(19) United States
(12) Patent Application Publication
(10) Pub. No.: US 2023/0265484 A1
(43) Pub. Date: Aug. 24, 2023

(54) MULTIPLEXED METHODS FOR DETECTING TARGET RNAS

(71) Applicant: PRESIDENT AND FELLOWS OF HARVARD COLLEGE, Cambridge, MA (US)

(72) Inventors: Marc W. KIRSCHNER, Cambridge, MA (US); Mingjie DAI, Cambridge, MA (US); George M. CHURCH, Cambridge, MA (US)

(73) Assignee: PRESIDENT AND FELLOWS OF HARVARD COLLEGE, Cambridge, MA (US)

(21) Appl. No.: 17/913,589

(22) PCT Filed: Mar. 24, 2021
(86) PCT No.: PCT/US2021/023978

§ 371 Date: Sep. 22, 2022

Related U.S. Application Data
(60) Provisional application No. 63/159,033, filed on Mar. 10, 2021.
(60) Provisional application No. 63/040,790, filed on Jun. 18, 2020.
(60) Provisional application No. 62/994,072, filed on Mar. 24, 2020.

(51) Int. Cl. C12Q 1/6806; C12Q 1/70

(52) U.S. Cl. CPC C12Q 1/6806; C12Q 1/70

(57) ABSTRACT

The technology described herein is directed to methods, kits, compositions, and systems for detecting a target RNA, such as a small amount of viral RNA. In one aspect, described herein are methods of detecting the target RNA, using primers comprising at least one barcode region. In other aspects, described herein are kits, compositions, and systems suitable to practice the methods described herein to detect the target RNA.

### CROSS-REFERENCE TO RELATED APPLICATIONS

This application claims benefit under 35 U.S.C. § 119(e) of U.S. Provisional Application No. 62/994,072 filed Mar. 24, 2020, U.S. Provisional Application No. 63/040,790 filed Jun. 18, 2020, and U.S. Provisional Application No. 63/159,033 filed Mar. 10, 2021, the contents of each of which are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Mar. 24, 2021, is named 002806-097220WOPT_SL.txt and is 302,231 bytes in size.

### TECHNICAL FIELD

The technology described herein relates to multiplexed methods, kits, and compositions for detecting target RNAs, such as viral RNAs.

### BACKGROUND

Highly -scalable and highly-sensitive viral diagnostics (e.g. for SARS-CoV-2) are critical for both pandemic response and long-term epidemiological surveillance. During a pandemic, population-wide testing can provide effective control and monitoring of the viral spread and allow safe return to work. In the long term, regular and population-wide monitoring promises a “bio-weather map” to identify and forecast new viral infection hotspots, preventing the “next outbreak”. Furthermore, the ability to sequence and identify emerging viral variants (e.g. B.1.1.7, B 1.427 for SARS-CoV-2), also on the population scale, allows real-time monitoring of the rate of transmission and pathogenicity, as well as informing public health policies and vaccine development. Current diagnostic methods fall short of these requirements, as they are limited in either sample processing throughput, testing sensitivity and reliability, or the ability to identify different viral variants.

At present, molecular tests using “gold standard” reverse transcription polymerase chain reaction (RT-qPCR) in central laboratory facilities have demonstrated high detection sensitivity (down to 200 gce/mL-1,000 gce/mL of SARS-CoV-2 (by the FDA’s comparison panel results), but they are limited in throughput by the requirements of RNA extraction and PCR thermocycling on each sample individually, as well as other liquid handling operations; see e.g., Vandenberg et al. Nat Rev Microbiol 19, 171-183 (Oct. 14, 2020); MacKay et al. Nat Biotechnol 38, 1021-1024 (Aug. 20, 2020); Esbin et al., RNA 26, 771-783 (May 1, 2020); Arnaout et al. SARS-CoV2 Testing: The Limit of Detection Matters (bioRxiv, Jun. 4, 2020); the contents of each of which are incorporated herein by reference in their entireties. As a result, it is challenging for most current clinical labs to perform more than 10,000 diagnostic tests per day, even with the help of automation; see e.g., Cobas SARS-CoV-2 Instructions for Use (Mar. 12, 2020), available on the world wide web at fda.gov/media/136049/download; the content of which is incorporated herein by reference in its entirety. By re-purposing large-scale liquid handling and sample automation, up to 100,000 tests per day can be achieved, but this approach requires heavy upfront capital investment and personnel costs.

Next-generation sequencing (NGS) based methods have long been attractive alternatives to RT-qPCR in two ways: (i) the intrinsic high-throughput readout for multiplexed diagnostics, and (ii) the ability to obtain viral genome sequences for variant identification. In principle the very high-throughput (up to 10¹⁰ reads per session, on an Illumina NovaSeq™ machine) allows a single testing lab to process up to a million patient samples per day with pooled analysis, if they could avoid the handling of individual samples. Since the beginning of the COVID-19 pandemic, several methods for NGS-based multiplexed testing have been proposed and developed. See e.g., Bloom et al., Swab-Seq: A high-throughput platform for massively scaled up SARS-CoV-2 testing, medRxiv (Aug. 6, 2020); Illumina™ COVIDSeq Test Instructions for Use (May 1, 2020); Hossain et al. A massively parallel COVID-19 diagnostic assay for simultaneous testing of 19200 patient samples. Google Docs (Mar. 20, 2020); Schmid-Burgk et al. LAMP-Seq: Population-Scale COVID-19 Diagnostics Using a Compressed Barcode Space bioRxiv (Apr. 8, 2020); Wu et al., INSIGHT: A population-scale COVID-19 testing strategy combining point-of-care diagnosis with centralized high-throughput sequencing. Sci Adv 7, (Feb. 12, 2021); Yelagandula et al. SARSeq, a robust and highly multiplexed NGS assay for parallel detection of SARS-CoV2 and other respiratory infections (medRxiv, Nov. 3, 2020); the contents of each of which are incorporated herein by reference in their entireties.

As expected, methods that achieved detection sensitivity close to the RT-qPCR tests (200-1000 gce/ml) mostly followed the traditional barcoding and sequencing workflows, which also required RNA extraction and PCR thermocycling steps, see e.g., supra, Bloom, Illumina, or Yelagandula (or used an extraction-free protocol but with ~10 x lower sensitivity, see e.g., Bloom supra; Bruce et al., PLoS Biol 18, e3000896 (Oct. 2, 2020); the contents of each of which are incorporated herein by reference in their entireties), which in practice hindered the maximum achievable sample throughput. Furthermore, current methods either do not report viral variant information, or perform whole genome sequencing (WGS), which further limits the achievable throughput due to the large number of sequencing reads required. As such, there is great need for sequencing-based methods that achieves high sensitivity, high throughput, and identification of viral variants.

### SUMMARY

The technology described herein is directed to multiplexed methods of detecting at least one target RNA in at least two samples. Specifically, the methods use primers comprising at least one barcode region. Also described herein are kits, compositions, and system associated with such methods. Such multiplexed methods, also referred to herein as “One-Seq,” exhibit at least the following advantages compared to existing detection methods: (1) the workflow permits barcoding of 50-5,000 samples per batch, with up to ~100,000 total samples per sequencing run; (2) the workflow permits pre-amplification pooling of reverse transcription products; (3) the method can be used to detect multiple loci on one target RNA molecule in one test; (4) the method can be used to detect multiple RNA target molecules, e.g., multiple viruses, in one test; (5) the method exhibits high sensitivity, e.g., as the number of RNA targets that are on one RNA molecule increases, the level of sensitivity increases (e.g., the sensitivity of the SARS-CoV-2 detection method approaches 50-150 genome copy equivalents per mL (gce/mL), compared to other sequencing-based tests that detect over 1000 gce/mL; (6) the method exhibits high efficiency, with reduced labor (e.g., no upfront extraction step, a one-pot reverse transcription step, reduced liquid-handling steps, etc.) and reduced cost per test; (7) the protector nucleic acid described herein can be used to reduce or eliminate barcode crosstalk that can result from reverse transcription primer carry-over into the amplification step; and (8) specially-designed primers can be used to detect variations of interest in the target RNA.

Accordingly, in one aspect described herein is a multiplexed method of detecting at least one target RNA in at least two samples, comprising: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; (c) contacting the pooled reverse transcription product mixture with a DNA polymerase and a set of second primers under conditions permitting the generation of amplification products; and (d) sequencing the amplification products, thereby detecting at least one target RNA, if present, in the at least two samples.

In some embodiments of any of the aspects, step (b) is performed before step (c).

In some embodiments of any of the aspects, steps (a)-(d) are performed sequentially.

In some embodiments of any of the aspects, the detection method has a limit of detection of at least 500 target RNA copies per mL for a given target RNA.

In some embodiments of any of the aspects, the detection method has a limit of detection of at least 1000 target RNA copies per mL for a given target RNA.

In some embodiments of any of the aspects, the detection method has a dynamic range of at least 3 logs.

In some embodiments of any of the aspects, at least 2 target RNAs in a single sample are detected.

In some embodiments of any of the aspects, the at least 2 target RNAs are on the same RNA molecule.

In some embodiments of any of the aspects, the at least 2 target RNAs are on different RNA molecules.

In some embodiments of any of the aspects, at least one target RNA is a viral RNA.

In some embodiments of any of the aspects, at least 2 target RNAs are from the same virus.

In some embodiments of any of the aspects, at least 2 target RNAs are from at least 2 different viruses.

In some embodiments of any of the aspects, at least one viral RNA is a SARS-CoV-2 RNA.

In some embodiments of any of the aspects, target RNAs from at least 50 samples are detected in a single performance of steps (a) - (d).

In some embodiments of any of the aspects, prior to step (a), the at least one target RNA is not extracted from the sample.

In some embodiments of any of the aspects, the reverse transcriptase (RT) is an engineered or recombinant version of an Moloney Murine Leukemia Virus (MMLV) RT, Avian Myeloblastosis Virus (AMV) RT, or another naturally occurring RT.

In some embodiments of any of the aspects, the first primer or each primer in the first set of primers comprises, from 5′ to 3′: (a) an adaptor region; (b) a first barcode region; and (c) a target-binding region that is complementary or substantially complementary to and permits hybridization to at least one target RNA.

In some embodiments of any of the aspects, the first primer or each primer in the first set of primers comprises, from 5′ to 3′: (a) an adaptor region; (b) a first barcode region; (c) a second barcode region; and (d) a target-binding region that is complementary or substantially complementary to and permits hybridization to at least one target RNA.

In some embodiments of any of the aspects, the barcode region of a first primer in the first set of barcoded primers is a Hamming distance of at least 10 from each other barcode region of any other primer in the first set of barcoded primers.

In some embodiments of any of the aspects, the first or second barcode region on the first primer or set of first primers comprises one of SEQ ID NOs: 18-989.

In some embodiments of any of the aspects, at least one barcode region on the first primer or set of first primers corresponds to and is different for each of the at least two samples.

In some embodiments of any of the aspects, at least one barcode region on the first primer or set of first primers corresponds to and is different for each of the target RNAs.

In some embodiments of any of the aspects, the target-binding region of a primer in the first set of primers binds at most 5 nucleotides away from a variation of interest in the target RNA.

In some embodiments of any of the aspects, the variation of interest is selected from the group consisting of: a single-nucleotide variation; a point mutation; a substitution; an insertion; and a deletion.

In some embodiments of any of the aspects, the target RNA is SARS-CoV-2 S gene and the variation of interest is selected from the group consisting of: del69-70, del144, K417N, K417T, L452R, E484K, N501Y, D614G, P681H, and A701V.

In some embodiments of any of the aspects, step (a) further comprises contacting the sample with a detergent.

In some embodiments of any of the aspects, the detergent lyses viral particles or cells in the sample.

In some embodiments of any of the aspects, the detergent releases target RNA from the sample.

In some embodiments of any of the aspects, the detergent is a nonionic surfactant.

In some embodiments of any of the aspects, the detergent is Triton X-100.

In some embodiments of any of the aspects, step (a) further comprises contacting the sample with carrier nucleic acid.

In some embodiments of any of the aspects, the carrier nucleic acid reduces loss of the target RNA.

In some embodiments of any of the aspects, the carrier nucleic acid is poly-A60 DNA oligonucleotide or E. coli tRNA.

In some embodiments of any of the aspects, step (a) further comprises contacting the sample with a positive control nucleic acid.

In some embodiments of any of the aspects, the positive control nucleic acid is a primer comprising from 5′ to 3′: (a) an adaptor region; (b) a first barcode region; and (c) a target-binding region that is complementary to or substantially complementary to a sample nucleic acid.

In some embodiments of any of the aspects, the positive control nucleic acid comprises, from 5′ to 3′: (a) a region that is not identical or substantially identical to any target RNA being assayed; and (b) a region that is identical or substantially identical to at least one target RNA.

In some embodiments of any of the aspects, the region of the positive control nucleic acid that is identical or substantially identical to at least one target RNA is complementary or substantially complementary to the target-binding region of at one least primer from the first set of primers.

In some embodiments of any of the aspects, the positive control nucleic acid comprises SEQ ID NO: 11.

In some embodiments of any of the aspects, the sample is contacted with at least 100-10⁴ copies/ul of positive control nucleic acid.

In some embodiments of any of the aspects, step (a) further comprises contacting the samples with a stabilization agent.

In some embodiments of any of the aspects, the stabilization agent prevents degradation of the RNA target and/or reverse transcriptase for at least 6 hours at room temperature.

In some embodiments of any of the aspects, the stabilization agent prevents degradation of the RNA target and/or reverse transcriptase for at least 24 hours at room temperature.

In some embodiments of any of the aspects, the stabilization agent is an RNA-preserving agent or a reverse-transcriptase-preserving agent.

In some embodiments of any of the aspects, the RNA-preserving agent is an RNase inhibitor, a metal-chelating agent, or a reducing agent.

In some embodiments of any of the aspects, the RNase inhibitor is murine RNase inhibitor or a thermostable RNase inhibitor.

In some embodiments of any of the aspects, the metal-chelating agent is ethylenediaminetetraacetic acid (EDTA).

In some embodiments of any of the aspects, the reducing agent is dithiothreitol (DTT).

In some embodiments of any of the aspects, the reverse-transcriptase-preserving agent is an antibiotic, an antimycotic, or a protease inhibitor.

In some embodiments of any of the aspects, step (a) comprises a reverse transcription reaction.

In some embodiments of any of the aspects, step (a) comprises: (i) incubating the sample, reverse transcriptase, and first primer or first set of primers comprising at least one barcode at a temperature of at least 50° C. for at least 30 minutes; and (ii) inactivating the reverse transcription reaction at a temperature of at least 95° C. for at least 5 minutes.

In some embodiments of any of the aspects, the reverse transcription products from step (a) comprise a barcoded DNA comprising a region that is complementary to a portion of at least one target RNA.

In some embodiments of any of the aspects, reverse transcription products from step (a) from at least 5 different samples are combined in one container.

In some embodiments of any of the aspects, prior to step (c) the first set of barcoded primers is substantially removed.

In some embodiments of any of the aspects, prior to step (c) the target RNA and/or sample is substantially removed.

In some embodiments of any of the aspects, prior to step (c) the first set of barcoded primers or the RNA target is substantially removed using a bead-based purification method or a spin-column-based purification method.

In some embodiments of any of the aspects, the DNA polymerase is a thermostable DNA polymerase I.

In some embodiments of any of the aspects, the DNA polymerase is a Thermus aquaticus (Taq) DNA polymerase.

In some embodiments of any of the aspects, the second set of primers comprises forward and reverse amplification primers.

In some embodiments of any of the aspects, the forward primer in the second set of primers comprises from 5′ to 3′: (a) an adaptor region; and (b) an adaptor-binding region that is identical or substantially identical to the adaptor region of a primer in the first set of barcoded primers.

In some embodiments of any of the aspects, a forward primer in the second set of primers comprises from 5′ to 3′: (a) an adaptor region; (b) a third barcode region; and (c) an adaptor-binding region that is identical or substantially identical to the adaptor region of a primer in the first set of barcoded primers.

In some embodiments of any of the aspects, a reverse primer in the second set of primers comprises, from 5′ to 3′: (a) an adaptor region; (b) a second barcode region; and (c) a target-binding region that is identical or substantially identical to at least one target RNA.

In some embodiments of any of the aspects, a reverse primer in the second set of primers comprises, from 5′ to 3′: (a) an adaptor region; and (b) a region that is identical or substantially identical to at least one target RNA.

In some embodiments of any of the aspects, the barcode region of a first primer in the second set of barcoded primers is a Hamming distance of at least 5 from each other barcode region of any other primer in the second set of barcoded primers.

In some embodiments of any of the aspects, the second or third barcode region in the second set of primers comprises one of SEQ ID NOs: 18-989.

In some embodiments of any of the aspects, step (c) further comprises contacting the reverse transcription product with Uracil-DNA Glycosylase (UDG) enzyme.

In some embodiments of any of the aspects, step (c) further comprises contacting the reverse transcription product or amplification product thereof with a protector nucleic acid.

In some embodiments of any of the aspects, the protector nucleic acid comprises single stranded DNA.

In some embodiments of any of the aspects, the protector nucleic acid comprises, from 5′ to 3′: (a) a region complementary or substantially complementary to a region of at least one target RNA or amplification product thereof, comprising: (i) a 5′ region that is identical or substantially identical to the target-binding region of at least one primer in the first set of primers; and (ii) a 3′ region that is complementary to the target RNA sequence downstream of the target-binding region of at least one primer in the first set of primers; and (b) a 3′ nucleic acid modification that inhibits synthesis of a complementary strand by a polymerase.

In some embodiments of any of the aspects, the 3′ complementary region of the protector nucleic acid is at least 15 nucleotides long.

In some embodiments of any of the aspects, the 3′ complementary region of the protector nucleic acid is at most 30 nucleotides long

In some embodiments of any of the aspects, the 3′ nucleic acid modification is selected from the group consisting of: (a) an inverted base; (b) a spacer; (c) a dideoxynucleotide; (d) a base that is not complementary to the target RNA; and (e) a non-canonical base.

In some embodiments of any of the aspects, the protector nucleic acid displaces a primer from the first set of primers from an amplification product of the reverse transcription product.

In some embodiments of any of the aspects, the protector nucleic acid inhibits or substantially inhibits a primer from the first set of primers from being extended by the DNA polymerase.

In some embodiments of any of the aspects, the protector nucleic acid has a higher binding affinity to an amplification product of the reverse transcription product than the target-binding region of the at least one primer from the first set of primers.

In some embodiments of any of the aspects, the protector nucleic acid has a higher Tm than the target-binding region of the at least one primer from the first set of primers.

In some embodiments of any of the aspects, the protector nucleic acid inhibits or substantially inhibits a primer from the first set of primers from binding to an amplification product of the reverse transcription product.

In some embodiments of any of the aspects, the protector nucleic acid is at least 15 nucleotides long.

In some embodiments of any of the aspects, the protector nucleic acid is at least 30 nucleotides long.

In some embodiments of any of the aspects, the protector nucleic acid is present at a concentration that is greater than the concentration of the primers in the first set of primers.

In some embodiments of any of the aspects, the protector nucleic acid is present at a concentration of at least 0.5 uM.

In some embodiments of any of the aspects, the protector nucleic acid is present at a concentration of at least 2.0 uM.

In some embodiments of any of the aspects, step (c) comprises a nucleic acid amplification method.

In some embodiments of any of the aspects, the amplification method comprises polymerase chain reaction amplification (PCR).

In some embodiments of any of the aspects, step (c) comprises: (i) a denaturation step; (ii) an annealing step; (iii) and an extension step, wherein steps (i)-(iii) are repeated at least 30 times.

In some embodiments of any of the aspects, step (c) further comprises an initial denaturation step before the first step (i) at least 95° C. for at least 60 seconds.

In some embodiments of any of the aspects, step (i) is performed at a temperature of at least 95° C. for at least 15 seconds.

In some embodiments of any of the aspects, step (ii) is performed at a temperature of at least 60° C. for at least 30 seconds.

In some embodiments of any of the aspects, the first two iterations of step (ii) are performed at a temperature of at least 52° C.

In some embodiments of any of the aspects, the iterations of step (ii) after the first two iterations of step (ii) are performed at a temperature of at least 68° C.

In some embodiments of any of the aspects, step (iii) is performed at a temperature of at least 72° C. for at least 30 seconds.

In some embodiments of any of the aspects, step (c) further comprises contacting at least one reverse transcription product with a protector nucleic acid, and wherein step (ii) is performed at a temperature of at least 64° C.

In some embodiments of any of the aspects, step (c) further comprises contacting at least one reverse transcription product with a protector nucleic acid, and wherein step (ii) is performed at a temperature of at least 72° C.

In some embodiments of any of the aspects, step (c) further comprises contacting at least one reverse transcription product with a protector nucleic acid, and at least one of the following: (I) step (ii) is performed at a temperature of at least 64° C.; (II) the 3′ complementary region of the protector nucleic acid is at least 20 nucleotides long; and/or (III) the protector nucleic acid is present at a concentration of at least 0.5 uM.

In some embodiments of any of the aspects, step (c) further comprises contacting at least one reverse transcription product with a protector nucleic acid, and at least one of the following: (I) step (ii) is performed at a temperature of at least 68° C.; (II) the 3′ complementary region of the protector nucleic acid is at least 30 nucleotides long; and/or (III) the protector nucleic acid is present at a concentration of at least 2.0 uM.

In some embodiments of any of the aspects, at least 10 amplification product sets from step (c) are combined in one container.

In some embodiments of any of the aspects, prior to step (d) the second set of barcoded primers are substantially removed.

In some embodiments of any of the aspects, prior to step (d) the second set of barcoded primers are substantially removed using a bead-based purification method or a spin-column-based purification method.

In some embodiments of any of the aspects, the sequencing method is a high-throughput sequencing method.

In some embodiments of any of the aspects, the sequencing method is selected from the group consisting of: sequencing by synthesis, dideoxy chain termination sequencing, pyrosequencing, sequencing by ligation and detection, polony sequencing, ion semiconductor sequencing, sequencing by hybridization, and nanopore sequencing.

In some embodiments of any of the aspects, the sequencing method is sequencing by synthesis.

In some embodiments of any of the aspects, the sequencing method comprises contacting the amplification products with a third set of primers, comprising at least first and second sequencing primers.

In some embodiments of any of the aspects, the first and second sequencing primers comprise an adaptor-binding region that is complementary or substantially complementary to the adaptor region of a primer in the first or second set of primers.

In some embodiments of any of the aspects, the sequencing method produces a sequencing read from the first or second sequencing primer.

In some embodiments of any of the aspects, the sequencing read from the first sequencing primer comprises the sequence of the first barcode region from a primer in the first primer set.

In some embodiments of any of the aspects, the sequencing read from the second sequencing primer comprises the sequence of the first and second barcode regions from a primer in the first primer set.

In some embodiments of any of the aspects, the sequencing read from the second sequencing primer comprises the sequence of the second barcode region from a primer in the second primer set.

In some embodiments of any of the aspects, the sequencing read from the first or second sequencing primer comprises sequence from the target RNA.

In some embodiments of any of the aspects, the sequencing read from the first or second sequencing primer comprises at least one variation of interest in the target RNA.

In some embodiments of any of the aspects, the target RNA is detected in the sample if a first and second barcode region associated with the specific target RNA is detected in the sequencing read of the amplification product.

In some embodiments of any of the aspects, the target RNA is not detected in the sample if a first or second barcode region associated with the specific target RNA is not detected in the sequencing read of the amplification product.

In some embodiments of any of the aspects, at least n target RNAs in a single sample are detected, and the at least n target RNAs are on the same assayed RNA molecule.

In some embodiments of any of the aspects, the assayed RNA molecule is: (i) determined to be present in the sample if at least one of the n target RNAs are detected; or (ii) determined to not be present in the sample if none of the n target RNAs are detected.

In one aspect described herein is a method of preparing at least two pooled barcoded amplification sets from at least one target RNA in at least two samples, comprising the sequential steps of: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; and (c) contacting the pooled reverse transcription product mixture with a DNA polymerase and a set of second primers under conditions permitting the generation of amplification products.

In one aspect described herein is a reverse transcription solution comprising: (a) a reverse transcriptase; (b) a first set of primers comprising at least one barcode; (c) a detergent; (d) carrier nucleic acid; (e) at least one positive control nucleic acid; (f) at least one stabilization agent; and/or (g) reverse transcription reaction buffer.

In one aspect described herein is a collection tube containing a reverse transcription solution as described herein.

In one aspect described herein is a kit for detecting a target RNA in a sample, comprising: (a) a reverse transcriptase; (b) a first set of primers comprising at least one barcode; (c) a detergent; (d) a carrier nucleic acid; (e) a positive control nucleic acid; (f) at least one stabilization agent; (g) at least two containers; (h) a DNA polymerase; (i) a second set of primers; (j) Uracil-DNA Glycosylase (UDG) enzyme; (k) a protector nucleic acid; and/or a third set of primers.

In one aspect described herein is a composition comprising: (a) a target RNA; (b) a reverse transcriptase; (c) a first primer or a first set of primers comprising at least one barcode; (d) a detergent; (e) a carrier nucleic acid; (f) a positive control nucleic acid; and/or (g) at least one stabilization agent.

In one aspect described herein is a composition comprising: (a) a barcoded reverse transcription product; (b) a second set of primers; (c) DNA polymerase; (d) Uracil-DNA Glycosylase (UDG) enzyme; and/or (e) a protector nucleic acid.

### BRIEF DESCRIPTION OF THE DRAWINGS

FIGS. 1A-1C is a series of schematics showing the workflow for highly-multiplexed viral RNA detection by high-throughput sequencing. Schematics are illustrated with 1000 patient samples (labelled as #1-#1000) and 20 locus-specific probes (labelled as (1)-(20)). FIG. 1A is a schematic showing that samples are converted to cDNA (first strand) with a set of barcoded forward primers, which can encode the sample ID as well as locus ID. FIG. 1B is a schematic showing that cDNA strands from many samples (e.g., 1,000) are pooled and a second strand is synthesized with a common, backward primer. Barcoded and pooled samples are purified, amplified with a limited number of PCR cycles, then captured on a surface. FIG. 1C is a schematic showing that barcodes (e.g., sample and locus ID) are amplified by bridge PCR and read out by high-throughput sequencing.

FIG. 2 is a flowchart showing an exemplary detection method.

FIG. 3 shows a reverse transcription efficiency assay (e.g., in saliva). “qPCR” indicates quantitative polymerase chain reaction; “Cq value” is the PCR cycle number at which the sample’s reaction curve intersects the threshold line; the x-axis shows “cps/rxn” or copies per reaction. FIG. 3 is a line graph showing the reverse transcription efficiency assay with double-stranded DNA (dsDNA) spike-in (N=3). For FIG. 3, qPCR detection (e.g., dye-based) sensitivity was as follows: <20 molecules in buffer; <200 molecules in saliva (e.g., 50%); ΔΔCq ~ 0.1, indicating close to quantitative RT reaction efficiency.

FIG. 4 is a line graph of the reverse transcription efficiency assay, showing the reverse transcription reaction and qPCR sensitivity for RNA or DNA, with or without saliva (N=2).

FIGS. 5A-5C is a series of graphs, schematics, and tables showing the reverse transcription efficiency assay (e.g., in saliva). FIG. 5A is a line graph showing RT reaction sensitivity (N=3). FIG. 5B is a line graph showing qPCR sensitivity (from DNA) (N=3). The average exponent slope was 3.3 (c.f. log2(10) = 3.32), i.e., close to perfect doubling. The starting concentration difference (RNA/DNA) was 3.5x. The expected ΔCq (RNA - DNA) was -0.87 (-1.87 + 1). The observed ΔCq (RNA - DNA) was -0.98, i.e. ΔΔCq was ~ 0.1; thus, the RT conversion was close to quantitative. FIG. 5C includes a schematic showing the RT primers and a table showing multiplexed RT Efficiency; “*” indicates that the Cq values were at 1.8e4 mRNA load.

FIGS. 6A-6B is a series of bar graphs showing reaction buffer and saliva sample stability at 0 hr, 7 hr, 24 hr, or 72 hr after sample acquisition (bars from left to right for each sample). FIG. 6A shows buffer conditions 0 and 351; FIG. 6B shows buffer conditions 651, 353, and 35301. Saliva samples A-H were tested. As targeted, the buffer mixture and sample demonstrated stabilization for 24-48 hours, which is compatible with methods comprising viral lysis and/or reverse transcription. Factors that can influence stability include: RNase activity, protease activity, mucus levels, bacteria and/or fungi growth, food residues in the saliva, etc.

FIG. 7 is a schematic showing a 96-sample set sequencing test. SEQ ID NO: 1 shows an exemplary primer. Bolded black text (e.g., nucleotides (nt) 1-16 of SEQ ID NO: 1) indicates the barcode region; grey text (e.g., nt 17-35 of SEQ ID NO: 1) indicates the RT primer region; and bold italicized text (e.g., nt 36-64 of SEQ ID NO: 1) indicates the region that is complementary to a target RNA (i.e., viral genome). The middle panel shows an exemplary plate map with a dilution factor of 1e4x, 1e5x, 1e6x, or 1e7x; “-ve” indicates no viral sample negative control; “-RT” indicates no reverse transcriptase negative control. The bottom panel shows the dilution factor, saliva concentration, and number of mRNA per reaction.

FIGS. 8A-8D is a series of graphs and tables showing results of the 96-sample set sequencing test (see e.g., FIG. 7 for test set-up). FIG. 8A is a dot plot showing the number of reads vs. mRNA copies of the first test. FIG. 8B is a dot plot showing the number of reads vs. mRNA copies of the second test. FIG. 8C is a table showing the maximum background (max (bg)) and limit of detection (LoD) of the first test. The limit of detection for the first test was 127 mRNA copies. FIG. 8D is a table showing the max (bg) and LoD of the second test. The limit of detection for the second test was 178 mRNA copies. “-ve” indicates no viral sample negative control; “-RT” indicates no reverse transcriptase negative control; “bg” indicates background; “stdev” indicates standard deviation.

FIGS. 9A-9C is a series of schematics, graphs and tables showing the protector strategy for reduced barcode swapping. FIG. 9A is a schematic showing the protector strategy. FIG. 9B is a dot plot showing the number of reads vs. mRNA copies for the protector strategy test. FIG. 9C is a table showing the max (bg) and LoD of the protector strategy test. Without protector, the limit of detection was 127 mRNA molecules. With protector, the limit of detection was 26 mRNA molecules. Thus, the protector strategy lowers the limit of detection.

FIGS. 10A-10B is a series of schematics and graphs showing the sub-pooling strategy for increased dynamic range. SEQ ID NO: 2 shows an exemplary primer. Unformatted black text (e.g., nucleotides (nt) 1-15 of SEQ ID NO: 2) indicates the sub-pool primer region; bolded black text (e.g., nucleotides (nt) 16-31 of SEQ ID NO: 2) indicates the barcode region; grey text (e.g., nt 32-50 of SEQ ID NO: 2) indicates the RT primer region; and bold italicized text (e.g., nt 51-79 of SEQ ID NO: 2) indicates the region that is complementary to a target RNA (e.g., viral genome). FIG. 10B is a bar graph showing a first test of dynamic range reduction by sub-pooling. The left-right order of bars for each mRNA concentration in FIG. 10B is the same as the top-bottom order of the legend.

FIG. 11 shows an exemplary schematic of a system as described herein.

FIGS. 12A-12D is a series of schematics showing the principle and workflow of One-Seq for highly-scalable viral detection and variant identification. FIG. 12A is an illustration of One-Seq “early pooling” strategy in comparison with “late pooling” methods. FIG. 12B is a schematic showing the clinical workflow of One-Seq. Early pooling allows up to 100,000 patient samples to be pooled and analyzed together. FIG. 12C is a schematic showing the molecular workflow of One-Seq. One-Seq uses upfront sample barcoding and a “protector” strategy to permit early sample pooling, and uses a two-stage pooling strategy to support highly scalable testing. FIG. 12D is an illustration of One-Seq reaction components. One-Seq uses multiple RT primers for viral diagnostic and sequencing, one human gene RT primer and one synthetic RNA as positive controls. In one embodiment, One-Seq uses the forward read to demultiplex the sample barcode, primer identity, and/or positive controls. In one embodiment, One-Seq uses the reverse read to demultiplex the batch barcode.

FIGS. 13A-13G is a series of schematics and graphs showing an extraction-free, one-pot reaction for efficient viral reverse transcription and sample preservation. FIG. 13A is a schematic of the RT efficiency test in contrived clinical samples, using pooled negative specimen and inactivated virus spike-in. FIG. 13B is an example RT sensitivity test. The top dot plot shows Ct values (3x repeats) plotted against different viral loads in genome copy equivalent (gce). The bottom table shows the detection rate and limit of detection (LoD) determination. FIGS. 13C-13E is a series of bar graphs showing the RT sensitivity test under different conditions: FIG. 13C shows a comparison of different RT primer concentrations; FIG. 13D shows a comparison of different RT primers and validation with different virus reference standards; FIG. 13E shows a comparison of single-primer vs dual-primer detection. FIGS. 13F-13G is a series of bar graphs showing the effect of sample preservation buffer after incubation for 0 hr or 24 hr room temperature in a clean reaction buffer (FIG. 13F) or contrived patient samples (FIG. 13G). AA: antibiotic and antimycotic; PI: protease inhibitor; D: DTT; E: EDTA; VTM: viral transport medium.

FIGS. 14A-14C is a series of schematics and graphs showing barcode design and the multiplexed sequencing sensitivity test. FIG. 14A is a schematic of unique sample barcode construction (see e.g., SEQ ID NO: 30 (UDPX001) and Table 5). FIG. 14A is a schematic showing 960 sample pooling and barcode selection. FIG. 14C is a dot plot showing an example multiplexed sequencing sensitivity test and LoD determination, plotted as sequencing read count +1 against expected viral loads. cDNA purification allows efficient library amplification after pooling.

FIGS. 15A-15G is a series of schematics and graphs showing a “protector” strategy that suppresses barcode crosstalk and preserves large sample dynamic range. FIG. 15A is a schematic showing the barcode crosstalk and dynamic range test by qPCR and multiplexed sequencing readout. FIG. 15B is a bar graph showing on-target and off-target sequencing read counts and fraction of crosstalk without using the protector strategy. FIG. 15C is a schematic showing two approaches to suppress barcode crosstalk: top panel shows dynamic strand displacement with a protector strand; bottom shows a naive approach with complementary strand hybridization. FIGS. 15D-15E is a series of bar graphs showing the crosstalk and dynamic range test with on-target amplification and 1 off-target primer, assayed by qPCR under different conditions. “≥” indicates lower bounds. FIG. 15D shows the effect of different protector strand design and annealing temperature; FIG. 15E shows the effect of off-target primer and protector strand concentrations. 15F-15G is a series of bar graphs showing the crosstalk and dynamic range test with 1 high-load sample and 95 off-target RT primers, assayed by multiplexed sequencing under different conditions. FIG. 15F shows the effect of supplementing extra off-target primers (+L, low amount, +H, high amount), with and without using the protector strategy. FIG. 15G shows a comparison of different cDNA purification methods. Q-PCR, QIAquick™ PCR purification kit (QIAGEN); Q-Nuc, QIAquick nucleotide removal kit (QIAGEN); T-MM, MagMax™ viral/pathogen nucleic acid isolation kit (ThermoFisher™); AP-XP, AmPure™ XP PCR purification beads (Beckman Coulter™).

FIGS. 16A-16C is a series of schematics and graphs showing validation of One-Seq on clinical SARS-CoV-2 specimens. FIG. 16A is a schematic of the One-Seq test with remnant clinical specimens. FIG. 16B shows an example of One-Seq testing results, plotted as One-Seq sequencing read counts (summed) +1 vs clinical Ct values by RT-qPCR and estimated viral load (calculated according to manufacturer’s specification). One-Seq results showed 6 logs of linear dynamic range with respect to patient viral load, and correctly detected samples down to 360 gce/ml. “*” indicates that for samples without a valid Ct(N) value, Ct(orf1ab) is used for plotting. FIG. 16C is a beeswarm plot of One-Seq results for positive (2x), positive (1x), and negative clinical samples, where positive (2x) refers to samples for which clinical RT-qPCR test returned positive results for both N and orflab amplicons, and positive (1x) refers to samples for which only one of the two amplicons were clinically detected (and Ct>36).

FIGS. 17A-17E is a series of schematics, tables, and graphs showing multi-primer testing and variant sequencing. FIG. 17A is a schematic showing RT primer design targeting a viral mutation hotspot. FIG. 17B is a schematic showing an example of strong local secondary structure in the viral genome that prevents efficient RT. Arrow indicates the mutated nucleotide. FIG. 17C is a table showing confirmatory sensitivity test results in contrived clinical samples for all four primer pairs (two in SARS-CoV-2 N gene and two in SARS-CoV2 S gene for mutation sequencing) designed for One-Seq. FIG. 17D is a bar graph showing a comparison of detection sensitivity with different numbers and combinations of primers. Combining more primers allows higher detection sensitivity, down to LoD = 2-5 gce with all four primers. Bars in each viral copy grouping are in the same order left-right as in the order of the legend top-bottom. FIG. 17E is a table showing exemplary test results. Viral sequencing showed that all positive clinical SARS-CoV-2 samples tested had the D614G mutation; however, none of the clinical samples had the del6970 mutation, indicating they were not related to the B.1.1.7 variant. Raw sequencing reads from four exemplary specimens as well as the virus standard sample (ATCC) were listed.

FIGS. 18A-18B is a series of schematics showing clinical implementations for One-Seq. FIG. 18A shows schematics for two clinical implementations: (v1) with pre-collected clinical specimen in viral transport medium, and (v2) with specimen collection directly into purpose-manufactured One-Seq collection tubes containing pre-assigned and uniquely identifiable sequence barcodes. FIG. 18B is a schematic showing that, compared with pre-collection (v1), direct collection (v2) completely avoids any liquid handling step and allows even higher scalability.

FIG. 19 is a schematic showing a comparison of One-Seq workflow with other related methods. The schematic compares the sample processing workflow for (i) RT-qPCR (i.e., the “gold standard”), (ii) Swab-Seq, and (iii) One-Seq. One-Seq uses a one-step reaction to circumvent the need for RNA extraction and PCR amplification steps. Dark grey blocks indicate sample processing steps that require high equipment usage and automation; light grey blocks indicate processing steps that are highly scalable.

FIGS. 20A-20B is a series of schematics showing the One-Seq sequencing construct and read structure. FIG. 20A is an illustration of a One-Seq sequencing construct and example sequences. Each viral amplicon consists of a patient ID, RT primer, viral sequence, reverse primer, and batch ID (see e.g., SEQ ID NO: 990). Sequences are illustrated with N#1 RT (e.g., SEQ ID NO: 3) and PCRprimers (e.g., SEQ ID NO: 4), patient ID barcode UDPX001 (e.g., SEQ ID NO: 30) and batch barcode S01 (e.g., SEQ ID NO: 992). “*” indicates the reverse complement of the indicated SEQ ID NO. FIG. 20A is an illustration of One-Seq sequencing read structure. Read 1 (see e.g., SEQ ID NO: 993) is used to decode patient ID (1000x), RT primer identity (4x) and amplicons from positive controls; read 2 (see e.g., SEQ ID NO: 993) is used to decode batch ID (100x).

FIG. 21 is a line graph showing a comparison of reverse transcriptase efficiency. Reverse transcription (RT) efficiency of different RT enzymes were compared using two-step RT-qPCR and the CDC’s N gene primer and probe set (N1), in the presence of human saliva background (50% v/v) and RNAse inhibitor (Murine, 10% v/v). SSIV showed the best RT efficiency in saliva-containing samples, and the assay detected 3 copies of mRNA spike-in. AMV, Avian Myeloblastosis Virus RT (New England Biolabs™, M0277); MMLV, Moloney Murine Leukemia Virus RT (New England Biolabs™, M0253); SSIV, SuperScript™ IV RT (ThermoFisher™, 18090010); RDF, RapiDxFire™ (Lucigen™, 30250).

FIGS. 22A-22F is a series of graphs and tables showing Ct and limit of detection data for tests in FIGS. 13C-13E. FIGS. 22A-22B show Ct and limit of detection (LoD) data for FIG. 13C, showing the effect of RT primer concentration. FIGS. 22C-22D show Ct and LoD data for FIG. 13D, showing validation using different virus standard materials. FIGS. 22E-22F show Ct and LoD data for FIG. 13E, showing the effect of multi-primer detection. FIG. 22A, FIG. 22C, and FIG. 22E are tables showing the limit of detection (LoD) determination. FIG. 22B, FIG. 22D, and FIG. 22F are raw Ct data plots; each condition was repeated three times.

FIGS. 23A-23D is a series of graphs and tables showing Ct and limit of detection data for tests in FIG. 13F, showing the effect of different sample preservatory buffers. FIG. 23A and FIG. 23C are tables showing the limit of detection (LoD) determination. FIG. 23B and FIG. 23D are raw Ct data plots; each condition was repeated three times.

FIGS. 24A-24D is a series of graphs and tables showing Ct and limit of detection data for tests in FIG. 13G, showing the effect of sample preservatory buffers in VTM and saliva samples. FIG. 24A and FIG. 24C are tables showing the limit of detection (LoD) determination. FIG. 24B and FIG. 24D are raw Ct data plots; each condition was repeated three times.

FIGS. 25A-25B is a series of graphs showing 960x barcode QC and selection (see e.g., Table 5). FIG. 25A is a bar graph showing the distribution of sequencing reads from all 960x sample barcodes; barcodes with reads above median were selected for subsequent tests. FIG. 25B is a box and whisker plot showing a linearity and dynamic range test with 200x selected barcodes. Sequencing reads showed linear response at higher viral load conditions and dynamic range of ~10⁴.

FIG. 26 is a bar graph showing a barcode crosstalk and dynamic range test in 10-plex settings. Barcode crosstalk and dynamic range was tested with 10 high-load samples, in the presence of ~86x off-target primers, amplified in the presence of protector strand, and assayed by sequencing. Four conditions were tested, using two different cDNA purification methods (Q-PCR and T-MM) and with or without supplementation of extra off-target primers (-, without supplementation, +L, with low amount supplementation). Reads were normalised by on-target samples (average) to 10⁶ reads per barcode. Q-PCR, QIAquick™ PCR purification kit (QIAGEN); T-MM, MagMax™ viral/pathogen nucleic acid isolation kit (ThermoFisher™).

FIGS. 27A-27B is a series of schematics and graphs showing the design of RT and PCR primers targeting viral hotspot mutations and the RT sensitivity test. FIG. 27A shows the Sequence design of RT and PCR primers targeting two SARS-CoV-2 hotspot mutations, S:del69-70 and S:D614G. Nucleotides affected by these mutations are indicated. See e.g., SEQ ID NOs: 7-10, SEQ ID NOs: 995-997, and SEQ ID NO: 1004. FIG. 27B is a table showing the RT sensitivity assay by dye-based qPCR assay, using the primer sets shown in FIG. 27A. LoD was determined to be 5 gce for both targets.

FIGS. 28A-28E is a series of graphs showing confirmatory clinical sensitivity studies in a 96x multiplexed test. Confirmatory clinical sensitivity studies were performed in pooled negative remnant clinical specimen background with different concentration of inactivated virus spike-in. All tests were performed with 96x multiplexed sample processing workflow. Each testing condition was repeated 20-22 times using unique barcodes (i.e. not repeated 20-22 times with the same barcode). Each primer was tested multiple times with different batch barcode on the reverse side. LoD was determined using 95% detection rate criteria (i.e., 19/20 detection). FIGS. 28A-28C show confirmatory clinical sensitivity studies for single-primer detection. FIGS. 28D-28E show confirmatory clinical sensitivity studies for multi-primer detection. For FIG. 28A, FIG. 28B, and FIG. 28D, each test condition was repeated 20-22 times with unique barcodes. Dot plots showing sequencing reads for each barcode and each test condition. Solid lines indicate 3-σ threshold values. S number indicates batch barcode. FIG. 28C and FIG. 28E are bar graphs showing the detection rate at different viral load conditions and LoD values determined for each primer; bars in each viral copy grouping is in the same order left-right as in the order of the legend top-bottom.

FIGS. 29A-29D is a series of dot plots showing raw sequencing reads and breakdown for multi-primer clinical sample test in a 96 x multiplexed test. Raw sequencing reads were plotted against clinical Ct values for N gene or Orflab gene (e.g., if N gene was not detected). FIGS. 29A-29B show sequencing read scatters plot for all samples, including clinical samples, standards and negative controls, and all four viral targeting primers. Note that del6970 and D614 targets were amplified in the absence of protector strand, and showed a limited dynamic range as a result. FIGS. 29C-29D show a breakdown of sequencing read for N#1 and N#2 primers, individually (FIG. 29C) or summed together (FIG. 29D). Positive (2x) refers to samples for which clinical RT-qPCR test returned positive results for both N and orflab amplicons, and positive (1x) refers to samples for which only one of the two amplicons were clinically detected (and Ct>36).

### DETAILED DESCRIPTION

The technology described herein is directed to multiplexed methods of detecting at least one target RNA in at least two samples. Specifically, the methods use primers comprising at least one barcode region. Also described herein are kits, compositions, and system associated with such methods. Such multiplexed methods, also referred to herein as “One-Seq,” exhibit at least the following advantages compared to existing detection methods: (1) the workflow permits barcoding of 50-5,000 samples per batch, with up to ~100,000 total samples per sequencing run; (2) the workflow permits pre-amplification pooling of reverse transcription products; (3) the method can be used to detect multiple loci on one target RNA molecule in one test; (4) the method can be used to detect multiple RNA target molecules, e.g., multiple viruses, in one test; (5) the method exhibits high sensitivity, e.g., as the number of RNA targets that are on one RNA molecule increases, the level of sensitivity increases (e.g., the sensitivity of the SARS-CoV-2 detection method approaches 50-150 genome copy equivalents per mL (gce/mL), compared to other sequencing-based tests that detect over 1000 gce/mL; (6) the method exhibits high efficiency, with reduced labor (e.g., no upfront extraction step, a one-pot reverse transcription step, reduced liquid-handling steps, etc.) and reduced cost per test; (7) the protector nucleic acid described herein can be used to reduce or eliminate barcode crosstalk that can result from reverse transcription primer carry-over into the amplification step; and (8) specially-designed primers can be used to detect variations of interest in the target RNA. The following discusses considerations to permit those of ordinary skill in the art to make and practice the compositions and methods described herein.

### Methods

In multiple aspects, described herein are methods of detecting a target RNA. The target RNA can be detected at the single molecular level using the methods, kits, and systems as described herein. In one aspect described herein is a multiplexed method of detecting at least one target RNA in at least two samples, comprising: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; (c) contacting the pooled reverse transcription product mixture with a DNA polymerase and a set of second primers under conditions permitting the generation of amplification products; and (d) sequencing the amplification products, thereby detecting at least one target RNA, if present, in the at least two samples. In some embodiments, step (a) is performed before step (b). In some embodiments, step (b) is performed before step (c). In some embodiments, step (c) is performed before step (d). In some embodiments, steps (a)-(d) are performed sequentially.

In one aspect described herein is a multiplexed method of detecting at least one target RNA in at least two samples, comprising the sequential steps of: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; (c) contacting the pooled reverse transcription product mixture with a DNA polymerase and a set of second primers under conditions permitting the generation of amplification products; and (d) sequencing the amplification products, thereby detecting at least one target RNA, if present, in the at least two samples.

In one aspect described herein is a multiplexed method of detecting at least one target RNA in at least two samples, consisting of: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; (c) contacting the pooled reverse transcription product mixture with a DNA polymerase and a set of second primers under conditions permitting the generation of amplification products; and (d) sequencing the amplification products, thereby detecting at least one target RNA, if present, in the at least two samples.

In one aspect described herein is a multiplexed method of detecting at least one target RNA in at least two samples, comprising: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; (c) contacting the pooled reverse transcription product mixture with a DNA polymerase, at least one protector nucleic acid, and a set of second primers under conditions permitting the generation of amplification products; and (d) sequencing the amplification products, thereby detecting at least one target RNA, if present, in the at least two samples.

In one aspect, described herein is a method of preparing at least two pooled barcoded amplification sets from at least one target RNA in at least two samples, comprising the sequential steps of: (a) contacting the at least two samples with a reverse transcriptase and a first primer or first set of primers comprising at least a first barcode, under conditions permitting the generation of reverse transcription products; (b) combining reverse transcription products from samples in step (a) in one container to form a pooled reverse transcription product mixture; and (c) contacting the pooled reverse transcription product mixture with a DNA polymerase and a set of second primers under conditions permitting the generation of amplification products. In some embodiments of any of the aspects, at least one target RNA in the at least two pooled barcoded amplification sets is detected using a sequencing method.

The detection methods as described herein are highly multiplexed. In some embodiments of any of the aspects, the multiplexed method detects at least one target RNA in at least two samples or as many as 100,000 samples in one sequencing run. In some embodiments of any of the aspects, at least one target RNA from at least 50 samples is/are detected, e.g., in a single performance of steps (a) - (d). In some embodiments of any of the aspects, at least one target RNA from at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, at least 850, at least 860, at least 870, at least 880, at least 890, at least 900, at least 910, at least 920, at least 930, at least 940, at least 950, at least 960, at least 970, at least 980, at least 990, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500, at least 10000, at least 15000, at least 20000, at least 25000, at least 30000, at least 35000, at least 40000, at least 45000, at least 50000, at least 55000, at least 60000, at least 65000, at least 70000, at least 75000, at least 80000, at least 85000, at least 90000, at least 95000, at least 100000 or more samples is/are detected. This improved workflow, facilitated for example by pre-amplification barcoding and pooling ahead of next generation sequencing permits highly increased throughput without sacrificing sensitivity.

In some embodiments of any of the aspects, at least one target RNA from at least 50 samples are detected per batch, e.g., in a single performance of steps (a) - (c). In some embodiments of any of the aspects, at least one target RNA from at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, at least 850, at least 860, at least 870, at least 880, at least 890, at least 900, at least 910, at least 920, at least 930, at least 940, at least 950, at least 960, at least 970, at least 980, at least 990, at least 1000 samples are detected per batch.

The detection methods as described herein are highly sensitive. In some embodiments of any of the aspects, the detection method has a limit of detection of at least 500 target RNAs per mL for a given target RNA. As used herein, the term “limit of detection” (LoD or detection limit) refers to the lowest quantity of the target RNA that can be distinguished from the absence of target RNA with a predetermined confidence level (e.g., 90% or 95% detection rate). In some embodiments of any of the aspects, the detection method has a limit of detection of at least 1000 target RNA copies per mL for a given target RNA. In some embodiments of any of the aspects, the detection method, e.g., using one primer per target RNA molecule, has a limit of detection of at least 500 target RNA copies per mL for a given target RNA. In some embodiments of any of the aspects, the detection method, e.g., using four primers per target RNA molecule, has a limit of detection of at least 100 target RNA copies per mL for a given target RNA. In some embodiments of any of the aspects, the limit of detection of the target RNA decreases and the sensitivity increases as the number of primers specific for a given target RNA molecule increases.

In some embodiments of any of the aspects, the detection method has a limit of detection of at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, at least 850, at least 860, at least 870, at least 880, at least 890, at least 900, at least 910, at least 920, at least 930, at least 940, at least 950, at least 960, at least 970, at least 980, at least 990, or at least 1000 or more target RNA copies per mL for a given target RNA.

In some embodiments of any of the aspects, the detection method has a dynamic range of at least 3 logs. As used herein, the term “dynamic range” refers to the variation of target RNA concentrations detectable by the methods described herein. Dynamic range can be calculated as the base-10 logarithmic value (“logs”) of the difference between the smallest and largest signal values. In some embodiments of any of the aspects, the detection method has a dynamic range of at least 5 logs. In some embodiments of any of the aspects, the detection method has a dynamic range of at least 6 logs. In some embodiments of any of the aspects, the detection method has a dynamic range of at least 3 logs, at least 3.25 logs, at least 3.5 logs, at least 3.75 logs, at least 4 logs, at least 4.25 logs, at least 4.5 logs, at least 4.75 logs, at least 5 logs, at least 5.25 logs, at least 5.5 logs, at least 5.75 logs, at least 6 logs, at least 6.25 logs, at least 6.5 logs, at least 6.75 logs, at least 7 logs or more.

In some embodiments of any of the aspects, between any of the steps, the reaction product is diluted before being added to the next reaction step. In some embodiments of any of the aspects, the reaction product of step (a) (e.g., the RT step) is diluted prior to being added to step (b) (e.g., the pooling step). In some embodiments of any of the aspects, the pooled mixture of step (b) (e.g., the pooling step) is diluted prior to being added to step (c) (e.g., the amplification step). In some embodiments of any of the aspects, the reaction product of step (c) (e.g., the amplification step) is diluted prior to being added to step (d) (e.g., the sequencing step). In some embodiments, such a dilution step reduces the level of components (e.g., primers, stabilization agents, metal-chelating agents, etc.) that can inhibit subsequent enzymatic reaction(s).

In some embodiments of any of the aspects, the diluent comprises the reaction buffer of the next reaction or an aqueous solution. In some embodiments of any of the aspects, the dilution comprises a ratio of at least 4:5, at least 2:3, at least 1:2, at least 1:3, at least 1:4, at least 1:5, at least 1:6, at least 1:7, at least 1:8, at least 1:9, at least 1:10, at least 1:20, at least 1:30, at least 1:40, at least 1:50, at least 1:60, at least 1:70, at least 1:80, at least 1:90, at least 1:10, at least 1:100, least 1:200, least 1:300, least 1:400, least 1:500, least 1:600, least 1:700, least 1:800, least 1:900, at least 1:10³, at least 1:10⁴, or at least 1:10⁵, of reaction product to diluent.

In some embodiments of any of the aspects, steps (a), (b), (c), and/or (d), or a sub-part thereof, are performed between 12° C. and 72° C. As a non-limiting example, steps (a), (b), (c), and/or (d), or a sub-part thereof, are performed at a temperature of at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., at least 71° C., at least 72° C. or more. In some embodiments of any of the aspects, steps (a), (b), (c), and/or (d) further comprise a step of heat-inactivation, e.g., heat-inactivation of an enzyme (reverse transcriptase; UDG; etc.). Such heat inactivation can be performed at least 50° C., at least 55° C., at least 60° C., at least 65° C., at least 70° C., at least 75° C., at least 80° C., at least 85° C., at least 90° C., at least 91° C., at least 92° C., at least 93° C., at least 94° C., at least 95° C., at least 96° C., at least 97° C., at least 98° C., at least 99° C., at least 99° C., or at least 99.5° C.

In some embodiments of any of the aspects, steps (a), (b), (c), and/or (d), or a sub-part thereof, are performed at a temperature of at most 20° C., at most 21° C., at most 22° C., at most 23° C., at most 24° C., at most 25° C., at most 26° C., at most 27° C., at most 28° C., at most 29° C., at most 30° C., at most 31° C., at most 32° C., at most 33° C., at most 34° C., at most 35° C., at most 36° C., at most 37° C., at most 38° C., at most 39° C., at most 40° C., at most 41° C., at most 42° C., at most 43° C., at most 44° C., at most 45° C., at most 46° C., at most 47° C., at most 48° C., at most 49° C., at most 50° C., at most 51° C., at most 52° C., at most 53° C., at most 54° C., at most 55° C., at most 56° C., at most 57° C., at most 58° C., at most 59° C., at most 60° C., at most 61° C., at most 62° C., at most 63° C., at most 64° C., at most 65° C., at most 66° C., at most 67° C., at most 68° C., at most 69° C., at most 70° C., at most 71° C., or at most 72° C.

In some embodiments of any of the aspects, steps (a), (b), (c), and/or (d) are performed at room temperature. As used herein, the term “room temperature” refers to the ambient temperature of a space, which is typically 20° C.-22° C. In some embodiments of any of the aspects, steps (a), (b), (c), and/or (d) are performed at body temperature. As used herein, the term “body temperature” refers to the temperature of the subject such as that of a human subject, which is typically 37° C. In some embodiments of any of the aspects, steps (a), (b), (c), and/or (d) are performed on a heat block or an incubator capable of maintaining a stable temperature. In some embodiments of any of the aspects, the heat block or incubator is set to approximately 50° C. In some embodiments of any of the aspects, steps (a), (b), (c), and/or (d) are performed in a thermocycler.

In some embodiments of any of the aspects, steps (a), (b), (c), and/or (d) are performed in at most 30 minutes. As a non-limiting example, steps (a), (b), (c), and/or (d) are performed in at most 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

In some embodiments of any of the aspects, steps (a), (b), and (c) are performed in at most 60 minutes. In some embodiments of any of the aspects, steps (a), (b), and (c) are performed in at most 60 minutes, at most 65 minutes, at most 70 minutes, at most 75 minutes, at most 80 minutes, at most 85 minutes, at most 90 minutes, at most 95 minutes, at most 100 minutes, at most 105 minutes, at most 110 minutes, at most 115 minutes, at most 120 minutes, at most 2.5 hours, at most 3 hours, at most 3.5 hours, at most 4 hours, at most 4.5 hours, at most 5 hours, at most 5.5 hours, at most 6 hours, at most 6.5 hours, at most 7 hours, at most 7.5 hours, at most 8 hours, at most 8.5 hours, at most 9 hours, at most 9.5 hours, at most 10 hours, at most 10.5 hours, at most 11 hours, at most 11.5 hours, at most 12 hours, at most 12.5 hours, at most 13 hours, at most 13.5 hours, at most 14 hours, at most 14.5 hours, at most 15 hours, at most 15.5 hours, at most 16 hours, at most 16.5 hours, at most 17 hours, at most 17.5 hours, or at most 18 hours.

In some embodiments of any of the aspects, steps (a), (b), (c), and (d) are performed in at most 180 minutes. In some embodiments of any of the aspects, steps (a), (b), (c), and (d) are performed in at most 2 hours, at most 2.5 hours, at most 3 hours, at most 3.5 hours, at most 4 hours, at most 4.5 hours, at most 5 hours, at most 5.5 hours, at most 6 hours, at most 6.5 hours, at most 7 hours, at most 7.5 hours, at most 8 hours, at most 8.5 hours, at most 9 hours, at most 9.5 hours, at most 10 hours, at most 10.5 hours, at most 11 hours, at most 11.5 hours, at most 12 hours, at most 12.5 hours, at most 13 hours, at most 13.5 hours, at most 14 hours, at most 14.5 hours, at most 15 hours, at most 15.5 hours, at most 16 hours, at most 16.5 hours, at most 17 hours, at most 17.5 hours, or at most 18 hours.

### Sample Preparation

Described herein are methods, kits, and systems permitting detection of a target RNA from a sample. The term “sample” or “test sample” as used herein denotes a sample taken or isolated from a biological organism, e.g., a subject in need of testing. In some embodiments of any of the aspects, the technology described herein encompasses several examples of a biological sample, including but not limited to a saliva sample, sputum sample, a nasopharyngeal sample, a pharyngeal sample, or a nasal sample. In some embodiments of any of the aspects, the sample is a saliva sample. In some embodiments of any of the aspects, the sample is obtained using a swab or another collection tool. In some embodiments of any of the aspects, the biological sample is cells, or tissue, or peripheral blood, or bodily fluid. Depending on the type of target RNA to be detected, exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample, etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the sample is contacted with a transport medium, such as a viral transport medium (VTM). In some embodiments of any of the aspects, transport medium preserves the target RNA between the time of sample collection and assaying the sample for the detection of the target RNA. The constituents of suitable viral transport media are designed to provide an isotonic solution containing protective agents, including protein protective agents, antibiotics to control microbial contamination, and one or more buffers to control the pH. Isotonicity, however, is not an absolute requirement; some highly successful transport media contain hypertonic solutions of sucrose. Liquid transport media are used primarily for transporting swabs or materials released into the medium from a collection swab. Liquid media can be added to other specimens when inactivation of the viral agent is likely and when the resultant dilution is acceptable. An exemplary VTM comprises FBS (e.g., 2%; heat-inactivated at 56° C. for 30 min, Gibco™ 26140079), 1x Antibiotic-Antimycotic (Gibco™, 15240096) and phenol red (e.g., 11 mg/L), in 1 x Hank’s balanced salt solution. In some embodiments of any of the aspects, the VTM further comprises a detergent, in an amount that does not interfere with subsequent enzymatic reactions; the detergent can allow for viral lysis without the need for a nucleic-acid extraction step. Another exemplary VTM suitable for use in collecting throat and nasal swabs from human patients is prepared as follows: (1) add 10 g veal infusion broth and 2 g bovine albumin fraction V to sterile distilled water (to 400 ml); (2) add 0.8 ml gentamicin sulfate solution (50 mg/ml) and 3.2 ml amphotericin B (250 µg/ml); and (3) sterilize by filtration. Additional non-limiting examples of viral transport media include COPAN Universal Transport Medium; Eagle Minimum Essential Medium (E-MEM); Transport medium 199; and PBS-Glycerol transport medium. see e.g., Johnson, Transport of Viral Specimens, CLINICAL MICROBIOLOGY REVIEWS, April 1990, p. 120-131; Collecting, preserving and shipping specimens for the diagnosis of avian influenza A(H5N1) virus infection, Guide for field operations, October 2006. In some embodiments of any of the aspects, viral transport media does not inhibit the detection methods as described herein.

In some embodiments of any of the aspects, prior to the reverse transcription (RT) step total RNA is not isolated from the sample. In some embodiments of any of the aspects, prior to the RT step, the at least one target RNA is not extracted from the sample. In some embodiments of any of the aspects, prior to the RT step, a standard RNA isolation method or kit is not used. Non-limiting examples of standard RNA extraction methods, which are not necessary to be used herein, include: (1) organic extraction, such as phenol-Guanidine Isothiocyanate (GITC)-based solutions (e.g., TRIZOL and TRI reagent); (2) silica-membrane based spin column technology (e.g., RNeasy and its variants); (3) paramagnetic particle technology (e.g., DYNABEADS mRNA DIRECT MICRO); (4) density gradient centrifugation using cesium chloride or cesium trifluoroacetate; (5) lithium chloride and urea isolation; (6) oligo(dt)-cellulose column chromatography; and (7) non-column poly (A)+ purification/isolation. In some embodiments of any of the aspects, prior to the RT step the sample is not heat-inactivated.

In some embodiments of any of the aspects, prior to the RT step, the sample is contacted with a detergent, in an amount that does not interfere with subsequent enzymatic reactions; the detergent can allow for viral lysis without the need for a nucleic-acid extraction step. Alternatively, the sample can be contacted with a detergent in an amount that facilitates release of viral nucleic acids, but that may be high enough to impact subsequent enzymatic steps; in this instance, dilution of the detergent-containing sample prior to enzymatic reaction (e.g., RT reaction, amplification reaction, or both) can reduce the detergent to a level that permits efficient enzyme activity. Non-limiting examples of detergents include Triton X-100, sodium tri-isopropyl naphthalene sulfonate, lithium dodecyl sulfate (LDS); sodium dodecyl sulfate (SDS), NP-40; lecithin, a Span group (e.g., Span 20, or 80), or a Tween group (e.g., Tween 20, 21, 40, 60, 60 K, 61, 65, 80, 80 K, 81, or 85), a sugar amide (e.g. polysaccharide amide), or an alkyl polyglucocide. In some embodiments of any of the aspects, the detergent is Triton X-100 (2-[4-(2,4,4-trimethylpentan-2-yl)phenoxy]ethanol).

In some embodiments of any of the aspects, the test sample can be an untreated test sample. As used herein, the phrase “untreated test sample” refers to a test sample that has not had any prior sample pre-treatment except for dilution and/or suspension in a solution. While pre-treatment is not required, and the lack of such requirement provides an advantage for assay workflow and throughput, in some embodiments the test sample can be treated prior to performing the RNA detection methods as described herein. Exemplary methods for treating a test sample include, but are not limited to, centrifugation, filtration, sonication, homogenization, heating, freezing and thawing, and combinations thereof. In some embodiments of any of the aspects, the test sample can be a frozen test sample. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, homogenization, sonication, filtration, thawing, purification, and any combinations thereof. In some embodiments of any of the aspects, the test sample can be treated with a chemical and/or biological reagent. Chemical and/or biological reagents can be employed, for example, to protect and/or maintain the stability of the sample, including biomolecules (e.g., nucleic acid and protein) therein, during processing. The skilled artisan is well aware of methods and processes appropriate for pre-processing of biological samples required for detection of a nucleic acid as described herein.

### Target RNA

Described herein are methods, kits, and systems that can be used to detect a target RNA, which can also be referred to as “an RNA of interest.” Ribonucleic acid (RNA) is a polymeric nucleic acid molecule essential in various biological roles in coding, decoding, regulation and expression of genes. Each nucleotide in RNA contains a ribose sugar, with carbons numbered 1′ through 5′. A base is attached to the 1′ position, in general, adenine (A), cytosine (C), guanine (G), or uracil (U). A phosphate group is attached to the 3′ position of one ribose and the 5′ position of the next. The phosphate groups have a negative charge each, making RNA a charged molecule (polyanion). An important structural component of RNA that distinguishes it from DNA is the presence of a hydroxyl group at the 2′ position of the ribose sugar. In some embodiments of any of the aspects, the target RNA can be any known type of RNA. In some embodiments of any of the aspects, the target RNA comprises an RNA selected from Table 11.

TABLE 11

Non-limiting Examples of Target RNAs
RNAs involved in protein synthesis

| Type | Abbr. | Function | Distribution |
|---|---|---|---|
| Messenger RNA | mRNA | Codes for protein | All organisms |
| Ribosomal RNA | rRNA | Translation | All organisms |
| Signal recognition particle RNA | 7SL RNA or SRP RNA | Membrane integration | All organisms |
| Transfer RNA | tRNA | Translation | All organisms |
| Transfer-messenger RNA | tmRNA | Rescuing stalled ribosomes | Bacteria |
| RNAs involved in post-transcriptional modification or DNA replication | | | |
| Type | Abbr. | Function | Distribution |
| Small nuclear RNA | snRNA | Splicing and other functions | Eukaryotes and archaea |
| Small nucleolar RNA | snoRNA | Nucleotide modification of RNAs | Eukaryotes and archaea |
| SmY RNA | SmY | mRNA trans-splicing | Nematodes |
| Small Cajal body-specific RNA | scaRNA | Type of snoRNA; Nucleotide modification of RNAs | |
| Guide RNA | gRNA | mRNA nucleotide modification | Kinetoplastid mitochondria |
| Ribonuclease P | RNase P | tRNA maturation | All organisms |
| Ribonuclease MRP | RNase MRP | rRNA maturation, DNA replication | Eukaryotes |
| Y RNA | | RNA processing, DNA replication | Animals |
| Telomerase RNA Component | TERC | Telomere synthesis | Most eukaryotes |
| Spliced Leader RNA | SL RNA | mRNA trans-splicing, RNA processing | |
| Regulatory RNAs | | | |
| Type | Abbr. | Function | Distribution |
| Antisense RNA | aRNA, asRNA | Transcriptional attenuation / mRNA degradation / mRNA stabilisation / Translation block | All organisms |
| Cis-natural antisense transcript | cis-NAT | Gene regulation | |
| CRISPR RNA | crRNA | Resistance to parasites, by targeting their DNA | Bacteria and archaea |
| Long noncoding RNA | lncRNA | Regulation of gene transcription, epigenetic regulation | Eukaryotes |
| MicroRNA | miRNA | Gene regulation | Most eukaryotes |
| Piwi-interacting RNA | piRNA | Transposon defense, maybe other functions | Most animals |
| Small interfering RNA | siRNA | Gene regulation | Most eukaryotes |
| Short hairpin RNA | shRNA | Gene regulation | Most eukaryotes |
| Trans-acting siRNA | tasiRNA | Gene regulation | Land plants |
| Repeat associated siRNA | rasiRNA | Type of piRNA; transposon defense | Drosophila |
| 7SK RNA | 7SK | negatively regulating CDK9/cyclin T complex | |
| Enhancer RNA | eRNA | Gene regulation | |
| Parasitic RNAs | | | |
| Type | Abbr. | Function | Distribution |
| Retrotransposon | | Self-propagating | Eukaryotes and some bacteria |
| Viral genome | | Information carrier | Double-stranded RNA viruses, positive-sense RNA viruses, negative-sense RNA viruses, many satellite viruses and reverse transcribing viruses |
| Viroid | | Self-propagating | Infected plants |
| Satellite RNA | | Self-propagating | Infected cells |
| Other RNAs | | | |
| Type | Abbr. | Function | Distribution |
| Vault RNA | vRNA, vtRNA | Expulsion of xenobiotics (conjectured) | |

In some embodiments of any of the aspects, at least 2 target RNAs in a single sample are detected, which can be on the same RNA molecule or different RNA molecules. Targeting more than one sequence on an RNA molecule, including but not limited to more than one sequence on a viral genomic RNA can permit increased sensitivity for the assay. This is especially true of longer RNA molecules, which can be subject to some degree of degradation - an assay designed to detect any of a number of sequences on the RNA molecule can improve the chances for detection by increasing the number of possible targets for detection. If one target site has been disrupted by cleavage or other degradation, other sites may remain intact, permitting detection. In some embodiments of any of the aspects, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, at least 850, at least 860, at least 870, at least 880, at least 890, at least 900, at least 910, at least 920, at least 930, at least 940, at least 950, at least 960, at least 970, at least 980, at least 990, at least 1000 target RNAs in a single sample are detected, which can be on the same RNA molecule or different RNA molecules.

In some embodiments of any of the aspects, at least one target RNA is a viral RNA. In some embodiments of any of the aspects, at least 2 target RNAs are from the same virus, which can be an RNA virus, a retrovirus, or a DNA virus. In some embodiments of any of the aspects, at least 2 target RNAs are from at least 2 different viruses, non-limiting examples of which are provided herein. Accordingly, in one aspect described herein is a method of detecting an RNA virus in a sample from a subject, comprising: obtaining a sample from the subject; and performing the methods as described herein (e.g., One-Seq) to detect the target viral RNA.

As used herein, the term “RNA virus” refers to a virus comprising an RNA genome. In some embodiments of any of the aspects, the RNA virus is a double-stranded RNA virus, a positive-sense RNA virus, a negative-sense RNA virus, or a reverse transcribing virus (e.g., retrovirus).

In some embodiments of any of the aspects, the RNA virus is a Group III (i.e., double stranded RNA (dsRNA)) virus. In some embodiments of any of the aspects, the Group III RNA virus belongs to a viral family selected from the group consisting of: Amalgaviridae, Birnaviridae, Chrysoviridae, Cystoviridae, Endomaviridae, Hypoviridae, Megabirnaviridae, Partitiviridae, Picobirnaviridae, Reoviridae (e.g., Rotavirus), Totiviridae, Quadriviridae. In some embodiments of any of the aspects, the Group III RNA virus belongs to the Genus Botybirnavirus. In some embodiments of any of the aspects, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some embodiments of any of the aspects, the RNA virus is a Group IV (i.e., positive-sense single stranded (ssRNA)) virus. In some embodiments of any of the aspects, the Group IV RNA virus belongs to a viral order selected from the group consisting of: Nidovirales, Picomavirales, and Tymovirales. In some embodiments of any of the aspects, the Group IV RNA virus belongs to a viral family selected from the group consisting of: Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus),

Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some embodiments of any of the aspects, the Group IV RNA virus belongs to a viral genus selected from the group consisting of: Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some embodiments of any of the aspects, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some embodiments of any of the aspects, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some embodiments of any of the aspects, the RNA virus is a Group V (i.e., negative-sense ssRNA) virus. In some embodiments of any of the aspects, the Group V RNA virus belongs to a viral phylum or subphylum selected from the group consisting of: Negarnaviricota, Haploviricotina, and Polyploviricotina. In some embodiments of any of the aspects, the Group V RNA virus belongs to a viral class selected from the group consisting of: Chunqiuviricetes, Ellioviricetes, Insthoviricetes, Milneviricetes, Monjiviricetes, and Yunchangviricetes. In some embodiments of any of the aspects, the Group V RNA virus belongs to a viral order selected from the group consisting of: Articulavirales, Bunyavirales, Goujianvirales, Jingchuvirales, Mononegavirales, Muvirales, and Serpentovirales. In some embodiments of any of the aspects, the Group V RNA virus belongs to a viral family selected from the group consisting of: Amnoonviridae (e.g., Taastrup virus), Arenaviridae (e.g., Lassa virus), Aspiviridae, Bornaviridae (e.g., Borna disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some embodiments of any of the aspects, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some embodiments of any of the aspects, the RNA virus is a Group VI RNA virus, which comprise a virally encoded reverse transcriptase. In some embodiments of any of the aspects, the Group VI RNA virus belongs to the viral order Ortervirales. In some embodiments of any of the aspects, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some embodiments of any of the aspects, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus). In some embodiments of any of the aspects, the RNA virus is any known RNA virus.

In some embodiments of any of the aspects, the RNA virus is a coronavirus. The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals - and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the coronavirus is selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKu1. In some embodiments of any of the aspects, the coronavirus is severe acute respiratory syndrome coronavirus 2 (SARS-CoV-2), which causes coronavirus disease of 2019 (COVID19 or simply COVID). In some embodiments of any of the aspects, the coronavirus is severe acute respiratory syndrome coronavirus (SARS-CoV or SARS-CoV-1), which causes SARS. In some embodiments of any of the aspects, the coronavirus is Middle East respiratory syndrome-related coronavirus (MERS-CoV), which causes MERS.

In some embodiments of any of the aspects, the RNA virus is severe acute respiratory syndrome coronavirus 2 (SARS-CoV-2). In some embodiments of any of the aspects, at least one viral RNA is a SARS-CoV-2 RNA. In some embodiments of any of the aspects, the target nucleic acid comprises at least a portion of Severe acute respiratory syndrome coronavirus 2 isolate SARS-CoV-2, (see e.g., complete genome, SARS-CoV-2 Jan. 2020/NC_045512.2 Assembly (wuhCor1)). In some embodiments of any of the aspects, the target nucleic acid comprises any gene from SARS-CoV-2, such as the N gene, the S gene, or the ORF1ab gene. In some embodiments of any of the aspects, the target nucleic acid comprises SEQ ID NO: 1001 (Severe acute respiratory syndrome coronavirus 2 isolate SARS-CoV-2, N gene). In some embodiments of any of the aspects, the target nucleic acid comprises SEQ ID NO: 1002 (Severe acute respiratory syndrome coronavirus 2 isolate SARS-CoV-2, S gene). In some embodiments of any of the aspects, the target nucleic acid comprises SEQ ID NO: 1018 (Severe acute respiratory syndrome coronavirus 2 isolate SARS-CoV-2, ORF1ab gene). In some embodiments of any of the aspects, the target nucleic acid comprises one of SEQ ID NOs: 1001-1002 or SEQ ID NO: 1018, or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NO: 1001-1002 or SEQ ID NO: 1018 that maintains the same function or a codon-optimized version of SEQ ID NOs: 1001-1002. In some embodiments of any of the aspects, the target nucleic acid comprises one of SEQ ID NOs: 1001-1002 or SEQ ID NO: 1018, or a nucleic acid sequence that is at least 95% identical to one of SEQ ID NOs: 1001-1002 or SEQ ID NO: 1018 that maintains the same function.

SEQ ID NO: 1001, Severe acute respiratory syndrome coronavirus 2 isolate Wuhan-Hu-1, N nucleocapsid phosphoprotein, Gene ID: 43740575, 1260 bp ss-RNA, NC_045512 REGION: 28274-29533

ATGTCTGATAATGGACCCCAAAATCAGCGAAATGCACCCCGCATTACGTT
TGGTGGACCCTCAGATTCAACTGGCAGTAACCAGAATGGAGAACGCAGTG
GGGCGCGATCAAAACAACGTCGGCCCCAAGGTTTACCCAATAATACTGCG
TCTTGGTTCACCGCTCTCACTCAACATGGCAAGGAAGACCTTAAATTCCC
TCGAGGACAAGGCGTTCCAATTAACACCAATAGCAGTCCAGATGACCAAA
TTGGCTACTACCGAAGAGCTACCAGACGAATTCGTGGTGGTGACGGTAAA
ATGAAAGATCTCAGTCCAAGATGGTATTTCTACTACCTAGGAACTGGGCC
AGAAGCTGGACTTCCCTATGGTGCTAACAAAGACGGCATCATATGGGTTG
CAACTGAGGGAGCCTTGAATACACCAAAAGATCACATTGGCACCCGCAAT
CCTGCTAACAATGCTGCAATCGTGCTACAACTTCCTCAAGGAACAACATT
GCCAAAAGGCTTCTACGCAGAAGGGAGCAGAGGCGGCAGTCAAGCCTCTT
CTCGTTCCTCATCACGTAGTCGCAACAGTTCAAGAAATTCAACTCCAGGC
AGCAGTAGGGGAACTTCTCCTGCTAGAATGGCTGGCAATGGCGGTGATGC
TGCTCTTGCTTTGCTGCTGCTTGACAGATTGAACCAGCTTGAGAGCAAAA
TGTCTGGTAAAGGCCAACAACAACAAGGCCAAACTGTCACTAAGAAATCT
GCTGCTGAGGCTTCTAAGAAGCCTCGGCAAAAACGTACTGCCACTAAAGC
ATACAATGTAACACAAGCTTTCGGCAGACGTGGTCCAGAACAAACCCAAG
GAAATTTTGGGGACCAGGAACTAATCAGACAAGGAACTGATTACAAACAT
TGGCCGCAAATTGCACAATTTGCCCCCAGCGCTTCAGCGTTCTTCGGAAT
GTCGCGCATTGGCATGGAAGTCACACCTTCGGGAACGTGGTTGACCTACA
CAGGTGCCATCAAATTGGATGACAAAGATCCAAATTTCAAAGATCAAGTC
ATTTTGCTGAATAAGCATATTGACGCATACAAAACATTCCCACCAACAGA
GCCTAAAAAGGACAAAAAGAAGAAGGCTGATGAAACTCAAGCCTTACCGC
AGAGACAGAAGAAACAGCAAACTGTGACTCTTCTTCCTGCTGCAGATTTG
GATGATTTCTCCAAACAATTGCAACAATCCATGAGCAGTGCTGACTCAAC
TCAGGCCTAA

SEQ ID NO: 1002, Severe acute respiratory syndrome coronavirus 2 isolate Wuhan-Hu-1, S surface glycoprotein, Gene ID: 43740568, 3822 bp ss-RNA, NC_045512 REGION: 21563-25384

ATGTTTGTTTTTCTTGTTTTATTGCCACTAGTCTCTAGTCAGTGTGTTAA
TCTTACAACCAGAACTCAATTACCCCCTGCATACACTAATTCTTTCACAC
GTGGTGTTTATTACCCTGACAAAGTTTTCAGATCCTCAGTTTTACATTCA
ACTCAGGACTTGTTCTTACCTTTCTTTTCCAATGTTACTTGGTTCCATGC
TATACATGTCTCTGGGACCAATGGTACTAAGAGGTTTGATAACCCTGTCC
TACCATTTAATGATGGTGTTTATTTTGCTTCCACTGAGAAGTCTAACATA
ATAAGAGGCTGGATTTTTGGTACTACTTTAGATTCGAAGACCCAGTCCCT
ACTTATTGTTAATAACGCTACTAATGTTGTTATTAAAGTCTGTGAATTTC
AATTTTGTAATGATCCATTTTTGGGTGTTTATTACCACAAAAACAACAAA
AGTTGGATGGAAAGTGAGTTCAGAGTTTATTCTAGTGCGAATAATTGCAC
TTTTGAATATGTCTCTCAGCCTTTTCTTATGGACCTTGAAGGAAAACAGG
GTAATTTCAAAAATCTTAGGGAATTTGTGTTTAAGAATATTGATGGTTAT
TTTAAAATATATTCTAAGCACACGCCTATTAATTTAGTGCGTGATCTCCC
TCAGGGTTTTTCGGCTTTAGAACCATTGGTAGATTTGCCAATAGGTATTA
ACATCACTAGGTTTCAAACTTTACTTGCTTTACATAGAAGTTATTTGACT
CCTGGTGATTCTTCTTCAGGTTGGACAGCTGGTGCTGCAGCTTATTATGT
GGGTTATCTTCAACCTAGGACTTTTCTATTAAAATATAATGAAAATGGAA
CCATTACAGATGCTGTAGACTGTGCACTTGACCCTCTCTCAGAAACAAAG
TGTACGTTGAAATCCTTCACTGTAGAAAAAGGAATCTATCAAACTTCTAA
CTTTAGAGTCCAACCAACAGAATCTATTGTTAGATTTCCTAATATTACAA
ACTTGTGCCCTTTTGGTGAAGTTTTTAACGCCACCAGATTTGCATCTGTT
TATGCTTGGAACAGGAAGAGAATCAGCAACTGTGTTGCTGATTATTCTGT
CCTATATAATTCCGCATCATTTTCCACTTTTAAGTGTTATGGAGTGTCTC
CTACTAAATTAAATGATCTCTGCTTTACTAATGTCTATGCAGATTCATTT
GTAATTAGAGGTGATGAAGTCAGACAAATCGCTCCAGGGCAAACTGGAAA
GATTGCTGATTATAATTATAAATTACCAGATGATTTTACAGGCTGCGTTA
TAGCTTGGAATTCTAACAATCTTGATTCTAAGGTTGGTGGTAATTATAAT
TACCTGTATAGATTGTTTAGGAAGTCTAATCTCAAACCTTTTGAGAGAGA
TATTTCAACTGAAATCTATCAGGCCGGTAGCACACCTTGTAATGGTGTTG
AAGGTTTTAATTGTTACTTTCCTTTACAATCATATGGTTTCCAACCCACT
AATGGTGTTGGTTACCAACCATACAGAGTAGTAGTACTTTCTTTTGAACT
TCTACATGCACCAGCAACTGTTTGTGGACCTAAAAAGTCTACTAATTTGG
TTAAAAACAAATGTGTCAATTTCAACTTCAATGGTTTAACAGGCACAGGT
GTTCTTACTGAGTCTAACAAAAAGTTTCTGCCTTTCCAACAATTTGGCAG
AGACATTGCTGACACTACTGATGCTGTCCGTGATCCACAGACACTTGAGA
TTCTTGACATTACACCATGTTCTTTTGGTGGTGTCAGTGTTATAACACCA
GGAACAAATACTTCTAACCAGGTTGCTGTTCTTTATCAGGATGTTAACTG
CACAGAAGTCCCTGTTGCTATTCATGCAGATCAACTTACTCCTACTTGGC
GTGTTTATTCTACAGGTTCTAATGTTTTTCAAACACGTGCAGGCTGTTTA
ATAGGGGCTGAACATGTCAACAACTCATATGAGTGTGACATACCCATTGG
TGCAGGTATATGCGCTAGTTATCAGACTCAGACTAATTCTCCTCGGCGGG
CACGTAGTGTAGCTAGTCAATCCATCATTGCCTACACTATGTCACTTGGT
GCAGAAAATTCAGTTGCTTACTCTAATAACTCTATTGCCATACCCACAAA
TTTTACTATTAGTGTTACCACAGAAATTCTACCAGTGTCTATGACCAAGA
CATCAGTAGATTGTACAATGTACATTTGTGGTGATTCAACTGAATGCAGC
AATCTTTTGTTGCAATATGGCAGTTTTTGTACACAATTAAACCGTGCTTT
AACTGGAATAGCTGTTGAACAAGACAAAAACACCCAAGAAGTTTTTGCAC
AAGTCAAACAAATTTACAAAACACCACCAATTAAAGATTTTGGTGGTTTT
AATTTTTCACAAATATTACCAGATCCATCAAAACCAAGCAAGAGGTCATT
TATTGAAGATCTACTTTTCAACAAAGTGACACTTGCAGATGCTGGCTTCA
TCAAACAATATGGTGATTGCCTTGGTGATATTGCTGCTAGAGACCTCATT
TGTGCACAAAAGTTTAACGGCCTTACTGTTTTGCCACCTTTGCTCACAGA
TGAAATGATTGCTCAATACACTTCTGCACTGTTAGCGGGTACAATCACTT
CTGGTTGGACCTTTGGTGCAGGTGCTGCATTACAAATACCATTTGCTATG
CAAATGGCTTATAGGTTTAATGGTATTGGAGTTACACAGAATGTTCTCTA
TGAGAACCAAAAATTGATTGCCAACCAATTTAATAGTGCTATTGGCAAAA
TTCAAGACTCACTTTCTTCCACAGCAAGTGCACTTGGAAAACTTCAAGAT
GTGGTCAACCAAAATGCACAAGCTTTAAACACGCTTGTTAAACAACTTAG
CTCCAATTTTGGTGCAATTTCAAGTGTTTTAAATGATATCCTTTCACGTC
TTGACAAAGTTGAGGCTGAAGTGCAAATTGATAGGTTGATCACAGGCAGA
CTTCAAAGTTTGCAGACATATGTGACTCAACAATTAATTAGAGCTGCAGA
AATCAGAGCTTCTGCTAATCTTGCTGCTACTAAAATGTCAGAGTGTGTAC
TTGGACAATCAAAAAGAGTTGATTTTTGTGGAAAGGGCTATCATCTTATG
TCCTTCCCTCAGTCAGCACCTCATGGTGTAGTCTTCTTGCATGTGACTTA
TGTCCCTGCACAAGAAAAGAACTTCACAACTGCTCCTGCCATTTGTCATG
ATGGAAAAGCACACTTTCCTCGTGAAGGTGTCTTTGTTTCAAATGGCACA
CACTGGTTTGTAACACAAAGGAATTTTTATGAACCACAAATCATTACTAC
AGACAACACATTTGTGTCTGGTAACTGTGATGTTGTAATAGGAATTGTCA
ACAACACAGTTTATGATCCTTTGCAACCTGAATTAGACTCATTCAAGGAG
GAGTTAGATAAATATTTTAAGAATCATACATCACCAGATGTTGATTTAGG
TGACATCTCTGGCATTAATGCTTCAGTTGTAAACATTCAAAAAGAAATTG
ACCGCCTCAATGAGGTTGCCAAGAATTTAAATGAATCTCTCATCGATCTC
CAAGAACTTGGAAAGTATGAGCAGTATATAAAATGGCCATGGTACATTTG
GCTAGGTTTTATAGCTGGCTTGATTGCCATAGTAATGGTGACAATTATGC
TTTGCTGTATGACCAGTTGCTGTAGTTGTCTCAAGGGCTGTTGTTCTTGT
GGATCCTGCTGCAAATTTGATGAAGACGACTCTGAGCCAGTGCTCAAAGG
AGTCAAATTACATTACACATAA

SEQ ID NO: 1003, Severe acute respiratory syndrome coronavirus 2 isolate Wuhan-Hu-1, S surface glycoprotein, Gene ID: 43740568, 1273 aa

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQKQIYKTPPIKDFGGFN
FSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDV
VNQNAQALNTLVKQLSSNFGAISSVLNDILSRLDKVEAEVQIDRLITGRL
QSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTH
WFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCG
SCCKFDEDDSEPVLKGVKLHYT

SEQ ID NO: 1018, ORF1ab polyprotein, Severe acute respiratory syndrome coronavirus 2, isolate Wuhan-Hu-1, NCBI Reference Sequence: NC_045512.2 region: 266-21555, 21290 nt

atggagagccttgtccctggtttcaacgagaaaacacacgtccaactcag
tttgcctgttttacaggttcgcgacgtgctcgtacgtggctttggagact
ccgtggaggaggtcttatcagaggcacgtcaacatcttaaagatggcact
tgtggcttagtagaagttgaaaaaggcgttttgcctcaacttgaacagcc
ctatgtgttcatcaaacgttcggatgctcgaactgcacctcatggtcatg
ttatggttgagctggtagcagaactcgaaggcattcagtacggtcgtagt
ggtgagacacttggtgtccttgtccctcatgtgggcgaaataccagtggc
ttaccgcaaggttcttcttcgtaagaacggtaataaaggagctggtggcc
atagttacggcgccgatctaaagtcatttgacttaggcgacgagcttggc
actgatccttatgaagattttcaagaaaactggaacactaaacatagcag
tggtgttacccgtgaactcatgcgtgagcttaacggaggggcatacactc
gctatgtcgataacaacttctgtggccctgatggctaccctcttgagtgc
attaaagaccttctagcacgtgctggtaaagcttcatgcactttgtccga
acaactggactttattgacactaagaggggtgtatactgctgccgtgaac
atgagcatgaaattgcttggtacacggaacgttctgaaaagagctatgaa
ttgcagacaccttttgaaattaaattggcaaagaaatttgacaccttcaa
tggggaatgtccaaattttgtatttcccttaaattccataatcaagacta
ttcaaccaagggttgaaaagaaaaagcttgatggctttatgggtagaatt
cgatctgtctatccagttgcgtcaccaaatgaatgcaaccaaatgtgcct
ttcaactctcatgaagtgtgatcattgtggtgaaacttcatggcagacgg
gcgattttgttaaagccacttgcgaattttgtggcactgagaatttgact
aaagaaggtgccactacttgtggttacttaccccaaaatgctgttgttaa
aatttattgtccagcatgtcacaattcagaagtaggacctgagcatagtc
ttgccgaataccataatgaatctggcttgaaaaccattcttcgtaagggt
ggtcgcactattgcctttggaggctgtgtgttctcttatgttggttgcca
taacaagtgtgcctattgggttccacgtgctagcgctaacataggttgta
accatacaggtgttgttggagaaggttccgaaggtcttaatgacaacctt
cttgaaatactccaaaaagagaaagtcaacatcaatattgttggtgactt
taaacttaatgaagagatcgccattattttggcatctttttctgcttcca
caagtgcttttgtggaaactgtgaaaggtttggattataaagcattcaaa
caaattgttgaatcctgtggtaattttaaagttacaaaaggaaaagctaa
aaaaggtgcctggaatattggtgaacagaaatcaatactgagtcctcttt
atgcatttgcatcagaggctgctcgtgttgtacgatcaattttctcccgc
actcttgaaactgctcaaaattctgtgcgtgttttacagaaggccgctat
aacaatactagatggaatttcacagtattcactgagactcattgatgcta
tgatgttcacatctgatttggctactaacaatctagttgtaatggcctac
attacaggtggtgttgttcagttgacttcgcagtggctaactaacatctt
tggcactgtttatgaaaaactcaaacccgtccttgattggcttgaagaga
agtttaaggaaggtgtagagtttcttagagacggttgggaaattgttaaa
tttatctcaacctgtgcttgtgaaattgtcggtggacaaattgtcacctg
tgcaaaggaaattaaggagagtgttcagacattctttaagcttgtaaata
aatttttggctttgtgtgctgactctatcattattggtggagctaaactt
aaagccttgaatttaggtgaaacatttgtcacgcactcaaagggattgta
cagaaagtgtgttaaatccagagaagaaactggcctactcatgcctctaa
aagccccaaaagaaattatcttcttagagggagaaacacttcccacagaa
gtgttaacagaggaagttgtcttgaaaactggtgatttacaaccattaga
acaacctactagtgaagctgttgaagctccattggttggtacaccagttt
gtattaacgggcttatgttgctcgaaatcaaagacacagaaaagtactgt
gcccttgcacctaatatgatggtaacaaacaataccttcacactcaaagg
cggtgcaccaacaaaggttacttttggtgatgacactgtgatagaagtgc
aaggttacaagagtgtgaatatcacttttgaacttgatgaaaggattgat
aaagtacttaatgagaagtgctctgcctatacagttgaactcggtacaga
agtaaatgagttcgcctgtgttgtggcagatgctgtcataaaaactttgc
aaccagtatctgaattacttacaccactgggcattgatttagatgagtgg
agtatggctacatactacttatttgatgagtctggtgagtttaaattggc
ttcacatatgtattgttctttctaccctccagatgaggatgaagaagaag
gtgattgtgaagaagaagagtttgagccatcaactcaatatgagtatggt
actgaagatgattaccaaggtaaacctttggaatttggtgccacttctgc
tgctcttcaacctgaagaagagcaagaagaagattggttagatgatgata
gtcaacaaactgttggtcaacaagacggcagtgaggacaatcagacaact
actattcaaacaattgttgaggttcaacctcaattagagatggaacttac
accagttgttcagactattgaagtgaatagttttagtggttatttaaaac
ttactgacaatgtatacattaaaaatgcagacattgtggaagaagctaaa
aaggtaaaaccaacagtggttgttaatgcagccaatgtttaccttaaaca
tggaggaggtgttgcaggagccttaaataaggctactaacaatgccatgc
aagttgaatctgatgattacatagctactaatggaccacttaaagtgggt
ggtagttgtgttttaagcggacacaatcttgctaaacactgtcttcatgt
tgtcggcccaaatgttaacaaaggtgaagacattcaacttcttaagagtg
cttatgaaaattttaatcagcacgaagttctacttgcaccattattatca
gctggtatttttggtgctgaccctatacattctttaagagtttgtgtaga
tactgttcgcacaaatgtctacttagctgtctttgataaaaatctctatg
acaaacttgtttcaagctttttggaaatgaagagtgaaaagcaagttgaa
caaaagatcgctgagattcctaaagaggaagttaagccatttataactga
aagtaaaccttcagttgaacagagaaaacaagatgataagaaaatcaaag
cttgtgttgaagaagttacaacaactctggaagaaactaagttcctcaca
gaaaacttgttactttatattgacattaatggcaatcttcatccagattc
tgccactcttgttagtgacattgacatcactttcttaaagaaagatgctc
catatatagtgggtgatgttgttcaagagggtgttttaactgctgtggtt
atacctactaaaaaggctggtggcactactgaaatgctagcgaaagcttt
gagaaaagtgccaacagacaattatataaccacttacccgggtcagggtt
taaatggttacactgtagaggaggcaaagacagtgcttaaaaagtgtaaa
agtgccttttacattctaccatctattatctctaatgagaagcaagaaat
tcttggaactgtttcttggaatttgcgagaaatgcttgcacatgcagaag
aaacacgcaaattaatgcctgtctgtgtggaaactaaagccatagtttca
actatacagcgtaaatataagggtattaaaatacaagagggtgtggttga
ttatggtgctagattttacttttacaccagtaaaacaactgtagcgtcac
ttatcaacacacttaacgatctaaatgaaactcttgttacaatgccactt
ggctatgtaacacatggcttaaatttggaagaagctgctcggtatatgag
atctctcaaagtgccagctacagtttctgtttcttcacctgatgctgtta
cagcgtataatggttatcttacttcttcttctaaaacacctgaagaacat
tttattgaaaccatctcacttgctggttcctataaagattggtcctattc
tggacaatctacacaactaggtatagaatttcttaagagaggtgataaaa
gtgtatattacactagtaatcctaccacattccacctagatggtgaagtt
atcacctttgacaatcttaagacacttctttctttgagagaagtgaggac
tattaaggtgtttacaacagtagacaacattaacctccacacgcaagttg
tggacatgtcaatgacatatggacaacagtttggtccaacttatttggat
ggagctgatgttactaaaataaaacctcataattcacatgaaggtaaaac
attttatgttttacctaatgatgacactctacgtgttgaggcttttgagt
actaccacacaactgatcctagttttctgggtaggtacatgtcagcatta
aatcacactaaaaagtggaaatacccacaagttaatggtttaacttctat
taaatgggcagataacaactgttatcttgccactgcattgttaacactcc
aacaaatagagttgaagtttaatccacctgctctacaagatgcttattac
agagcaagggctggtgaagctgctaacttttgtgcacttatcttagccta
ctgtaataagacagtaggtgagttaggtgatgttagagaaacaatgagtt
acttgtttcaacatgccaatttagattcttgcaaaagagtcttgaacgtg
gtgtgtaaaacttgtggacaacagcagacaacccttaagggtgtagaagc
tgttatgtacatgggcacactttcttatgaacaatttaagaaaggtgttc
agataccttgtacgtgtggtaaacaagctacaaaatatctagtacaacag
gagtcaccttttgttatgatgtcagcaccacctgctcagtatgaacttaa
gcatggtacatttacttgtgctagtgagtacactggtaattaccagtgtg
gtcactataaacatataacttctaaagaaactttgtattgcatagacggt
gctttacttacaaagtcctcagaatacaaaggtcctattacggatgtttt
ctacaaagaaaacagttacacaacaaccataaaaccagttacttataaat
tggatggtgttgtttgtacagaaattgaccctaagttggacaattattat
aagaaagacaattcttatttcacagagcaaccaattgatcttgtaccaaa
ccaaccatatccaaacgcaagcttcgataattttaagtttgtatgtgata
atatcaaatttgctgatgatttaaaccagttaactggttataagaaacct
gcttcaagagagcttaaagttacatttttccctgacttaaatggtgatgt
ggtggctattgattataaacactacacaccctcttttaagaaaggagcta
aattgttacataaacctattgtttggcatgttaacaatgcaactaataaa
gccacgtataaaccaaatacctggtgtatacgttgtctttggagcacaaa
accagttgaaacatcaaattcgtttgatgtactgaagtcagaggacgcgc
agggaatggataatcttgcctgcgaagatctaaaaccagtctctgaagaa
gtagtggaaaatcctaccatacagaaagacgttcttgagtgtaatgtgaa
aactaccgaagttgtaggagacattatacttaaaccagcaaataatagtt
taaaaattacagaagaggttggccacacagatctaatggctgcttatgta
gacaattctagtcttactattaagaaacctaatgaattatctagagtatt
aggtttgaaaacccttgctactcatggtttagctgctgttaatagtgtcc
cttgggatactatagctaattatgctaagccttttcttaacaaagttgtt
agtacaactactaacatagttacacggtgtttaaaccgtgtttgtactaa
ttatatgccttatttctttactttattgctacaattgtgtacttttacta
gaagtacaaattctagaattaaagcatctatgccgactactatagcaaag
aatactgttaagagtgtcggtaaattttgtctagaggcttcatttaatta
tttgaagtcacctaatttttctaaactgataaatattataatttggtttt
tactattaagtgtttgcctaggttctttaatctactcaaccgctgcttta
ggtgttttaatgtctaatttaggcatgccttcttactgtactggttacag
agaaggctatttgaactctactaatgtcactattgcaacctactgtactg
gttctataccttgtagtgtttgtcttagtggtttagattctttagacacc
tatccttctttagaaactatacaaattaccatttcatcttttaaatggga
tttaactgcttttggcttagttgcagagtggtttttggcatatattcttt
tcactaggtttttctatgtacttggattggctgcaatcatgcaattgttt
ttcagctattttgcagtacattttattagtaattcttggcttatgtggtt
aataattaatcttgtacaaatggccccgatttcagctatggttagaatgt
acatcttctttgcatcattttattatgtatggaaaagttatgtgcatgtt
gtagacggttgtaattcatcaacttgtatgatgtgttacaaacgtaatag
agcaacaagagtcgaatgtacaactattgttaatggtgttagaaggtcct
tttatgtctatgctaatggaggtaaaggcttttgcaaactacacaattgg
aattgtgttaattgtgatacattctgtgctggtagtacatttattagtga
tgaagttgcgagagacttgtcactacagtttaaaagaccaataaatccta
ctgaccagtcttcttacatcgttgatagtgttacagtgaagaatggttcc
atccatctttactttgataaagctggtcaaaagacttatgaaagacattc
tctctctcattttgttaacttagacaacctgagagctaataacactaaag
gttcattgcctattaatgttatagtttttgatggtaaatcaaaatgtgaa
gaatcatctgcaaaatcagcgtctgtttactacagtcagcttatgtgtca
acctatactgttactagatcaggcattagtgtctgatgttggtgatagtg
cggaagttgcagttaaaatgtttgatgcttacgttaatacgttttcatca
acttttaacgtaccaatggaaaaactcaaaacactagttgcaactgcaga
agctgaacttgcaaagaatgtgtccttagacaatgtcttatctactttta
tttcagcagctcggcaagggtttgttgattcagatgtagaaactaaagat
gttgttgaatgtcttaaattgtcacatcaatctgacatagaagttactgg
cgatagttgtaataactatatgctcacctataacaaagttgaaaacatga
caccccgtgaccttggtgcttgtattgactgtagtgcgcgtcatattaat
gcgcaggtagcaaaaagtcacaacattgctttgatatggaacgttaaaga
tttcatgtcattgtctgaacaactacgaaaacaaatacgtagtgctgcta
aaaagaataacttaccttttaagttgacatgtgcaactactagacaagtt
gttaatgttgtaacaacaaagatagcacttaagggtggtaaaattgttaa
taattggttgaagcagttaattaaagttacacttgtgttcctttttgttg
ctgctattttctatttaataacacctgttcatgtcatgtctaaacatact
gacttttcaagtgaaatcataggatacaaggctattgatggtggtgtcac
tcgtgacatagcatctacagatacttgttttgctaacaaacatgctgatt
ttgacacatggtttagccagcgtggtggtagttatactaatgacaaagct
tgcccattgattgctgcagtcataacaagagaagtgggttttgtcgtgcc
tggtttgcctggcacgatattacgcacaactaatggtgactttttgcatt
tcttacctagagtttttagtgcagttggtaacatctgttacacaccatca
aaacttatagagtacactgactttgcaacatcagcttgtgttttggctgc
tgaatgtacaatttttaaagatgcttctggtaagccagtaccatattgtt
atgataccaatgtactagaaggttctgttgcttatgaaagtttacgccct
gacacacgttatgtgctcatggatggctctattattcaatttcctaacac
ctaccttgaaggttctgttagagtggtaacaacttttgattctgagtact
gtaggcacggcacttgtgaaagatcagaagctggtgtttgtgtatctact
agtggtagatgggtacttaacaatgattattacagatctttaccaggagt
tttctgtggtgtagatgctgtaaatttacttactaatatgtttacaccac
taattcaacctattggtgctttggacatatcagcatctatagtagctggt
ggtattgtagctatcgtagtaacatgccttgcctactattttatgaggtt
tagaagagcttttggtgaatacagtcatgtagttgcctttaatactttac
tattccttatgtcattcactgtactctgtttaacaccagtttactcattc
ttacctggtgtttattctgttatttacttgtacttgacattttatcttac
taatgatgtttcttttttagcacatattcagtggatggttatgttcacac
ctttagtacctttctggataacaattgcttatatcatttgtatttccaca
aagcatttctattggttctttagtaattacctaaagagacgtgtagtctt
taatggtgtttcctttagtacttttgaagaagctgcgctgtgcacctttt
tgttaaataaagaaatgtatctaaagttgcgtagtgatgtgctattacct
cttacgcaatataatagatacttagctctttataataagtacaagtattt
tagtggagcaatggatacaactagctacagagaagctgcttgttgtcatc
tcgcaaaggctctcaatgacttcagtaactcaggttctgatgttctttac
caaccaccacaaacctctatcacctcagctgttttgcagagtggttttag
aaaaatggcattcccatctggtaaagttgagggttgtatggtacaagtaa
cttgtggtacaactacacttaacggtctttggcttgatgacgtagtttac
tgtccaagacatgtgatctgcacctctgaagacatgcttaaccctaatta
tgaagatttactcattcgtaagtctaatcataatttcttggtacaggctg
gtaatgttcaactcagggttattggacattctatgcaaaattgtgtactt
aagcttaaggttgatacagccaatcctaagacacctaagtataagtttgt
tcgcattcaaccaggacagactttttcagtgttagcttgttacaatggtt
caccatctggtgtttaccaatgtgctatgaggcccaatttcactattaag
ggttcattccttaatggttcatgtggtagtgttggttttaacatagatta
tgactgtgtctctttttgttacatgcaccatatggaattaccaactggag
ttcatgctggcacagacttagaaggtaacttttatggaccttttgttgac
aggcaaacagcacaagcagctggtacggacacaactattacagttaatgt
tttagcttggttgtacgctgctgttataaatggagacaggtggtttctca
atcgatttaccacaactcttaatgactttaaccttgtggctatgaagtac
aattatgaacctctaacacaagaccatgttgacatactaggacctctttc
tgctcaaactggaattgccgttttagatatgtgtgcttcattaaaagaat
tactgcaaaatggtatgaatggacgtaccatattgggtagtgctttatta
gaagatgaatttacaccttttgatgttgttagacaatgctcaggtgttac
tttccaaagtgcagtgaaaagaacaatcaagggtacacaccactggttgt
tactcacaattttgacttcacttttagttttagtccagagtactcaatgg
tctttgttcttttttttgtatgaaaatgcctttttaccttttgctatggg
tattattgctatgtctgcttttgcaatgatgtttgtcaaacataagcatg
catttctctgtttgtttttgttaccttctcttgccactgtagcttatttt
aatatggtctatatgcctgctagttgggtgatgcgtattatgacatggtt
ggatatggttgatactagtttgtctggttttaagctaaaagactgtgtta
tgtatgcatcagctgtagtgttactaatccttatgacagcaagaactgtg
tatgatgatggtgctaggagagtgtggacacttatgaatgtcttgacact
cgtttataaagtttattatggtaatgctttagatcaagccatttccatgt
gggctcttataatctctgttacttctaactactcaggtgtagttacaact
gtcatgtttttggccagaggtattgtttttatgtgtgttgagtattgccc
tattttcttcataactggtaatacacttcagtgtataatgctagtttatt
gtttcttaggctatttttgtacttgttactttggcctcttttgtttactc
aaccgctactttagactgactcttggtgtttatgattacttagtttctac
acaggagtttagatatatgaattcacagggactactcccacccaagaata
gcatagatgccttcaaactcaacattaaattgttgggtgttggtggcaaa
ccttgtatcaaagtagccactgtacagtctaaaatgtcagatgtaaagtg
cacatcagtagtcttactctcagttttgcaacaactcagagtagaatcat
catctaaattgtgggctcaatgtgtccagttacacaatgacattctctta
gctaaagatactactgaagcctttgaaaaaatggtttcactactttctgt
tttgctttccatgcagggtgctgtagacataaacaagctttgtgaagaaa
tgctggacaacagggcaaccttacaagctatagcctcagagtttagttcc
cttccatcatatgcagcttttgctactgctcaagaagcttatgagcaggc
tgttgctaatggtgattctgaagttgttcttaaaaagttgaagaagtctt
tgaatgtggctaaatctgaatttgaccgtgatgcagccatgcaacgtaag
ttggaaaagatggctgatcaagctatgacccaaatgtataaacaggctag
atctgaggacaagagggcaaaagttactagtgctatgcagacaatgcttt
tcactatgcttagaaagttggataatgatgcactcaacaacattatcaac
aatgcaagagatggttgtgttcccttgaacataatacctcttacaacagc
agccaaactaatggttgtcataccagactataacacatataaaaatacgt
gtgatggtacaacatttacttatgcatcagcattgtgggaaatccaacag
gttgtagatgcagatagtaaaattgttcaacttagtgaaattagtatgga
caattcacctaatttagcatggcctcttattgtaacagctttaagggcca
attctgctgtcaaattacagaataatgagcttagtcctgttgcactacga
cagatgtcttgtgctgccggtactacacaaactgcttgcactgatgacaa
tgcgttagcttactacaacacaacaaagggaggtaggtttgtacttgcac
tgttatccgatttacaggatttgaaatgggctagattccctaagagtgat
ggaactggtactatctatacagaactggaaccaccttgtaggtttgttac
agacacacctaaaggtcctaaagtgaagtatttatactttattaaaggat
taaacaacctaaatagaggtatggtacttggtagtttagctgccacagta
cgtctacaagctggtaatgcaacagaagtgcctgccaattcaactgtatt
atctttctgtgcttttgctgtagatgctgctaaagcttacaaagattatc
tagctagtgggggacaaccaatcactaattgtgttaagatgttgtgtaca
cacactggtactggtcaggcaataacagttacaccggaagccaatatgga
tcaagaatcctttggtggtgcatcgtgttgtctgtactgccgttgccaca
tagatcatccaaatcctaaaggattttgtgacttaaaaggtaagtatgta
caaatacctacaacttgtgctaatgaccctgtgggttttacacttaaaaa
cacagtctgtaccgtctgcggtatgtggaaaggttatggctgtagttgtg
atcaactccgcgaacccatgcttcagtcagctgatgcacaatcgttttta
aacgggtttgcggtgtaagtgcagcccgtcttacaccgtgcggcacaggc
actagtactgatgtcgtatacagggcttttgacatctacaatgataaagt
agctggttttgctaaattcctaaaaactaattgttgtcgcttccaagaaa
aggacgaagatgacaatttaattgattcttactttgtagttaagagacac
actttctctaactaccaacatgaagaaacaatttataatttacttaagga
ttgtccagctgttgctaaacatgacttctttaagtttagaatagacggtg
acatggtaccacatatatcacgtcaacgtcttactaaatacacaatggca
gacctcgtctatgctttaaggcattttgatgaaggtaattgtgacacatt
aaaagaaatacttgtcacatacaattgttgtgatgatgattatttcaata
aaaaggactggtatgattttgtagaaaacccagatatattacgcgtatac
gccaacttaggtgaacgtgtacgccaagctttgttaaaaacagtacaatt
ctgtgatgccatgcgaaatgctggtattgttggtgtactgacattagata
atcaagatctcaatggtaactggtatgatttcggtgatttcatacaaacc
acgccaggtagtggagttcctgttgtagattcttattattcattgttaat
gcctatattaaccttgaccagggctttaactgcagagtcacatgttgaca
ctgacttaacaaagccttacattaagtgggatttgttaaaatatgacttc
acggaagagaggttaaaactctttgaccgttattttaaatattgggatca
gacataccacccaaattgtgttaactgtttggatgacagatgcattctgc
attgtgcaaactttaatgttttattctctacagtgttcccacctacaagt
tttggaccactagtgagaaaaatatttgttgatggtgttccatttgtagt
ttcaactggataccacttcagagagctaggtgttgtacataatcaggatg
taaacttacatagctctagacttagttttaaggaattacttgtgtatgct
gctgaccctgctatgcacgctgcttctggtaatctattactagataaacg
cactacgtgcttttcagtagctgcacttactaacaatgttgcttttcaaa
ctgtcaaacccggtaattttaacaaagacttctatgactttgctgtgtct
aagggtttctttaaggaaggaagttctgttgaattaaaacacttcttctt
tgctcaggatggtaatgctgctatcagcgattatgactactatcgttata
atctaccaacaatgtgtgatatcagacaactactatttgtagttgaagtt
gttgataagtactttgattgttacgatggtggctgtattaatgctaacca
agtcatcgtcaacaacctagacaaatcagctggttttccatttaataaat
ggggtaaggctagactttattatgattcaatgagttatgaggatcaagat
gcacttttcgcatatacaaaacgtaatgtcatccctactataactcaaat
gaatcttaagtatgccattagtgcaaagaatagagctcgcaccgtagctg
gtgtctctatctgtagtactatgaccaatagacagtttcatcaaaaatta
ttgaaatcaatagccgccactagaggagctactgtagtaattggaacaag
caaattctatggtggttggcacaacatgttaaaaactgtttatagtgatg
tagaaaaccctcaccttatgggttgggattatcctaaatgtgatagagcc
atgcctaacatgcttagaattatggcctcacttgttcttgctcgcaaaca
tacaacgtgttgtagcttgtcacaccgtttctatagattagctaatgagt
gtgctcaagtattgagtgaaatggtcatgtgtggcggttcactatatgtt
aaaccaggtggaacctcatcaggagatgccacaactgcttatgctaatag
tgtttttaacatttgtcaagctgtcacggccaatgttaatgcacttttat
ctactgatggtaacaaaattgccgataagtatgtccgcaatttacaacac
agactttatgagtgtctctatagaaatagagatgttgacacagactttgt
gaatgagttttacgcatatttgcgtaaacatttctcaatgatgatactct
ctgacgatgctgttgtgtgtttcaatagcacttatgcatctcaaggtcta
gtggctagcataaagaactttaagtcagttctttattatcaaaacaatgt
ttttatgtctgaagcaaaatgttggactgagactgaccttactaaaggac
ctcatgaattttgctctcaacatacaatgctagttaaacagggtgatgat
tatgtgtaccttccttacccagatccatcaagaatcctaggggccggctg
ttttgtagatgatatcgtaaaaacagatggtacacttatgattgaacggt
tcgtgtctttagctatagatgcttacccacttactaaacatcctaatcag
gagtatgctgatgtctttcatttgtacttacaatacataagaaagctaca
tgatgagttaacaggacacatgttagacatgtattctgttatgcttacta
atgataacacttcaaggtattgggaacctgagttttatgaggctatgtac
acaccgcatacagtcttacaggctgttggggcttgtgttctttgcaattc
acagacttcattaagatgtggtgcttgcatacgtagaccattcttatgtt
gtaaatgctgttacgaccatgtcatatcaacatcacataaattagtcttg
tctgttaatccgtatgtttgcaatgctccaggttgtgatgtcacagatgt
gactcaactttacttaggaggtatgagctattattgtaaatcacataaac
cacccattagttttccattgtgtgctaatggacaagtttttggtttatat
aaaaatacatgtgttggtagcgataatgttactgactttaatgcaattgc
aacatgtgactggacaaatgctggtgattacattttagctaacacctgta
ctgaaagactcaagctttttgcagcagaaacgctcaaagctactgaggag
acatttaaactgtcttatggtattgctactgtacgtgaagtgctgtctga
cagagaattacatctttcatgggaagttggtaaacctagaccaccactta
accgaaattatgtctttactggttatcgtgtaactaaaaacagtaaagta
caaataggagagtacacctttgaaaaaggtgactatggtgatgctgttgt
ttaccgaggtacaacaacttacaaattaaatgttggtgattattttgtgc
tgacatcacatacagtaatgccattaagtgcacctacactagtgccacaa
gagcactatgttagaattactggcttatacccaacactcaatatctcaga
tgagttttctagcaatgttgcaaattatcaaaaggttggtatgcaaaagt
attctacactccagggaccacctggtactggtaagagtcattttgctatt
ggcctagctctctactacccttctgctcgcatagtgtatacagcttgctc
tcatgccgctgttgatgcactatgtgagaaggcattaaaatatttgccta
tagataaatgtagtagaattatacctgcacgtgctcgtgtagagtgtttt
gataaattcaaagtgaattcaacattagaacagtatgtcttttgtactgt
aaatgcattgcctgagacgacagcagatatagttgtctttgatgaaattt
caatggccacaaattatgatttgagtgttgtcaatgccagattacgtgct
aagcactatgtgtacattggcgaccctgctcaattacctgcaccacgcac
attgctaactaagggcacactagaaccagaatatttcaattcagtgtgta
gacttatgaaaactataggtccagacatgttcctcggaacttgtcggcgt
tgtcctgctgaaattgttgacactgtgagtgctttggtttatgataataa
gcttaaagcacataaagacaaatcagctcaatgctttaaaatgttttata
agggtgttatcacgcatgatgtttcatctgcaattaacaggccacaaata
ggcgtggtaagagaattccttacacgtaaccctgcttggagaaaagctgt
ctttatttcaccttataattcacagaatgctgtagcctcaaagattttgg
gactaccaactcaaactgttgattcatcacagggctcagaatatgactat
gtcatattcactcaaaccactgaaacagctcactcttgtaatgtaaacag
atttaatgttgctattaccagagcaaaagtaggcatactttgcataatgt
ctgatagagacctttatgacaagttgcaatttacaagtcttgaaattcca
cgtaggaatgtggcaactttacaagctgaaaatgtaacaggactctttaa
agattgtagtaaggtaatcactgggttacatcctacacaggcacctacac
acctcagtgttgacactaaattcaaaactgaaggtttatgtgttgacata
cctggcatacctaaggacatgacctatagaagactcatctctatgatggg
ttttaaaatgaattatcaagttaatggttaccctaacatgtttatcaccc
gcgaagaagctataagacatgtacgtgcatggattggcttcgatgtcgag
gggtgtcatgctactagagaagctgttggtaccaatttacctttacagct
aggtttttctacaggtgttaacctagttgctgtacctacaggttatgttg
atacacctaataatacagatttttccagagttagtgctaaaccaccgcct
ggagatcaatttaaacacctcataccacttatgtacaaaggacttccttg
gaatgtagtgcgtataaagattgtacaaatgttaagtgacacacttaaaa
atctctctgacagagtcgtatttgtcttatgggcacatggctttgagttg
acatctatgaagtattttgtgaaaataggacctgagcgcacctgttgtct
atgtgatagacgtgccacatgcttttccactgcttcagacacttatgcct
gttggcatcattctattggatttgattacgtctataatccgtttatgatt
gatgttcaacaatggggttttacaggtaacctacaaagcaaccatgatct
gtattgtcaagtccatggtaatgcacatgtagctagttgtgatgcaatca
tgactaggtgtctagctgtccacgagtgctttgttaagcgtgttgactgg
actattgaatatcctataattggtgatgaactgaagattaatgcggcttg
tagaaaggttcaacacatggttgttaaagctgcattattagcagacaaat
tcccagttcttcacgacattggtaaccctaaagctattaagtgtgtacct
caagctgatgtagaatggaagttctatgatgcacagccttgtagtgacaa
agcttataaaatagaagaattattctattcttatgccacacattctgaca
aattcacagatggtgtatgcctattttggaattgcaatgtcgatagatat
cctgctaattccattgtttgtagatttgacactagagtgctatctaacct
taacttgcctggttgtgatggtggcagtttgtatgtaaataaacatgcat
tccacacaccagcttttgataaaagtgcttttgttaatttaaaacaatta
ccatttttctattactctgacagtccatgtgagtctcatggaaaacaagt
agtgtcagatatagattatgtaccactaaagtctgctacgtgtataacac
gttgcaatttaggtggtgctgtctgtagacatcatgctaatgagtacaga
ttgtatctcgatgcttataacatgatgatctcagctggctttagcttgtg
ggtttacaaacaatttgatacttataacctctggaacacttttacaagac
ttcagagtttagaaaatgtggcttttaatgttgtaaataagggacacttt
gatggacaacagggtgaagtaccagtttctatcattaataacactgttta
cacaaaagttgatggtgttgatgtagaattgtttgaaaataaaacaacat
tacctgttaatgtagcatttgagctttgggctaagcgcaacattaaacca
gtaccagaggtgaaaatactcaataatttgggtgtggacattgctgctaa
tactgtgatctgggactacaaaagagatgctccagcacatatatctacta
ttggtgtttgttctatgactgacatagccaagaaaccaactgaaacgatt
tgtgcaccactcactgtcttttttgatggtagagttgatggtcaagtaga
cttatttagaaatgcccgtaatggtgttcttattacagaaggtagtgtta
aaggtttacaaccatctgtaggtcccaaacaagctagtcttaatggagtc
acattaattggagaagccgtaaaaacacagttcaattattataagaaagt
tgatggtgttgtccaacaattacctgaaacttactttactcagagtagaa
atttacaagaatttaaacccaggagtcaaatggaaattgatttcttagaa
ttagctatggatgaattcattgaacggtataaattagaaggctatgcctt
cgaacatatcgtttatggagattttagtcatagtcagttaggtggtttac
atctactgattggactagctaaacgttttaaggaatcaccttttgaatta
gaagattttattcctatggacagtacagttaaaaactatttcataacaga
tgcgcaaacaggttcatctaagtgtgtgtgttctgttattgatttattac
ttgatgattttgttgaaataataaaatcccaagatttatctgtagtttct
aaggttgtcaaagtgactattgactatacagaaatttcatttatgctttg
gtgtaaagatggccatgtagaaacattttacccaaaattacaatctagtc
aagcgtggcaaccgggtgttgctatgcctaatctttacaaaatgcaaaga
atgctattagaaaagtgtgaccttcaaaattatggtgatagtgcaacatt
acctaaaggcataatgatgaatgtcgcaaaatatactcaactgtgtcaat
atttaaacacattaacattagctgtaccctataatatgagagttatacat
tttggtgctggttctgataaaggagttgcaccaggtacagctgttttaag
acagtggttgcctacgggtacgctgcttgtcgattcagatcttaatgact
ttgtctctgatgcagattcaactttgattggtgattgtgcaactgtacat
acagctaataaatgggatctcattattagtgatatgtacgaccctaagac
taaaaatgttacaaaagaaaatgactctaaagagggttttttcacttaca
tttgtgggtttatacaacaaaagctagctcttggaggttccgtggctata
aagataacagaacattcttggaatgctgatctttataagctcatgggaca
cttcgcatggtggacagcctttgttactaatgtgaatgcgtcatcatctg
aagcatttttaattggatgtaattatcttggcaaaccacgcgaacaaata
gatggttatgtcatgcatgcaaattacatattttggaggaatacaaatcc
aattcagttgtcttcctattctttatttgacatgagtaaatttcccctta
aattaaggggtactgctgttatgtctttaaaagaaggtcaaatcaatgat
atgattttatctcttcttagtaaaggtagacttataattagagaaaacaa
cagagttgttatttctagtgatgttcttgttaacaactaa

In some embodiments of any of the aspects, the target RNA comprises a variation of interest. In some embodiments of any of the aspects, the variation of interest is selected from the group consisting of: a single-nucleotide variation; a point mutation; a substitution; an insertion; and a deletion. In some embodiments of any of the aspects, the variation of interest is associated with a variant of SARS-CoV-2. In some embodiments of any of the aspects, the SARS-CoV-2 variant is selected from the group consisting of: B.1.1.7 (also referred to as the United Kingdom variant, 20I/501Y.V1, or VOC 202012/01); B.1.351 (also referred to as the South African variant, or 20H/501Y.V2); P.1 (also referred to as the Brazilian variant); and CAL.20C (also referred to as the California variant). Non-limiting examples of variations of interest include del69-70, del144, K417N, K417T, L452R, E484K, N501Y, D614G, P681H, or A701V in the SARS-CoV-2 S protein (see e.g., SEQ ID NO: 1003). In some embodiments of any of the aspects, variations associated with the B.1.1.7 variant include del69-70, del144, N501Y, D614G, P681H, and/or A701V in the SARS-CoV-2 S protein. In some embodiments of any of the aspects, variations associated with the B.1.351 variant include K417N, E484K, and/or N501Y in the SARS-CoV-2 S protein. In some embodiments of any of the aspects, variations associated with the P.1 variant include K417T, E484K, and/or N501Y in the SARS-CoV-2 S protein. In some embodiments of any of the aspects, a variations associated with the CAL.20C variant includes L452R in the SARS-CoV-2 S protein. See e.g., Table 12 for exemplary variations of interest in the SARS-CoV-2 S gene and associated nucleic acid mutations in the target nucleic acid (T (thymine) and U (uracil) are used interchangeably).

TABLE 12

Exemplary Variations of Interest in SARS-CoV-2 S gene

| Mutation (see e.g., SEQ ID NO: 1003) | nt in SEQ ID NO: 1002 | WT nt in SEQ ID NO: 1002 | Exemplary mutant nt in SEQ ID NO: 1002 | B.1.1.7 | B.1.351 | P.1 | CAL.20C |
|---|---|---|---|---|---|---|---|
| del69-70 (delHV) | 205-210 | 205 - CAT GTC - 210 | (del) | X | | | |
| del144 (delY) | 430-432 | 430 - TAT - 432 | (del) | X | | | |
| K417N | 1249-1251 | 1249 - AAG - 1251 | 1249-AAT-1251 1249-AAC-1251 | | X | | |
| K417T | 1249-1251 | 1249 - AAG - 1251 | 1249-ACG-1251 1249-ACA-1251 1249-ACT-1251 1249-ACC-1251 | | | X | |
| L452R | 1354-1356 | 1354 - CTG - 1356 | 1354-CGG-1356 1354-CGA-1356 1354-CGT-1356 1354-CGC-1356 | | | | X |
| E484K | 1450-1452 | 1450-GAA-1452 | 1450-AAA-1452 1450-AAG-1452 | | X | X | |
| N501Y | 1501-1503 | 1501-AAT-1503 | 1501-TAT-1503 1501-TAC-1503 | X | X | X | |
| D614G | 1840-1842 | 1840-GAT-1842 | 1840-GGT-842 1840-GGA-842 1840-GGC-842 1840-GGG-842 | X | | | |
| P681H | 2041-2043 | 2041-CCT-2043 | 2041-CAT-2043 2041-CAC-2043 | X | | | |
| A701V | 2101-2103 | 2101-GCA-2103 | 2101-GTA-2103 2101-GTT-2103 2101-GTC-2103 2101-GTG-2103 | X | | | |

In some embodiments of any of the aspects, the viral RNA is an RNA produced by a virus with a DNA genome, i.e., a DNA virus. As a non-limiting example the DNA virus is a Group I (dsDNA) virus, a Group II (ssDNA) virus, or a Group VII (dsDNA-RT) virus. In some embodiments of any of the aspects, the RNA produced by a DNA virus comprises an RNA transcript of the DNA genome.

### Reverse Transcription

Described are methods, kits, and systems that can be used to detect a target RNA. In some embodiments of any of the aspects, the target RNA is reverse transcribed to a complementary DNA (cDNA) that is thereafter amplified and detected. Accordingly, the methods described herein comprise a step (a) (i.e., the RT step) of contacting the sample with a reverse transcriptase and a first primer or first set of primers. In some embodiments of any of the aspects, the method comprises contacting the at least two samples with a reverse transcriptase and a first primer or first set of primers comprising at least a first barcode, under conditions permitting the generation of reverse transcription products. As used herein, the phrase “conditions permitting the generation of reverse transcription products” refers to temperature(s), time(s), and/or reagent(s) that allow the reverse transcriptase to reverse-transcribe a cDNA from the target RNA using at least one primer from the first set of primers; non-limiting examples of such conditions are described herein. In some embodiments of any of the aspects, prior to step (a) (i.e., the RT step) the at least one target RNA is not extracted from the sample, as described herein with regard to sample preparation.

### Reverse Transcriptase

The term “reverse transcriptase” (RT) refers to an RNA-dependent DNA polymerase used to generate complementary DNA (cDNA) from an RNA template. In some embodiments of any of the aspects, the cDNA is single-stranded DNA (ssDNA) or double-stranded DNA (dsDNA). Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Reverse transcriptases are also used in the synthesis of extrachromosomal DNA/RNA chimeric elements called multicopy single-stranded DNA (msDNA) in bacteria. Retroviral RT has three sequential biochemical activities: RNA-dependent DNA polymerase activity, ribonuclease H (RNAse H), and/or DNA-dependent DNA polymerase activity. Collectively, these activities permit the enzyme to convert single-stranded RNA into single-stranded cDNA or double-stranded cDNA.

In some embodiments of any of the aspects, the reverse transcriptase can be any enzyme that can produce cDNA from an RNA transcript. In some embodiments of any of the aspects, the reverse transcriptase comprises an HIV-1 reverse transcriptase from human immunodeficiency virus type 1. In some embodiments of any of the aspects, the reverse transcriptase comprises M-MuLV reverse transcriptase from the Moloney murine leukemia virus (referred to as M-MuLV, M-MLV, or MMLV). In some embodiments of any of the aspects, the reverse transcriptase comprises AMV reverse transcriptase from the avian myeloblastosis virus (AMV). In some embodiments of any of the aspects, the reverse transcriptase comprises telomerase reverse transcriptase that maintains the telomeres of eukaryotic chromosomes. In some embodiments of any of the aspects, the reverse transcriptase is selected from those expressed by any Group VI or Group VII virus. In some embodiments of any of the aspects, the reverse transcriptase is a naturally occurring RT selected from the group consisting of: an M-MLV RT, an AMV RT, a retrotransposon RT, a telomerase reverse transcriptase, and an HIV-1 reverse transcriptase.

In some embodiments of any of the aspects, the reverse transcriptase (RT) is an engineered or recombinant version of, for example, a Moloney Murine Leukemia Virus (MMLV) RT, Avian Myeloblastosis Virus (AMV) RT, or another naturally occurring RT. In some embodiments of any of the aspects, the reverse transcriptase is ProtoScript® II Reverse Transcriptase, which is also referred to herein as ProtoScript® II RT or Protoscriptase II. ProtoScript® II RT is a recombinant Moloney Murine Leukemia Virus (M-MuLV) reverse transcriptase, e.g., a fusion of the Escherichia coli trpE gene with the central region of the M-MuLV pol gene.

In some embodiments of any of the aspects, the reverse transcriptase is selected from the group consisting of: Maxima® RT (e.g., Maxima H Minus® RT); Omniscript® RT; PowerScript® RT; Sensiscript® RT (SES); SuperScript® II (SSII or SS2); SuperScript® III (SSIII or SS3); SuperScript® IV (SSIV); Accuscript® RT (ACC); a recombinant HIV RT; imProm-II® (IP2) RT; M-MLV RT (MML); Protoscript® RT (PRS); Smart MMLV (SML) RT; ThermoScript® (TSR) RT; RapiDxFire™ RT; (see e.g., Levesque-Sergerie et al., BMC Molecular Biology volume 8, Article number: 93 (2007); Okello et al., PLoS One. 2010 Nov 10;5(11):e13931). Non limiting examples of RTs derived from MMLV include PowerScript®, ACC, MML, SML, SS2, SS3, and SS4. Non limiting examples of RTs derived from AMV include PRS and TSR. Non limiting examples of RTs derived from proprietary sources include IP2, SES, Omniscript®, RapiDxFire™ RT (derived from viral DNA isolated from hot springs). In some embodiments of any of the aspects, the reverse transcriptase exhibits increased thermostability (e.g., up to 48° C.) compared to the wild type RT.

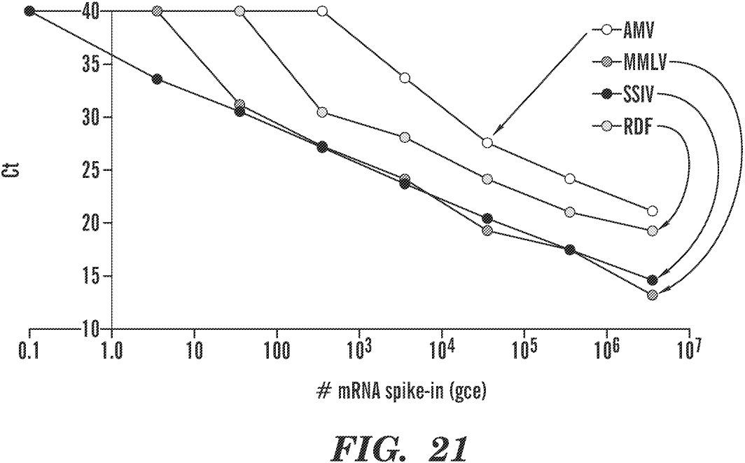

In some embodiments of any of the aspects, the reverse transcriptase is SuperScript® IV (see e.g., FIG. 21). In some embodiments of any of the aspects, the reverse transcriptase is Avian Myeloblastosis Virus RT. In some embodiments of any of the aspects, the reverse transcriptase is Moloney Murine Leukemia Virus RT. In some embodiments of any of the aspects, the reverse transcriptase is RapiDxFire™.

As used herein, one unit (“U”) of reverse transcriptase (e.g., SuperScript® IV RT) is defined as the amount of enzyme that will incorporate 1 nmol of dTTP into acid-insoluble material in a total reaction volume of 50 µl in 10 minutes at 37° C. using poly(rA)•oligo(dT)₁₈ (“(dT)₁₈” disclosed as SEQ ID NO: 1017) as template. In some embodiments of any of the aspects, the reverse transcriptase is provided at a concentration of at least 1 U/µL, at least 2 U/µL, at least 3 U/µL, at least 4 U/µL, at least 5 U/µL, at least 6 U/µL, at least 7 U/µL, at least 8 U/µL, at least 9 U/µL, at least 10 U/µL, at least 20 U/µL, at least 30 U/µL, at least 40 U/µL, at least 50 U/µL, at least 60 U/µL, at least 70 U/µL, at least 80 U/µL, at least 90 U/µL, at least 100 U/µL, at least 110 U/µL, at least 120 U/µL, at least 130 U/µL, at least 140 U/µL, at least 150 U/µL, at least 160 U/µL, at least 170 U/µL, at least 180 U/µL, at least 190 U/µL, at least 200 U/µL, at least 210 U/µL, at least 220 U/µL, at least 230 U/µL, at least 240 U/µL, at least 250 U/µL, at least 260 U/µL, at least 270 U/µL, at least 280 U/µL, at least 290 U/µL, at least 300 U/µL, at least 310 U/µL, at least 320 U/µL, at least 330 U/µL, at least 340 U/µL, at least 350 U/µL, at least 360 U/µL, at least 370 U/µL, at least 380 U/µL, at least 390 U/µL, at least 400 U/µL, at least 410 U/µL, at least 420 U/µL, at least 430 U/µL, at least 440 U/µL, at least 450 U/µL, at least 460 U/µL, at least 470 U/µL, at least 480 U/µL, at least 490 U/µL, or at least 500 U/µL. In some embodiments of any of the aspects, the reverse transcriptase is provided at a concentration of 20 U/µL. In some embodiments of any of the aspects, the reverse transcriptase is provided at a concentration of 200 U/µL.

### First Set of Primers

In some embodiments of any of the aspects, the sample is contacted with a first primer or first set of primers comprising at least a first barcode. In some embodiments of any of the aspects, the sample is contacted with a first primer comprising at least a first barcode. In some embodiments of any of the aspects, the sample is contacted with a first set of primers comprising at least a first barcode. In some embodiments of any of the aspects, the first primer or first set of primers comprises one barcode region. In some embodiments of any of the aspects, the first primer or first set of primers comprises 1, 2, 3, 4, 5, or more barcode regions.

As used herein, the term “primer” denotes a single-stranded nucleic acid that hybridizes to a nucleic acid region of interest and provides a starting point for nucleic acid synthesis, i.e. for enzymatic synthesis of a nucleic acid strand complementary to a template, e.g., a target RNA. In some embodiments of any of the aspects, the primer can be DNA, RNA, modified DNA, modified RNA, synthetic DNA, synthetic RNA, or another synthetic nucleic acid that serves as a substrate for extension when hybridized to a target RNA template. In some embodiments, the primer, e.g., in the first set of primers is about 60 nucleotides long. In some embodiments, the primer, e.g., in the first set of primers is about 40-80 nucleotides long. As a non-limiting example, the primer is 40 nucleotides (nt) long, 41 nt, 42 nt, 43 nt, 44 nt, 45 nt, 46 nt, 47 nt, 48 nt, 49 nt, 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt or more. In some embodiments of any of the aspects, at least one primer, e.g., from the first set of primers, comprises sequences selected from Table 4.

In some embodiments of any of the aspects, the first primer or each primer in the first set of primers comprises, from 5′ to 3′: (a) an adaptor region; (b) a first barcode region; and (c) a target-binding region that is complementary or substantially complementary to and permits hybridization to at least one target RNA. In some embodiments of any of the aspects, the first primer or each primer in the first set of primers comprises, from 5′ to 3′: (a) an adaptor region; (b) a first barcode region; (c) a second barcode region; and (d) a target-binding region that is complementary or substantially complementary to and permits hybridization to at least one target RNA.

In some embodiments of any of the aspects, the adaptor region, e.g., of the first primer or each primer in the first set of primers, comprises an amplification adaptor region such as a PCR adaptor region. The adaptor region provides a hybridization or binding site for an amplification primer to be used after reverse transcription and pooling of reverse-transcription products. Inclusion of an adaptor thus permits amplification of an entire pooled population of cDNA products with, for example, a common forward amplification primer or one pair of forward and reverse amplification primers. In some embodiments of any of the aspects, the adaptor region, e.g., of the first primer or each primer in the first set of primers, is complementary or substantially complementary to an adaptor binding region of a primer in a second or subsequent set of primers. In some embodiments of any of the aspects, the adaptor region, e.g., of the first primer or each primer in the first set of primers, comprises SEQ ID NO: 13 or a nucleic acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 13 that maintains the same function (e.g., amplification adaptor or binding to amplification primer).

In some embodiments of any of the aspects, the first or second barcode region on the first primer or set of first primers is at least 25 nucleotides long. As a non-limiting example, the barcode region can be 10 nucleotides (nt) long, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt long or more. In some embodiments of any of the aspects, the barcode region of a first primer in the first set of barcoded primers is a Hamming distance of at least 10 from each other barcode region of any other primer in the first set of barcoded primers. As used herein, the term “Hamming distance” refers to the number of positions (e.g., base pairs) at which the corresponding sequences are different. In some embodiments of any of the aspects, the barcode region of a first primer in the first set of barcoded primers is a Hamming distance of at least 12 from each other barcode region of any other primer in the first set of barcoded primers. In some embodiments of any of the aspects, the barcode region of a first primer in the first set of barcoded primers is a Hamming distance of at least 5, at least 6, at least 7, at least 8, at least 9, at least 10, at least 11, at least 12, at least 13, at least 14, at least 15, at least 16, at least 17, at least 18, at least 19, at least 20 or more from each other barcode region of any other primer in the first set of barcoded primers (or barcode region in a second, third, fourth, etc. set of barcoded primers).

In some embodiments of any of the aspects, the first or second barcode region on the first primer or set of first primers comprises one of SEQ ID NOs: 18-989 or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 18-989 that maintains the same function (e.g., identification). In some embodiments of any of the aspects, the first barcode region on the first primer or set of first primers comprises one of SEQ ID NOs: 30-989 (see e.g., Table 5 or Table 6); such barcodes are also referred to herein as “sample barcode,” “sample ID”, “patient barcode,” or “patient ID.” In some embodiments of any of the aspects, at least one barcode region on the first primer or set of first primers corresponds to and is different for each of the at least two samples. In some embodiments of any of the aspects, at least one barcode region on the first primer or set of first primers corresponds to and is different for each of the target RNAs.

In some embodiments of any of the aspects, a target-binding region is complementary or substantially complementary to and permits hybridization to at least one target RNA. In some embodiments of any of the aspects, the target-binding region permits hybridization to at least one target RNA under conditions permitting the generation of a reverse transcription product. In some embodiments of any of the aspects, the target-binding region, e.g., of a primer in the first set of primers, is about 20 nucleotides long. In some embodiments, the target-binding region, e.g., of a primer in the first set of primers, is about 15-35 nucleotides long. As a non-limiting example, the target-binding region can be 15 nucleotides (nt) long, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt long or more. In some embodiments, the target-binding region, e.g., of a primer in the first set of primers, has a Tm of about 53° C.-62° C., e.g., at least 53° C., at least 54° C., at least 55° C., at least 56° C., at least 57° C., at least 58° C., at least 59° C., at least 60° C., at least 61° C., at least 62° C. or more.

In some embodiments of any of the aspects, the target-binding region of a primer in the first set of primers binds to a region of SARS-CoV-2 N gene or S gene (see e.g., SEQ ID NO: 1001-1002). In some embodiments of any of the aspects, the target-binding region of a primer in the first set of primers comprises one of SEQ ID NO: 3 (N#1_RT), SEQ ID NO: 5 (N#2_RT), SEQ ID NO: 7 (del6970_RT), SEQ ID NO: 9 (D614_RT), or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 3, 5, 7, or 9 that maintains the same function (e.g., binding to the target RNA or positive control RNA) (see e.g., Table 4).

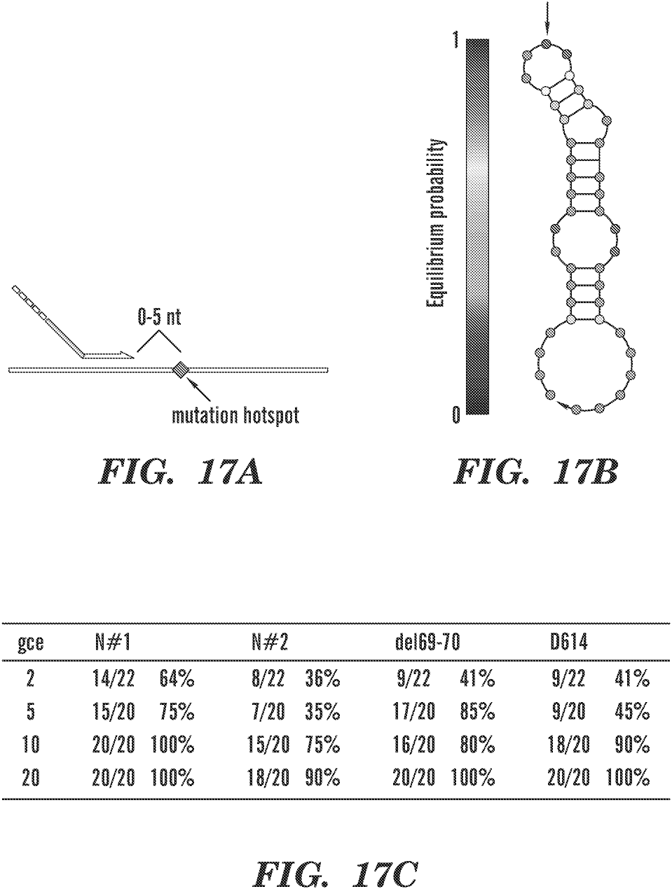

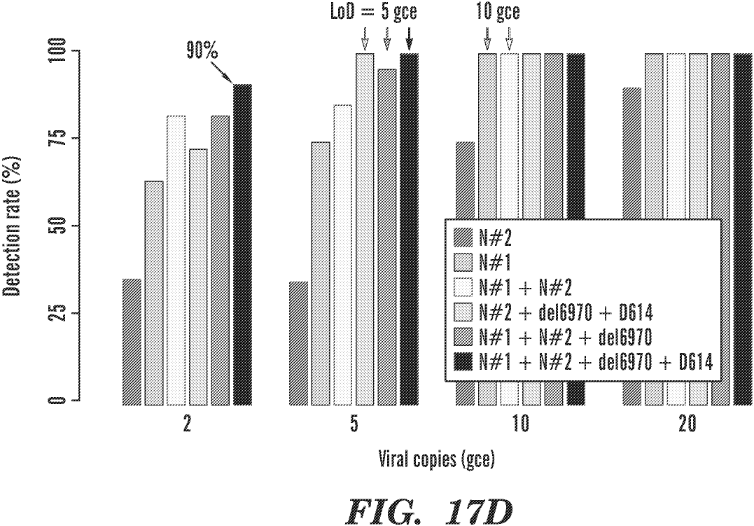

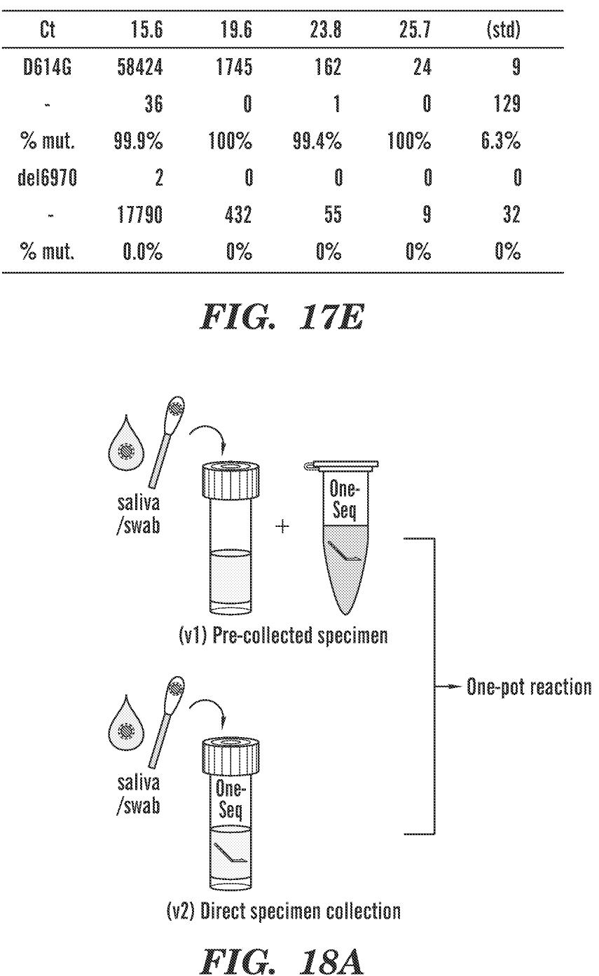

In some embodiments of any of the aspects, the target-binding region, e.g., of a primer in the first set of primers, binds at most 5 nucleotides away from, e.g., between the 3′ end of the primer and the 5′ end of, a variation of interest in the target RNA. In some embodiments of any of the aspects, the target-binding region, e.g., of a primer in the first set of primers, binds 0 nt, 1 nt, 2 nt, 3 nt, 4 nt, 5 nt, 6 nt, 7 nt, 8 nt, 9 nt, or 10 nt away from a variation of interest in the target RNA (see e.g., FIG. 17A). In some embodiments of any of the aspects, the variation of interest is selected from the group consisting of: a single-nucleotide variation; a point mutation; a substitution; an insertion; and a deletion. In some embodiments of any of the aspects, the target RNA is SARS-CoV-2 S gene and the variation of interest is selected from the group consisting of: del69-70, de1144, K417N, K417T, L452R, E484K, N501Y, D614G, P681H, and A701V (see e.g., Table 12). In some embodiments of any of the aspects, the target RNA is SARS-CoV-2 S gene, the variation of interest is del69-70 in the S gene, and the target-binding region of a primer in the first set of primers is SEQ ID NO: 7. In some embodiments of any of the aspects, the target RNA is SARS-CoV-2 S gene, the variation of interest is D614G in the S gene, and the target-binding region of a primer in the first set of primers is SEQ ID NO: 9.

In some embodiments of any of the aspects, the target-binding region, e.g., of a primer in the first set of primers, comprises at most 1 nucleotide mismatch (i.e., non-complementary nucleotide) compared to a target RNA (see e.g., Table 7). In some embodiments of any of the aspects, the target-binding region, e.g., of a primer in the first set of primers, does not specifically bind to a non-target nucleic acid, e.g., a nucleic acid that is not a target RNA. In some embodiments of any of the aspects, the target-binding region, e.g., of a primer in the first set of primers, is at most 80% identical to a non-target nucleic acid (see e.g., Table 8 for non-limiting examples of non-target microbial nucleic acids). In some embodiments of any of the aspects, the target-binding region, e.g., of a primer in the first set of primers, is at most 40%, at most 45%, at most 50%, at most 55%, at most 60%, at most 65%, at most 70%, at most 75%, or at most 80% identical to a non-target nucleic acid.

In some embodiments of any of the aspects, the first primer or each primer in the first set of primers comprises, from 5′ to 3′: (a) an adaptor region (e.g., SEQ ID NO: 13); (b) a first barcode region (e.g., one of SEQ ID NOs: 30-989); and (c) a target-binding region that is complementary or substantially complementary to and permits hybridization to at least one target RNA (e.g., one of SEQ ID NOs: 3, 5, 7, or 9). SEQ ID NO: 1005 is an exemplary primer from the first set of primers, comprising from 5′ to 3′: SEQ ID NO: 13 (bolded), SEQ ID NO: 30, and SEQ ID NO: 3 (bold italicized).

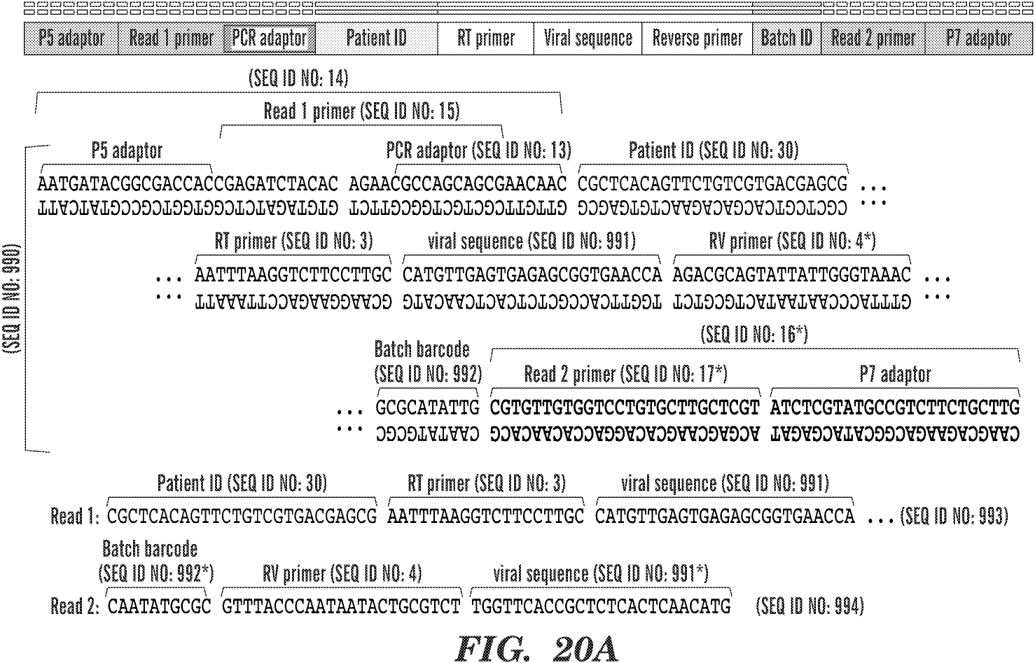

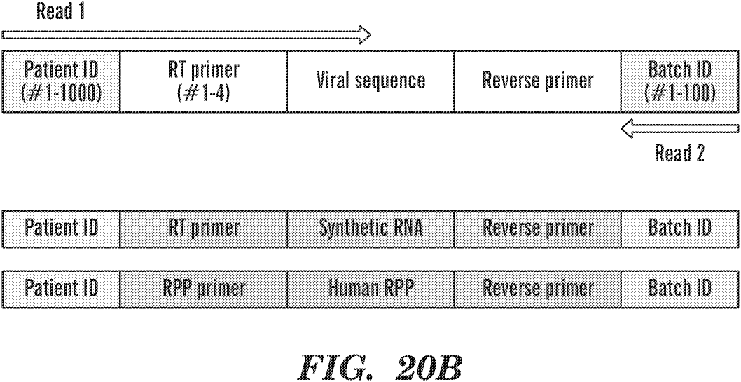

SEQ ID NO: 1005, 61 nt (see e.g., FIG. 20A) CGCCAGCAGCGAACAACGCTCACAGTTCTGTCGTGACGAGCGAATTTAAGGTCTTCCTTGC

In some embodiments of any of the aspects, the first primer or each primer in the first set of primers is present in the RT reaction at a concentration of at least 125 nM. In some embodiments of any of the aspects, the first primer or each primer in the first set of primers is present in the RT reaction at a concentration of at least 25 nM, at least 30 nM, at least 35 nM, at least 40 nM, at least 45 nM, at least 50 nM, at least 55 nM, at least 60 nM, at least 65 nM, at least 70 nM, at least 75 nM, at least 80 nM, at least 85 nM, at least 90 nM, at least 95 nM, at least 100 nM, at least 105 nM, at least 110 nM, at least 115 nM, at least 120 nM, at least 125 nM, at least 130 nM, at least 135 nM, at least 140 nM, at least 145 nM, at least 150 nM, at least 160 nM, at least 170 nM, at least 180 nM, at least 190 nM, at least 200 nM, at least 210 nM, at least 220 nM, at least 230 nM, at least 240 nM, at least 250 nM, at least 260 nM, at least 270 nM, at least 280 nM, at least 290 nM, at least 300 nM, at least 310 nM, at least 320 nM, at least 330 nM, at least 340 nM, at least 350 nM, at least 360 nM, at least 370 nM, at least 380 nM, at least 390 nM, at least 400 nM, at least 410 nM, at least 420 nM, at least 430 nM, at least 440 nM, at least 450 nM, at least 460 nM, at least 470 nM, at least 480 nM, at least 490 nM, at least 500 nM.

### Detergent

In some embodiments of any of the aspects, step (a) (the RT step) further comprises contacting the sample with a detergent (also referred to as a surfactant). Such detergent can be included in the viral transport medium, or added thereafter, e.g., in a diluent or RT solution. In some embodiments of any of the aspects, the detergent lyses viral particles or cells in the sample. In some embodiments of any of the aspects, the detergent allows target RNA detection in extraction-free samples, i.e., without the need for a nucleic acid-extraction step. In some embodiments of any of the aspects, the detergent releases target RNA from the sample. In some embodiments of any of the aspects, the detergent releases at least 70%, at least 75%, at least 80%, at least 85%, at least 90%, at least 91%, at least 92%, at least 93%, at least 94%, at least 95%, at least 96%, at least 97%, at least 98%, at least 99% or more of the target RNA from the sample. Non-limiting examples of detergents include anionic surfactants, cationic surfactants, nonionic surfactants, amphoteric/zwitterionic surfactants, and co-surfactants or mixtures thereof.

In some embodiments of any of the aspects, the detergent is a nonionic surfactant. Non-limiting examples of nonionic surfactants include Triton X-100, sodium tri-isopropyl naphthalene sulfonate, LDS, SDS, NP-40; lecithin, a Span group (e.g., Span 20, or 80), or a Tween group (e.g., Tween 20, 21, 40, 60, 60 K, 61, 65, 80, 80 K, 81, or 85), a sugar amide (e.g. polysaccharide amide), or an alkyl polyglucocide. In some embodiments of any of the aspects, the detergent is Triton X-100 (2-[4-(2,4,4-trimethylpentan-2-yl)phenoxy]ethanol). Non-limiting examples of anionic surfactant include alkyl sulfosuccinate, sodium dioctyl sulfosuccinate (AOT), sodium dihexyl sulfosuccinate (AMA), ammonium or sodium lauryl ether sulfate, alkyl or acyl taurates, alkyl or acyl sarcosinates, alyl ether sulfates, alkyl ether sulfonates, or alkyl ether carboxylates (e.g., counterion can be sodium, ammonium, or potassium). Alkyl sulfosuccinate can include a mono or dialkyl sulfosuccinate or a C6-C22 sulfosuccinate. Non limiting examples of cationic surfactants include a quaternary ammonium compound (e.g., an alkyldimethylammonium haloginide), alkyl pyridinium chlorides or bromides, or other hydrogenides. Non-limiting examples of amphoteric surfactants include, for example, a quaternary amino acid, an alkyl amine oxide, or an alkyl betaine.

In some embodiments of any of the aspects, the detergent is present in an amount that does not interfere with subsequent enzymatic reactions (e.g., the RT step, the amplification step, and/or the sequencing step). If the detergent concentration can interfere with subsequent enzymatic reactions then it is diluted or the reaction product is isolated prior to the subsequent enzymatic reactions. In some embodiments of any of the aspects, the detergent (e.g., Triton X-100) is present in the RT reaction at a concentration of at least 0.1%. In some embodiments of any of the aspects, the detergent (e.g., Triton X-100) is present in the RT reaction at a concentration of at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1% or more.

### Carrier Nucleic Acid

In some embodiments of any of the aspects, step (a) (the RT step) further comprises contacting the sample with carrier nucleic acid. Such carrier nucleic acid can be included, for example, in the viral transport medium, or added thereafter. In some embodiments of any of the aspects, carrier nucleic acid reduces loss of the target RNA, e.g., preserves at least 70%, at least 75%, at least 80%, at least 85%, at least 90%, at least 91%, at least 92%, at least 93%, at least 94%, at least 95%, at least 96%, at least 97%, at least 98%, at least 99% or more of the target RNA in the sample. In some embodiments of any of the aspects, the carrier nucleic acid is poly-A60 DNA oligonucleotide (e.g., a DNA comprising at least 60 adenosines; (“(dA)₆₀” disclosed as SEQ ID NO: 1025) or E. coli tRNA (e.g., E. coli MRE 600; see e.g., Sigma™, 10109541001).

In some embodiments of any of the aspects, the carrier nucleic acid (e.g., poly-A60 DNA oligonucleotide) is present at a concentration of at least 0.5 uM in the RT reaction. In some embodiments of any of the aspects, the carrier nucleic acid (e.g., poly-A60 DNA oligonucleotide) is present at a concentration of at least 0.01 uM, at least 0.02 uM, at least 0.03 uM, at least 0.04 uM, at least 0.05 uM, at least 0.06 uM, at least 0.07 uM, at least 0.08 uM, at least 0.09 uM, at least 0.1 uM, at least 0.2 uM, at least 0.3 uM, at least 0.4 uM, at least 0.5 uM, at least 0.6 uM, at least 0.7 uM, at least 0.8 uM, at least 0.9 uM, at least 1 uM, at least 2 uM, at least 3 uM, at least 4 uM, at least 5 uM, at least 6 uM, at least 7 uM, at least 8 uM, at least 9 uM, at least 10 uM or more in the RT reaction.

In some embodiments of any of the aspects, the carrier nucleic acid (e.g., E. coli tRNA) is present at a concentration of at least 15 ug/ml in the RT reaction. In some embodiments of any of the aspects, the carrier nucleic acid (e.g., E. coli tRNA) is present at a concentration of at least 1 ug/ml, at least 2 ug/ml, at least 3 ug/ml, at least 4 ug/ml, at least 5 ug/ml, at least 6 ug/ml, at least 7 ug/ml, at least 8 ug/ml, at least 9 ug/ml, at least 10 ug/ml, at least 11 ug/ml, at least 12 ug/ml, at least 13 ug/ml, at least 14 ug/ml, at least 15 ug/ml, at least 16 ug/ml, at least 17 ug/ml, at least 18 ug/ml, at least 19 ug/ml, at least 20 ug/ml, at least 21 ug/ml, at least 22 ug/ml, at least 23 ug/ml, at least 24 ug/ml, at least 25 ug/ml or more in the RT reaction.

### Positive Control Nucleic Acids

In some embodiments of any of the aspects, step (a) (the RT step) further comprises contacting the sample with a positive control nucleic acid. In some embodiments of any of the aspects, the positive control nucleic acid is a positive sample control nucleic acid or a positive enzymatic control nucleic acid. As discussed further below, a sample control tests for the presence of a host (e.g., human) gene transcript to control for the integrity of the sample nucleic acid. In some embodiments of any of the aspects, the reverse transcription reaction comprises a positive sample control nucleic acid. In some embodiments of any of the aspects, the reverse transcription reaction comprises a positive enzymatic control nucleic acid. The enzymatic control tests for the activity or activities of the RT and amplification enzymes used in the reaction. In some embodiments of any of the aspects, the reverse transcription reaction comprises both a positive sample control nucleic acid or a positive enzymatic control nucleic acid.

In some embodiments of any of the aspects, the detection methods described herein comprise a “split amplification” step, e.g., in order to allow optimal detection of the positive control nucleic acids during the sequencing step. In such a split amplification, the pooled reverse transcription product mixture from step (b) is divided into at least two portions, e.g., a “positive control portion” and a “target portion,” and a separate step (c) (e.g., the amplification step) is performed for each portion. In some embodiments, the positive control portion (e.g., the smaller portion) is used to amplify the positive control nucleic acids, e.g., using forward and reverse amplification primers specific for the positive control nucleic acids. The positive control portion can be used to amplify the sample control and/or the enzymatic control. In some embodiments, the target portion (e.g., the larger portion) is used to amplify the target RNAs, e.g., using forward and reverse amplification primers specific for the target cDNAs (e.g., viral targets). After the split amplification step, the at least two portions comprising amplification products from the positive controls and target nucleic acids are combined in the one container for step (d) (e.g., the sequencing step). In some embodiments, before step (d) (e.g., the sequencing step), the amplified portions are combined at the same ratio as before the split amplification. In some embodiments, before step (d) (e.g., the sequencing step), the amplified portions are combined at a new ratio, e.g., with a higher proportion of the positive control amplification products to the target amplification products than before the split, in order to allocate more sequencing reads for the positive control sequences. In some embodiments, the pooled reverse transcription product mixture from step (b) is split 1:10, e.g., into 1 part positive control portion and 10 parts target portion. In some embodiments, before step (d) (e.g., the sequencing step), the amplification products are combined 1:10, e.g., 1 part positive control amplification product and 10 parts target amplification product. In some embodiments, before step (d) (e.g., the sequencing step), the amplification products are combined at a ratio higher than 1:10, e.g., 2, 3, 4, 5, 6, 7, 8, 9, 10 or more parts positive control amplification product and 10 parts target amplification product.

### Sample Control

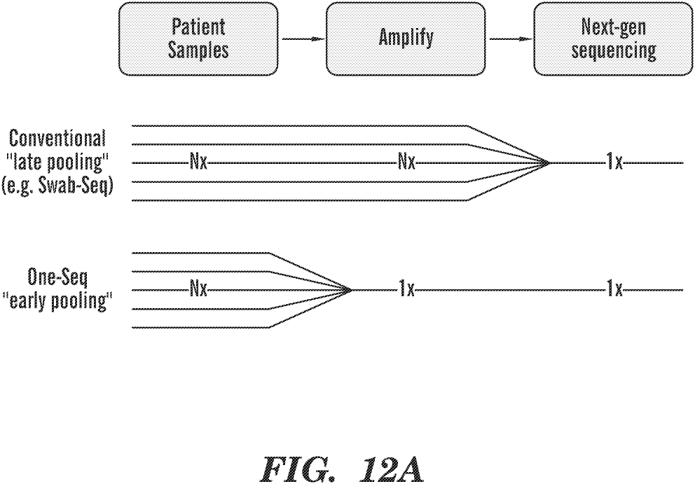

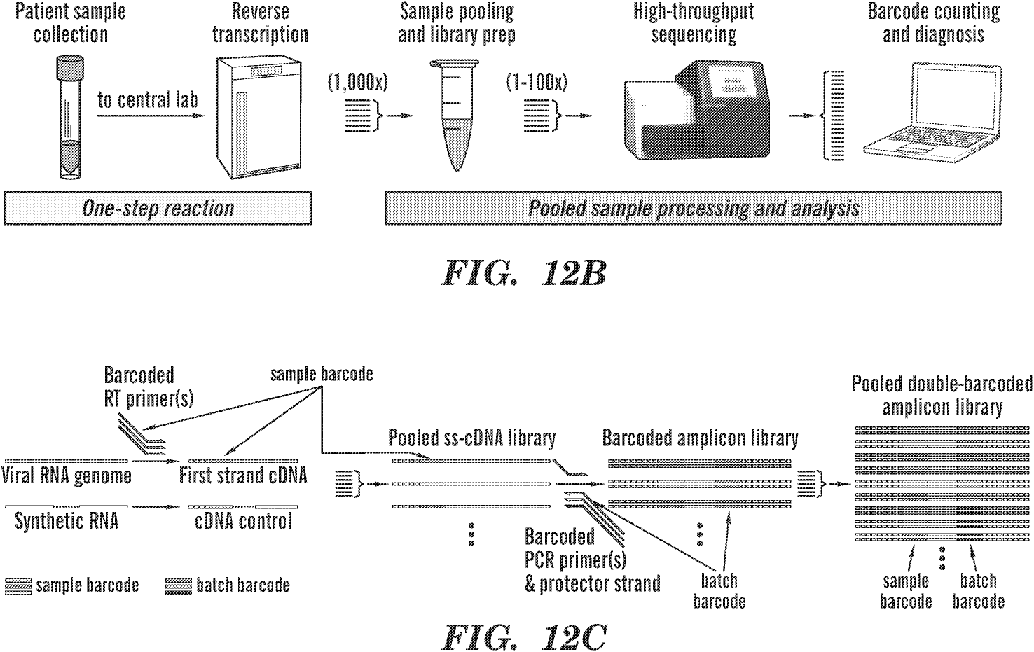

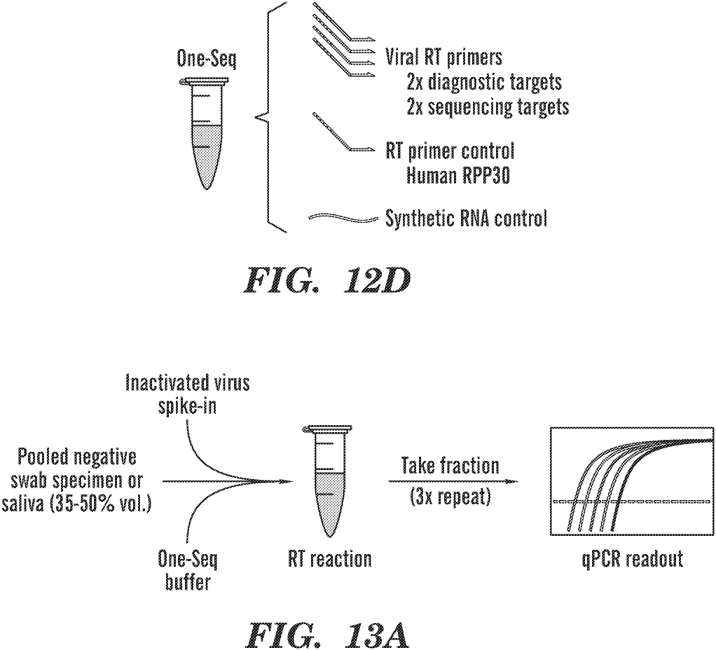

In some embodiments of any of the aspects, the positive control nucleic acid (e.g., “positive sample control nucleic acid” or “sample control”) is a primer comprising from 5′ to 3′: (a) an adaptor region; (b) a first barcode region; and (c) a target-binding region that is complementary to or substantially complementary to a sample nucleic acid (e.g., RPP30). The “positive sample control nucleic acid” targets a nucleic acid that is present in the sample, e.g., a “sample nucleic acid,” e.g., a nucleic acid from the subject species or patient, e.g., a human nucleic acid. In some embodiments of any of the aspects, the sample control targets human Ribonuclease P protein subunit p30 (hRPP30 or RPP30 or RPP) gene. RPP30 is a single copy gene present in the human genome. In some embodiments, the sample control targets an RNA (e.g., a specific mRNA) present in the sample. In some embodiments of any of the aspects, the sample control (e.g., primer binding to hRPP30) functions as a control to indicate presence or absence of sample (see e.g., FIG. 12D) and can also indicate the integrity thereof. In other words, the sample control is a reverse transcription primer (i.e., a primer in the first set of primers) specific for a nucleic acid in the sample, not the specific RNA target (e.g., viral RNA).

In some embodiments of any of the aspects, the forward primer in the second set of primers (i.e., FW PCR primer) for the reverse transcription product of the sample control (e.g., SEQ ID NO: 11) is SEQ ID NO: 14. In some embodiments of any of the aspects, the reverse primer in the second set of primers (i.e., RV PCR primer) for the reverse transcription product of the sample control comprises a target-binding region that is complementary or substantially complementary to the sample nucleic acid. In some embodiments of any of the aspects, the first and second sequencing primers in the third set of primers for the sample control are SEQ ID NO: 15 and SEQ ID NO: 17. If a sequencing signal is detected from the sample control, then the RT reaction comprised a sample that included RNA that could be reverse transcribed and amplified for detection. If a sequencing signal is not detected from the sample control, then the RT reaction did not comprise a sample that included such RNA.

In some embodiments of any of the aspects, the sample control is present in the RT reaction at a concentration of at least 125 nM. In some embodiments of any of the aspects, the sample control is present in the RT reaction at a concentration of at least 25 nM, at least 30 nM, at least 35 nM, at least 40 nM, at least 45 nM, at least 50 nM, at least 55 nM, at least 60 nM, at least 65 nM, at least 70 nM, at least 75 nM, at least 80 nM, at least 85 nM, at least 90 nM, at least 95 nM, at least 100 nM, at least 105 nM, at least 110 nM, at least 115 nM, at least 120 nM, at least 125 nM, at least 130 nM, at least 135 nM, at least 140 nM, at least 145 nM, at least 150 nM, at least 160 nM, at least 170 nM, at least 180 nM, at least 190 nM, at least 200 nM, at least 210 nM, at least 220 nM, at least 230 nM, at least 240 nM, at least 250 nM, at least 260 nM, at least 270 nM, at least 280 nM, at least 290 nM, at least 300 nM, at least 310 nM, at least 320 nM, at least 330 nM, at least 340 nM, at least 350 nM, at least 360 nM, at least 370 nM, at least 380 nM, at least 390 nM, at least 400 nM, at least 410 nM, at least 420 nM, at least 430 nM, at least 440 nM, at least 450 nM, at least 460 nM, at least 470 nM, at least 480 nM, at least 490 nM, at least 500 nM.

In some embodiments of any of the aspects, the target-binding region of the sample control comprises a 15 nt - 25 nt sequence that is complementary to or substantially complementary to SEQ ID NO: 1006, or a 15 nt - 25 nt sequence that is complementary to or substantially complementary to a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1006 that maintains the same function (e.g., specifically binding a nucleic acid in the sample; e.g., specifically binding hRPP30 mRNA). In some embodiments of any of the aspects, the target-binding region of the sample control comprises SEQ ID NO: 1019 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1019 that maintains the same function (e.g., specifically binding hRPP30 mRNA).

SEQ ID NO: 1006, Homo sapiens ribonuclease P/MRP subunit p30 (RPP30), transcript variant 1, mRNA, 4521 nt

ATGGGACTTCAGCATGGCGGTGTTTGCAGATTTGGACCTGCGAGCGGGTT
CTGACCTGAAGGCTCTGCGCGGACTTGTGGAGACAGCCGCTCACCTTGGC
TATTCAGTTGTTGCTATCAATCATATCGTTGACTTTAAGGAAAAGAAACA
GGAAATTGAAAAACCAGTAGCTGTTTCTGAACTCTTCACAACTTTGCCAA
TTGTACAGGGAAAATCAAGACCAATTAAAATTTTAACTAGATTAACAATT
ATTGTCTCGGATCCATCTCACTGCAATGTTTTGAGAGCAACTTCTTCAAG
GGCCCGGCTCTATGATGTTGTTGCAGTTTTTCCAAAGACAGAAAAGCTTT
TTCATATTGCTTGCACACATTTAGATGTGGATTTAGTCTGCATAACTGTA
ACAGAGAAACTACCATTTTACTTCAAAAGACCTCCTATTAATGTGGCGAT
TGACCGAGGCCTGGCTTTTGAACTTGTCTATAGCCCTGCTATCAAAGACT
CCACAATGAGAAGGTATACAATTTCCAGTGCCCTCAATTTGATGCAAATC
TGCAAAGGAAAGAATGTAATTATATCTAGTGCTGCAGAAAGGCCTTTAGA
AATAAGAGGGCCATATGACGTGGCAAATCTAGGCTTGCTGTTTGGGCTCT
CTGAAAGTGACGCCAAGGCTGCGGTGTCCACCAACTGCCGAGCAGCGCTT
CTCCATGGAGAAACTAGAAAAACTGCTTTTGGAATTATCTCTACAGTGAA
GAAACCTCGGCCATCAGAAGGAGATGAAGATTGTCTTCCAGCTTCCAAGA
AAGCCAAGTGGTCTCACTCTGTCACCCAGGCTGGAGTGCAGTGGCACAAT
CTCGGCTCACTGCAACCTTTGCCTCTTGGGCTCAAGCCATCCTCCCACCT
CAGCCTCCCAAGAACTAGAATTCAACAAAGACAACTTTTGATCTCTCATC
AGAGAGATCATACTCCCAAGAACAGGCTTTGACCCTTCTTTAAAAGAGGA
TTGTCCTGGGCTGATGAGAGTCACTTTACCTGAAGATACCTGGGAAGTTT
TGTCTCCTCTGAGGTTGGCCCATGGCCAGTGACTGATGCAGGACATTACA
GCCTGGCCCACTGGCCTCGGTGGTACAATTTATGCTCCTGAGCACCCCAT
GGGATTACGCTGTGTGTGGATTTCTCCTGAAACCACATATTTGCCTCTTC
TGCCCTGTTCTGTTTTTTCTCATTCCCTTATAGAGGACTCTCAGTAAGTC
ACTTACACAAGAATCCTAATTTGAAATGCTGCTTCCAGGAGACCTGACTT
AGAAAATTGGACAAATAAAGTTGATTTTTTTAAATGTCCAGTAACATGAA
GATGCTGAACTTTCCTAGTCATTTAGGGGGAAATCACCACAAATATATCT
GGCTGATCAGGTTGAAAGTTAAAAGAAAAAAAGATTTATAAAGTGGGTAT
TTTCAAGATGGTGTGGAGGGAGATACAATTTGATGCAAGTCTTATACTTT
TGATGTCAATTTATTCTTCAGAAATAACTGGTTAATTATAAAGGGTGGAT
GGATAAGGATATTCACTGCAACAATGTCTTAAATGTGAAAATGGAAACAA
CCTAAATACCCAATAATAACAGGATTAAATAATTCATTGTACATTAAAAG
AATACTGTCTATAAAGATGTCTAGAATAAGTTGTTCAGTTGAAGTTGTAA
AGCTAAATACACAATACCCATAATGTGCACAGAAATAATCAGAATGTCAT
GAAACCAGGATTATTGGTGATGTGTTGCTTCCTTTGTTACTCATTTCTGT
ATTGGCATAATGAGTATTGGGTGTTCAAGAGGAGGGGGAAGGAAGTATGA
CAGATGTTATGGGGAAAAAGCAAAGTACAACAGGAAGACACCTTGGGGGA
ACTAATAGAATCTAAGGACTCAAGGATGGCTTCCTGGAGGAAATACAGCT
AGAACAAAGGGGAGGAATGAGAAGTGATGTGATGGCGTGGAGTGGGCTGT
AGTGGGAAGAAGAGTCTTCCAGGGAGCTGGCACAGTATGTGAAAACAGTA
AAGCAAGTGCCTGGATTTTTTAAGGAACTGAAAATTTAGTTGAGTTGAAA
TTTAGAGTTTGGCTAGGAAGGTTATGAGAGATAAGAATAAAGAGTTAACA
GCAGCCAGATTTTAAGGATTTTATAAGACATTTTTAGGAGTTTTTATTTC
ATCCTGAGAGAAATGTGAAGCCATCGAAGGGTTGAAAGAGGAGAGTGAGT
TGATCAGCATTGCATTTTAGAAAAATCCCTCTATCTGCAACTTGAAAAAC
ATTCTGGAGGTAAGCAAGCCTGGAGGCCAGGAGCCTAGGAGGGCTATTTG
ATCCAGATGAGAAGTAATGGTGACCTGAACTAGGGCAGAGGCACCTAGGA
TTGGAAAACATGGACAGATCACAGCACTACTTATGTAGTATACTTGGTAA
GACCTGGTTGTTTAAAAGAGAAGGATGAGGGAAAGAAGGTCAAAAACAAC
TTCTAGGACTCTCCATTGGCCAGTGTGGTGTGCCCTTCACTGAAGAGCAA
ACACAAAATGAAAGATTGTGGGCAAAGAGTTGAGTCAGTGAGAAGGCAAG
GAGAGAACCTTATAAAAAAATTGACTATGTGATTAAAAACTTAAAAATTT
CCCCCAACGTGTTTATCTTTTCCATTAGCAGAAATAACTAAGAGTTGTCT
TAATTCTAATGGGATTTATTCCATATTGTCTCTCATGCCCTCTACCTAGT
TATTAGTGCAAATATTTATATGTGGCAACATAAAACTTTTTAACTCTTTA
TTCTCTTCTCTCGTGTACCCTCCCAGCTCTTTAGGGGAGGTGGATTTGAG
GCAGATACCATAAAGAAAAGTTGGTCACATGGTGGTAACACGTTGAAGTT
ATGCCACATGAGACATCAGCACTGGCAAGAGAAATGTCTGTGTTGTAGAT
GTTTCACTTGGAAGAAATTGAAGGACCCTGAGCCTTAAAAGTCTGACAAA
CTTAAGCCAGGACCCCTGTGGGGAAGGTAGAGGGGCCAACAAACAAGATT
GGGAGTCAGAGAGATAACAATGAAATCCCCAATGCCTGTGGGAGGTGGAC
TCCCTGGATTAGTACTAGACAGAAAAGGTACAAAAATATTTCAAACCATT
CTCACAACTCTATATGTGTCTATGACCAGATAACTGGAAGACCTTCTGGT
TATGGACTATGCGTATACACTCTCCCAGATAGTTAGAGGCATATCTAAGA
GGTTAACATATATGATCTTATCCAAAATGGGTCTCTTGGTGCTAGTGTTT
TACATCAGACTTCACTGGCTTTCATGTATTTCCACAAGTGCCAAACATTT
CTCATATCCTTGCTGTATTCCATAGAGCAGTGTTCCTGCTACCTGGAACA
CTTGATTCTTGAATAACTCCTGTTTACCTTTCAGACAAACCCTAAAGGTT
ACCACCTCAAAGAAGTCTTTATAGAAGCCTCATCATCTTAGACACTCTGT
ATTGTTTCCTTCATCGTATTTACAACAGACAGATACTGTGCACTTACTGC
CTCACTTAACGACAGGGATACGTTCTGAAAGGTGCATCATTAGGCGGTTT
TGTTGTGTGAACATCACAGAGTGTTACTTACACAAACCTAAATGATACAG
CCTACTAAACACCTAGGCTATATGAGCAATACAGCCTATTGCTCTTAGGC
TTCAAACTTGTACGACATGTCACTGTACTGAATACTGTAGGCAACTATAA
CACAGTGGTAAGTATATTGTGTATCTAAACAAACATAGAAAAGGTAATGC
ACTGTACTATGATGTTACAACAGCTAGGATGTTGCTATCAATAGAAATTT
TTCAGCTTCATTTTATTTTTATGGGACCACCTTTGTATATGTGGTTCATT
GTTGGCCGAAACACCATTCTGTGGCACATGACTATGTATTTATTCCTCAT
TATTCCTTTAATATTCATCTCTTCCAGGAGGGCATGTCATGGACAATCTC
TTTTTCTTACCACAGGTCTTAGGACCTGGCCTAGCACCTGGCCAAGAACT
ACTGGCATACCTCCTTTTATTGTGCTTCAATTTATTGTGCTTTGCAAATA
CTGAATTTTTTACAAGTTGAAGATTTGTGGCACCTCTGTAACCAGCAAGT
CTATTGGTGCCATTTTTTCAACATCATGTGCCTGTTTCCTGTCTCGCTCA
TGTCACATTTTGGTAATTTTCACAATATTAAAAACTTTTTCATTATTATT
A

SEQ ID NO: 1019, RPP30 RT primer, target-binding region, 20 nt GAGCGGCTGTCTCCACAAGT

SEQ ID NO: 1020, RPP30 RV amplification primer, target-binding region, 20 nt GTGTTTGCAGATTTGGACCT

In some embodiments of any of the aspects, the primer in the first step of primers (i.e., RT primer) for the sample control (e.g., RPP30, SEQ ID NO: 1006) comprises SEQ ID NO: 1019. In some embodiments of any of the aspects, the forward primer in the second set of primers (i.e., FW PCR primer) for the sample control (e.g., RPP30, SEQ ID NO: 1006) is SEQ ID NO: 14. In some embodiments of any of the aspects, the reverse primer in the second set of primers (i.e., RV PCR primer) for the sample control (e.g., RPP30, SEQ ID NO: 1006) comprises SEQ ID NO: 1020. In some embodiments of any of the aspects, the first and second sequencing primers in the third set of primers for the enzymatic control are SEQ ID NO: 15 and SEQ ID NO: 17 (see e.g., Table 15).

### Enzymatic Control

In some embodiments of any of the aspects, the positive control nucleic acid (e.g., a “positive enzymatic control nucleic acid” or “enzymatic control”) comprises, from 5′ to 3′: (a) a region that is not identical or substantially identical to any target RNA being assayed; and (b) a region that is identical or substantially identical to at least one target RNA region. In some embodiments of any of the aspects, the positive control nucleic acid (e.g., a “positive enzymatic control nucleic acid”) comprises, from 5′ to 3′: (a) a region that is not identical or substantially identical to any target RNA being assayed; and (b) a region that is complementary or substantially complementary to the target-binding region of at one least primer from the first set of primers. In some embodiments of any of the aspects, the region of the positive control nucleic acid that is identical or substantially identical to at least one target RNA is complementary or substantially complementary to the target-binding region of at one least primer from the first set of primers. In some embodiments of any of the aspects, the enzymatic control comprises SEQ ID NO: 11 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 11 that maintains the same function (e.g., specific binding to at least one primer in the first set of primers).

In some embodiments of any of the aspects, the enzymatic control functions as a control for the enzymatic reactions (e.g., the RT step, the amplification step, and/or the sequencing step). In some embodiments of any of the aspects, the primer in the first step of primers (i.e., RT primer) for the enzymatic control (e.g., SEQ ID NO: 11) comprises SEQ ID NO: 3, or e.g., SEQ ID NO: 1005. In some embodiments of any of the aspects, the forward primer in the second set of primers (i.e., FW PCR primer) for the enzymatic control (e.g., SEQ ID NO: 11) is SEQ ID NO: 14. In some embodiments of any of the aspects, the reverse primer in the second set of primers (i.e., RV PCR primer) for the enzymatic control (e.g., SEQ ID NO: 11) comprises SEQ ID NO: 12. In some embodiments of any of the aspects, the first and second sequencing primers in the third set of primers for the enzymatic control are SEQ ID NO: 15 and SEQ ID NO: 17 (see e.g., Table 15).

If a sequencing signal is detected from the enzymatic control (e.g., SEQ ID NO: 11), then all of the enzymatic reactions were completed successfully. If a sequencing signal is not detected from the enzymatic control (e.g., SEQ ID NO: 11), then at least one of the enzymatic reactions (e.g., the RT step, the amplification step, and/or the sequencing step) were not completed successfully.

In some embodiments of any of the aspects, the sample is contacted with at least 100 copies/ul of enzymatic control (e.g., SEQ ID NO: 11). In some embodiments of any of the aspects, the sample is contacted with at least 10⁴ copies/ul of enzymatic control (e.g., SEQ ID NO: 11). In some embodiments of any of the aspects, the sample is contacted with at least 10¹ copies/ul, at least 10² copies/ul, at least 10³ copies/ul, at least 10⁴ copies/ul, at least 10⁵ copies/ul, at least 10⁶ copies/ul, at least 10⁷ copies/ul, at least 10⁸ copies/ul, at least 10⁹ copies/ul, at least 10¹⁰ copies/ul or more of enzymatic control. In some embodiments of any of the aspects, the sample is contacted with both a sample control (e.g., primer specific to hRPP30) and an enzymatic control (e.g., SEQ ID NO: 11).

### Stabilization Agent

In some embodiments of any of the aspects, step (a) (e.g., the RT step) further comprises contacting the samples with a stabilization agent. In some embodiments of any of the aspects, the stabilization agent prevents degradation of the RNA target and/or reverse transcriptase for at least 6 hours at room temperature. The stabilization agent or agents can be present, for example, in the viral transport medium, such that RNA is protected as soon as the sample is placed in the medium. In some embodiments of any of the aspects, the stabilization agent prevents degradation of the RNA target and/or reverse transcriptase for at least 24 hours at room temperature. In some embodiments of any of the aspects, the stabilization agent prevents degradation of the RNA target and/or reverse transcriptase for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours, at least 49 hours, at least 50 hours, at least 51 hours, at least 52 hours, at least 53 hours, at least 54 hours, at least 55 hours, at least 56 hours, at least 57 hours, at least 58 hours, at least 59 hours, at least 60 hours, at least 61 hours, at least 62 hours, at least 63 hours, at least 64 hours, at least 65 hours, at least 66 hours, at least 67 hours, at least 68 hours, at least 69 hours, at least 70 hours, at least 71 hours, at least 72 hours or more, e.g., at room temperature.

In some embodiments of any of the aspects, the stabilization agent is an RNA-preserving agent and/or a reverse-transcriptase-preserving agent. In some embodiments of any of the aspects, the reverse transcription reaction comprises an RNA-preserving agent. In some embodiments of any of the aspects, the reverse transcription reaction comprises a reverse-transcriptase-preserving agent. In some embodiments of any of the aspects, the reverse transcription reaction comprises both an RNA-preserving agent and a reverse-transcriptase-preserving agent.

In some embodiments of any of the aspects, the RNA-preserving agent is an RNase inhibitor, a metal-chelating agent, and/or a reducing agent. In some embodiments of any of the aspects, the reverse transcription reaction comprises an RNase inhibitor. In some embodiments of any of the aspects, the reverse transcription reaction comprises a metal-chelating agent. In some embodiments of any of the aspects, the reverse transcription reaction comprises a reducing agent. In some embodiments of any of the aspects, the reverse transcription reaction comprises an RNase inhibitor and a metal-chelating agent. In some embodiments of any of the aspects, the reverse transcription reaction comprises an RNase inhibitor and a reducing agent. In some embodiments of any of the aspects, the reverse transcription reaction comprises a metal-chelating agent and a reducing agent. In some embodiments of any of the aspects, the reverse transcription reaction comprises an RNase inhibitor, a metal-chelating agent, and a reducing agent.

In some embodiments of any of the aspects, the reverse-transcriptase-preserving agent is an antibiotic, an antimycotic, and/or a protease inhibitor. In some embodiments of any of the aspects, the reverse transcription reaction comprises an antibiotic. In some embodiments of any of the aspects, the reverse transcription reaction comprises an antimycotic. In some embodiments of any of the aspects, the reverse transcription reaction comprises a protease inhibitor. In some embodiments of any of the aspects, the reverse transcription reaction comprises an antibiotic and an antimycotic. In some embodiments of any of the aspects, the reverse transcription reaction comprises an antibiotic and a protease inhibitor. In some embodiments of any of the aspects, the reverse transcription reaction comprises an antimycotic and a protease inhibitor. In some embodiments of any of the aspects, the reverse transcription reaction comprises an antibiotic, an antimycotic, and a protease inhibitor.

In some embodiments of any of the aspects, the viral transport medium or reverse transcription reaction comprises contacting the sample with at least one of the following stabilization agents: (a) an RNase inhibitor; (b) a metal-chelating agent; (c) a reducing agent; d) an antibiotic; (e) an antimycoctic; and/or (f) a protease inhibitor. Table 13 provides exemplary combinations of such stabilization agents. In some embodiments, if the reverse transcription reaction does not comprise a specific stabilization agent, it can be added in a subsequent step.

Table 13: Non-Limiting Examples of Stabilization Agents in the RT Reaction; “RI” indicates an RNase inhibitor; “MC: indicates a metal-chelating agent; “RA” indicates a reducing agent; “AB” indicates an antibiotic; “AM” indicates an antimycoctic; and “PI” indicates a protease inhibitor.

TABLE 13

Non-Limiting Examples of Stagilization Agents in the RT Reaction

| | | | | | | | | | | | |
|---|---|---|---|---|---|---|---|---|---|---|---|
| RI | MC | RA | AB | AM | PI | RI | MC | RA | AB | AM | PI |
| | | | | | | | | | | | X |
| X | | | | | | X | | | | | X |
| | X | | | | | | X | | | | X |
| X | X | | | | | X | X | | | | X |
| | | X | | | | | | X | | | X |
| X | | X | | | | X | | X | | | X |
| | X | X | | | | | X | X | | | X |
| X | X | X | | | | X | X | X | | | X |
| | | | X | | | | | | X | | X |
| X | | | X | | | X | | | X | | X |
| | X | | X | | | | X | | X | | X |
| X | X | | X | | | X | X | | X | | X |
| | | X | X | | | | | X | X | | X |
| X | | X | X | | | X | | X | X | | X |
| | X | X | X | | | | X | X | X | | X |
| X | X | X | X | | | X | X | X | X | | X |
| | | | | X | | | | | | X | X |
| X | | | | X | | X | | | | X | X |
| | X | | | X | | | X | | | X | X |
| X | X | | | X | | X | X | | | X | X |
| | | X | | X | | | | X | | X | X |
| X | | X | | X | | X | | X | | X | X |
| | X | X | | X | | | X | X | | X | X |
| X | X | X | | X | | X | X | X | | X | X |
| | | | X | X | | | | | X | X | X |
| X | | | X | X | | X | | | X | X | X |
| | X | | X | X | | | X | | X | X | X |
| X | X | | X | X | | X | X | | X | X | X |
| | | X | X | X | | | | X | X | X | X |
| X | | X | X | X | | X | | X | X | X | X |
| | X | X | X | X | | | X | X | X | X | X |
| X | X | X | X | X | | X | X | X | X | X | X |

### RNase Inhibitor

In some embodiments of any of the aspects, the RNase inhibitor is murine RNase inhibitor or a thermostable RNase inhibitor. In some embodiments of any of the aspects, the RNase inhibitor specifically inhibits RNases A, B and C, which specifically cleave ssRNA or dsRNA. RNase A and RNase B are an endoribonuclease that specifically degrades single-stranded RNA at C and U residues. RNase C recognizes dsRNA and cleaves it at specific targeted locations to transform them into mature RNAs. In some embodiments of any of the aspects, the RNase inhibitor is present in the reverse transcription reaction at a concentration of at least 10% (e.g., volume per volume, v/v, percent). In some embodiments of any of the aspects, the RNase inhibitor is present in the reverse transcription reaction at a concentration of at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%.

Exemplary RNase inhibitors include, but are not limited to, mammalian ribonuclease inhibitor proteins such as porcine ribonuclease inhibitor and human ribonuclease inhibitor (e.g., human placenta ribonuclease inhibitor and recombinant human ribonuclease inhibitor), vanadyl ribonucleoside complexes, proteinase K, phenylglyoxal, p-hydroxyphenylglyoxal, polyamines, spermidine, 9-aminoacridine, iodoacetate, bentonite, poly[2′-O-(2,4-dinitrophenyl)]poly(adenyhlic acid), zinc sulfate, bromopyruvic acid, formamide, dimethylformamide, copper, zinc, aurintricarboxylic acid (ATA) and salts thereof such as triammonium aurintricarboxylate (aluminon), adenosine 5′-pyrophosphate, 2′-cytidine monophosphate free acid (2′-CMP), 5′-diphosphoadenosine 3′-phosphate (ppA-3′-p), 5′-diphosphoadenosine 2′-phosphate (ppA-2′-p), leucine, oligovinysulfonic acid, poly(aspartic acid), tyrosine-glutamic acid polymer, 5′-phospho-2′-deoxyuridine 3′-pyrophosphate P′→5′-ester with adenosine 3′-phosphate (pdUppAp), and analogs, derivatives and salts thereof.

In some embodiments of any of the aspects, the RNase inhibitor is a ribonuclease inhibitor protein, such as a recombinant RNase inhibitor, e.g., a recombinant mammalian RNase inhibitor. In some embodiments of any of the aspects, the RNase inhibitor is murine RNase inhibitor or RNasin® Plus (Promega™). In some embodiments of any of the aspects, the RNase inhibitor is murine RNase inhibitor or a thermostable RNase inhibitor. In some embodiments of any of the aspects, the RNase inhibitor is a thermostable RNase inhibitor, e.g., RNasin® Plus. One unit is defined as the amount of RNase inhibitor (e.g., RNasin®) required to inhibit the activity of 5 ng of ribonuclease A by 50%; activity is measured by the inhibition of hydrolysis of cytidine 2,3′-cyclic monophosphate by ribonuclease A.

In some embodiments of any of the aspects, the RNase inhibitor, i.e., a ribonuclease inhibitor protein, is added to a final concentration of at least 0.01 U/µL, at least 0.02 U/µL, at least 0.03 U/µL, at least 0.04 U/µL, at least 0.05 U/µL, at least 0.06 U/µL, at least 0.07 U/µL, at least 0.08 U/µL, at least 0.09 U/µL, at least 0.1 U/µL, at least 0.2 U/µL, at least 0.3 U/µL, at least 0.4 U/µL, at least 0.5 U/µL, at least 0.6 U/µL, at least 0.7 U/µL, at least 0.8 U/µL, at least 0.9 U/µL, at least 1.0 U/µL, at least 1.1 U/µL, at least 1.2 U/µL, at least 1.3 U/µL, at least 1.4 U/µL, at least 1.5 U/µL, at least 1.6 U/µL, at least 1.7 U/µL, at least 1.8 U/µL, at least 1.9 U/µL, at least 2.0 U/µL, at least 2.1 U/µL, at least 2.2 U/µL, at least 2.3 U/µL, at least 2.4 U/µL, at least 2.5 U/µL, at least 2.6 U/µL, at least 2.7 U/µL, at least 2.8 U/µL, at least 2.9 U/µL, at least 3.0 U/µL, at least 3.1 U/µL, at least 3.2 U/µL, at least 3.3 U/µL, at least 3.4 U/µL, at least 3.5 U/µL, at least 3.6 U/µL, at least 3.7 U/µL, at least 3.8 U/µL, at least 3.9 U/µL, at least 4.0 U/µL, at least 4.1 U/µL, at least 4.2 U/µL, at least 4.3 U/µL, at least 4.4 U/µL, at least 4.5 U/µL, at least 4.6 U/µL, at least 4.7 U/µL, at least 4.8 U/µL, at least 4.9 U/µL, at least 5.0 U/µL, at least 5.1 U/µL, at least 5.2 U/µL, at least 5.3 U/µL, at least 5.4 U/µL, at least 5.5 U/µL, at least 5.6 U/µL, at least 5.7 U/µL, at least 5.8 U/µL, at least 5.9 U/µL, at least 6.0 U/µL, at least 6.1 U/µL, at least 6.2 U/µL, at least 6.3 U/µL, at least 6.4 U/µL, at least 6.5 U/µL, at least 6.6 U/µL, at least 6.7 U/µL, at least 6.8 U/µL, at least 6.9 U/µL, at least 7.0 U/µL, at least 7.1 U/µL, at least 7.2 U/µL, at least 7.3 U/µL, at least 7.4 U/µL, at least 7.5 U/µL, at least 7.6 U/µL, at least 7.7 U/µL, at least 7.8 U/µL, at least 7.9 U/µL, at least 8.0 U/µL, at least 8.1 U/µL, at least 8.2 U/µL, at least 8.3 U/µL, at least 8.4 U/µL, at least 8.5 U/µL, at least 8.6 U/µL, at least 8.7 U/µL, at least 8.8 U/µL, at least 8.9 U/µL, at least 9.0 U/µL, at least 9.1 U/µL, at least 9.2 U/µL, at least 9.3 U/µL, at least 9.4 U/µL, at least 9.5 U/µL, at least 9.6 U/µL, at least 9.7 U/µL, at least 9.8 U/µL, at least 9.9 U/µL, at least 10 U/µL, at least 20 U/µL, at least 30 U/µL, at least 40 U/µL, or at least 50 U/µL.

### Metal-Chelating Agent

In some embodiments of any of the aspects, the metal-chelating agent is selected from the group consisting of ethylenediaminetetraacetic acid (EDTA), ethylene glycol-bis(β-aminoethyl ether)-N,N,N′,N′-tetraacetic acid (EGTA), 2,3-dimercapto-1-propanesulfonic acid sodium (DMPS), dimercaptosuccinic acid (DMSA), metallothionin, and desferroxamine. Chelation is the binding of ions and molecules to metal ions, involving the formation or presence of two or more separate coordinate bonds between a polydentate (multiple bonded) ligand and a single central metal atom. In some embodiments of any of the aspects, the metal-chelating agent is EDTA. In some embodiments of any of the aspects, the metal-chelating agent (e.g., EDTA) is present in the reverse transcription reagent at a concentration of at least 0.5 mM. In some embodiments of any of the aspects, the metal-chelating agent (e.g., EDTA) is present in the reverse transcription reagent at a concentration of at least 0.01 mM, at least 0.02 mM, at least 0.03 mM, at least 0.04 mM, at least 0.05 mM, at least 0.06 mM, at least 0.07 mM, at least 0.08 mM, at least 0.09 mM, at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1 mM or more.

It should be noted that metal-chelating agents, e.g., EDTA, can inhibit polymerase function as well as nuclease activities. In some embodiments of any of the aspects, the metal-chelating agent is diluted out or removed from the solution prior to the RT and/or amplification reactions.

### Reducing Agent

In some embodiments of any of the aspects, the reducing agent is selected from the group consisting of: tris-(2-carboxyethyl)-phosphine (TCEP), cysteine, dithionite, dithioerythritol, dithiothreitol (DTT), dysteine, 2- mercaptoethanol, mercaptoethylene, bisulfite, sodium metabisulfite, pyrosulfite, pentaerythritol, thioglycolic acid, urea, uric acid, vitamin C, vitamin E, superoxide dismutases, and analogs, derivatives and salts thereof. In some embodiments of any of the aspects, the reducing agent is dithiothreitol (DTT). Dithiothreitol (DTT) is a redox reagent used to stabilize proteins which possess free sulfhydryl groups (e.g., RT).

The reducing agent can be added to any desired amount. In some embodiments of any of the aspects, the reducing agent is present in the reverse transcription reaction at a concentration of at least 5 mM. For example, the reducing agent can be added to a final concentration of at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1 mM, at least 2 mM, at least 3 mM, at least 4 mM, at least 5 mM, at least 6 mM, at least 7 mM, at least 8 mM, at least 10 mM, at least 11 mM, at least 12 mM, at least 13 mM, at least 14 mM, at least 15, mM, at least 16 mM, at least 17 mM, at least 18 mM, at least 19 mM, at least 20 mM, at least 25 mM, at least 30 mM, at least 35 mM, at least 40 mM, at least 45 mM, at least 50 mM, at least 55 mM, at least 60 mM, at least 65 mM, at least 70 mM, at least 75 mM, at least 80 mM, at least 85 mM, at least 90 mM, at least 95 mM, at least 100 mM or more.

### Antibiotic and Antimycotic

In some embodiments of any of the aspects, the reverse transcription reaction comprises an antibiotic (i.e., anti-bacterial) and/or an antimycoctic (i.e., anti-fungal), which permits stabilization of the reverse transcriptase and prevents bacterial or fungal contamination of the sample (e.g., during incubation at room temperature for 6-24 hours). In some embodiments of any of the aspects, the antibiotic is penicillin (e.g., 10,000 units/mL) and/or streptomycin (e.g., 10,000 µg/mL). Penicillin was originally purified from the fungus Penicillium and acts by interfering directly with the turnover of the bacterial cell wall and indirectly by triggering the release of enzymes that further alter the cell wall. Penicillin inhibits gram-positive bacteria. Streptomycin was originally purified from Streptomyces griseus. Streptomycin acts by binding to the 30S subunit of the bacterial ribosome leading to inhibition of protein synthesis and death in susceptible bacteria. Streptomycin inhibits gram-positive and gram-negative bacteria.

In some embodiments of any of the aspects, the antibiotic (also referred to as anti-bacterial) is selected from the group consisting of: aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof. In some embodiments of any of the aspects, the antimicrobial agent can comprise an antibiotic.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

In some embodiments of any of the aspects, the antimycotic is Amphotericin B (e.g., 25 µg/mL). Amphotericin B is an antifungal agent that prevents the growth of fungi and yeast by causing an increase in fungal plasma membrane permeability. In some embodiments of any of the aspects, the antimycotic (also referred to as anti-fungal) is selected from the group consisting of: polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles, Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani’s paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

In some embodiments of any of the aspects, the antibiotic(s) and/or antimycoctic(s) is present in the reverse transcription reaction at a concentration of at least 10 ug/mL, at least 15 ug/mL, at least 20 ug/mL, at least 25 ug/mL, at least 30 ug/mL, at least 35 ug/mL, at least 40 ug/mL, at least 45 ug/mL, at least 50 ug/mL, at least 60 ug/mL, at least 70 ug/mL, at least 80 ug/mL, at least 90 ug/mL, at least 100 ug/mL, at least 110 ug/mL, at least 120 ug/mL, at least 130 ug/mL, at least 140 ug/mL, at least 150 ug/mL, at least 160 ug/mL, at least 170 ug/mL, at least 180 ug/mL, at least 190 ug/mL, at least 200 ug/mL, at least 210 ug/mL, at least 220 ug/mL, at least 230 ug/mL, at least 240 ug/mL, at least 250 ug/mL, at least 260 ug/mL, at least 270 ug/mL, at least 280 ug/mL, at least 290 ug/mL, at least 300 ug/mL, at least 310 ug/mL, at least 320 ug/mL, at least 330 ug/mL, at least 340 ug/mL, at least 350 ug/mL, at least 360 ug/mL, at least 370 ug/mL, at least 380 ug/mL, at least 390 ug/mL, at least 400 ug/mL, at least 410 ug/mL, at least 420 ug/mL, at least 430 ug/mL, at least 440 ug/mL, at least 450 ug/mL, at least 460 ug/mL, at least 470 ug/mL, at least 480 ug/mL, at least 490 ug/mL, at least 500 ug/mL, at least 510 ug/mL, at least 520 ug/mL, at least 530 ug/mL, at least 540 ug/mL, at least 550 ug/mL, at least 560 ug/mL, at least 570 ug/mL, at least 580 ug/mL, at least 590 ug/mL, at least 600 ug/mL, at least 610 ug/mL, at least 620 ug/mL, at least 630 ug/mL, at least 640 ug/mL, at least 650 ug/mL, at least 660 ug/mL, at least 670 ug/mL, at least 680 ug/mL, at least 690 ug/mL, at least 700 ug/mL, at least 710 ug/mL, at least 720 ug/mL, at least 730 ug/mL, at least 740 ug/mL, at least 750 ug/mL, at least 760 ug/mL, at least 770 ug/mL, at least 780 ug/mL, at least 790 ug/mL, at least 800 ug/mL, at least 810 ug/mL, at least 820 ug/mL, at least 830 ug/mL, at least 840 ug/mL, at least 850 ug/mL, at least 860 ug/mL, at least 870 ug/mL, at least 880 ug/mL, at least 890 ug/mL, at least 900 ug/mL, at least 910 ug/mL, at least 920 ug/mL, at least 930 ug/mL, at least 940 ug/mL, at least 950 ug/mL, at least 960 ug/mL, at least 970 ug/mL, at least 980 ug/mL, at least 990 ug/mL, at least 1000 ug/mL, at least 1500 ug/mL, at least 2000 ug/mL, at least 2500 ug/mL, at least 3000 ug/mL, at least 3500 ug/mL, at least 4000 ug/mL, at least 4500 ug/mL, at least 5000 ug/mL, at least 5500 ug/mL, at least 6000 ug/mL, at least 6500 ug/mL, at least 7000 ug/mL, at least 7500 ug/mL, at least 8000 ug/mL, at least 8500 ug/mL, at least 9000 ug/mL, at least 9500 ug/mL, at least 10,000 ug/mL or more.

In some embodiments of any of the aspects, the reverse transcription reaction does not comprise an antiviral. Non-limiting examples of antivirals include Abacavir, Acyclovir, Adefovir, Amantadine, Ampligen, Amprenavir, antiretroviral, Arbidol, Atazanavir, Atripla, Cidofovir, Combivir, Darunavir, Delavirdine, Didanosine, Docosanol, Dolutegravir, Ecoliever, Edoxudine, Efavirenz, Emtricitabine, Enfuvirtide, Entecavir, Famciclovir, Fomivirsen, Fosamprenavir, Foscarnet, Fosfonet, Fusion inhibitor, Ibacitabine, Idoxuridine, Imiquimod, Imunovir, Indinavir, Inosine, Integrase inhibitor, Interferon, Interferon type I, Interferon type II, Interferon type III, Lamivudine, Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Penciclovir, Peramivir, Pleconaril, Podophyllotoxin, viral protease inhibitor, Pyramidine, Raltegravir, Reverse transcriptase inhibitor, Ribavirin, Rimantadine, Ritonavir, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine.

### Protease Inhibitor

Protease inhibitors inhibit peptide degradation, e.g., degradation of the reverse transcriptase. Non-limiting classes of protease inhibitors include reversible or irreversible inhibitors of substrate (e.g., peptide) binding to the protease. Particular non-limiting classes of protease inhibitors include serine and cysteine protease inhibitors. Specific non-limiting examples of protease inhibitors include PMSF, PMSF Plus, APMSF, antithrombin I11, Amastatin, Antipain, aprotinin, Bestatin, Benzamidine, Chymostatin, calpain inhibitor I and II, E-64,3,4-dichloroisocoumarin, DFP, Elastatinal, Leupeptin, Pepstatin, 1,10-Phenanthroline, Phosphoramidon, TIMP-2, TLCK, TPCK, trypsin inhibitor (soybean or chicken egg white), hirustasin, alpha-2-macroglobulin, 4-(2-aminoethyl)-benzenesulfonyl fluoride hydrochloride (AEBSF) and Kunitz-type protease inhibitors.

In some embodiments of any of the aspects, the protease inhibitor is a protease inhibitor cocktail (e.g., cOmplete™ tablets). Such protease inhibitor tablets inhibit a broad spectrum of serine, cysteine, and metalloproteases, as well as calpains. Due to the composition of the tablets, they show excellent inhibition effects, and are well suited for the protection of proteins isolated from animal tissues, plants, yeast, and bacteria. Such protease inhibitor tablets comprise both irreversible and reversible protease inhibitors. Such protease inhibitor tablets can be substantially free of metal-chelating agents, such as EDTA.

In some embodiments of any of the aspects, the protease inhibitor is present at a concentration of one tablet per 10 mL of reverse transcriptase reaction buffer. In some embodiments of any of the aspects, the protease inhibitor is present at a concentration of at least 1, at least 2, at least 3, at least 4, at least 5 or more tablets per 10 mL of reverse transcriptase reaction buffer. In some embodiments of any of the aspects, the protease inhibitor is present at a concentration of one tablet for at least 1 mL, at least 2 mL, at least 3 mL, at least 4 mL, at least 5 mL, at least 6mL, at least 7 mL, at least 8 mL, at least 9 mL, at least 10 mL, at least 11 mL, at least 12 mL, at least 13 mL, at least 14 mL, at least 15 mL, at least 16 mL, at least 17 mL, at least 18 mL, at least 19 mL, at least 20 mL or more of reverse transcriptase reaction buffer.

### Reverse Transcription Reaction

In some embodiments of any of the aspects, step (a) comprises a reverse transcription reaction. In some embodiments of any of the aspects, the RT step comprises one round of polymerization, wherein the target RNA is reverse-transcribed into a single-stranded cDNA. In some embodiments of any of the aspects, the reverse transcription products from step (a) (the RT step) comprise a barcoded DNA comprising a region that is complementary to a portion of at least one target RNA.

In some embodiments of any of the aspects, the reverse transcription step comprises contacting the sample with a reverse transcriptase, a first primer or a first set of primers, and a reverse transcription reaction buffer. In some embodiments, the RT reaction buffer comprises at least one of the following: water, magnesium acetate (or another magnesium compound such as magnesium chloride), and/or dNTPs. In some embodiments of any of the aspects, the reaction buffer maintains the reaction at specific optimal pH (e.g., 7-9; e.g., 8.1) and can include such components as Tris, KCl, MgCl2, and other buffers or salts. Magnesium ions (Mg2+) can function as a cofactor for polymerases, increasing their activity. Deoxynucleoside triphosphate (dNTPs) are free nucleoside triphosphates comprising deoxyribose as the sugar (e.g., dATP, dGTP, dCTP, and dTTP) that are used in the polymerization of the cDNA.

In one aspect, described herein is a reverse transcription solution comprising at least one of the following: (a) a reverse transcriptase; (b) a first primer or a first set of primers comprising at least one barcode; (c) a detergent; (d) carrier nucleic acid; (e) at least one positive control nucleic acid; (f) at least one stabilization agent; and/or (g) a RT reaction buffer. Table 14 provides exemplary combinations of such reverse transcription solution components. In some embodiments, if the reverse transcription solution does not comprise a specific component, it can be added in a subsequent step.

“RT” indicates reverse transcriptase; “FP” indicates first primer or a first set of primers comprising at least one barcode; “Det.” indicates a detergent; “CN” indicates carrier nucleic acid; “PC” indicates at least one positive control nucleic acid; “SA” indicates at least one stabilization agent; and “Buf.” indicates a RT reaction buffer.

TABLE 14

Non-Limiting Examples of Reverse Transcription Solutions

| | | | | | | | | | | | | | |
|---|---|---|---|---|---|---|---|---|---|---|---|---|---|
| RT | FP | Det. | CN | PC | SA | Buf. | RT | FP | Det. | CN | PC | SA | Buf. |
| | | | | | | | X | | | | | X | |
| X | | | | | | | | X | | | | X | |
| | X | | | | | | X | X | | | | X | |
| X | X | | | | | | | | X | | | X | |
| | | X | | | | | X | | X | | | X | |
| X | | X | | | | | | X | X | | | X | |
| | X | X | | | | | X | X | X | | | X | |
| X | X | X | | | | | | | | X | | X | |
| | | | X | | | | X | | | X | | X | |
| X | | | X | | | | | X | | X | | X | |
| | X | | X | | | | X | X | | X | | X | |
| X | X | | X | | | | | | X | X | | X | |
| | | X | X | | | | X | | X | X | | X | |
| X | | X | X | | | | | X | X | X | | X | |
| | X | X | X | | | | X | X | X | X | | X | |
| X | X | X | X | | | | | | | | X | X | |
| | | | | X | | | X | | | | X | X | |
| X | | | | X | | | | X | | | X | X | |
| | X | | | X | | | X | X | | | X | X | |
| X | X | | | X | | | | | X | | X | X | |
| | | X | | X | | | X | | X | | X | X | |
| X | | X | | X | | | | X | X | | X | X | |
| | X | X | | X | | | X | X | X | | X | X | |
| X | X | X | | X | | | | | | X | X | X | |
| | | | X | X | | | X | | | X | X | X | |
| X | | | X | X | | | | X | | X | X | X | |
| | X | | X | X | | | X | X | | X | X | X | |
| X | X | | X | X | | | | | X | X | X | X | |
| | | X | X | X | | | X | | X | X | X | X | |
| X | | X | X | X | | | | X | X | X | X | X | |
| | X | X | X | X | | | X | X | X | X | X | X | |
| X | X | X | X | X | | | | | | | | | X |
| | | | | | X | | X | | | | | | X |
| | X | | | | | X | X | | | | | X | X |
| X | X | | | | | X | | X | | | | X | X |
| | | X | | | | X | X | X | | | | X | X |
| X | | X | | | | X | | | X | | | X | X |
| | X | X | | | | X | X | | X | | | X | X |
| X | X | X | | | | X | | X | X | | | X | X |
| | | | X | | | X | X | X | X | | | X | X |
| X | | | X | | | X | | | | X | | X | X |
| | X | | X | | | X | X | | | X | | X | X |
| X | X | | X | | | X | | X | | X | | X | X |
| | | X | X | | | X | X | X | | X | | X | X |
| X | | X | X | | | X | | | X | X | | X | X |
| | X | X | X | | | X | X | | X | X | | X | X |
| X | X | X | X | | | X | | X | X | X | | X | X |
| | | | | X | | X | X | X | X | X | | X | X |
| X | | | | X | | X | | | | | X | X | X |
| | X | | | X | | X | X | | | | X | X | X |
| X | X | | | X | | X | | X | | | X | X | X |
| | | X | | X | | X | X | X | | | X | X | X |
| X | | X | | X | | X | | | X | | X | X | X |
| | X | X | | X | | X | X | | X | | X | X | X |
| X | X | X | | X | | X | | X | X | | X | X | X |
| | | | X | X | | X | X | X | X | | X | X | X |
| X | | | X | X | | X | | | | X | X | X | X |
| | X | | X | X | | X | X | | | X | X | X | X |
| X | X | | X | X | | X | | X | | X | X | X | X |
| | | X | X | X | | X | X | X | | X | X | X | X |
| X | | X | X | X | | X | | | X | X | X | X | X |
| | X | X | X | X | | X | X | | X | X | X | X | X |
| X | X | X | X | X | | X | | X | X | X | X | X | X |
| | | | | | X | X | X | X | X | X | X | X | X |

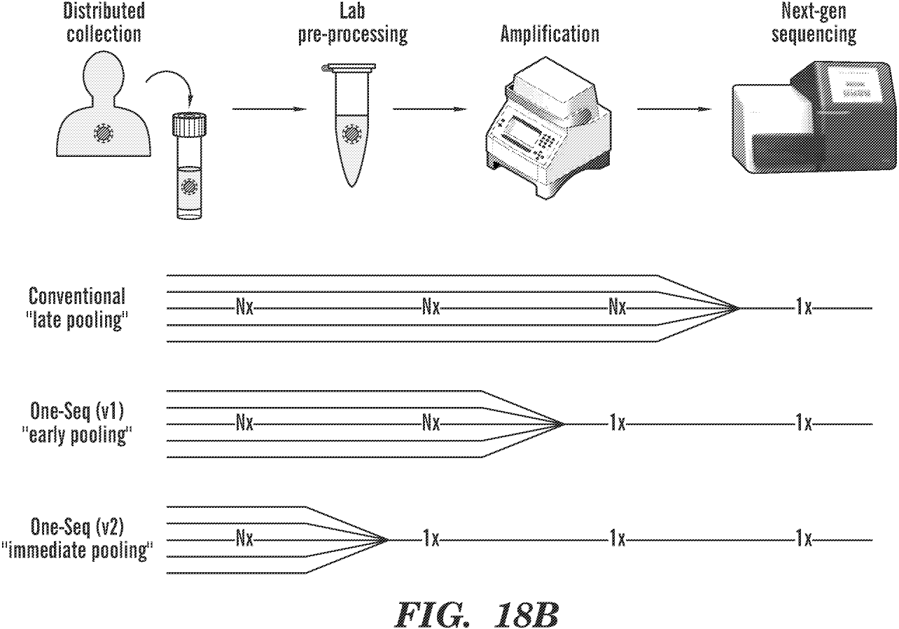

In one aspect, described herein is a collection container (e.g., a collection tube) containing a reverse transcription solution as described herein. In some embodiments of any of the aspects, the sample collection container further contains viral transport media, as described further herein. In some embodiments of any of the aspects, a sample from the subject can be added directly to the collection container, reducing the number of liquid handling steps (see e.g., FIGS. 18A-18B). In some embodiments of any of the aspects, the reverse transcription step is performed in the collection container.

In some embodiments of any of the aspects, step (a) (the RT step) comprises: (i) incubating the sample, reverse transcriptase, and first primer or first set of primers comprising at least one barcode at a temperature of at least 50° C. for at least 30 minutes; and (ii) inactivating the reverse transcription reaction at a temperature of at least 95° C. for at least 5 minutes. In some embodiments of any of the aspects, step (i) further comprises incubating the sample in a RT reaction solution as described herein (see e.g. Table 13 and Table 14).

In some embodiments of any of the aspects, step (i) (e.g., the incubation step) of the RT reaction comprises incubating the reaction at a temperature of at least 50° C. In some embodiments of any of the aspects, step (i) (e.g., the incubation step) of the RT reaction comprises incubating the reaction at a temperature of at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C. or more. In some embodiments of any of the aspects, the RT step is performed at body temperature (e.g., 37° C.). In some embodiments of any of the aspects, the RT step is performed on a heat block set to approximately 50° C. or an incubator set to approximately 50° C.

In some embodiments of any of the aspects, step (i) (e.g., the incubation step) of the RT reaction comprises incubating the reaction for at least 30 minutes. In some embodiments of any of the aspects, step (i) (e.g., the incubation step) of the RT reaction comprises incubating the reaction for at least 15 minutes, at least 20 minutes, at least 25 minutes, at least 30 minutes, at least 40 minutes, at least 50 minutes, at least 60 minutes, at least 70 minutes, at least 80 minutes, at least 90 minutes, or at least 100 minutes. The specific conditions, e.g., of temperature, time, and buffer conditions can be varied as necessary to accommodate different RT enzymes.

In some embodiments of any of the aspects, step (ii) (e.g., the inactivation step) of the RT reaction comprises inactivating the reaction at a temperature of at least 95° C. In some embodiments of any of the aspects, step (ii) (e.g., the inactivation step) of the RT reaction comprises inactivating the reaction at a temperature of at least 50° C., at least 55° C., at least 60° C., at least 65° C., at least 70° C., at least 75° C., at least 80° C., at least 85° C., at least 90° C., at least 91° C., at least 92° C., at least 93° C., at least 94° C., at least 95° C., at least 96° C., at least 97° C., at least 98° C., at least 99° C., at least 99° C., or at least 99.5° C. In some embodiments of any of the aspects, step (ii) (e.g., the inactivation step) of the RT reaction comprises inactivating the reaction for at least 5 minutes. In some embodiments of any of the aspects, step (ii) (e.g., the inactivation step) of the RT reaction comprises inactivating the reaction for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 20 minutes, at least 25 minutes, or at least 30 minutes.

In some embodiments of any of the aspects, the reverse transcription products from step (a) for different samples are combined in one container to form a pooled reverse transcription product mixture. Such a step is in contrast to other methods, in which products can only be combined after the amplification step, not the reverse transcription step. Contacting the sample with a first primer or a first set of primers comprising at least one barcode, which produces individually barcoded cDNAs, allows for pre-amplification pooling of the reverse transcription products. In some embodiments of any of the aspects, reverse transcription products from step (a) (the RT step) of at least 5 samples are combined in one container. In some embodiments of any of the aspects, reverse transcription products from step (a) (the RT step) of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, at least 600, at least 700, at least 800, at least 900, at least 1000, at least 1500, at least 2000, at least 2500, at least 3000, at least 3500, at least 4000, at least 4500, at least 5000 or more samples are combined in one container.

In some embodiments of any of the aspects, the reverse transcription step is performed in at most 30 minutes. As a non-limiting example, the reverse transcription step is performed in at most 20 minutes, at most 25 minutes, at most 30 minutes, at most 40 minutes, at most 50 minutes, at most 60 minutes, at most 70 minutes, at most 80 minutes, at most 90 minutes, at most 100 minutes, at most 110 minutes, or at most 120 minutes.

In another aspect, provided herein are compositions useful in detecting an RNA target. The composition can comprise any of the reagents discussed herein. In one aspect, described herein is a reverse transcription composition comprising at least two of the following: (a) a target RNA; (b) a reverse transcriptase; (c) a first primer or a first set of primers comprising at least one barcode; (d) a detergent; (e) a carrier nucleic acid; (f) a positive control nucleic acid; and/or (g) at least one stabilization agent. It is noted that a composition can comprise any one, two, three, four, five, six, or all seven of the components listed above.

### Amplification

Described are methods, kits, and systems that can be used to detect a target RNA. In some embodiments of any of the aspects, the cDNA resulting from the RT step is amplified to detectable levels. In some embodiments, the target RNA is present at a low starting amount, such that amplification is needed in order to detect the RNA. As used herein, “amplification” is defined as the production of additional copies of a nucleic acid sequence, i.e., for example, amplicons or amplification products. Methods of amplifying nucleic acid sequences are well known in the art. Such methods include, but are not limited to, polymerase chain reaction (PCR) and variants of PCR such as Rapid amplification of cDNA ends (RACE); ligase chain reaction (LCR); multiplex RT-PCR; immuno-PCR; Sequence-Independent, Single-Primer-Amplification (SSIPA); Real Time RT-qPCR; nanofluidic digital PCR; or isothermal amplification methods. Accordingly, the methods described herein comprise an amplification step (e.g., step (c)) of contacting the pooled reverse transcription product mixture with a DNA polymerase and a second set of primers, e.g., under conditions permitting the generation of amplification products. As used herein, the phrase “conditions permitting the generation of amplification products” refers to temperature(s), time(s), and/or reagent(s) that allow the DNA polymerase to catalyze the generation of dsDNA from the cDNA using at least one primer (e.g., at least two primers) from the second set of primers. In some embodiments of any of the aspects, the second set of primers comprises at least 2 primers and comprises a forward primer and reverse primer that together amplify a target of 15 base pairs (bp) - 50,000 bp, unless indicated otherwise.

In some embodiments of any of the aspects, the amplification step permits an amplification reaction, such as a polymerase chain reaction. In general, the PCR procedure relates to a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary or include sequence complementary to a strand of the template (e.g., target cDNA) to be amplified. In an alternative embodiment, mRNA level of gene expression products described herein can be determined by reverse-transcription (RT) PCR or quantitative RT-PCR (QRT-PCR) or real-time PCR methods. Methods of RT-PCR and QRT-PCR are well known in the art.

In some embodiments of any of the aspects, the amplification method comprises isothermal amplification, which permits rapid and specific amplification of DNA at a constant temperature. In general, isothermal amplification is comprised of (i) sequence-specific hybridization of primers to specific genes or sequences within a nucleic acid sample or library, (ii) subsequent amplification involving multiple rounds of primer annealing, elongation, and strand displacement (as a non-limiting example, using a combination of recombinase, single-stranded binding proteins, and DNA polymerase), and (iii) detection of the product. In some embodiments of any of the aspects, the isothermal amplification produce can be detected through such methods as sequencing to confirm the identity of the amplified product or general assays such as turbidity. In some types of isothermal amplification, turbidity results from pyrophosphate byproducts produced during the reaction; these byproducts form a white precipitate that increases the turbidity of the solution. The primers used in isothermal amplification are oligonucleotides of sufficient length and appropriate sequence to provide initiation of polymerization, i.e. each primer is specifically designed to be complementary or include sequence complementary to a strand of the template (e.g., target cDNA) to be amplified. In contrast to the polymerase chain reaction (PCR) technology in which the reaction is carried out with a series of alternating temperature steps or cycles, isothermal amplification is carried out at one temperature, and does not require a thermal cycler or thermostable enzymes.

Non-limiting examples of isothermal amplification include: Recombinase Polymerase Amplification (RPA), nested RPA, Loop Mediated Isothermal Amplification (LAMP), Helicase-dependent isothermal DNA amplification (HDA), thermophilic helicase-dependent amplification (tHDA), Rolling Circle Amplification (RCA), strand displacement amplification (SDA), ligase chain reaction (LCR), nicking enzyme amplification reaction (NEAR), polymerase Spiral Reaction (PSR), polymerase cross-linking spiral reaction (PCLSR), and transcription-based amplification systems (TAS) such as nucleic acid sequence based amplification (NASBA), Rolling Circle Amplification (RCA), “RACE” and “one-sided PCR.” See e.g., Yan et al., Isothermal amplified detection of DNA and RNA, March 2014, Molecular BioSystems 10(5), DOI: 10.1039/c3mb70304e, the content of which is incorporated herein by reference in its entirety. In some embodiments of any of the aspects, the isothermal amplification reaction is Recombinase Polymerase Amplification (RPA) or Loop Mediated Isothermal Amplification (LAMP).

In some embodiments of any of the aspects, the isothermal amplification reaction is Recombinase Polymerase Amplification (RPA). RPA is a low temperature DNA and RNA amplification technique. The RPA process employs three core enzymes - a recombinase, a single-stranded DNA-binding protein (SSB) and strand-displacing polymerase. Recombinases are capable of pairing oligonucleotide primers with homologous sequence in duplex DNA. SSB bind to displaced strands of DNA and prevent the primers from being displaced. Finally, the strand displacing polymerase begins DNA synthesis where the primer has bound to the target DNA. By using two opposing primers, much like PCR, if the target sequence is indeed present, an exponential DNA amplification reaction is initiated. No other sample manipulation such as thermal or chemical melting is required to initiate amplification. At optimal temperatures (e.g., 37-42° C.), the RPA reaction progresses rapidly and results in specific DNA amplification from just a few target copies to detectable levels, typically within 10 minutes, for rapid detection of the target nucleic acid. In some embodiments of any of the aspects, the single-stranded DNA-binding protein is a gp32 SSB protein. In some embodiments of any of the aspects, the recombinase is a uvsX recombinase. See e.g., U.S. Pat. 7,666,598, the content of which is incorporated herein by reference in its entirety. In some embodiments of any of the aspects, RPA can also be referred to as Recombinase Aided Amplification (RAA). Accordingly, in some embodiments of any of the aspects, the amplification step comprises contacting the pooled reverse transcription product mixture from step (b) with a recombinase and single-stranded DNA binding protein. In some embodiments of any of the aspects, the amplification step(s) comprises contacting the pooled reverse transcription product mixture from step (b) with a DNA polymerase, a second set of primers, a recombinase, and single-stranded DNA binding protein.

In some embodiments of any of the aspects, the isothermal amplification reaction is Loop Mediated Isothermal Amplification (LAMP). LAMP is a single tube technique for the amplification of DNA; LAMP uses 4-6 primers, which form loop structures to facilitate subsequent rounds of amplification. Accordingly, in some embodiments of the aspects, the amplification step(s) comprises contacting the pooled reverse transcription product mixture from step (b) with a DNA polymerase and a set of primers, wherein the set of primers comprises 4, 5, or 6 loop-forming primers.

In some embodiments of any of the aspects, prior to step (c) (the amplification step) the first set of barcoded primers is substantially removed, e.g., from the pooled reverse transcription product mixture. In some embodiments of any of the aspects, prior to step (c) the target RNA is substantially removed, e.g., from the pooled reverse transcription product mixture. In some embodiments of any of the aspects, prior to step (c) the sample (e.g., the patient sample; e.g., the viral sample) is substantially removed, e.g., from the pooled reverse transcription product mixture. In some embodiments of any of the aspects, prior to step (c) the first set of barcoded primers, the RNA target, and/or the sample is substantially removed using a bead-based purification method. In some embodiments of any of the aspects, prior to step (c) the first set of barcoded primers, the RNA target, and/or the sample is substantially removed using a spin-column-based purification method.

Spin column-based nucleic acid purification is a solid phase extraction method to quickly purify nucleic acids. This method relies on the fact that nucleic acid will bind to the solid phase of silica under certain conditions. Magnetic bead/particle-based purification methods also employ a bind-wash-elute process. However, instead of using centrifugation or vacuum manifolds to remove the aqueous phase from contact with the silica matrix, these workflows use magnetic beads or particles functionalized with silica surfaces to allow selective binding of DNA in the presence of high concentrations of salt. DNA bound to a magnetic bead can be easily separated from the aqueous phase using a magnet; thereby allowing rapid sample processing and fine control of solution volumes. Magnetic-based methods are ideal for automation of high throughput processing, as they eliminate the need for centrifugation and other time-consuming steps.

### DNA Polymerase

In some embodiments of any of the aspects, the DNA polymerase used in the amplification step is a DNA-dependent DNA polymerase. DNA polymerases catalyze the synthesis of DNA molecules from nucleoside triphosphates, the molecular precursors of DNA, using a DNA or cDNA template. In some embodiments of any of the aspects, the DNA polymerase is a thermostable DNA polymerase, e.g., capable of withstanding (i.e., not irreversibly denaturing at) the high temperatures used in the amplification step. In some embodiments of any of the aspects, the DNA polymerase is a thermostable DNA polymerase I. DNA polymerase I (Pol I) is a prokaryotic polymerase, which is encoded by the po1A gene and ubiquitous among prokaryotes. This repair polymerase is involved in excision repair with both 3′-5′ and 5′-3′ exonuclease activity and processing of Okazaki fragments generated during lagging strand synthesis. Pol I is the most abundant polymerase in most prokaryotes.

Non-limiting examples of thermostable DNA polymerases include: Taq DNA polymerase from Thermus aquaticus; AmpliTaq™ Gold from Thermus aquaticus; HotTub™ from Thermus flavus; rTth from Thermus thermophilus; DNA polymerase from Thermotoga maritima (Ultma); Pwo DNA polymerase (Pyrococcus woesei); Tfl DNA polymerase (Thermus flavus); Tli DNA polymerase (Thermus litoralis); see e.g., Al-Soud et al., Appl Environ Microbiol. 1998 Oct; 64(10): 3748-3753. In some embodiments of any of the aspects, the DNA polymerase is a Thermus aquaticus (Taq) DNA polymerase or variant thereof (see e.g., SEQ ID NO: 1007). Taq polymerase is a heat-stable enzyme of this family that lacks proofreading ability. In some embodiments of any of the aspects, the DNA polymerase comprises SEQ ID NO: 1007 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1007 that maintains the same function (e.g., DNA-dependent DNA polymerase).

SEQ ID NO: 1007, DNA polymerase I, thermostable, po1A, Thermus aquaticus, UniProtKB - P19821, 832 aa

MRGMLPLFEPKGRVLLVDGHHLAYRTFHALKGLTTSRGEPVQAVYGFAKS
LLKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLLS
DRIHVLHPEGYLITPAWLWEKYGLRPDQWADYRALTGDESDNLPGVKGIG
EKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWP
PPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTE
EAGERAALSERLFANLWGRLEGEERLLWLYREVERPLSAVLAHMEATGVR
LDVAYLRALSLEVAEEIARLEAEVFRLAGHPFNLNSRDQLERVLFDELGL
PAIGKTEKTGKRSTSAAVLEALREAHPIVEKILQYRELTKLKSTYIDPLP
DLIHPRTGRLHTRFNQTATATGRLSSSDPNLQNIPVRTPLGQRIRRAFIA
EEGWLLVALDYSQIELRVLAHLSGDENLIRVFQEGRDIHTETASWMFGVP
REAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFN
MPVQGTAADLMKLAMVKLFPRLEEMGARMLLQVHDELVLEAPKERAEAVA
RLAKEVMEGVYPLAVPLEVEVGIGEDWLSAKE

In some embodiments of any of the aspects, the DNA polymerase is provided (i.e., added to the reaction mixture) at a sufficient concentration to promote polymerization, e.g., 0.1 U/µL to 100 U/µL. As used herein, one unit (“U”) of DNA polymerase (e.g., Taq) is defined as the amount of enzyme that incorporates 10 nmol of total deoxyribonucleoside triphosphates into acid precipitable DNA within 60 min at +65° C. In some embodiments of any of the aspects, the DNA polymerase is provided at a concentration of at least 0.1 U/µL, at least 0.2 U/µL, at least 0.3 U/µL, at least 0.4 U/µL, at least 0.5 U/µL, at least 0.6 U/µL, at least 0.7 U/µL, at least 0.8 U/µL, at least 0.9 U/µL, at least 1 U/µL, at least 2 U/µL, at least 3 U/µL, at least 4 U/µL, at least 5 U/µL, at least 6 U/µL, at least 7 U/µL, at least 8 U/µL, at least 9 U/µL, at least 10 U/µL, at least 20 U/µL, at least 30 U/µL, at least 40 U/µL, at least 50 U/µL, at least 60 U/µL, at least 70 U/µL, at least 80 U/µL, at least 90 U/µL, at least 100 U/µL or more.

### Second Set of Primers

In some embodiments of any of the aspects, the sample is contacted with a second set of primers (i.e., after the first set of RT primers). In some embodiments of any of the aspects, the second set of primers is specific to the target RNA. In some embodiments of any of the aspects, the second set of primers is specific (i.e., binds specifically through complementarity) to cDNA, in other words, the DNA produced in the RT step that is complementary to the target RNA. The second set of primers can be specific to any region of the target RNA. In some embodiments of any of the aspects, the second set of primers comprises at least one barcode region. In some embodiments of any of the aspects, the second set of primers comprises 1, 2, 3, 4, 5, or more barcode regions.

In some embodiments, a forward primer, e.g., in the second set of primers is about 50 nucleotides long. In some embodiments, a reverse primer, e.g., in the second set of primers is about 80 nucleotides long. In some embodiments, a primer, e.g., in the second set of primers is about 40-100 nucleotides long. As a non-limiting example, the primer is 40 nucleotides (nt) long, 41 nt, 42 nt, 43 nt, 44 nt, 45 nt, 46 nt, 47 nt, 48 nt, 49 nt, 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, 85 nt, 86 nt, 87 nt, 88 nt, 89 nt, 90 nt, 91 nt, 92 nt, 93 nt, 94 nt, 95 nt, 96 nt, 97 nt, 98 nt, 99 nt, 100 nt or more. In some embodiments of any of the aspects, at least one primer, e.g., from the second set of primers, comprises sequences selected from Table 4. In some embodiments of any of the aspects, the second set of primers comprises forward and reverse amplification primers.

In some embodiments of any of the aspects, a forward primer in the second set of primers comprises from 5′ to 3′: (a) an adaptor region; and (b) an adaptor-binding region that is identical or substantially identical to the adaptor region of a primer in the first set of barcoded primers. In some embodiments of any of the aspects, a forward primer in the second set of primers comprises from 5′ to 3′: (a) an adaptor region; (b) a third barcode region; and (c) an adaptor-binding region that is identical or substantially identical to the adaptor region of a primer in the first set of barcoded primers.

In some embodiments of any of the aspects, the adaptor region, e.g., of a forward primer in the second set of primers, comprises a sequencing adaptor region that allows for a high throughput sequencing method (e.g., P5 adaptor or P7 adaptor). In some embodiments of any of the aspects, the adaptor-binding region, e.g., of a forward primer in the second set of primers, specifically binds to the reverse complement of the adaptor region (e.g., PCR adaptor) of a primer in the first set of primers. In some embodiments of any of the aspects, the PCR adaptor-binding region, e.g., of a forward primer in the second set of primers, comprises SEQ ID NO: 13. In some embodiments of any of the aspects, a forward primer in the second set of primers, e.g., comprising the adaptor region and the adaptor-binding region, comprises SEQ ID NO: 14 or a nucleic acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 14 that maintains the same function (e.g., amplification adaptor and/or sequencing adaptor). In some embodiments of any of the aspects, a forward primer in the second set of primers allows the amplification product to specifically bind to a sequencing primer (e.g., read 1 primer, SEQ ID NO: 15).

In some embodiments of any of the aspects, a reverse primer in the second set of primers comprises, from 5′ to 3′:(a) an adaptor region; (b) a second barcode region; and (c) a target-binding region that is identical or substantially identical to at least one target RNA. In some embodiments of any of the aspects, a reverse primer in the second set of primers comprises, from 5′ to 3′:(a) an adaptor region; and (b) a region that is identical or substantially identical to at least one target RNA. In some embodiments of any of the aspects, the adaptor region, e.g., of a reverse primer in the second set of primers, comprises a sequencing adaptor region that allows for a high throughput sequencing method (e.g., P7 adaptor or P5 adaptor).

In some embodiments of any of the aspects, the adaptor region, e.g., of a reverse primer in the second set of primers, comprises SEQ ID NO: 16 or a nucleic acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 16 that maintains the same function (e.g., sequencing adaptor). In some embodiments of any of the aspects, a reverse primer in the second set of primers allows the amplification product to specifically bind to a sequencing primer (e.g., read 2 primer, SEQ ID NO: 17).

In some embodiments of any of the aspects, a barcode region on a primer in the second set of primers is shorter than the barcode region on a primer in the first set of primers. In some embodiments of any of the aspects, a barcode region on a primer in the second set of primers is at least 8 nucleotides long. As a non-limiting example, the barcode region can be 10 nucleotides (nt) long, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt long or more. In some embodiments of any of the aspects, the barcode region of a first primer in the second set of barcoded primers is a Hamming distance of at least 5 from each other barcode region of any other primer in the second set of barcoded primers. In some embodiments of any of the aspects, the barcode region of a first primer in the second set of barcoded primers is a Hamming distance of 4-6 from each other barcode region of any other primer in the second set of barcoded primers. In some embodiments of any of the aspects, the barcode region of a first primer in the second set of barcoded primers is a Hamming distance of at least 1, at least 2, at least 3, at least 4, at least 5, at least 6, at least 7, at least 8, at least 9, or at least 10, or more from each other barcode region of any other primer in the second set of barcoded primers (or barcode region in a first, third, fourth, etc. set of barcoded primers).

In some embodiments of any of the aspects, the second or third barcode region on a primer in the second set of primers comprises one of SEQ ID NOs: 18-989 or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 18-989 that maintains the same function (e.g., identification). In some embodiments of any of the aspects, the first barcode region on the first primer or set of first primers comprises one of SEQ ID NOs: 18-29 or SEQ ID NO: 992 (see e.g., Table 4 or FIG. 20A); such barcodes are also referred to herein as “batch barcode” or batch ID.” In some embodiments of any of the aspects, the at least one barcode region on a primer in the second set of primers corresponds to and is different for each of the at least two batches (e.g., batched by RT reaction; e.g., batched by local community, organization, or department).

In some embodiments of any of the aspects, a target-binding region is complementary or substantially complementary to and permits hybridization to at least one target RNA. In some embodiments of any of the aspects, the target-binding region permits hybridization to at least one target RNA under conditions permitting the generation of a reverse transcription product. In some embodiments of any of the aspects, the target-binding region, e.g., of a primer in the second set of primers, is about 20 nucleotides long. In some embodiments, the target-binding region, e.g., of a primer in the second set of primers, is about 15-35 nucleotides long. As a non-limiting example, the target-binding region can be 15 nucleotides (nt) long, 16 nt, 17 nt,18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt long or more. In some embodiments, the target-binding region, e.g., of a primer in the second set of primers, has a Tm of about 60° C.-62° C., e.g., at least 60° C., at least 60.5° C., at least 61° C., at least 61.5° C., at least 62° C. or more.

In some embodiments of any of the aspects, the target-binding region of a primer in the second set of primers binds to a region of SARS-CoV-2 N gene or S gene (see e.g., SEQ ID NO: 1001-1002). In some embodiments of any of the aspects, the target-binding region of a primer in the first set of primers comprises one of SEQ ID NO: 4 (N#1 _PCR), SEQ ID NO: 6 (N#2 _PCR), SEQ ID NO: 8 (del6970_PCR), SEQ ID NO: 10 (D614 _PCR), SEQ ID NO: 12 (positive control PCR) or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 4, 6, 8, 10, or 12 that maintains the same function (e.g., binding to the target RNA or positive control RNA) (see e.g., Table 4).

In some embodiments of any of the aspects, the reverse primer in the second set of primers comprises, from 5′ to 3′: (a) an adaptor region (e.g., SEQ ID NO: 16); (b) optionally, a second barcode region (e.g., one of 18-29 or SEQ ID NO: 992 or reverse complement thereof); and (c) a target-binding region that is identical or identical complementary to and permits hybridization to at least one target RNA (e.g., one of SEQ ID NOs: 4, 6, 8, 10, or 12). SEQ ID NO: 1008 is an exemplary reverse primer from the second set of primers, comprising from 5′ to 3′: SEQ ID NO: 16 (bolded), the reverse complement of SEQ ID NO: 992, and SEQ ID NO: 4 (bold italicized).

SEQ ID NO: 1008, 85 nt (see e.g., FIG. 20A) CAAGCAGAAGACGGCATACGAGATACGAGCAAGCACAGGACCACAACACGcaatatatgcgc GTTTACCCAATAATACTGCGTCT

In some embodiments of any of the aspects, the forward and/or reverse primers of the second set of primers are present in the amplification reaction at a concentration of at least 0.125 uM. In some embodiments of any of the aspects, the forward and/or reverse primers of the second set of primers are present in the amplification reaction at a concentration of at least 0.25 uM. In some embodiments of any of the aspects, the forward and/or reverse primers of the second set of primers are present in the amplification reaction at a concentration of at least 0.5 uM. In some embodiments of any of the aspects, the forward and/or reverse primers of the second set of primers are present in the amplification reaction at a concentration of at least 25 nM, at least 30 nM, at least 35 nM, at least 40 nM, at least 45 nM, at least 50 nM, at least 55 nM, at least 60 nM, at least 65 nM, at least 70 nM, at least 75 nM, at least 80 nM, at least 85 nM, at least 90 nM, at least 95 nM, at least 100 nM, at least 105 nM, at least 110 nM, at least 115 nM, at least 120 nM, at least 125 nM, at least 130 nM, at least 135 nM, at least 140 nM, at least 145 nM, at least 150 nM, at least 160 nM, at least 170 nM, at least 180 nM, at least 190 nM, at least 200 nM, at least 210 nM, at least 220 nM, at least 230 nM, at least 240 nM, at least 250 nM, at least 260 nM, at least 270 nM, at least 280 nM, at least 290 nM, at least 300 nM, at least 310 nM, at least 320 nM, at least 330 nM, at least 340 nM, at least 350 nM, at least 360 nM, at least 370 nM, at least 380 nM, at least 390 nM, at least 400 nM, at least 410 nM, at least 420 nM, at least 430 nM, at least 440 nM, at least 450 nM, at least 460 nM, at least 470 nM, at least 480 nM, at least 490 nM, at least 500 nM.

In some embodiments of any of the aspects, specific combinations of primers in the first and second set of primers are used for the reverse transcription and amplification reactions. In some embodiments of any of the aspects, the same set of sequencing primers (i.e., the third set of primers) can be used for sequencing the amplification products (see e.g., Table 15).

For the RT primer and RV PCR primers, the SEQ ID NOs correspond to the target-binding regions of the specific primers; as described herein, the full primers can also comprise adaptor regions and/or barcode regions. For the FW PCR primer and sequencing primers, the SEQ ID NOs correspond to the full-length primer, or a portion thereof.

TABLE 15

| | | | | | |
|---|---|---|---|---|---|
| Name | Target | RT primer | FW PCR | RV PCR | Sequencing primers |
| N#1 | SARS-CoV2 N gene (e.g., nt 131-197 of SEQ ID NO: 1001; see e.g., SEQ ID NO: 1009) | SEQ ID NO: 3 | SEQ ID NO: 14 | SEQ ID NO: 4 | SEQ ID NOs: 15 and 17 |
| N#2 | SARS-CoV-2 N gene (e.g., nt 876-1002 of SEQ ID NO: 1001; see e.g., SEQ ID NO: 1010) | SEQ ID NO: 5 | SEQ ID NO: 14 | SEQ ID NO: 6 | SEQ ID NOs: 15 and 17 |
| del6970 | SARS-CoV-2 S gene (e.g., nt 163-233 of SEQ ID NO: 1002; see e.g., SEQ ID NO: 1011) | SEQ ID NO: 7 | SEQ ID NO: 14 | SEQ ID NO: 8 | SEQ ID NOs: 15 and 17 |
| D614 | SARS-CoV-2 S gene (e.g., nt 1785-1861 of SEQ ID NO: 1002; see e.g., SEQ ID NO: 1012) | SEQ ID NO: 9 | SEQ ID NO: 14 | SEQ ID NO: 10 | SEQ ID NOs: 15 and 17 |
| positive control (enzymatic control) | SEQ ID NO: 11 | SEQ ID NO: 3 | SEQ ID NO: 14 | SEQ ID NO: 12 | SEQ ID NOs: 15 and 17 |
| RPP30 | Human RPP30 gene (e.g., nt 20-93 of SEQ ID NO: 1006) | SEQ ID NO: 1019 | SEQ ID NO: 14 | SEQ ID NO: 1020 | SEQ ID NOs: 15 and 17 |

### Protector Nucleic Acid

Described herein are protector nucleic acids (or simply “protectors”) that are capable of reducing barcode crosstalk. Such barcode crosstalk can arise due to binding of primers from the first set of the primers (i.e., RT primers) to amplification products of the RT product during the amplification step. As used herein, the term “protector nucleic acid” denotes a single-stranded nucleic acid that hybridizes to a region of an amplification product of the reverse transcription product (or RT primers) and prevents extension of the RT primer during the amplification step. Specifically, the protector nucleic acid can hybridize to an amplification product that is identical, or the same sense, as the target RNA, and comprises a region that is complementary to the target-binding region of an RT primer from the first set of primers. In some embodiments of any of the aspects, the protector nucleic acid can be DNA, RNA, modified DNA, modified RNA, synthetic DNA, synthetic RNA, or another synthetic nucleic acid.

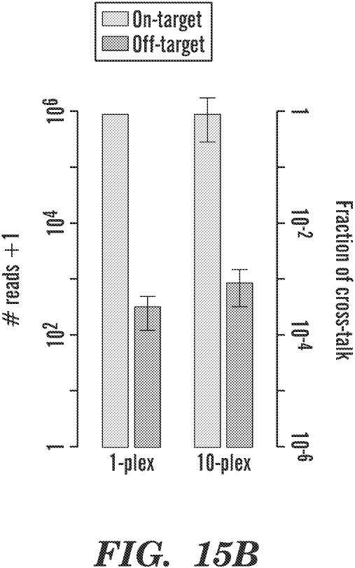

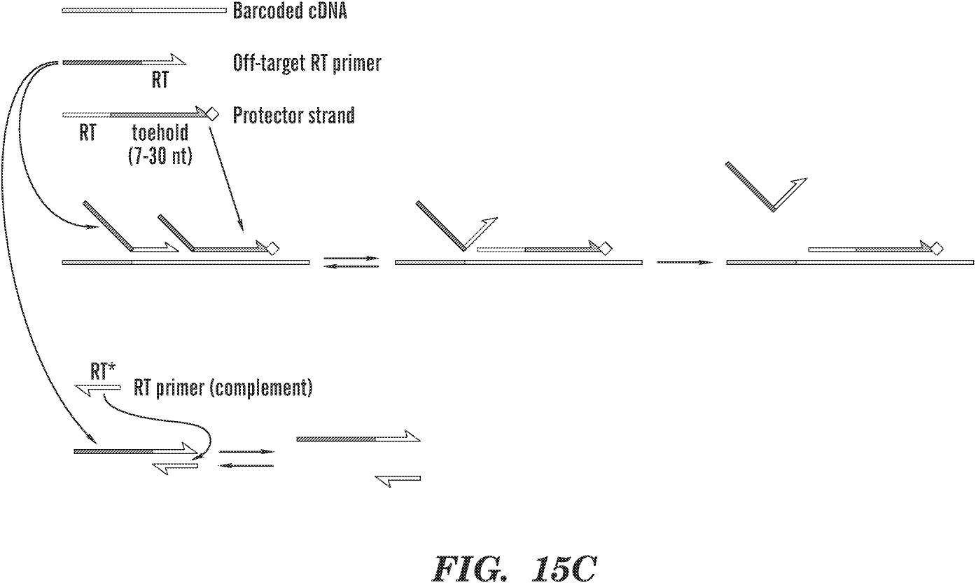

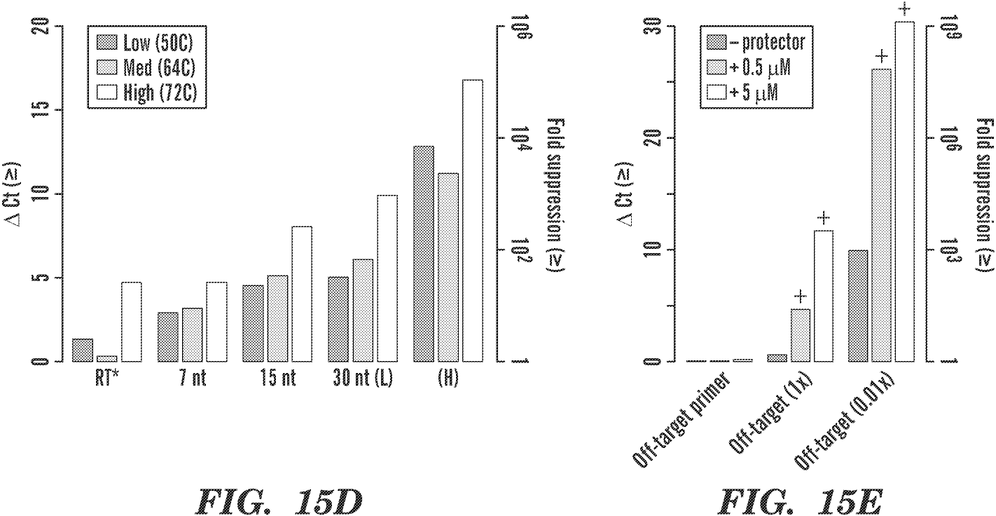

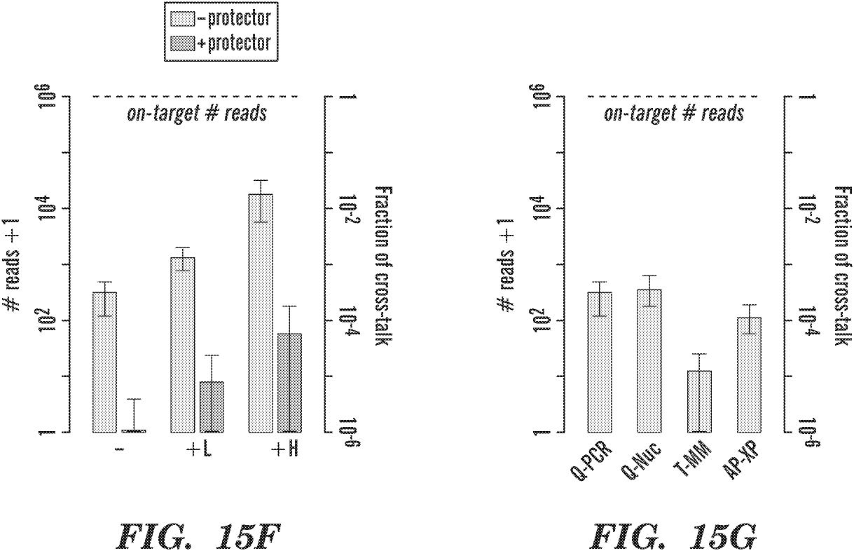

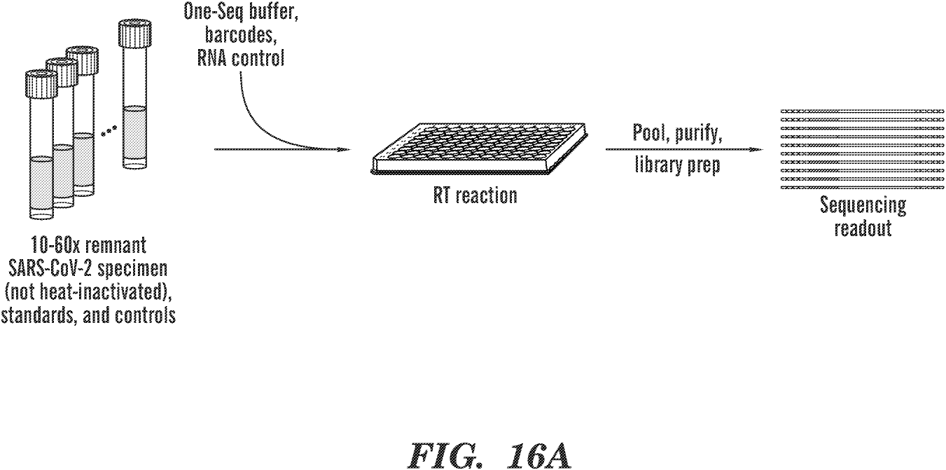

In some embodiments of any of the aspects, step (c) (amplification step) further comprises adding a protector nucleic acid to the amplification reaction mixture. In this way, the amplification reaction of step (c) comprises contacting the reverse transcription product (or pooled reverse transcription product mixture or amplification product thereof) with at least one protector nucleic acid (see e.g., upper panel of FIG. 15C). In some embodiments of any of the aspects, the protector nucleic acid comprises single stranded DNA. In some embodiments of any of the aspects, the protector nucleic acid comprises, from 5′ to 3′:(a) a region complementary or substantially complementary to a region of at least one target RNA or amplification product thereof, comprising (i) a 5′ region that is identical or substantially identical to the target-binding region of at least one primer in the first set of primers; and (ii) a 3′ region that is complementary to target RNA sequence downstream of the target-binding region of at least one primer in the first set of primers; and (b) a 3′ nucleic acid modification that inhibits synthesis of a complementary strand by a polymerase.

In some embodiments of any of the aspects, region (a)(ii) of the protector nucleic acid (also known as the “toe-hold region” or “3′ complementary region”) is at least 15 nucleotides long. In some embodiments of any of the aspects, the 3′ complementary region of the protector nucleic acid is at most 30 nucleotides long. In some embodiments of any of the aspects, the 3′ complementary region of the protector nucleic acid is at least 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt or more long.

In some embodiments of any of the aspects, an amplification product of the reverse transcription product comprises one of SEQ ID NOs: 1009-1012 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 1009-1012 that maintains the same function (RNA target region).

SEQ ID NO: 1009, N#1 target amplification product (showing only the RNA target region, e.g., nt 131-197 of SEQ ID NO: 1001); bolded text indicates where the target-binding region of the RT primer (e.g., SEQ ID NO: 3) binds (nt 49-67 of SEQ ID NO: 1009); double-underlined text indicates where an exemplary protector nucleic acid binds (e.g., SEQ ID NO: 1013); the N#1 reverse transcription product corresponds to the reverse complement of SEQ ID NO: 1009.

GTTTACCCAATAATACTGCGTCTTGGTTCACCGCTCTCACTCAACATGGC
AAGGAAGACCTTAAATT

SEQ ID NO: 1010, N#2 target amplification product (showing only the RNA target region, e.g., nt 876-1002 of SEQ ID NO: 1001); bolded text indicates where the target-binding region of the RT primer (e.g., SEQ ID NO: 5) binds (nt 111-127 of SEQ ID NO: 1010); double-underlined text indicates where an exemplary protector nucleic acid binds (e.g., SEQ ID NO: 1014); the N#2 reverse transcription product corresponds to the reverse complement of SEQ ID NO: 1010.

CAGACAAGGAACTGATTACAAACATTGGCCGCAAATTGCACAATTTGCCC
CCAGCGCTTCAGCGTTCTTCGGAATGTCGCGCATTGGCATGGAAGTCACA
CCTTCGGGAACGTGGTTGACCTACACA

SEQ ID NO: 1011, del6970 target amplification product (showing only the RNA target region, e.g., nt 163-233 of SEQ ID NO: 1002); bolded text indicates where the target-binding region of the RT primer (e.g., SEQ ID NO: 7) binds (nt 53-71 of SEQ ID NO: 1011); double-underlined text indicates where an exemplary protector nucleic acid binds (e.g., SEQ ID NO: 1015); the del6970 reverse transcription product corresponds to the reverse complement of SEQ ID NO: 1011.

TTCTTACCTTTCTTTTCCAATGTTACTTGGTTCCATGCTATACATGTCTC
TGGGACCAATGGTACTAAGAG

SEQ ID NO: 1012, D614 target amplification product showing only the RNA target region, (e.g., nt 1785-1861 of SEQ ID NO: 1002); bolded text indicates where the target-binding region of the RT primer (e.g., SEQ ID NO: 9) binds (nt 59-77 of SEQ ID NO: 1012); double-underlined text indicates where an exemplary protector nucleic acid binds (e.g., SEQ ID NO: 1016); the D614 reverse transcription product corresponds to the reverse complement of SEQ ID NO: 1012.

CAGTGTTATAACACCAGGAACAAATACTTCTAACCAGGTTGCTGTTCTTT
ATCAGGATGTTAACTGCACAGAAGTCC

In some embodiments of any of the aspects, the protector nucleic acid is complementary or substantially complementary to a region of at least one of SEQ ID NOs: 1009-1012. In some embodiments of any of the aspects, the protector nucleic acid is complementary or substantially complementary to a 3′ region of at least one of SEQ ID NOs: 1009-1012. In some embodiments of any of the aspects, the protector nucleic acid is complementary or substantially complementary to a region of at least one of SEQ ID NOs: 1009-1012 that overlaps with the region bound by the target-binding region of an RT primer (e.g., the bolded regions of SEQ ID NOs: 1009-1012).

SEQ ID NOs: 1021-1024 represent exemplary protector nucleic acids comprising: (i) a 5′ region that is identical to the target-binding region of a primer in the first set of primers (e.g., one of SEQ ID NOs: 3, 5, 7, 9); and (ii) a 30-nt-long 3′ region (i.e., toe-hold region) that is complementary to the target RNA sequence downstream of the target-binding region of the primer in the first set of primer (e.g., one of SEQ ID NOs: 3, 5, 7, 9) on the reverse transcription product.

SEQ ID NO: 1021, exemplary protector nucleic acid for the N#1 reverse transcription product (e.g., SEQ ID NO: 1009); bolded region indicates 5′ region that is identical to the target-binding region of a primer in the first set of primers (e.g., SEQ ID NO: 3) and unformatted text is the 30-nt-long toehold region: AATTTAAGGTCTTCCTTGCCATGTTGAGTGAGAGCGGTGAACCAAGACG

SEQ ID NO: 1022, exemplary protector nucleic acid for the N#2 reverse transcription product (e.g., SEQ ID NO: 1010); bolded region indicates 5′ region that is identical to the target-binding region of a primer in the first set of primers (e.g., SEQ ID NO: 5) and unformatted text is the 30-nt-long toehold region: TGTGTAGGTCAACCACGTTCCCGAAGGTGTGACTTCCATGCCAATGC

SEQ ID NO: 1023, exemplary protector nucleic acid for the del6970 reverse transcription product (e.g., SEQ ID NO: 1011); bolded region indicates 5′ region that is identical to the target-binding region of a primer in the first set of primers (e.g., SEQ ID NO: 7) and unformatted text is the 30-nt-long toehold region: CTCTTAGTACCATTGGTCCCAGAGACATGTATAGCATGGAACCAAGTAA

SEQ ID NO: 1024, exemplary protector nucleic acid for the D614 reverse transcription product (e.g., SEQ ID NO: 1012); bolded region indicates 5′ region that is identical to the target-binding region of a primer in the first set of primers (e.g., SEQ ID NO: 9) and unformatted text is the 30-nt-long toehold region: GGACTTCTGTGCAGTTAACATCCTGATAAAGAACAGCAACCTGGTTAGA

SEQ ID NOs: 1013-1016 represent exemplary protector nucleic acids comprising: (i) a 5′ region that is identical to the target-binding region of a primer in the first set of primers (e.g., one of SEQ ID NOs: 3, 5, 7, 9); and (ii) a 20-nt-long 3′ region (i.e., toe-hold region) that is complementary to the target RNA sequence downstream of the target-binding region of the primer in the first set of primer (e.g., one of SEQ ID NOs: 3, 5, 7, 9) on the reverse transcription product.

SEQ ID NO: 1013, exemplary protector nucleic acid for the N#1 reverse transcription product (e.g., SEQ ID NO: 1009); bolded region indicates 5′ region that is identical to the target-binding region of a primer in the first set of primers (e.g., SEQ ID NO: 3) and unformatted text is the 20-nt-long toehold region: AATTTAAGGTCTTCCTTGCCATGTTGAGTGAGAGCGGTG

SEQ ID NO: 1014, exemplary protector nucleic acid for the N#2 reverse transcription product (e.g., SEQ ID NO: 1010); bolded region indicates 5′ region that is identical to the target-binding region of a primer in the first set of primers (e.g., SEQ ID NO: 5) and unformatted text is the 20-nt-long toehold region: TGTGTAGGTCAACCACGTTCCCGAAGGTGTGACTTCC

SEQ ID NO: 1015, exemplary protector nucleic acid for the del6970 reverse transcription product (e.g., SEQ ID NO: 1011); bolded region indicates 5′ region that is identical to the target-binding region of a primer in the first set of primers (e.g., SEQ ID NO: 7) and unformatted text is the 20-nt-long toehold region: CTCTTAGTACCATTGGTCCCAGAGACATGTATAGCATGG

SEQ ID NO: 1016, exemplary protector nucleic acid for the D614 reverse transcription product (e.g., SEQ ID NO: 1012); bolded region indicates 5′ region that is identical to the target-binding region of a primer in the first set of primers (e.g., SEQ ID NO: 9) and unformatted text is the 20-nt-long toehold region: GGACTTCTGTGCAGTTAACATCCTGATAAAGAACAGCAA

In some embodiments of any of the aspects, the protector nucleic acid comprises one of SEQ ID NOs: 1013-1016 or SEQ ID NOs: 1021-1024 or functional fragment thereof or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 1013-1016 or SEQ ID NOs: 1021-1024 that maintains the same function (e.g., protector nucleic acid, reduction of barcode crosstalk during amplification step).

In some embodiments of any of the aspects, the protector nucleic acid comprises a nucleic acid modification capable of inhibiting synthesis of a complementary strand by a polymerase. In some embodiments of any of the aspects, the protector nucleic acid comprises a 3′ nucleic acid modification capable of inhibiting synthesis of a complementary strand by a polymerase. In some embodiments of any of the aspects, the 3′ nucleic acid modification is selected from the group consisting of: (a) an inverted base; (b) a spacer; (c) a dideoxynucleotide; (d) a base that is not complementary to the target RNA; and (e) a non-canonical base.

In some embodiments of any of the aspects, the nucleic acid modification capable of inhibiting synthesis of a complementary strand by a polymerase is an inverted nucleotide. As used herein, the term “inverted nucleotide” refers to a nucleotide that is inserted by a DNA polymerase inverted onto a DNA molecule; e.g., the 3′ OH group is used for polymerization, as opposed to the 5′ OH group. In some embodiments of any of the aspects, the inverted nucleotide is an inverted dT, inverted dA, inverted dG, or inverted dC. In some embodiments of any of the aspects, the inverted nucleotide is a 3′ Inverted dT. Inverted dT can be incorporated at the 3′-end of the protector nucleic acid, leading to a 3′-3′ linkage which inhibits both degradation by 3′ exonucleases and extension by DNA polymerases.

In some embodiments of any of the aspects, the nucleic acid modification capable of inhibiting synthesis of a complementary strand by a polymerase is a spacer. In some embodiments of any of the aspects, the spacer is located at an internal location of one or both primers. Non-limiting examples of spacers include the C3 spacer (phosphoramidite); hexanediol; 1′,2′-Dideoxyribose (dSpacer; e.g., an abasic site); Spacer 9 (a triethylene glycol spacer); and Spacer 18 (an 18-atom hexa-ethyleneglycol spacer).

In some embodiments of any of the aspects, the nucleic acid modification capable of inhibiting synthesis of a complementary strand by a polymerase is a dideoxynucleotide. Dideoxynucleotides are chain-elongating inhibitors of DNA polymerase, e.g., used in the Sanger method for DNA sequencing. The dideoxynucleotides, when attached or incorporated at the 3′ end of an oligonucleotide or a growing strand do not present a substrate for elongation by DNA polymerase. Dideoxynucleotides are also known as 2′,3′ because both the 2′ and 3′ positions on the ribose lack hydroxyl groups, and are abbreviated as ddNTPs (ddGTP, ddATP, ddTTP and ddCTP). In some embodiments of any of the aspects, the nucleic acid modification capable of inhibiting synthesis of a complementary strand by a polymerase is selected from the group consisting of ddGTP, ddATP, ddTTP and ddCTP.

In some embodiments of any of the aspects, the nucleic acid modification capable of inhibiting synthesis of a complementary strand by a polymerase is a base that is not complementary to the target RNA. As a non-limiting example, A-T and G-C represent proper base-pairing; as such, non-limiting examples of non-complementary base-paring include: A-G, A-C, A-A, G-T, G-G, C-A, T-T, T-C, T-G, C-C, C-T, or C-A. If the final 3′ nucleotide of an oligonucleotide is not complementary to the template, it cannot be extended.

In some embodiments of any of the aspects, the nucleic acid modification capable of inhibiting synthesis of a complementary strand by a polymerase is a non-canonical base. In some embodiments of any of the aspects, the non-canonical bases is isocytosine (iso-dC). In some embodiments of any of the aspects, the non-canonical bases is isoguanosine (iso-dG).

In some embodiments of any of the aspects, the protector nucleic acid displaces a primer from the first set of primers from an amplification product of the reverse transcription product. In some embodiments of any of the aspects, the protector nucleic acid inhibits or substantially inhibits a primer from the first set of primers from being extended by the DNA polymerase. In some embodiments of any of the aspects, the protector nucleic acid has a higher binding affinity to an amplification product of the reverse transcription product than the target-binding region of the at least one primer from the first set of primers.

In some embodiments of any of the aspects, the protector nucleic acid has a higher Tm than the target-binding region of the at least one primer from the first set of primers. In some embodiments of any of the aspects, the protector nucleic acid has a Tm that is at least 1° C., at least 2° C., at least 3° C., at least 4° C., at least 5° C., at least 6mL, at least 7° C., at least 8° C., at least 9° C., at least 10° C., at least 11° C., at least 12° C., at least 13° C., at least 14° C., at least 15° C., at least 16° C., at least 17° C., at least 18° C., at least 19° C., or at least 20° C. higher than the target-binding region of the at least one primer from the first set of primers.

In some embodiments of any of the aspects, step (c) (the amplification step) further comprises contacting at least one primer from the first set of primers, e.g., if present in the amplification reaction, with a protector nucleic acid (see e.g., lower panel of FIG. 15C). In some embodiments of any of the aspects, the protector nucleic acid comprises a region that is complementary or substantially complementary to the target-binding region of at least one primer from the first set of primers (e.g., complementary to at least a portion of one of SEQ ID NOs: 3, 5, 7, or 9). In some embodiments of any of the aspects, the protector nucleic acid inhibits or substantially inhibits a primer from the first set of primers from binding to the reverse transcription product.

In some embodiments of any of the aspects, the protector nucleic acid is at least 15 nucleotides long. In some embodiments of any of the aspects, the protector nucleic acid is at least 30 nucleotides long. In some embodiments of any of the aspects, the protector nucleic acid is at least 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt or more long.

In some embodiments of any of the aspects, the protector nucleic acid is present at a concentration that is greater than the concentration of the primers in the first set of primers. In some embodiments of any of the aspects, the protector nucleic acid is present at a concentration of at least 0.5 uM. In some embodiments of any of the aspects, the protector nucleic acid is present at a concentration of at least 2.0 uM. In some embodiments of any of the aspects, the protector nucleic acid is present, e.g., in the amplification reaction, at a concentration of at least 0.1 uM, at least 0.2 uM, at least 0.3 uM, at least 0.4 uM, at least 0.5 uM, at least 0.6 uM, at least 0.7 uM, at least 0.8 uM, at least 0.9 uM, at least 1 uM, at least 2 uM, at least 3 uM, at least 4 uM, at least 5 uM, at least 6 uM, at least 7 uM, at least 8 uM, at least 9 uM, at least 10 uM, or more.

In some embodiments of any of the aspects, prior to step (c) the first set of barcoded primers is substantially removed, for example, using a bead-based purification method or a spin-column-based purification method, and during step (c) the reverse transcription product or amplification product thereof is contacted with at least one protector nucleic acid.

### Amplification Reaction

In some embodiments of any of the aspects, step (c) comprises a nucleic acid amplification method. In some embodiments of any of the aspects, the amplification step comprises 35-50 rounds or cycles of amplification in which the DNA polymerase replicates the cDNA using forward and reverse primers in the second set of primers. In some embodiments of any of the aspects, the product of the amplification step comprises a barcoded dsDNA library, each comprising a region that is complementary to a portion of at least one target RNA.

In some embodiments of any of the aspects, the amplification step comprises contacting the pooled reverse transcription product mixture with a DNA polymerase, a second set of primers, optionally at least one protector nucleic acid, and an amplification reaction buffer. In some embodiments of any of the aspects, the amplification step further comprises contacting the reverse transcription product with carrier nucleic acid, e.g., poly-A60 DNA oligonucleotide and/or E. coli tRNA. In some embodiments of any of the aspects, the carrier nucleic acid can be provided at a similar concentration as in the RT step.

In some embodiments of any of the aspects, step (c) (the amplification step) further comprises contacting the reverse transcription product with Uracil-DNA Glycosylase (UDG or UNG) enzyme. UNG can be used to eliminate carryover polymerase chain reaction (PCR) products. This method modifies PCR products such that in a new reaction, any residual products from any previous PCR amplifications are digested and prevented from amplifying, but the true cDNA templates are unaffected. PCR synthesizes abundant amplification products each round, but contamination of further rounds of PCR with trace amounts of these products, called carry-over contamination (e.g., on surfaces of a laboratory), yields false positive results. Carry-over contamination from some previous PCR can be a significant problem, due both to the abundance of PCR products, and to the ideal structure of the contaminant material for re-amplification. In some embodiments, carry-over contamination can be controlled by the following two steps: (i) incorporating dUTP in all PCR products (e.g., by substituting dUTP for dTTP, either completely or partially, or by incorporating uracil during synthesis of primers); and (ii) treating all subsequent fully preassembled starting reactions with uracil DNA glycosylase (UDG), followed by thermal inactivation of UDG. UDG cleaves the uracil base from the phosphodiester backbone of uracil-containing DNA, but has no effect on natural (i.e., thymine-containing) DNA. The resulting apyrimidinic sites block replication by DNA polymerases, and are very labile to acid/base hydrolysis. Because UDG does not react with dTTP, and is also inactivated by heat denaturation prior to the actual PCR, carry-over contamination of PCRs can be controlled effectively if the contaminants contain uracils in place of thymines.

In some embodiments of any of the aspects, the amplification reaction buffer comprises dNTPs (e.g., dATP, dGTP, dCTP, and dTTP). In some embodiments of any of the aspects, the amplification reaction buffer comprises UNG and dNTPs (e.g., dATP, dGTP, dCTP, dUTP, and/or +/dTTP). In some embodiments of any of the aspects, the reaction buffer maintains the reaction at specific optimal pH (e.g., 8.3) and can include such components as water, Tris-HCl, KCl, MgCl2, and other buffers or salts.

In some embodiments of any of the aspects, the amplification reaction buffer comprises a detectable marker, e.g., for the presence of amplification product, e.g., dsDNA. In some embodiments of any of the aspects, the amount of amplification product can be determined by quantitative PCR (QPCR) or real-time PCR methods, e.g., using a set of primers specific to the amplification product and/or SYBR® GREEN, or an equivalent dye, or a detectable probe. Methods of qPCR and real-time qPCR are known in the art.

In some embodiments of any of the aspects, step (c) (the amplification step) comprises: (i) a denaturation step; and (ii) an annealing step; and (iii) an extension step. In some embodiments of any of the aspects, step (c) (e.g., the amplification step) is performed in a thermocycler. In some embodiments of any of the aspects, (i)-(iii) of the amplification (e.g., PCR) are repeated at least 30 times (e.g., 30-40 times). In some embodiments of any of the aspects, (i) and (ii) of the amplification (e.g., PCR) are repeated at least 30, 31, 32, 33, 34, 35, 36, 37, 38, 39, 40, 41, 42, 43, 44, 45, 46, 47, 48, 49, 50, or more times.

In some embodiments of any of the aspects, step (c) (the amplification step) further comprises an initial denaturation step before the first step (i) at least 95° C. for at least 60 seconds. Such an initial denaturation step can denature the cDNA, the UNG enzyme, and/or the reverse transcriptase. In some embodiments of any of the aspects, the initial denaturation step is performed at temperature of at least 90° C., at least 91° C., at least 92° C., at least 93° C., at least 94° C., at least 95° C., at least 96° C., at least 97° C., at least 98° C., at least 99° C., at least 99° C., or at least 99.5° C. In some embodiments of any of the aspects, the initial denaturation step is performed for at least 10 seconds, at least 20 second, at least 30 seconds, at least 40 seconds, at least 50 seconds, at least 1 minute, at least 2 minutes, at least 3 minutes, at least 4 minutes, at least 5 minutes, at least 6 minutes, at least 7 minutes, at least 8 minutes, at least 9 minutes, at least 10 minutes or more.

In some embodiments of any of the aspects, step (i) of the amplification (e.g., the denaturation step) is performed at a temperature of at least 95° C. for at least 15 seconds (sec). In some embodiments of any of the aspects, step (i) of the amplification (e.g., the denaturation step) is performed at a temperature of at least 90° C., at least 91° C., at least 92° C., at least 93° C., at least 94° C., at least 95° C., at least 96° C., at least 97° C., at least 98° C., at least 99° C., at least 99° C., or at least 99.5° C. In some embodiments of any of the aspects, step (i) of the amplification (e.g., the denaturation step) is performed for at least 5 sec, at least 6 sec, at least 7 sec, at least 8 sec, at least 9 sec, at least 10 sec, at least 11 sec, at least 12 sec, at least 13 sec, at least 14 sec, at least 15 sec, at least 16 sec, at least 17 sec, at least 18 sec, at least 19 sec, at least 20 sec, at least 21 sec, at least 22 sec, at least 23 sec, at least 24 sec, at least 25 sec, at least 26 sec, at least 27 sec, at least 28 sec, at least 29 sec, at least 30 sec or more.

In some embodiments of any of the aspects, step (ii) of the amplification (e.g., the annealing step) is performed at a temperature of at least 60° C. for at least 30 seconds. In some embodiments of any of the aspects, step (ii) of the amplification (e.g., the annealing step) is performed at a temperature of at least 60° C. In some embodiments of any of the aspects, step (ii) of the amplification (e.g., the annealing step) is performed at a temperature of at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., at least 71° C., at least 72° C., at least 73° C., at least 74° C., at least 75° C. or more. In some embodiments of any of the aspects, step (ii) of the amplification (e.g., the annealing step) is performed for at least 30 seconds. In some embodiments of any of the aspects, step (ii) of the amplification (e.g., the annealing step) is performed for at least 15 sec, at least 20 sec, at least 25 sec, at least 30 sec, at least 35 sec, at least 40 sec, at least 45 sec, at least 50 sec, at least 55 sec, at least 60 sec, at least 65 sec, at least 70 sec, at least 75 sec, at least 80 sec, at least 85 sec, at least 90 sec, at least 95 sec, at least 100 sec, at least 105 sec, at least 110 sec, at least 115 sec, or at least 120 sec or more.

In some embodiments of any of the aspects, the at least first iteration of step (ii) of the amplification (e.g., the annealing step) is performed at a lower temperature than subsequent iterations of step (ii). In some embodiments of any of the aspects, the first two iterations of step (ii) of the amplification (e.g., the annealing step) are performed at a temperature of at least 52° C. In some embodiments of any of the aspects, the first 1, 2, 3, 4, 5, or more iterations of step (ii) of the amplification (e.g., the annealing step) are performed at a temperature of at least 52° C. In some embodiments of any of the aspects, the first 1, 2, 3, 4, 5, or more iterations of step (ii) (e.g., the annealing step) of the amplification are performed at a temperature of at least 58° C. In some embodiments of any of the aspects, the first 1, 2, 3, 4, 5, or more iterations of step (ii) (e.g., the annealing step) of the amplification are performed at a temperature of at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., or at least 65° C.

In some embodiments of any of the aspects, the subsequent iterations of step (ii) (e.g., after the first two iterations of step (ii), e.g., the annealing step) are performed at a temperature of at least 68° C. In some embodiments of any of the aspects, the subsequent iterations of step (ii) (e.g., after the first 1, 2, 3, 4, 5, or more iterations of step (ii) of the amplification, e.g., the annealing step) are performed at a temperature of at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., at least 71° C., at least 72° C., at least 73° C., at least 74° C., or at least 75° C.

In some embodiments of any of the aspects, step (iii) of the amplification (e.g., the extension step) is performed at a temperature of at least 72° C. for at least 30 seconds. In some embodiments of any of the aspects, step (iii) of the amplification (e.g., the extension step) is performed at a temperature of at least 72° C. In some embodiments of any of the aspects, step (ii) of the amplification (e.g., the annealing step) is performed at a temperature of at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., at least 71° C., at least 72° C., at least 73° C., at least 74° C., at least 75° C., at least 76° C., at least 77° C., at least 78° C., at least 79° C., or at least 80° C. or more. In some embodiments of any of the aspects, step (iii) of the amplification (e.g., the extension step) is performed for at least 30 seconds. In some embodiments of any of the aspects, step (ii) of the amplification (e.g., the extension step) is performed for at least 15 sec, at least 20 sec, at least 25 sec, at least 30 sec, at least 35 sec, at least 40 sec, at least 45 sec, at least 50 sec, at least 55 sec, at least 60 sec, at least 65 sec, at least 70 sec, at least 75 sec, at least 80 sec, at least 85 sec, at least 90 sec, at least 95 sec, at least 100 sec, at least 105 sec, at least 110 sec, at least 115 sec, or at least 120 sec, at least 130 sec, at least 140 sec, at least 150 sec, at least 160 sec, at least 170 sec, at least 180 sec, at least 190 sec, at least 200 sec or more.

In some embodiments of any of the aspects, step (c) (the amplification step) further comprises contacting at least one reverse transcription product (or at least one primer from the first set of primers, if present) with a protector nucleic acid, and wherein step (ii) (e.g., the annealing step) is performed at a temperature of at least 64° C. In some embodiments of any of the aspects, step (c) (the amplification step) further comprises contacting at least one reverse transcription product (or at least one primer from the first set of primers, if present) with a protector nucleic acid, and wherein step (ii) (e.g., the annealing step) is performed at a temperature of at least 72° C. In some embodiments of any of the aspects, step (c) (the amplification step) further comprises contacting at least one reverse transcription product (or at least one primer from the first set of primers, if present) with a protector nucleic acid, and wherein step (ii) (e.g., the annealing step) is performed at a temperature of at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., at least 71° C., at least 72° C., at least 73° C., at least 74° C., or at least 75° C.

In some embodiments of any of the aspects, step (c) (the amplification step) further comprises contacting at least one reverse transcription product with a protector nucleic acid, and at least one of the following: (I) step (ii) (e.g., the annealing step) is performed at a temperature of at least 64° C.; (II) the 3′ complementary region (i.e., toe-hold region) of the protector nucleic acid is at least 20 nucleotides long; and/or (III) the protector nucleic acid is present at a concentration of at least 0.5 uM. In some embodiments of any of the aspects, (I) step (ii) is performed at a temperature of at least 64° C. In some embodiments of any of the aspects, (II) the 3′ complementary region of the protector nucleic acid is at least 20 nucleotides long. In some embodiments of any of the aspects, (III) the protector nucleic acid is present at a concentration of at least 0.5 uM. In some embodiments of any of the aspects, (I) step (ii) is performed at a temperature of at least 64° C.; and (II) the 3′ complementary region of the protector nucleic acid is at least 20 nucleotides long. In some embodiments of any of the aspects, (I) step (ii) is performed at a temperature of at least 64° C.; and (III) the protector nucleic acid is present at a concentration of at least 0.5 uM. In some embodiments of any of the aspects, (II) the 3′ complementary region of the protector nucleic acid is at least 20 nucleotides long; and (III) the protector nucleic acid is present at a concentration of at least 0.5 uM. In some embodiments of any of the aspects, (I) step (ii) is performed at a temperature of at least 64° C.; (II) the 3′ complementary region of the protector nucleic acid is at least 20 nucleotides long; and (III) the protector nucleic acid is present at a concentration of at least 0.5 uM.

In some embodiments of any of the aspects, step (c) (the amplification step) further comprises contacting at least one reverse transcription product with a protector nucleic acid, and at least one of the following: (I) step (ii) (e.g., the annealing step) is performed at a temperature of at least 68° C.; (II) the 3′ complementary region (i.e., toe-hold region) of the protector nucleic acid is at least 30 nucleotides long; and/or (III) the protector nucleic acid is present at a concentration of at least 2.0 uM. In some embodiments of any of the aspects, (I) step (ii) is performed at a temperature of at least 68° C. In some embodiments of any of the aspects, (II) the 3′ complementary region of the protector nucleic acid is at least 30 nucleotides long. In some embodiments of any of the aspects, (III) the protector nucleic acid is present at a concentration of at least 2.0 uM. In some embodiments of any of the aspects, (I) step (ii) is performed at a temperature of at least 68° C.; and (II) the 3′ complementary region of the protector nucleic acid is at least 30 nucleotides long. In some embodiments of any of the aspects, (I) step (ii) is performed at a temperature of at least 68° C.; and (III) the protector nucleic acid is present at a concentration of at least 2.0 uM. In some embodiments of any of the aspects, (II) the 3′ complementary region of the protector nucleic acid is at least 30 nucleotides long; and (III) the protector nucleic acid is present at a concentration of at least 2.0 uM. In some embodiments of any of the aspects, (I) step (ii) is performed at a temperature of at least 68° C.; (II) the 3′ complementary region of the protector nucleic acid is at least 30 nucleotides long; and (III) the protector nucleic acid is present at a concentration of at least 2.0 uM.

In some embodiments of any of the aspects, at least 2 batches of amplification products from step (c) (the amplification step) are combined in one container. As used herein, the term “batch” refers to the combined products from one reaction, e.g., the barcoded amplification products from a single amplification reaction. In some embodiments of any of the aspects, at least 10 amplification product batches from step (c) (the amplification step) are combined in one container. In some embodiments of any of the aspects, at least 2 batches, at least 3 batches, at least 4 batches, at least 5 batches, at least 6 batches, at least 7 batches, at least 8 batches, at least 9 batches, at least 10 batches, at least 15 batches, at least 20 batches, at least 25 batches, at least 30 batches, at least 35 batches, at least 40 batches, at least 45 batches, at least 50 batches, at least 55 batches, at least 60 batches, at least 65 batches, at least 70 batches, at least 75 batches, at least 80 batches, at least 85 batches, at least 90 batches, at least 95 batches, at least 100 batches or more of amplification products from step (c) are combined in one container.

In some embodiments of any of the aspects, the amplification step is performed in at most 30 minutes. As a non-limiting example, the amplification step is performed in at most 20 minutes, at most 25 minutes, at most 30 minutes, at most 40 minutes, at most 50 minutes, at most 60 minutes, at most 70 minutes, at most 80 minutes, at most 90 minutes, at most 100 minutes, at most 110 minutes, at most 120 minutes, at most 130 minutes, at most 140 minutes, at most 150 minutes, at most 160 minutes, at most 170 minutes, or at most 180 minutes. The specific conditions, e.g., of temperature, time, and buffer conditions can be varied as necessary to accommodate different DNA polymerases.

In one aspect, described herein is an amplification composition comprising at least two of the following: (a) a barcoded reverse transcription product; (b) a second set of primers; (c) DNA polymerase; (c) Uracil-DNA Glycosylase (UDG) enzyme; and/or (d) a protector nucleic acid. It is noted that a composition can comprise any one, two, three, or all four of the components listed above.

### Sequencing

In some embodiments as described further herein, nucleic acid samples (e.g., amplified nucleic acid samples) can be sequenced. Accordingly, the detection method comprises sequencing the amplification products, thereby detecting at least one target RNA, if present, in the at least two samples Sequencing is the process of determining the order of monomers in a polymer. For example, DNA or RNA sequencing is the process of determining a nucleic acid sequence - the order of nucleotides in DNA or RNA, respectively, from a sample. DNA or RNA sequencing can also be referred to herein as “nucleic acid sequencing” or simply “sequencing.”

In some embodiments of any of the aspects, prior to step (d) (the sequencing step) the second set of barcoded primers are substantially removed. In some embodiments of any of the aspects, prior to step (d) (the sequencing step) the second set of barcoded primers are substantially removed using, for example, a bead-based purification method or a spin-column-based purification method.

Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of hybridization of probes, or measuring one or more characteristics of a single molecule as it passes through a sensor (e.g. the modulation of an electrical field as a nucleic acid molecule passes through a nanopore).

In some embodiments as described herein, nucleic acid sequence data can be obtained from a sequencing platform. The term “sequencing platform” refers not only to a particular machine or device used for sequencing, but also to the particular chemical and/or physical approaches applied to extract or derive the sequence information from a sample. Exemplary methods of sequencing include, but are not limited to, Sanger sequencing, dideoxy chain termination, high-throughput sequencing, next generation sequencing, pyrosequencing (e.g., 454), sequencing by ligation and detection (SOLiD™), polony sequencing, sequencing by synthesis (e.g., Illumina™), ion semiconductor sequencing (e.g., Ion Torrent™), sequencing by hybridization, nanopore sequencing, HeliScope single molecule sequencing, single-molecule real-time sequencing (SMRT), RNAP sequencing, combinatorial probe anchor synthesis (cPAS), nanopore sequencing, chain termination sequencing, DNA nanoball sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Early methods of DNA sequencing, or “first generation sequencing,” included Sanger sequencing (also known as chain terminator sequencing) and Maxam-Gilbert sequencing (also known as chemical sequencing). High-throughput sequencing methods have significantly reduced the cost and time to sequence nucleic acid samples. High-throughput sequencing can also be referred to herein as “next-generation sequencing”, “second-generation sequencing”, “third-generation sequencing”, or “massively parallel signature sequencing (MPSS)”.

Non-limiting examples of ion semiconductor sequencing platforms include Ion Torrent™ sequencing platforms comprising Ion S5™, Ion AmpliSeq™, Ion Proton™, Ion PGM™ (e.g., PGM 314™, PGM 316™, PGM 318™, PI™, or PII™), or Ion Chef™ platforms, from ThermoFisher™ (see e.g., U.S. Pat. 7,785,785, US 8552771, US8692298B2, US8731847B2, US8742472B2, US8841217B1, US8912580B2, US8912005B1, US8962366B2, US8963216B2, US9116117B2, US9128044B2, US9194000B2, US9239313B2, US9404920B2, US9841398B2, US9927393B2, US9944981B2, US9958414B2, US9960253B2, which are incorporated herein by reference in their entireties).

Pyrosequencing, an example of sequencing by synthesis, can also be referred to as 454 Life Sciences™ sequencing, 454 sequencing, or 454 pyrosequencing. Non-limiting examples of 454 pyrosequencing platforms include Genome Sequencer FLX™, GS20™, or GS Junior™ sequencing platforms. Pyrosequencing can also be performed on any the following sequencing platforms from QIAGEN: PyroMark Q48 Autoprep™, PyroMark Q24 Advanced™, PyroMark Q24™, or PyroMark Q96 ID™ (see e.g., U.S. Pat. US 6,210,891, US 7,323,305, US 8,748,102, US 8,765,380, which are incorporated herein by reference in their entireties).

Sequencing by synthesis methods include, for example, Illumina™ sequencing or Solexa™ sequencing. Non-limiting examples of Illumina™ sequencing platforms include cBot™, Genome Analyzer (GA)™, MiniSeq™, NextSeq™, MiSeq™, HiSeq 2500™, HiSeq 3000™, HiSeq 4000™, HiSeq X™ (e.g., Hiseq Ten™), iSeq™ 100, HiScan™, and iScan™ Illumina platforms (see e.g., U.S. Pat. US 7,414,116, US 7,329,860, US 7,589,315, US 7,960,685, US 8,039,817, US 8,071,962, US 8,158,926, US 8,241,573, US 8,778,848, US 8,778,849, US 8,244,479, US 8,315,817, US 8,412,467, US 8,422,031, US 8,446,573, US 8,914,241, US 8,965,076, US 9,012,022, US 9,068,220, US 9,121,063, US 9,365,898, US 9,410,977, US 9,512,422, US 9,540,690, US 9,670,535, US 9,752,186, US 9,777,325, US 9,994,687, US 10,005,083, US 10,053,730, US 10,152,776, which are incorporated herein by reference in their entireties).

Additional non-limiting examples of sequencing by synthesis platforms can comprise GeneReader™ from QIAGEN or Mini-20™ from AZCO Biotech™, Inc.

Non-limiting examples of SMRT sequencing platforms include C1™, C2™, P4-XL™, P5-C3™, P6-C4™, RS™, RS II™, or Sequel™ platforms, all from PacBio™ sequencing. SMRT sequencing can also be referred to as PacBio™ sequencing.

Non-limiting examples of cPAS sequencing platforms includeBGISEQ-50™, MGISEQ 200™, BGISEQ-500™, or MGISEQ-2000™ cPAS platforms. cPas sequencing platforms can also utilize DNA nanoball sequencing methods (e.g., BGISEQ-500™, or MGISEQ-2000™).

Non-limiting examples of SOLiD™ sequencing platforms include 5500x1 SOLiD™, 5500 SOLiD™, SOLiD 5500xl Wildfire™, or SOLiD 5500 Wildfire™, from Thermo Fisher Scientific™.

Non-limiting examples of Nanopore sequencing platforms include SmidgeION™, MinION™, and PromethION™, all from Oxford Nanopore Technologies™.

Non-limiting examples of chain termination sequencing platforms can comprise Microfluidic Sanger sequencing platforms or the Apollo 100™ platform (Microchip Biotechnologies™, Inc.).

Non-limiting examples of Polony sequencing platforms include a Polonator™ platform (Dover™) or fluorescence microscope and a computer controlled flowcell.

Non-limiting examples of HeliScope single molecule sequencing platforms include Helicos® Genetic Analysis System platform or the HeliScope™ Sequencer.

Additional non-limiting examples of sequencing methods include tunneling currents DNA sequencing, sequencing by hybridization, sequencing with mass spectrometry, microscopy-based techniques, RNA polymerase (RNAP) sequencing, or in vitro virus high-throughput sequencing.

In some embodiments of any of the aspects, the sequencing method is sequencing by synthesis. In some embodiments of any of the aspects, the sequencing method is Illumina™ sequencing. In some embodiments of any of the aspects, the sequencing method comprises contacting the amplification products with a third set of primers, comprising at least first and second sequencing primers. In some embodiments of any of the aspects, the first and second sequencing primers comprise at least one of SEQ ID NOs: 15 and 17 or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 15 and 17 that maintains the same function (e.g., priming for sequencing by synthesis). In some embodiments of any of the aspects, the first and second sequencing primers comprise an adaptor-binding region that is complementary or substantially complementary to the adaptor region of a primer in the first or second set of primers.

In some embodiments of any of the aspects, the sequencing method produces a sequencing read from the first or second sequencing primer (see e.g., FIG. 20A, SEQ ID NOs: 993-994). In some embodiments of any of the aspects, the sequencing read from the first sequencing primer (e.g., SEQ ID NO: 15) comprises the sequence of the first barcode region (i.e., sample ID or patient ID) from a primer in the first primer set (see e.g., FIG. 20A, SEQ ID NO: 993). In some embodiments of any of the aspects, the sequencing read from the second sequencing primer (e.g., SEQ ID NO: 17) comprises the sequence of the first and second barcode regions from a primer in the first primer set. In some embodiments of any of the aspects, the sequencing read from the second sequencing primer (e.g., SEQ ID NO: 17) comprises the sequence of the second barcode region (i.e., batch barcode) from a primer in the second primer set (see e.g., FIG. 20A, SEQ ID NO: 994).

In some embodiments of any of the aspects, the sequencing read from the first or second sequencing primer (e.g., SEQ ID NOs: 15 or 17) comprises sequence from the target RNA (e.g., one of SEQ ID NOs: 1009-1012 or the reverse complement thereof). In some embodiments of any of the aspects, the sequencing read from the first or second sequencing primer comprises at least one variation of interest in the target RNA.

In some embodiments of any of the aspects, the target RNA is detected in the sample if a first and second barcode region associated with the specific target RNA is detected in the sequencing read of the amplification product. In some embodiments of any of the aspects, the target RNA is detected in the sample if at least one first barcode region associated with the specific target RNA is detected in the sequencing read of the amplification product. In some embodiments of any of the aspects, the target RNA is detected in the sample if at least one second barcode region associated with the specific target RNA is detected in the sequencing read of the amplification product. In some embodiments of any of the aspects, the target RNA is not detected in the sample if a first or second barcode region associated with the specific target RNA is not detected in the sequencing read of the amplification product. In some embodiments of any of the aspects, if the target RNA is not present in the sample, then no barcode regions associated with the specific target RNA is detected in the sequencing reads of the amplification product.

In some embodiments of any of the aspects, at least n target RNAs in a single sample are detected, and the at least n target RNAs are on the same assayed RNA molecule. In some embodiments of any of the aspects, the assayed RNA molecule is determined to be present in the sample if at least one of the n target RNAs are detected. In some embodiments of any of the aspects, the assayed RNA molecule is determined to not be present in the sample if none of the n target RNAs are detected.

### Kits

Another aspect of the technology described herein relates to kits for detecting a target RNA. Described herein are kit components that can be included in one or more of the kits described herein. In one aspect, described herein is a kit for detecting a target RNA in a sample, comprising: at least one of the following (a) a reverse transcriptase; (b) a first set of primers comprising at least one barcode; (c) a detergent; (d) a carrier nucleic acid; (e) a positive control nucleic acid; (f) at least one stabilization agent; (g) at least two containers; (h) a DNA polymerase; (i) a second set of primers; (j) Uracil-DNA Glycosylase (UDG) enzyme; (k) a protector nucleic acid; and/or (i) a third set of primers.

In some embodiments of any of the aspects, the kit comprises a reverse transcriptase. In some embodiments of any of the aspects, the kit is used to reverse transcribe target RNA into DNA, and to amplify the DNA to a detectable amplification product. In some embodiments of any of the aspects, the reverse transcriptase is selected from the group consisting of: a Moloney murine leukemia virus (M-MLV) reverse transcriptase (RT), an avian myeloblastosis virus (AMV) RT, a retrotransposon RT, a telomerase reverse transcriptase, an HIV-1 reverse transcriptase, or a recombinant version thereof. In some embodiments of any of the aspects, the reverse transcriptase is provided at a sufficient amount, such that, e.g., at least 200 U/µL, can be added to the RT reaction mixture.

In some embodiments of any of the aspects, the kit comprises a DNA polymerase. In some embodiments of any of the aspects, the DNA polymerase is a Thermus aquaticus (Taq) DNA polymerase or variant thereof. In some embodiments of any of the aspects, the DNA polymerase(s) is provided at a sufficient amount to be added to the amplification reaction mixture.

In some embodiments of any of the aspects, the kit comprises a first set of primers (e.g., for RT), comprising at least one barcode. In some embodiments of any of the aspects, the first set of primers comprises primers that bind to target RNA and provide an adaptor region (e.g., a PCR adaptor region). In some embodiments of any of the aspects, the kit comprises a second set of primers (e.g., for amplification). In some embodiments of any of the aspects, the second set of primers is specific (i.e., binds specifically through complementarity) to cDNA, in other words, the DNA produced in the RT step that is complementary to the target RNA. In some embodiments of any of the aspects, the second set of primers provides adaptors for sequencing. In some embodiments of any of the aspects, the kit comprises a third set of primers (e.g., for sequencing). In some embodiments of any of the aspects, the first, second, and/or third sets of primers are provided at a sufficient concentration, e.g., 25 uM to 500 uM, to be added to associated reaction mixture.

In some embodiments of any of the aspects, the kit comprises carrier nucleic acid, e.g., poly-A60 DNA oligonucleotide and/or E. coli tRNA, provided at a sufficient concentration to be added to the RT and/or amplification reaction. In some embodiments of any of the aspects, the kit comprises at least one positive control nucleic acid, provided at a sufficient concentration to be added to the RT reaction. In some embodiments of any of the aspects, the positive control nucleic acid is a positive sample control nucleic acid or a positive enzymatic control nucleic acid. In some embodiments of any of the aspects, the kit further comprises detergent, e.g., Triton-X10, provided at a sufficient concentration to be added to the RT reaction.

In some embodiments of any of the aspects, the kit comprises a stabilization agent, provided at a sufficient concentration to be added to the RT reaction. In some embodiments of any of the aspects, the kit comprises at least one of the following stabilization agents: (a) an RNase inhibitor; (b) a metal-chelating agent; (c) a reducing agent; d) an antibiotic; (e) an antimycoctic; and/or (f) a protease inhibitor (or any combination thereof, see e.g., Table 13).

In some embodiments of any of the aspects, the kit comprises at least one protector nucleic acid, provided at a sufficient concentration to be added to the amplification reaction. In some embodiments of any of the aspects, the at least one protector nucleic acid reduces or inhibits barcode crosstalk in the amplification reaction. In some embodiments of any of the aspects, the kit comprises Uracil-DNA Glycosylase (UDG) enzyme, provided at a sufficient concentration to be added to the amplification reaction, which can reduce or inhibit detection of amplification product contaminants.

In some embodiments of any of the aspects, the kit comprises at least two containers, such that at least two RT reactions can be combined into one amplification reaction, and/or at least two amplification reactions can be combined into one sequencing reaction. In some embodiments of any of the aspects, the container is a test tube, centrifuge tube, multi-well plate, and the like.

In some embodiments of any of the aspects, the kit further comprises a reaction buffer for the RT reaction and/or a reaction buffer for the amplification reaction. Such reaction buffers can comprise at least one of the following: diluent, water, magnesium acetate (or another magnesium compound such as magnesium chloride), and/or dNTPs. In some embodiments of any of the aspects, the kit further comprises a sample collection device, such a swab. In some embodiments of any of the aspects, the kit further comprises a sample collection container, optionally containing transport media. In some embodiments of any of the aspects, the kit further comprises reagents for a bead-based purification method or a spin-column-based purification method. In some embodiments of any of the aspects, the kit further comprises at least one negative control. Non-limiting examples of negative controls for SARS-CoV-2 include MERS, SARS, 229e, NL63, and hKul, which can be detected using specific primers.

In some embodiments, the kit comprises an effective amount of the reagents as described herein. As will be appreciated by one of skill in the art, the reagents can be supplied in a lyophilized form or a concentrated form that can diluted or suspended in liquid prior to use. The kit reagents described herein can be supplied in aliquots or in unit doses.

In some embodiments, the components described herein can be provided singularly or in any combination as a kit. Such a kit includes the components described herein and packaging materials thereof. In addition, a kit optionally comprises informational material.

In some embodiments, the compositions in a kit can be provided in a watertight or gas tight container which in some embodiments is substantially free of other components of the kit. For example, the reagents described herein can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The informational material can be descriptive, instructional, marketing or other material that relates to the methods described herein. The informational material of the kits is not limited in its form. In some embodiments, the informational material can include information about production of the reagents, concentration, date of expiration, batch or production site information, and so forth. In some embodiments, the informational material relates to methods for using or administering the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Systems

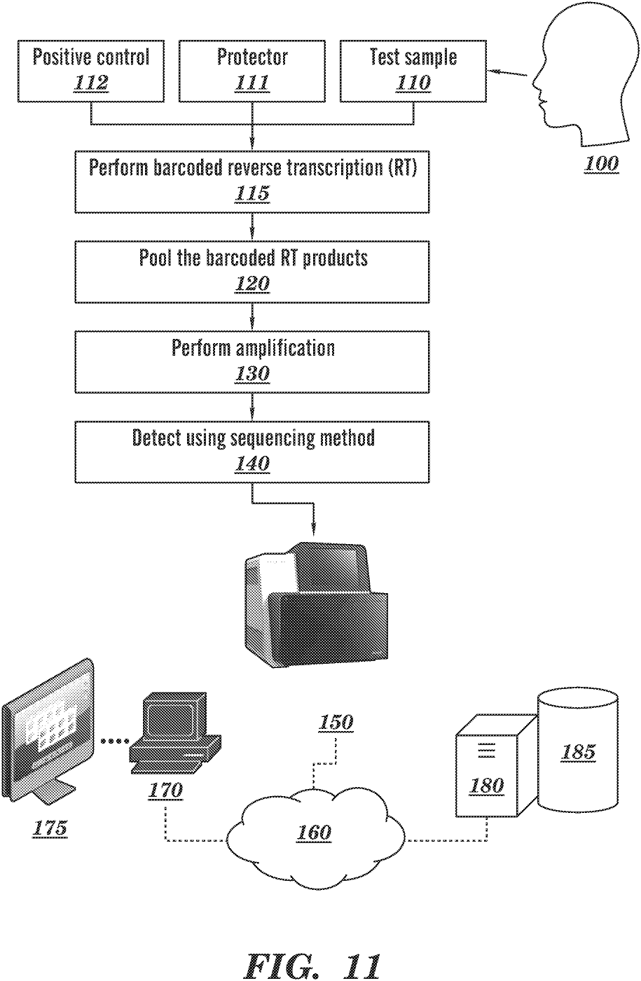

FIG. 11 shows an exemplary schematic of a system as described herein. In some embodiments of any of the aspects, a test sample 110 is collected from a subject. A protector nucleic acid 111 and/or a positive control nucleic acid 112 can also be provided. In separate or combined reactions, the barcoded RT reaction 115 is performed using the first set of primers comprising at least one barcode. Next, at least two barcoded RT products are pooled 120 into at least one container. The pooled reverse transcription product mixture is then subjected to an amplification reaction 130, which are optionally pooled following the amplification. The amplification products are then sequenced 140 using a high-throughput sequencer 150. The sequencer 150 outputs its results to a network 160.

The computing device 170 and server 180 can be connected by a network 160 and the network 160 can be connected to various other devices, servers, or network equipment for implementing the present disclosure. A computing device 170 can be connected to a display 175. Computing device 170 can be any suitable computing device, including a desktop computer, server (including remote servers), mobile device, or other suitable computing device. A computing device 170 can be used to view or process sequencer 150 data. Data output from the sequencer 150 can also be input into a program that can be stored in a database 185. In some examples, sequencing data as described herein and other associated software can be stored in database 185 and run on server 180. Additionally, sequencing data processed or produced by said programs can be stored in database 185.

It should initially be understood that the methods and systems described herein can be implemented with any type of hardware and/or software, and can include use of a pre-programmed general purpose computing device. For example, the system can be implemented using a server, a personal computer, a portable computer, a thin client, or any suitable device or devices. The kits, methods and/or components for the performance thereof can include the use of a single device at a single location, or multiple devices at a single, or multiple, locations that are connected together using any appropriate communication protocols over any communication medium such as electric cable, fiber optic cable, or in a wireless manner.

It should also be noted that the systems as described herein can be arranged or used in a format having a plurality of modules which perform particular functions. It should be understood that these modules are merely schematically illustrated based on their function for clarity purposes only, and do not necessary represent specific hardware or software. In this regard, these modules can be hardware and/or software implemented to substantially perform the particular functions discussed. Moreover, the modules can be combined together within the disclosure, or divided into additional modules based on the particular function desired. Thus, the disclosure should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be performed in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of these. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC as noted above.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from, or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. In other words, the term “hybridization” refers to the process in which two single-stranded polynucleotides bind non-covalently through hydrogen bonding to form a stable double-stranded polynucleotide. The term “hybridization” may also refer to triple-stranded hybridization. The resulting (usually) double-stranded polynucleotide is a “hybrid” or “duplex.”

As used herein, the term “complementary” refers to nucleic acid sequences that are capable of base-pairing according to the standard Watson-Crick complementary rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “substantial” refers to of ample or considerable amount, quantity, or size as determined by a user. As a non-limiting example, the term “substantially complementary” refers to a nucleic acid that is at least at least 90%, at least 91%, at least 92%, at least 93%, at least 94%, at least 95%, at least 96%, at least 97%, at least 98%, at least 99%, or more complementary to another nucleic acid. As another non-limiting example, the term “substantially identical” refers to a nucleic acid that is at least at least 90%, at least 91%, at least 92%, at least 93%, at least 94%, at least 95%, at least 96%, at least 97%, at least 98%, at least 99%, or more identical to another nucleic acid. The term “essentially complementary” can be used interchangeably with “substantially complementary.” The term “essentially identical” can be used interchangeably with “substantially identical.”

As used herein, a “barcode” is an artificial DNA sequence that provides an indication, e.g., of sample origin, target identity or other information regarding a sequencing target. In one embodiment, the presence of a barcode can be an indicator that a target sequence is or was present in a given starting sample. In general, a barcode should not be substantially identical to or substantially complementary to any sequence of the genome of a host or to the genome of, e.g., a virus one wishes to detect. Similarly, the barcodes used in a given method should not be substantially complementary to other barcodes used in that method, i.e., the barcodes are members of a minimally cross-hybridizing set. That is, the nucleotide sequence of each member of such a barcode set is sufficiently different from that of every other member of the set that no member can form a stable duplex with the complement of any other member under stringent hybridization conditions. Barcodes can vary in length, but will generally be at least 4 nucleotides in length. Longer barcodes are contemplated, but will generally be less than 36 nucleotides in length. In some embodiments, barcodes can each have a length within a range of from 4 to 36 nucleotides, or from 6 to 30 nucleotides, or from 8 to 20 nucleotides. For more details concerning barcode technologies, see e.g., U.S. Pat. US9902950, US10233490; U.S. Pat. Publications US20150298091, US2018032017, US20180216160; international patent publications WO2015164212, WO2013192292; Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101: 11046; and Brenner (2004) Genome Biol. 5:240; the contents of each of which are incorporated herein by reference in their entireties.

By adding a barcode to a primer with another region that specifically binds or hybridizes to a sequence one wishes to detect, detection of the barcode by sequencing becomes a surrogate for reading the actual signal of the target nucleic acid. When the only way to obtain an amplification product to sequence is to have a target nucleic acid present in an initial reverse-transcription and/or amplification reaction, one only needs to sequence the barcode to determine that the target sequence was present in the initial sample. Barcoding can also be used to indicate, for example, which sample a given sequence read belongs to. For example, when each sample is reverse transcribed using a primer that includes a barcode unique to that sample, detection of the sample-indicating barcode identifies which sample a given sequence read arose from. A combination of two or more barcodes can therefore provide significant information without the need to read into the actual target sequence, if so desired. For example, a primer including two barcodes (or a set or sets of primers including two barcodes), one correlating with target identity (indicating presence or absence of an RNA target) and one indicating which sample the read came from (a sample-specific barcode) can identify which sample, e.g., which individual subject, and which target nucleic acid is present in that sample without the need to sequence beyond the two barcodes, if so desired. As another example, a primer including two barcodes (or a set or sets of primers including two barcodes), one correlating with sample identity (a sample-specific barcode) and one correlating with batch identity (a batch-specific barcode indicating the reverse transcription batch) can identify the sample and reaction batch; sequencing in between the barcodes can determine the specific target sequence. In this manner, very high throughput diagnostics, e.g., viral diagnostics, can be realized. Of course, additional sequence information beyond just the barcodes can be and often is obtained using NGS approaches. In addition to simply obtaining more sequence beyond the barcodes through longer reads, reads beyond the barcodes can provide information on variants of a given target, for example.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of viral infection. A subject can be male or female.

A subject can be one who has been previously diagnosed with or identified as suffering from or having a condition in need of treatment (e.g. a viral infection) or one or more complications related to such a condition, and optionally, have already undergone treatment for a viral infection or the one or more complications related to a viral infection. Alternatively, a subject can also be one who has not been previously diagnosed as having a viral infection or one or more complications related to a viral infection. For example, a subject can be one who exhibits one or more risk factors for a viral infection or one or more complications related to a viral infection or a subject who does not exhibit risk factors. A “subject in need” of testing for a particular condition can be a subject having that condition, diagnosed as having that condition, or at risk of developing that condition.

In the various embodiments described herein, it is contemplated that variants (naturally occurring or otherwise), alleles, homologs, conservatively modified variants, and/or conservative substitution variants of any of the particular polypeptides described (e.g., reverse transcriptase, DNA polymerase, etc.) are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) nonpolar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, G1n; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into G1n or into His; Asp into Glu; Cys into Ser; G1n into Asn; Glu into Asp; Gly into Ala or into Pro; His into Asn or into Gln; Ile into Leu or into Val; Leu into Ile or into Val; Lys into Arg, into G1n or into Glu; Met into Leu, into Tyr or into Ile; Phe into Met, into Leu or into Tyr; Ser into Thr; Thr into Ser; Trp into Tyr; Tyr into Tip; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide’s activity according to the assays described herein. A functional fragment can comprise conservative substitutions of the sequences disclosed herein.

In some embodiments, a polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments, the methods described herein relate to measuring, detecting, or determining the level of at least one target, e.g., the target RNA. As used herein, the term “detecting” or “measuring” refers to observing a signal from, e.g. a sequencing read, a probe, label, or target molecule to indicate the presence of an analyte in a sample. Any method known in the art for detecting a particular label moiety can be used for detection. Exemplary detection methods include, but are not limited to, sequencing, spectroscopic, fluorescent, photochemical, biochemical, immunochemical, electrical, optical or chemical methods. In some embodiments of any of the aspects, measuring can be a quantitative observation. Sequence determination, e.g., that indicates or confirms the presence of a given barcode region is a form of detecting used herein.

In some embodiments of any of the aspects, a polypeptide or nucleic acid as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, “contacting” refers to any suitable means for delivering, or exposing, an agent to at least one component as described herein (e.g., sample, target RNA, cDNA, amplification product, etc.). In some embodiments, contacting comprises physical human activity, e.g., an injection; an act of dispensing, mixing, and/or decanting; and/or manipulation of a delivery device or machine.

As used herein, the term “specific binding” refers to a chemical or physical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%. In some embodiments of any of the aspects, the term “about” when used in connection with percentages can mean ±5%.

As used herein, the term “comprising” means that other elements can also be present in addition to the defined elements presented. The use of “comprising” indicates inclusion rather than limitation.

The term “consisting of refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), W. W. Norton & Company, 2016 (ISBN 0815345054, 978-0815345053); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments can perform functions in a different order, or functions can be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments can also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A multiplexed method of detecting at least one target RNA in at least two samples, comprising:
   a) contacting the at least two samples with a reverse transcriptase and a first primer or first set of primers comprising at least a first barcode, under conditions permitting the generation of reverse transcription products;
   b) combining reverse transcription products from samples in step (a) in one container to form a pooled reverse transcription product mixture;
   c) contacting the pooled reverse transcription product mixture with a DNA polymerase and a set of second primers under conditions permitting the generation of amplification products; and
   d) sequencing the amplification products, thereby detecting at least one target RNA, if present, in the at least two samples.
2. The method of paragraph 1, wherein step (b) is performed before step (c).
3. The method of paragraph 1 or paragraph 2, wherein steps (a)-(d) are performed sequentially.
4. The method of any one of paragraphs 1-3, wherein the detection method has a limit of detection of at least 500 target RNA copies per mL for a given target RNA.
5. The method of any one of paragraphs 1-4, wherein the detection method has a limit of detection of at least 1000 target RNA copies per mL for a given target RNA.
6. The method of any one of paragraphs 1-5, wherein the detection method has a dynamic range of at least 3 logs.
7. The method of any one of paragraphs 1-6, wherein at least 2 target RNAs in a single sample are detected.
8. The method of paragraph 7, wherein the at least 2 target RNAs are on the same RNA molecule.
9. The method of paragraph 7, wherein the at least 2 target RNAs are on different RNA molecules.
10. The method of any one of paragraphs 1-9, wherein at least one target RNA is a viral RNA.
11. The method of paragraph 10, wherein at least 2 target RNAs are from the same virus.
12. The method of paragraph 10, wherein at least 2 target RNAs are from at least 2 different viruses.
13. The method of paragraph 10, wherein at least one viral RNA is a SARS-CoV-2 RNA.
14. The method of any one of paragraphs 1-13, wherein target RNAs from at least 50 samples are detected in a single performance of steps (a) - (d).
15. The method of any one of paragraphs 1-14, wherein prior to step (a), the at least one target RNA is not extracted from the sample.
16. The method of any one of paragraphs 1-15, wherein the reverse transcriptase (RT) is an engineered or recombinant version of an Moloney Murine Leukemia Virus (MMLV) RT, Avian Myeloblastosis Virus (AMV) RT, or another naturally occurring RT.
17. The method of any one of paragraphs 1-16, wherein the first primer or each primer in the first set of primers comprises, from 5′ to 3′:
   a) an adaptor region;
   b) a first barcode region; and
   c) a target-binding region that is complementary or substantially complementary to and permits hybridization to at least one target RNA.
18. The method of any one of paragraphs 1-17, wherein the first primer or each primer in the first set of primers comprises, from 5′ to 3′:
   a) an adaptor region;
   b) a first barcode region;
   c) a second barcode region; and
   d) a target-binding region that is complementary or substantially complementary to and permits hybridization to at least one target RNA.
19. The method of any one of paragraphs 1-18, wherein the barcode region of a first primer in the first set of barcoded primers is a Hamming distance of at least 10 from each other barcode region of any other primer in the first set of barcoded primers.
20. The method of any one of paragraphs 1-19, wherein the first or second barcode region on the first primer or set of first primers comprises one of SEQ ID NOs: 18-989.
21. The method of any one of paragraphs 1-20, wherein at least one barcode region on the first primer or set of first primers corresponds to and is different for each of the at least two samples.
22. The method of any one of paragraphs 1-21, wherein at least one barcode region on the first primer or set of first primers corresponds to and is different for each of the target RNAs.
23. The method of any one of paragraphs 1-22, wherein the target-binding region of a primer in the first set of primers binds at most 5 nucleotides away from a variation of interest in the target RNA.
24. The method of paragraph 23, wherein the variation of interest is selected from the group consisting of: a single-nucleotide variation; a point mutation; a substitution; an insertion; and a deletion.
25. The method of paragraph 23 or 24, wherein the target RNA is SARS-CoV-2 S gene and the variation of interest is selected from the group consisting of: del69-70, del144, K417N, K417T, L452R, E484K, N501Y, D614G, P681H, and A701V.
26. The method of any one of paragraphs 1-25, wherein step (a) further comprises contacting the sample with a detergent.
27. The method of paragraph 26, wherein the detergent lyses viral particles or cells in the sample.
28. The method of paragraph 26 or 27, wherein the detergent releases target RNA from the sample.
29. The method of any one of paragraphs 26-28, wherein the detergent is a nonionic surfactant.
30. The method of any one of paragraphs 26-29, wherein the detergent is Triton X-100.
31. The method of any one of paragraphs 1-30, wherein step (a) further comprises contacting the sample with carrier nucleic acid.
32. The method of paragraph 31, wherein the carrier nucleic acid reduces loss of the target RNA.
33. The method of paragraph 31 or 32, wherein the carrier nucleic acid is poly-A60 DNA oligonucleotide or E. coli tRNA.
34. The method of any one of paragraphs 1-33, wherein step (a) further comprises contacting the sample with a positive control nucleic acid.
35. The method of paragraph 34, wherein the positive control nucleic acid is a primer comprising from 5′ to 3′:
   a) an adaptor region;
   b) a first barcode region; and
   c) a target-binding region that is complementary to or substantially complementary to a sample nucleic acid.
36. The method of paragraph 34, wherein the positive control nucleic acid comprises, from 5′ to 3′:
   a) a region that is not identical or substantially identical to any target RNA being assayed; and
   b) a region that is identical or substantially identical to at least one target RNA.
37. The method of paragraph 36, wherein the region of the positive control nucleic acid that is identical or substantially identical to at least one target RNA is complementary or substantially complementary to the target-binding region of at one least primer from the first set of primers.
38. The method of any one of paragraphs 34-37, wherein the positive control nucleic acid comprises SEQ ID NO: 11.
39. The method of any one of paragraphs 34-38, wherein the sample is contacted with at least 100-10⁴ copies/ul of positive control nucleic acid.
40. The method of any one of paragraphs 1-39, wherein step (a) further comprises contacting the samples with a stabilization agent.
41. The method of paragraph 40, wherein the stabilization agent prevents degradation of the RNA target and/or reverse transcriptase for at least 6 hours at room temperature.
42. The method of paragraph 40 or 41, wherein the stabilization agent prevents degradation of the RNA target and/or reverse transcriptase for at least 24 hours at room temperature.
43. The method of any one of paragraphs 40-42, wherein the stabilization agent is an RNA-preserving agent or a reverse-transcriptase-preserving agent.
44. The method of paragraph 43, wherein the RNA-preserving agent is an RNase inhibitor, a metal-chelating agent, or a reducing agent.
45. The method of paragraph 44, wherein the RNase inhibitor is murine RNase inhibitor or a thermostable RNase inhibitor.
46. The method of paragraph 44, wherein the metal-chelating agent is ethylenediaminetetraacetic acid (EDTA).
47. The method of paragraph 44, wherein the reducing agent is dithiothreitol (DTT).
48. The method of paragraph 43, wherein the reverse-transcriptase-preserving agent is an antibiotic, an antimycotic, or a protease inhibitor.
49. The method of any one of paragraphs 1-48, wherein step (a) comprises a reverse transcription reaction.
50. The method of any one of paragraphs 1-49, wherein step (a) comprises:
   i) incubating the sample, reverse transcriptase, and first primer or first set of primers comprising at least one barcode at a temperature of at least 50° C. for at least 30 minutes; and
   ii) inactivating the reverse transcription reaction at a temperature of at least 95° C. for at least 5 minutes.
51. The method of any one of paragraphs 1-50, wherein the reverse transcription products from step (a) comprise a barcoded DNA comprising a region that is complementary to a portion of at least one target RNA.
52. The method of any one of paragraphs 1-51, wherein reverse transcription products from step (a) from at least 5 different samples are combined in one container.
53. The method of any one of paragraphs 1-52, wherein prior to step (c) the first set of barcoded primers is substantially removed.
54. The method of any one of paragraphs 1-53, wherein prior to step (c) the target RNA and/or sample is substantially removed.
55. The method of any one of paragraphs 1-54, wherein prior to step (c) the first set of barcoded primers or the RNA target is substantially removed using a bead-based purification method or a spin-column-based purification method.
56. The method of any one of paragraphs 1-55, wherein the DNA polymerase is a thermostable DNA polymerase I.
57. The method of any one of paragraphs 1-56, wherein the DNA polymerase is a Thermus aquaticus (Taq) DNA polymerase.
58. The method of any one of paragraphs 1-57, wherein the second set of primers comprises forward and reverse amplification primers.
59. The method of any one of paragraphs 1-58, wherein the forward primer in the second set of primers comprises from 5′ to 3′:
   a) an adaptor region; and
   b) an adaptor-binding region that is identical or substantially identical to the adaptor region of a primer in the first set of barcoded primers.
60. The method of any one of paragraphs 1-58, wherein a forward primer in the second set of primers comprises from 5′ to 3′:
   a) an adaptor region;
   b) a third barcode region; and
   c) an adaptor-binding region that is identical or substantially identical to the adaptor region of a primer in the first set of barcoded primers.
61. The method of any one of paragraphs 1-60, wherein a reverse primer in the second set of primers comprises, from 5′ to 3′:
   a) an adaptor region;
   b) a second barcode region; and
   c) a target-binding region that is identical or substantially identical to at least one target RNA.
62. The method of any one of paragraphs 1-60, wherein a reverse primer in the second set of primers comprises, from 5′ to 3′:
   a) an adaptor region; and
   b) a region that is identical or substantially identical to at least one target RNA.
63. The method of any one of paragraphs 1-62, wherein the barcode region of a first primer in the second set of barcoded primers is a Hamming distance of at least 5 from each other barcode region of any other primer in the second set of barcoded primers.
64. The method of any one of paragraphs 1-63, wherein the second or third barcode region in the second set of primers comprises one of SEQ ID NOs: 18-989.
65. The method of any one of paragraphs 1-64, wherein step (c) further comprises contacting the reverse transcription product with Uracil-DNA Glycosylase (UDG) enzyme.
66. The method of any one of paragraphs 1-65, wherein step (c) further comprises contacting the reverse transcription product or amplification product thereof with a protector nucleic acid.
67. The method of paragraph 66, wherein the protector nucleic acid comprises single stranded DNA.
68. The method of paragraph 66 or 67, wherein the protector nucleic acid comprises, from 5′ to 3′:
   a) a region complementary or substantially complementary to a region of at least one target RNA or amplification product thereof, comprising
      i) a 5′ region that is identical or substantially identical to the target-binding region of at least one primer in the first set of primers; and
      ii) a 3′ region that is complementary to the target RNA sequence downstream of the target-binding region of at least one primer in the first set of primers; and
   b) a 3′ nucleic acid modification that inhibits synthesis of a complementary strand by a polymerase.
69. The method of paragraph 68, wherein the 3′ complementary region of the protector nucleic acid is at least 15 nucleotides long.
70. The method of paragraph 68, wherein the 3′ complementary region of the protector nucleic acid is at most 30 nucleotides long
71. The method of paragraph 68, wherein the 3′ nucleic acid modification is selected from the group consisting of:
   a) an inverted base;
   b) a spacer;
   c) a dideoxynucleotide;
   d) a base that is not complementary to the target RNA; and
   e) a non-canonical base.
72. The method of any one of paragraphs 66-71, wherein the protector nucleic acid displaces a primer from the first set of primers from an amplification product of the reverse transcription product.
73. The method of any one of paragraphs 66-72, wherein the protector nucleic acid inhibits or substantially inhibits a primer from the first set of primers from being extended by the DNA polymerase.
74. The method of any one of paragraphs 66-73, wherein the protector nucleic acid has a higher binding affinity to an amplification product of the reverse transcription product than the target-binding region of the at least one primer from the first set of primers.
75. The method of any one of paragraphs 66-74, wherein the protector nucleic acid has a higher Tm than the target-binding region of the at least one primer from the first set of primers.
76. The method of any one of paragraphs 66-75, wherein the protector nucleic acid inhibits or substantially inhibits a primer from the first set of primers from binding to an amplification product of the reverse transcription product.
77. The method of any one of paragraphs 66-76, wherein the protector nucleic acid is at least 15 nucleotides long.
78. The method of any one of paragraphs 66-77, wherein the protector nucleic acid is at least 30 nucleotides long.
79. The method of any one of paragraphs 66-78, wherein the protector nucleic acid is present at a concentration that is greater than the concentration of the primers in the first set of primers.
80. The method of any one of paragraphs 66-79, wherein the protector nucleic acid is present at a concentration of at least 0.5 uM.
81. The method of any one of paragraphs 66-80, wherein the protector nucleic acid is present at a concentration of at least 2.0 uM.
82. The method of any one of paragraphs 1-81, wherein step (c) comprises a nucleic acid amplification method.
83. The method of paragraph 82, wherein the amplification method comprises polymerase chain reaction amplification (PCR).
84. The method of paragraph 82 or 83, wherein step (c) comprises:
   i) a denaturation step;
   ii) an annealing step;
   iii) and an extension step wherein steps (i)-(iii) are repeated at least 30 times.
85. The method of 83 or 84, wherein step (c) further comprises an initial denaturation step before the first step (i) at least 95° C. for at least 60 seconds.
86. The method of paragraphs 84 or 85, wherein step (i) is performed at a temperature of at least 95° C. for at least 15 seconds.
87. The method of any one of paragraphs 84-86, wherein step (ii) is performed at a temperature of at least 60° C. for at least 30 seconds.
88. The method of any one of paragraphs 84-87, wherein the first two iterations of step (ii) are performed at a temperature of at least 52° C.
89. The method of any one of paragraphs 84-88, wherein the iterations of step (ii) after the first two iterations of step (ii) are performed at a temperature of at least 68° C.
90. The method of any one of paragraphs 84-89, wherein step (iii) is performed at a temperature of at least 72° C. for at least 30 seconds.
91. The method of any one of paragraphs 84-90, wherein step (c) further comprises contacting at least one reverse transcription product with a protector nucleic acid, and wherein step (ii) is performed at a temperature of at least 64° C.
92. The method of any one of paragraphs 84-91, wherein step (c) further comprises contacting at least one reverse transcription product with a protector nucleic acid, and wherein step (ii) is performed at a temperature of at least 72° C.
93. The method of any one of paragraphs 84-92, wherein step (c) further comprises contacting at least one reverse transcription product with a protector nucleic acid, and at least one of the following:
   I) step (ii) is performed at a temperature of at least 64° C.;
   II) the 3′ complementary region of the protector nucleic acid is at least 20 nucleotides long; and/or
   III) the protector nucleic acid is present at a concentration of at least 0.5 uM.
94. The method of any one of paragraphs 84-93, wherein step (c) further comprises contacting at least one reverse transcription product with a protector nucleic acid, and at least one of the following:
   I) step (ii) is performed at a temperature of at least 68° C.;
   II) the 3′ complementary region of the protector nucleic acid is at least 30 nucleotides long; and/or
   III) the protector nucleic acid is present at a concentration of at least 2.0 uM.
95. The method of any one of paragraphs 1-94, wherein at least 10 amplification product sets from step (c) are combined in one container.
96. The method of any one of paragraphs 1-95, wherein prior to step (d) the second set of barcoded primers are substantially removed.
97. The method of any one of paragraphs 1-96, wherein prior to step (d) the second set of barcoded primers are substantially removed using a bead-based purification method or a spin-column-based purification method.
98. The method of any one of paragraphs 1-97, wherein the sequencing method is a high-throughput sequencing method.
99. The method of any one of paragraphs 1-98, wherein the sequencing method is selected from the group consisting of: sequencing by synthesis, dideoxy chain termination sequencing, pyrosequencing, sequencing by ligation and detection, polony sequencing, ion semiconductor sequencing, sequencing by hybridization, and nanopore sequencing.
100. The method of any one of paragraphs 1-99, wherein the sequencing method is sequencing by synthesis.
101. The method of any one of paragraphs 1-100, wherein the sequencing method comprises contacting the amplification products with a third set of primers, comprising at least first and second sequencing primers.
102. The method of paragraph 101, wherein the first and second sequencing primers comprise an adaptor-binding region that is complementary or substantially complementary to the adaptor region of a primer in the first or second set of primers.
103. The method of paragraph 101 or 102, wherein the sequencing method produces a sequencing read from the first or second sequencing primer.
104. The method of any one of paragraphs 101-103, wherein the sequencing read from the first sequencing primer comprises the sequence of the first barcode region from a primer in the first primer set.
105. The method of any one of paragraphs 101-104, wherein the sequencing read from the second sequencing primer comprises the sequence of the first and second barcode regions from a primer in the first primer set.
106. The method of any one of paragraphs 101-105, wherein the sequencing read from the second sequencing primer comprises the sequence of the second barcode region from a primer in the second primer set.
107. The method of any one of paragraphs 101-106, wherein the sequencing read from the first or second sequencing primer comprises sequence from the target RNA.
108. The method of any one of paragraphs 101-107, wherein the sequencing read from the first or second sequencing primer comprises at least one variation of interest in the target RNA.
109. The method of any one of paragraphs 1-108, wherein the target RNA is detected in the sample if a first and second barcode region associated with the specific target RNA is detected in the sequencing read of the amplification product.
110. The method of any one of paragraphs 1-109, wherein the target RNA is not detected in the sample if a first or second barcode region associated with the specific target RNA is not detected in the sequencing read of the amplification product.
111. The method of any one of paragraphs 1-110, wherein at least n target RNAs in a single sample are detected, and the at least n target RNAs are on the same assayed RNA molecule.
112. The method of paragraph 111, wherein the assayed RNA molecule is:
   i) determined to be present in the sample if at least one of the n target RNAs are detected; or
   ii) determined to not be present in the sample if none of the n target RNAs are detected.
113. A method of preparing at least two pooled barcoded amplification sets from at least one target RNA in at least two samples, comprising the sequential steps of:
   a) contacting the at least two samples with a reverse transcriptase and a first primer or first set of primers comprising at least a first barcode, under conditions permitting the generation of reverse transcription products;
   b) combining reverse transcription products from samples in step (a) in one container to form a pooled reverse transcription product mixture; and
   c) contacting the pooled reverse transcription product mixture with a DNA polymerase and a set of second primers under conditions permitting the generation of amplification products.
114. A reverse transcription solution comprising:
   a) a reverse transcriptase;
   b) a first set of primers comprising at least one barcode;
   c) a detergent;
   d) carrier nucleic acid;
   e) at least one positive control nucleic acid;
   f) at least one stabilization agent; and/or
   g) reverse transcription reaction buffer.
115. A collection tube containing the reverse transcription solution of paragraph 114.
116. A kit for detecting a target RNA in a sample, comprising:
   a) a reverse transcriptase;
   b) a first set of primers comprising at least one barcode;
   c) a detergent;
   d) a carrier nucleic acid;
   e) a positive control nucleic acid;
   f) at least one stabilization agent;
   g) at least two containers;
   h) a DNA polymerase;
   i) a second set of primers;
   j) Uracil-DNA Glycosylase (UDG) enzyme;
   k) a protector nucleic acid; and/or
   l) a third set of primers.
117. A composition comprising:
   a) a target RNA;
   b) a reverse transcriptase;
   c) a first primer or a first set of primers comprising at least one barcode;
   d) a detergent;
   e) a carrier nucleic acid;
   f) a positive control nucleic acid; and/or
   g) at least one stabilization agent.
118. A composition comprising:
   a) a barcoded reverse transcription product;
   b) a second set of primers;
   c) DNA polymerase;
   d) Uracil-DNA Glycosylase (UDG) enzyme; and/or
   e) a protector nucleic acid.

### EXAMPLES

### Example 1: Highly-Multiplexed Viral RNA Detection by High-Throughput Sequencing

This project addresses the urgent need of high-throughput viral diagnostics. The rapid, exponential spread of the COVID-19 virus in the US and across the world has forced a switch from a containment to a mitigation strategy. A national-scale lockdown, while maybe effective in the short run, is neither sustainable nor economically affordable. Learning from the experience of countries like China and South Korea, one strategy for resolving this crisis is to perform viral screening (and regular monitoring) at the population level - isolate the infected; let the others go to work. In particular, in a situation where there are significant numbers of infected but asymptomatic individuals in the population, population-wide testing is of vital importance. However, such a strategy requires a tremendously high testing capacity (e.g., >100,000,000 tests). As of Mar. 23, 2020, 80,000 tests had been performed across the US, with a testing capacity (e.g., <10,000 per day) that was not even enough to test all symptomatic patients. Even with the introduction of the high-volume testing systems (e.g., 10x higher throughput than conventional RT-PCR), there is at least a 100- to 1000-fold gap in testing capacity relative to need.

Described herein is an approach that uses a DNA barcoding strategy for multiplexed sample detection, to allow for massively parallel viral detection in 1,000 or more patient samples and several viral species, simultaneously. To achieve this, the method takes advantage of the tremendously high throughput of next-generation sequencing (NGS) platforms (e.g., 10 million reads per run on an Illumina MiSeq™ machine, and 10 billion reads on a NovaSeq™). Importantly, hundreds of these sequencing machines are set up in academic institutes and centralized core facilities across the country, and are readily convertible to clinical testing centers to meet the current urgent diagnostic needs. The method described herein allows highly-multiplexed viral testing (e.g., COVID-19, SARS, H1N1) in thousands of patient samples in a few hours, with an amortized instrument and reagent cost of <$1 per test. Successful implementation of this method allows massive-scale viral surveillance at a population level and can immediately impact the course of an infectious disease, such as the COVID19 pandemic. As well as identifying asymptomatic carriers, these surveillance results provide critical data for better epidemiological understanding of the spatial and temporal dynamics of viral transmission. Apart from viral detection, the method further provides the ability of (e.g., partial) viral sequencing to allow monitoring of new subspecies and better understanding of its mutational and transmission dynamics. Combined, these results play a critical role in evaluating effective strategies (e.g., social isolation) and guiding public policy making for subsequent phases (e.g., months or years to come) in the battle against infectious disease, while reducing negative economic and social impacts.

### Highly-Multiplexed RNA Barcoding and Detection by High-Throughput Sequencing

Described herein is development of the workflow for highly-multiplexed RNA barcoding, sample pooling, library preparation and sequencing readout. Synthetic COVID-19 viral RNA (commercially available, e.g., from ATCC) is used as a test target. Tests involve multiplexing specificity and cross-talk and determination of the limit of detection, dynamic range, and uniformity of barcode detection sensitivity. One can test for and optimize different barcoding probes and reverse transcription primer designs, various reaction conditions (e.g., concentrations, temperature) and then test for large-scale (e.g., 1,000) multiplexed detection.

### RNA Extraction-Free Sample Processing and Highly-Multiplexed Viral Detection in Clinical Samples

Current gold-standard RT-PCR protocols rely on RNA extraction before cDNA conversion, which limits the overall assay throughput and makes testing dependent on the availability of RNA extraction kits, which can be in short supply during pandemics. The methods described herein comprise an efficient cDNA conversion and barcoding method without a separate RNA extraction step. Methods for nuclease inhibition and reverse transcription are also utilized; see e.g., Myhrvold et al., Science, 2018. 360(6387): 444-448, the contents of which are incorporated herein by reference in its entirety. Mimicked clinical samples (e.g., spiked-in synthetic targets in human cell background) are used to assay cDNA conversion efficiency and overall detection sensitivity and uniformity across different barcodes. Finally, the method for multiplexed detection is tested with patient samples, through collaboration with hospitals. Such tests are cross-checked with standard RT-PCR methods to validate the test results and further quantify our limit of detection, false positive, and false negative rates in patient samples.

### Approach

The principle of this approach is to use DNA barcoding to tag different patient samples (e.g., sample ID), as well as multiple viral species or genomic loci (e.g., locus ID) at the cDNA level, thus permitting highly-parallel readout by NGS sequencing. In contrast to traditional sequencing-based viral detection and assay methods, the approach does not sequence the viral genome. In some embodiments, it only reads out the two DNA barcodes. Additionally, the method uses limited pre-amplification in combination with bridge PCR to prevent the common problem of carryover contamination.

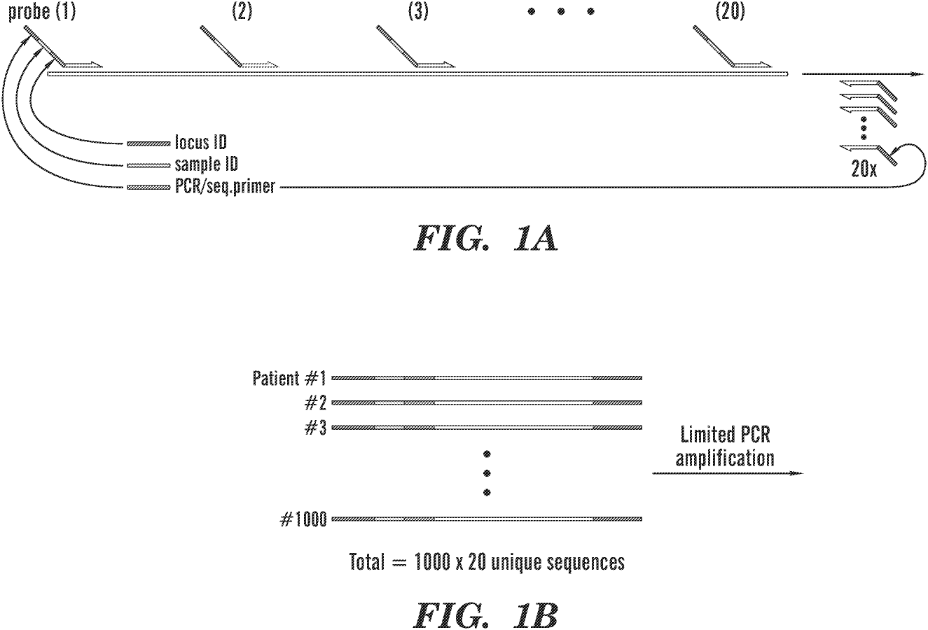

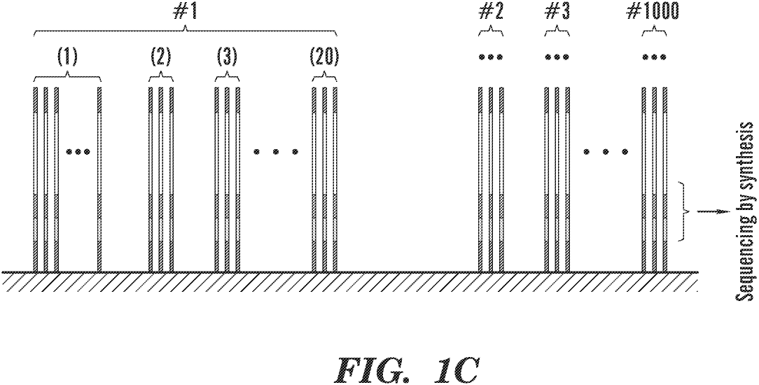

The molecular workflow for the method comprises four steps (see e.g., FIG. 1): (i) Patient samples are converted to cDNA (first strand) with a set of barcoded forward primers, which can encode the sample ID as well as locus ID (see e.g., FIG. 1A). (ii) cDNA strands from many samples (e.g., 1,000) are pooled and a second strand is synthesized with a common, backward primer (see e.g., FIG. 1B). (iii) Barcoded and pooled samples are purified, amplified with a limited number of PCR cycles, then captured on a surface. (iv) Barcodes (e.g., sample and locus ID) are amplified by bridge PCR and read out by high-throughput sequencing (see e.g., FIG. 1C).

A single sequencing run on a MiSeq machine (e.g., 20 million reads), for 1,000 patient samples and 20 genomic loci, gives an average of 1,000 reads per patient/locus pair. This matches well with the clinically observed dynamic range of viral load, and indicates that the method can not only report the existence or absence of virus, but can also provide quantitative information on the patient’s viral load (e.g., around the swab sampling area). The test result can be interpreted as positive when most of the 20 locus IDs (e.g., >15) are observed (e.g., associated with a particular patient ID); and negative when none or only a few are observed (e.g., <5). The assay is therefore highly robust against sample degradation and barcode cross-talk.

(a) Multiple viral pathogens can be tested simultaneously for differential diagnosis, by extending the pool of locus-specific probes to target different viral genomes (e.g., COVID-19, SARS, H1N1). (b) A unique molecular identifier (UMI) can be incorporated on the reverse primer, to allow digital counting of viral load. (c) A short segment of viral genome can be sequenced, immediately following the barcode regions, to provide viral sequence and mutation information at locations critical for the study of virus-host interaction and potentially vaccine development (e.g., the ACE2 binding site on the SARS-CoV-2 spike protein). (d) cDNA conversion and barcoding can be performed in one reaction, after heat inactivation of the virus and in the presence of nuclease inhibitor and viral transport medium (VTM).

The workflow for multiplexed viral RNA barcoding and detection can be used to detect 1,000 samples in a single sequencing run. A pilot test can be performed that multiplexes 100 samples. With demonstration of massively multiplexed viral detection in 1,000 patient samples, this workflow can be implemented in local hospitals.

In some embodiments, a sequencer can be used as a single molecule detector without amplification. Also, by employing DNA barcoding several steps in bulk biochemistry can be performed after pooling the individual molecules and recovering the identities of the individuals who contributed the samples. The assay can be expanded to multiple individuals and multiple viruses simultaneously. This technique can be immediately extended to as many viruses as one wished and used to look at the spread of genetic variants in the populations of many samples all at once, taken from tens of thousands to hundreds of thousands of individuals. With this information, epidemiologists can design optimal strategies for predicting the course of an epidemic and for designing a strategy to contain the epidemic by identifying the carriers and segregating them from a healthy population. This kind of technique can be used to measure the efficacy of anti-virals and vaccines in smaller populations in clinical trials.

### Example 2: “One-Step” Sequencing for Scalable Viral Diagnostics

The goal for the method described herein is to reduce or remove as many pre-processing steps as possible to cut down the labor and material requirement for scaling up; such pre-processing steps include, e.g., RNA extraction, pre-amplification, and the logistics of sample handling. Barcoding and sequencing methods allow for low-crosstalk, high-dynamic-range readout. Such methods are referred to herein as “one-step” and/or “one-Seq” methods, e.g., from the patient and logistic perspective. For the patient, such methods allow at-home sample collection and remove the burden of a heating step at home. For the testing facility, such methods remove any per-tube reaction (e.g., RNA extraction, PCR/thermocycling) and any nontrivial robotic pipetting.

See, e.g., Table 1 for exemplary advantages of the sequence-based detection methods as described herein. FIGS. 1A-1C shows an exemplary workflow for One-Seq. The One-Seq method allows for highly-multiplexed and highly-reliable viral detection and mutation tracing. With regard to biochemistry, the One-Seq method demonstrates: high-sensitivity, one-step viral lysis, and reverse transcription (including sample barcoding); the method is compatible with multiplexed RT primers and long-term (e.g., 24-48 hrs) sample stability. With regard to sequencing, the One-Seq method demonstrates sequence amplification with high barcode specificity (e.g., low barcode swapping) and a high dynamic range readout of a large number of patient samples (e.g., viral load can vary over 3-6 log).

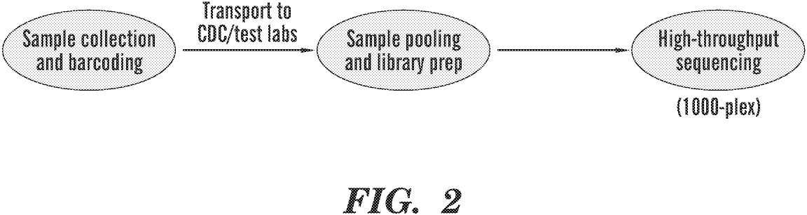

FIG. 2 shows a flowchart of an exemplary detection method as described herein.

TABLE 1

Exemplary advantages of sequencing-based detection methods

| | | |
|---|---|---|
| Criteria | Performance | Notes |
| Sensitivity | ++ | Can be influenced by viral load dynamic range |
| Specificity | +++ | Sequencing info provides extra specificity |
| Speed | + | Can be influenced by logistics |
| Scalability | +++ | Takes advantages of existing facilities |
| Identifiability | +++ | Can be influenced by barcode swapping |
| Material/reagent-sparing | +++ | Amortized |
| Quantitative | ++ | Can be influenced by viral load dynamic range |
| Multi-virus testing | +++ | Unlimited multiplexity |
| Cost | +++ | Amortized |

The workflow of the method described herein comprises barcoding at the first step. There is also no pre amplification before pooling, allowing for a simpler biochemistry reaction for complex environment, multiplexed detection, and semi-quantitative readout. The method also involves short amplicon sequencing.

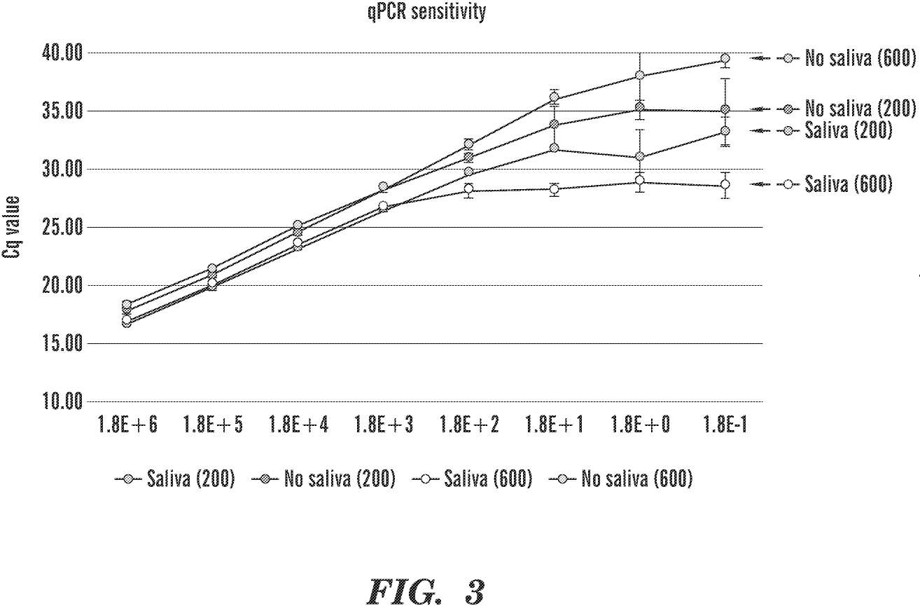

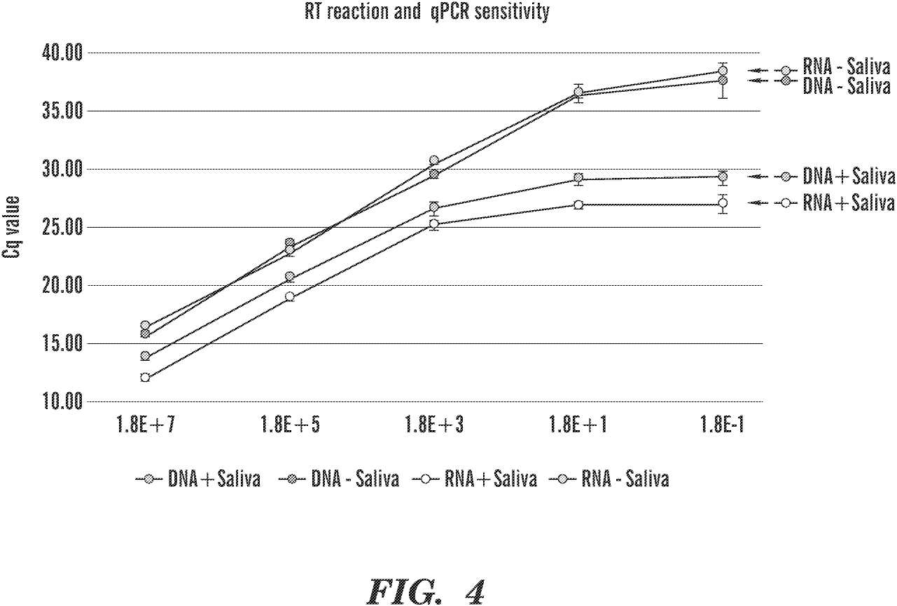

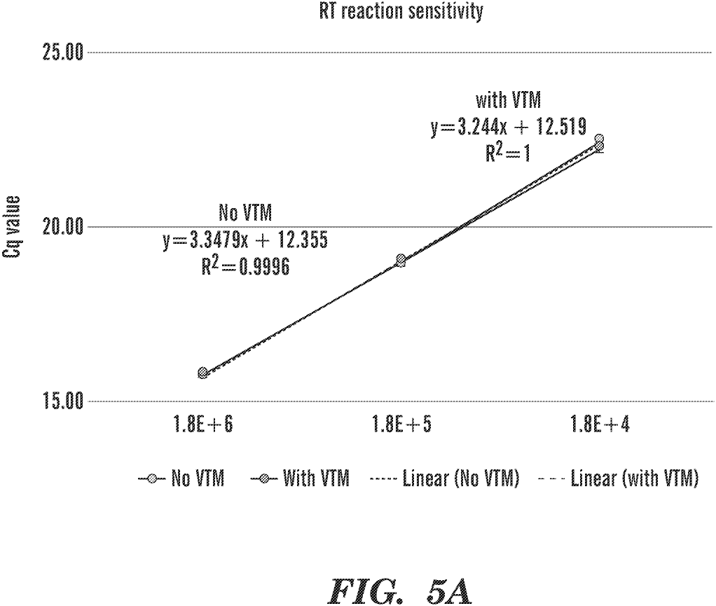

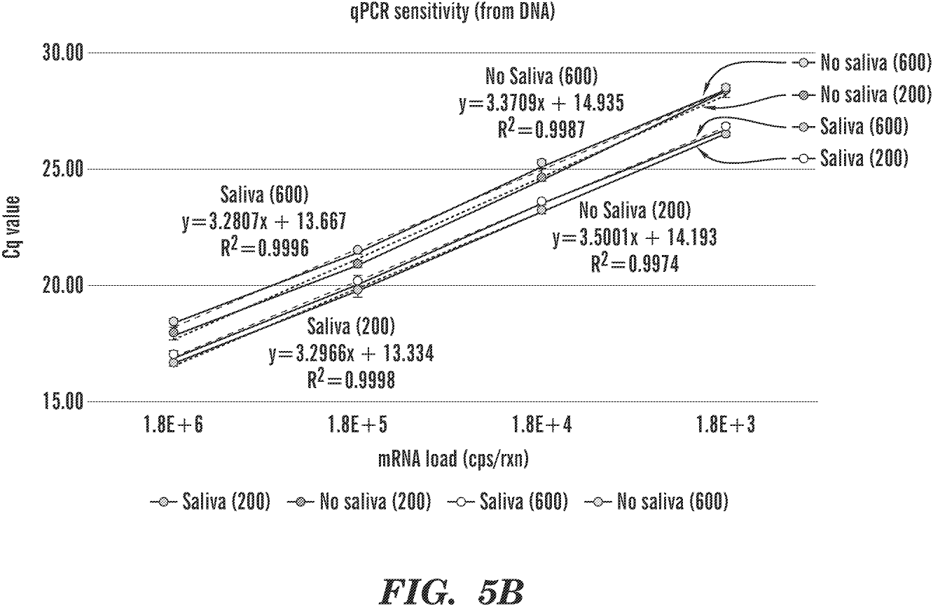

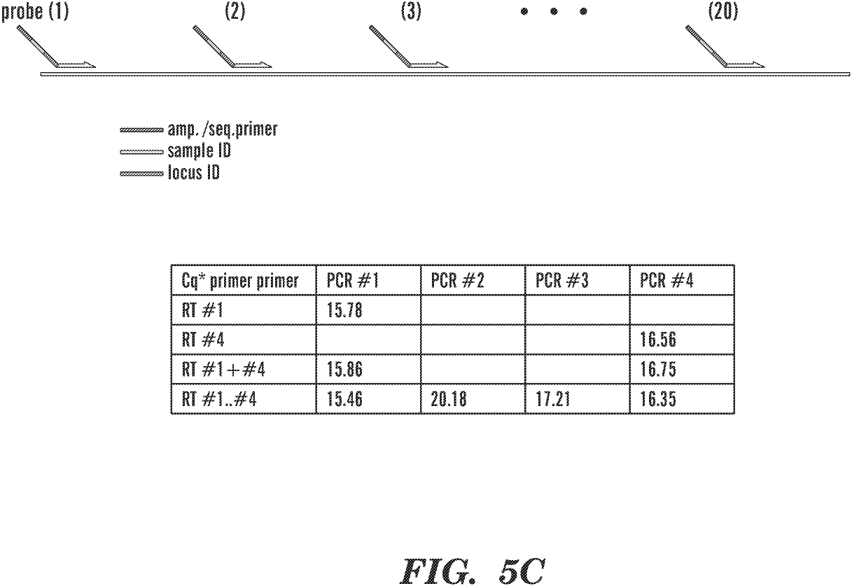

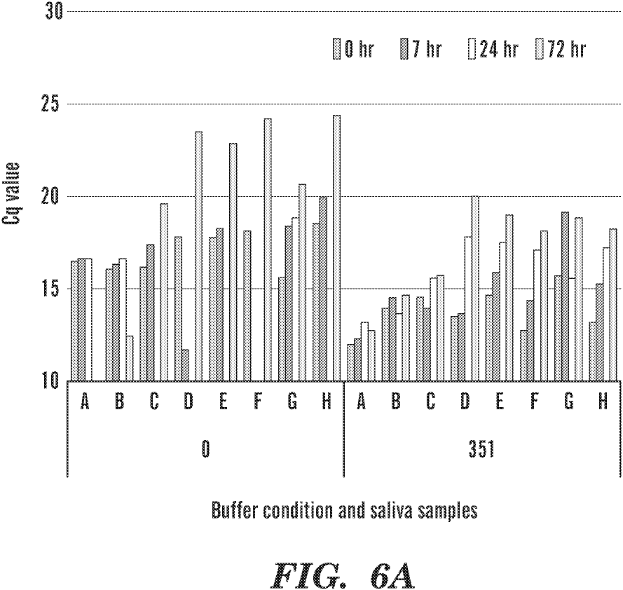

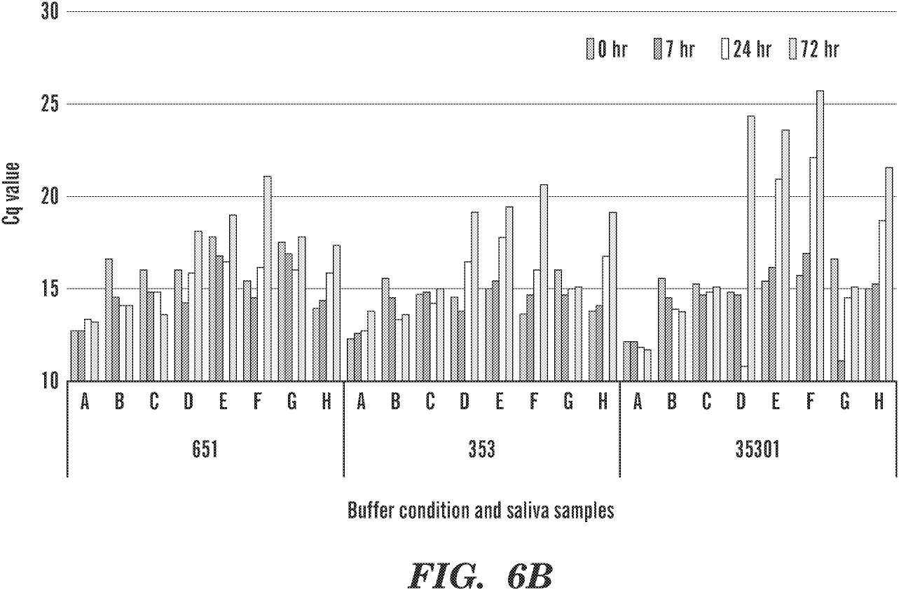

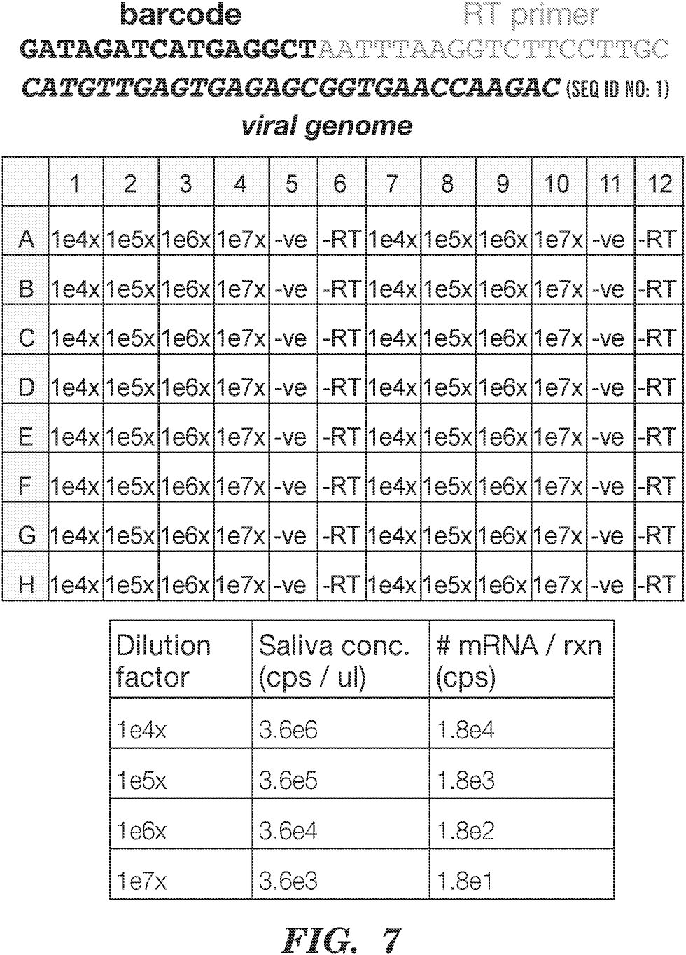

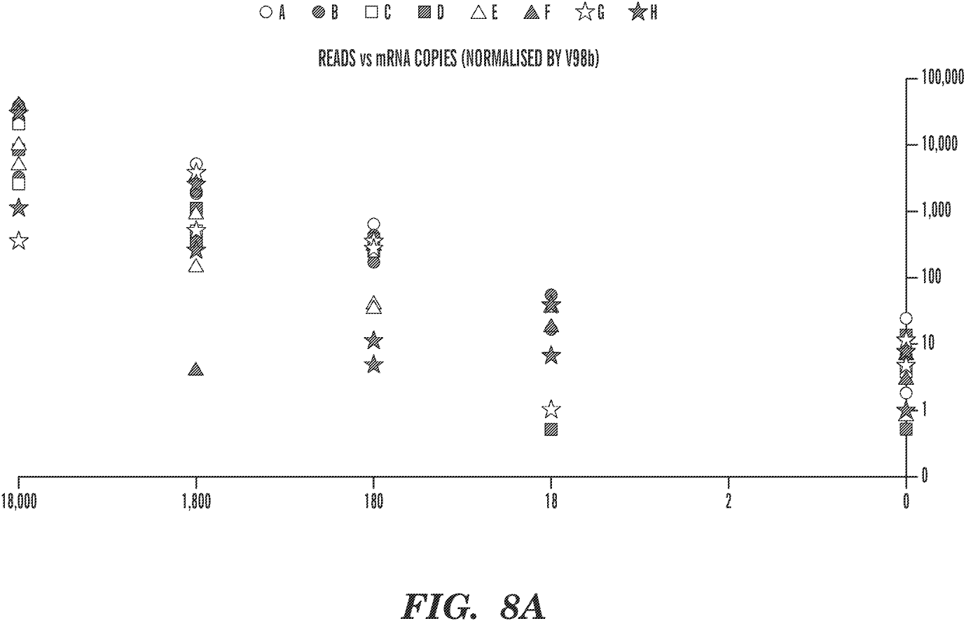

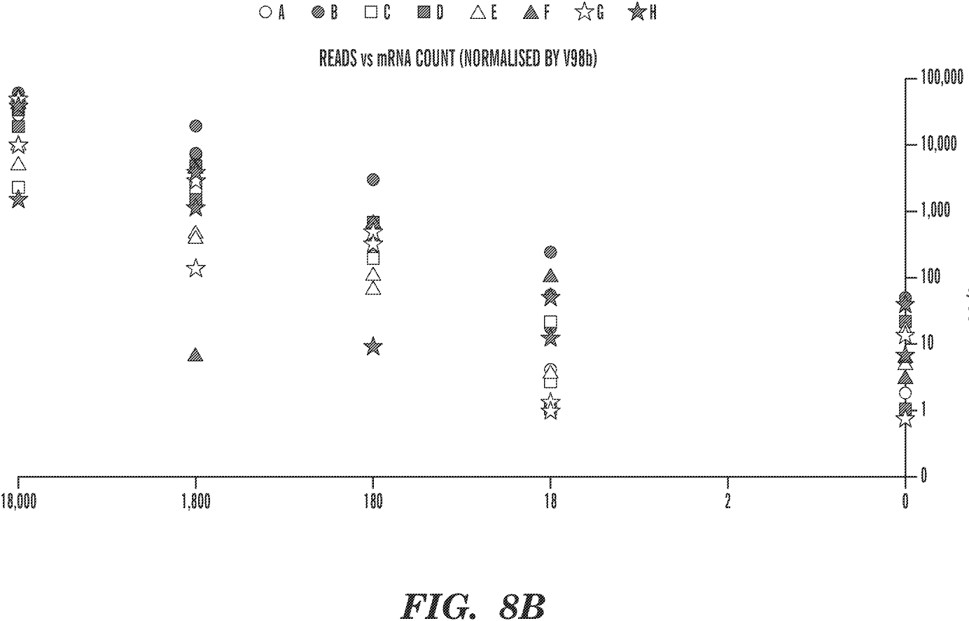

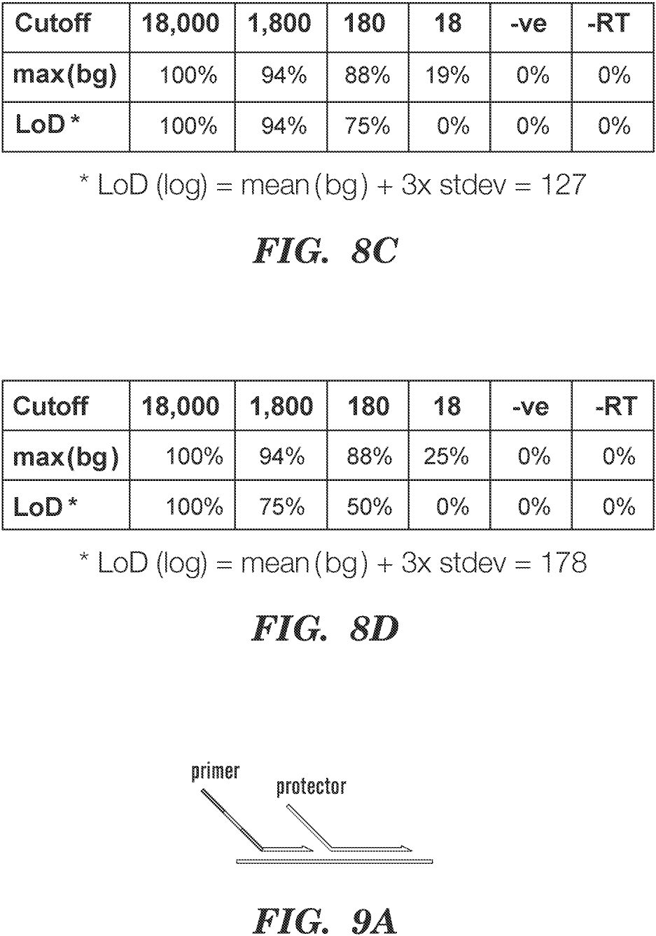

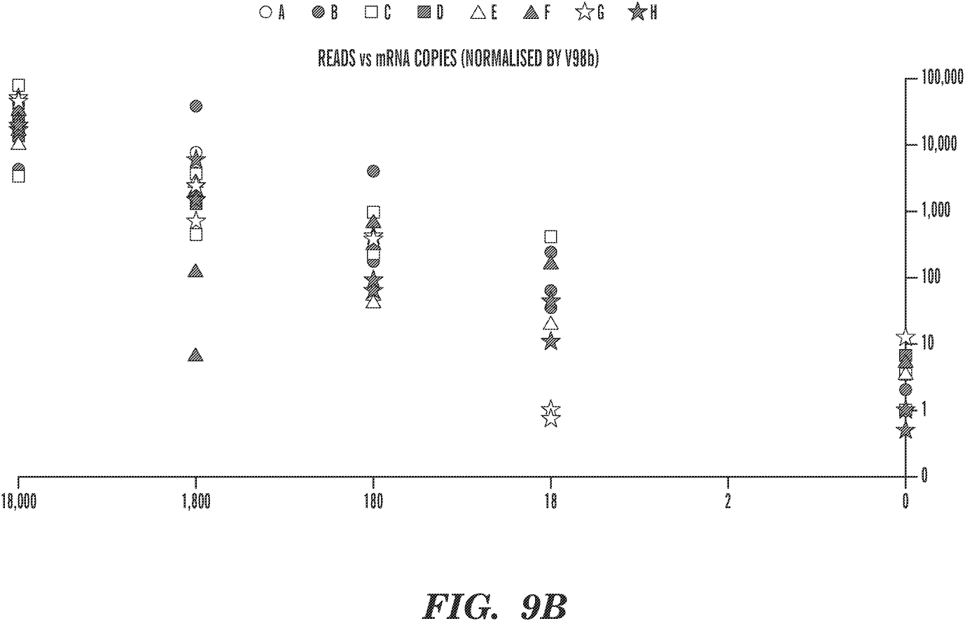

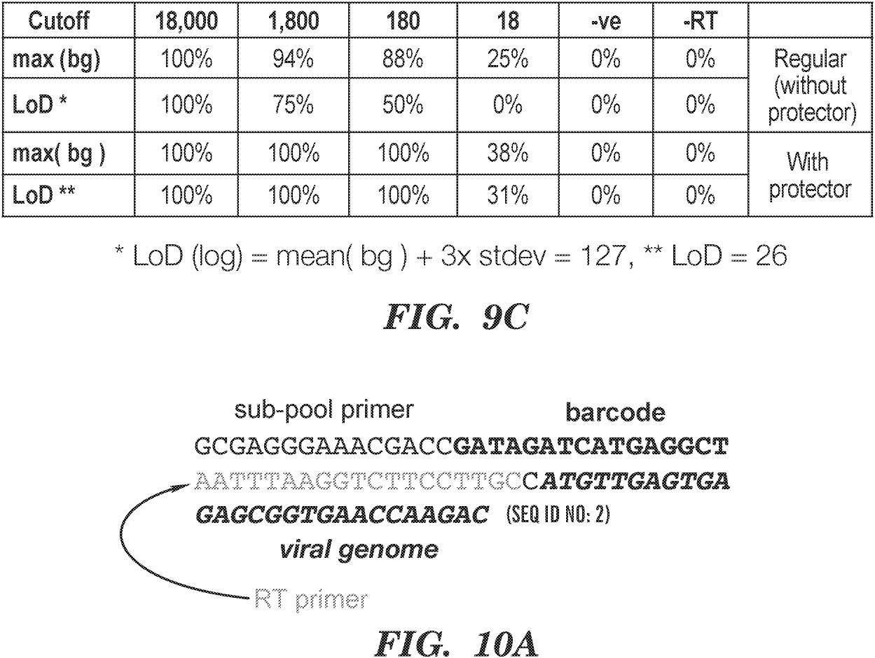

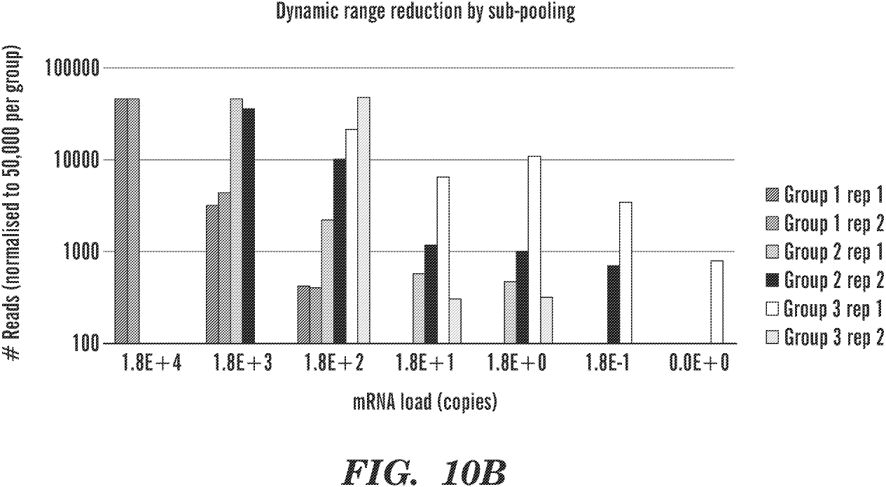

Biochemically, the methods described herein comprise: a one-step RT reaction, e.g., in the presence of viral media and/or saliva; a multiplexed RT reaction; sample preservation before reaching a central testing facility; and/or a positive control for sample quality, amount, and/or RT reaction. See e.g., FIGS. 3-8 for exemplary RT PCR results. Sample pooling and sequencing allows for high detection sensitivity and high dynamic range of viral load from different patients. The protector strand strategy as described herein (see e.g., FIGS. 9A-9C) can help eliminate a barcode swapping issue. FIGS. 10A-10B shows a sub-pooling strategy for increased dynamic range.

In summary, the one-step reaction system for viral lysis and efficient reverse transcription described herein is compatible with multiplexed RT reactions and sample storage at room temperature for up to 24 hrs; furthermore, the high-throughput sequencing readout method demonstrates a high dynamic range (see e.g., FIGS. 2, 3, 4, 5A-5C, 6A-6B, 7, 8A-8D). With the protector strategy, barcode crosstalk was reduced to <10^-4 (see e.g., FIGS. 9A-9C). With sub-pooling, 5+ logs dynamic range can be detected (see e.g., FIGS. 10A-10B).

Outlook, regulatory agencies, such as the FDA, have approved for Emergency Use Authorization (EUA) NGS-based COVID-19 diagnostic test (e.g., IDT). The methods described herein can also be used for COVID-19 diagnostics, using sequence-optimized primers and barcodes, as well as multiplexed viral and viral loci detection.

### Example 3: One-Seq, A Highly Scalable Sequencing-Based Diagnostic for SARS-CoV-2 and Other Single-Stranded Viruses

The management of pandemics, such as COVID-19, requires highly scalable and sensitive viral diagnostics, together with variant identification. Next-generation sequencing (NGS) has many attractive features for highly multiplexed testing, however current sequencing-based methods are limited in throughput by early processing steps on individual samples (e.g., RNA extraction and PCR amplification). Described herein is a method, “One-Seq”, that eliminates the bottlenecks in scalability, by permitting early pooling of samples, before any extraction or amplification steps. To permit early pooling, a one-pot reaction is used for efficient reverse transcription (RT) and upfront barcoding in extraction-free clinical samples, and a “protector” strategy in which carefully designed competing oligonucleotides prevent barcode crosstalk and preserve detection of the high dynamic range of viral load in clinical samples. One-Seq is highly sensitive, achieving a limit of detection (LoD) down to 2.5 genome copy equivalent (gce) in contrived RT samples, 10 gce in multiplexed sequencing, and 2-5 gce with multi-primer detection, indicating an LoD of 100-250 gce/ml for clinical testing. In clinical specimens, One-Seq showed quantitative viral detection against clinical Ct values with 6 logs of linear dynamic range and detection of SARS-CoV-2 positive samples down to ~300 gce/ml. In addition, One-Seq reports a number of hotspot viral mutations, allowing variant identification, at equal scalability with no extra cost. Scaling up One-Seq allows a throughput of 100,000-1,000,000 tests per day per single clinical lab, at an estimated amortized reagent cost of $3 per test and turn-around time (TAT) of 7.5-15 hr.

Highly-scalable and highly-sensitive viral diagnostics (e.g. for SARS-CoV-2) are critical for both pandemic response and long-term epidemiological surveillance. During a pandemic, population-wide testing can provide effective control and monitoring of the viral spread and allow safe return to work. In the long term, regular and population-wide monitoring promises a “bio-weather map” to identify and forecast new viral infection hotspots, preventing the “next outbreak”. Furthermore, the ability to sequence and identify emerging viral variants (e.g. B.1.1.7, B 1.427 for SARS-CoV-2), also on the population scale, allows real-time monitoring of the rate of transmission and pathogenicity, as well as informing public health policies and vaccine development. Current diagnostic methods fall short of these requirements, as they are limited in either sample processing throughput, testing sensitivity and reliability, or the ability to identify different viral variants.

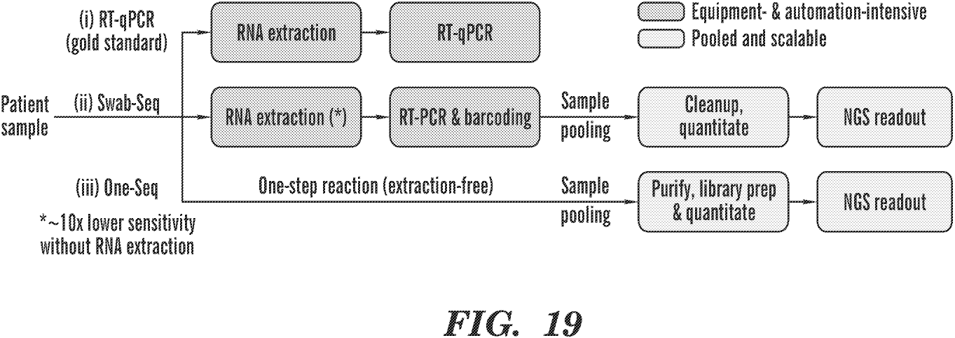

At present, molecular tests using “gold standard” reverse transcription polymerase chain reaction (RT-qPCR) in central laboratory facilities have demonstrated high detection sensitivity (down to 200 gce/mL-1,000 gce/mL of SARS-CoV-2 (by the FDA’s comparison panel results), but they are limited in throughput by the requirements of RNA extraction and PCR thermocycling on each sample individually, as well as other liquid handling operations; see e.g., FIG. 19; see e.g., Vandenberg et al. Nat Rev Microbiol 19, 171-183 (Oct. 14, 2020); MacKay et al. Nat Biotechnol 38, 1021-1024 (Aug. 20, 2020); Esbin et al., RNA 26, 771-783 (May 1, 2020); Arnaout et al. SARS-CoV2 Testing: The Limit of Detection Matters (bioRxiv, Jun. 4, 2020); the contents of each of which are incorporated herein by reference in their entireties. As a result, it is challenging for most current clinical labs to perform more than 10,000 diagnostic tests per day, even with the help of automation; see e.g., Cobas SARS-CoV-2 Instructions for Use (Mar. 12, 2020), available on the world wide web at fda.gov/media/136049/download; the content of which is incorporated herein by reference in its entirety. By re-purposing large-scale liquid handling and sample automation, up to 100,000 tests per day can be achieved, but this approach requires heavy upfront capital investment and personnel costs.

Next-generation sequencing (NGS) based methods have long been attractive alternatives to RT-qPCR in two ways: (i) the intrinsic high-throughput readout for multiplexed diagnostics, and (ii) the ability to obtain viral genome sequences for variant identification. In principle the very high-throughput (up to 10¹⁰ reads per session, on an Illumina NovaSeq™ machine) allows a single testing lab to process up to a million patient samples per day with pooled analysis, if they could avoid the handling of individual samples. Since the beginning of the COVID-19 pandemic, several methods for NGS-based multiplexed testing have been proposed and developed. See e.g., Bloom et al., Swab-Seq: A high-throughput platform for massively scaled up SARS-CoV-2 testing, medRxiv (Aug. 6, 2020); Illumina™ COVIDSeq Test Instructions for Use (May 1, 2020); Hossain et al. A massively parallel COVID-19 diagnostic assay for simultaneous testing of 19200 patient samples. Google Docs (Mar. 20, 2020); Schmid-Burgk et al. LAMP-Seq: Population-Scale COVID-19 Diagnostics Using a Compressed Barcode Space bioRxiv (Apr. 8, 2020); Wu et al., INSIGHT: A population-scale COVID-19 testing strategy combining point-of-care diagnosis with centralized high-throughput sequencing. Sci Adv 7, (Feb. 12, 2021); Yelagandula et al. SARSeq, a robust and highly multiplexed NGS assay for parallel detection of SARS-CoV2 and other respiratory infections (med Rxiv, Nov. 3, 2020); the contents of each of which are incorporated herein by reference in their entireties.

As expected, methods that reported detection sensitivity close to the RT-qPCR tests (200-1000 gce/ml) mostly followed the traditional barcoding and sequencing workflows and require individual RNA extraction and PCR thermocycling steps; see e.g., FIG. 19, see e.g., supra, Bloom, Illumina, Yelagandula) (or used an extraction-free protocol but with ~10x lower sensitivity, see e.g., Bloom supra; Bruce et al., PLoS Biol 18, e3000896 (Oct. 2, 2020); the contents of each of which are incorporated herein by reference in their entireties), which in practice hinders the maximum achievable sample throughput (see e.g., FIG. 12A). Furthermore, current methods either do not report viral variant information, or perform whole genome sequencing (WGS), which further limits the achievable throughput due to the large number of sequencing reads required.

To overcome these limitations, described herein is a sequencing-based method that achieves high sensitivity, high throughput, and identification of viral variants. To obtain high throughput a “pooling-before-amplification” strategy was implemented (see e.g., FIG. 12A, FIG. 19); the workflow performs an extraction-free, PCR-free, one-step processing from clinical sample to library pooling, thus allowing thousands of patient samples to be processed immediately after arrival at testing centers, with all further steps being done in bulk (see e.g., FIG. 12B). The method is referred to herein as “One-step” viral Sequencing, or “One-Seq”.

### Results

To overcome the bottleneck in throughput, One-Seq introduces a “pooling-before-amplification” strategy (see e.g., FIG. 12A), that postpones library amplification until after sample pooling and avoids the instrument- and liquid handling-intensive steps of RNA extraction and PCR thermocycling. The molecular workflow of One-Seq comprises the following four steps (see e.g., FIG. 12C, FIG. 20). (1) viral particles (e.g., from patient samples) are lysed and viral RNA is transcribed to a first strand cDNA using a barcoded RT primer, that includes the patient sample barcode, and an adaptor for library amplification; (2) barcoded single-stranded cDNAs are pooled (e.g., 100-1,000 samples) and purified to remove excess RT primers and buffer; (3) second strand cDNA synthesis and PCR library amplification from a common reverse primer and a common forward extension primer are performed together, optionally with a batch barcode on the reverse side; and (4) amplicon libraries are cleaned up and normalized, and optionally pooled again with different batches, and analyzed by next-generation sequencing. This workflow is further compatible with multiplexed viral detection and sequencing (see e.g., FIG. 12D), where several strands sharing the same patient barcode but with different RT primer sequences mixed together. Such a multi-primer strategy confers three benefits: (i) increased detection sensitivity (e.g., sensitivity increases linearly with number of primers); (ii) ability to sequence multiple viral loci to permit variant identification; and (iii) simultaneous detection of multiple different viruses (e.g. common cold, flu, hepatitis viruses), informing better diagnosis as well as providing a more comprehensive picture for epidemiological surveillance. On top of viral targets, One-Seq further incorporates two positive controls: one against a specially designed synthetic RNA fragment that shares the same RT primer as one of the viral targets but has a different sequence, and another against human RPP30 gene (see e.g., FIG. 12D).

Such a workflow involves at least two critical challenges. First, the one-step, extraction-free reaction has to perform three tasks simultaneously: viral lysis and release of viral RNA, an efficient reverse-transcription that allows high-sensitivity viral detection, and preservation of patient samples at room temperature for up to 24 hr during sample collection and transport to the central lab. Second, by performing pooling before amplification, the library amplification reaction must faithfully preserve the high dynamic range of viral load known to exist in clinical samples (e.g., up to 10⁶to 10⁷-fold range), and at the same time achieve high detection sensitivity. In particular, the method needs to stringently avoid any barcode crosstalk that can arise from amplification and sequencing steps, as this crosstalk would result in false positive diagnoses. The detection methods described herein overcome at least those challenges.

### A One-Pot Reaction for Efficient Viral Reverse Transcription and Sample Preservation

### An Optimized RT Reaction System Allows for Sensitive RNA Detection From Extraction-Free Virus Samples

Described herein is an extraction-free and high-sensitivity method for viral lysis and reverse transcription (RT), which can be performed in the presence of potential inhibitors in patient samples (e.g. NP swab or saliva). Since reverse transcriptases are in general more resistant to inhibitors than thermostable polymerases, there is an unappreciated advantage in separating the RT and PCR steps in the traditional RT-PCR workflow, since this allows more flexibility in formulating the RT reaction mix. To assay RT efficiency in the presence of inhibitors, contrived standard samples were prepared with human saliva collected from COVID-19 negative donors and viral RNA spike-in (e.g., synthetic RNA fragment by in vitro transcription (IVT), or full-length RNA genome from Twist Bio Sciences™). First, the RNA protection effects of different RNase inhibitors were compared, and Murine™ (New England Biolabs™) and RNAsin™ (Promega™) provided the best and similar protection at 25° C. to 50° C. The RT efficiency of various reverse transcriptases was then compared in saliva-containing samples (see e.g., FIG. 21), using qPCR as a readout with the CDC’s RT-PCR primer and probe set; see e.g., “Real-Time RT-PCR Panel for Detection, 2019-Novel Coronavirus - Instructions for Use.,” (Center for Disease Control and Prevention, Jan. 15, 2020), available on the world wide web at stacks.cdc.gov/view/cdc/84526, the content of which is incorporated herein by reference in its entirety. SuperScript IV™ reverse transcriptase detected 3 molecules of synthetic RNA in the presence of human saliva, such that it is sensitive enough to be used in the efficient, extraction-free reactions described herein.

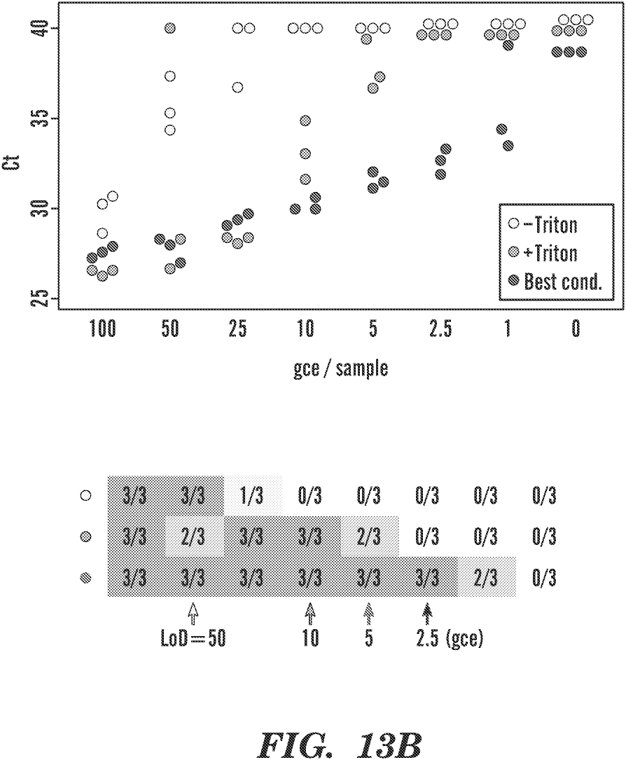

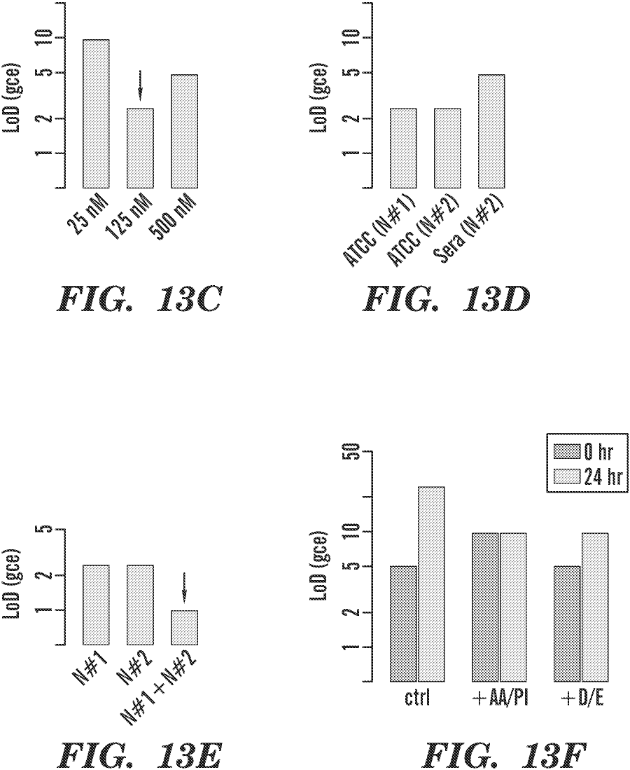

Contrived clinical samples were next prepared using pooled COVID-19 negative remnant clinical specimens (nasopharyngeal (NP) swab in viral transport medium (VTM), N=15), with spiked-in inactivated virus standard (heat-inactivated SARS-CoV-2 from ATCC, VR-1986HK; or AccuPlex™ SARS-CoV-2 verification panel from SeraCare™, 0505-0168) (see e.g., FIG. 13A). In contrast to a “naked” RNA spike-in, these inactivated virus samples allowed testing of the efficiency of viral lysis in patient samples.

To assay the analytical sensitivity of RT reaction, a roughly 2x dilution series was prepared of inactivated virus standard (ATCC) in contrived clinical samples, ranging from 100 genome copy equivalent (gce) to less than 1 gce per reaction. The RT product was assayed by qPCR in triplicate (see e.g., FIG. 13B). The RT samples indeed showed a significant inhibitory effect on PCR amplification, and PCR efficiency was restored only after a 40x-80x dilution.

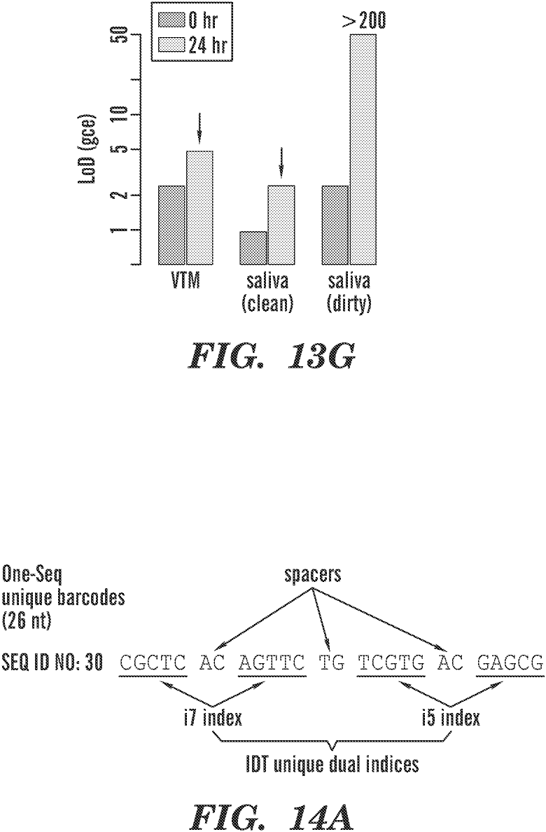

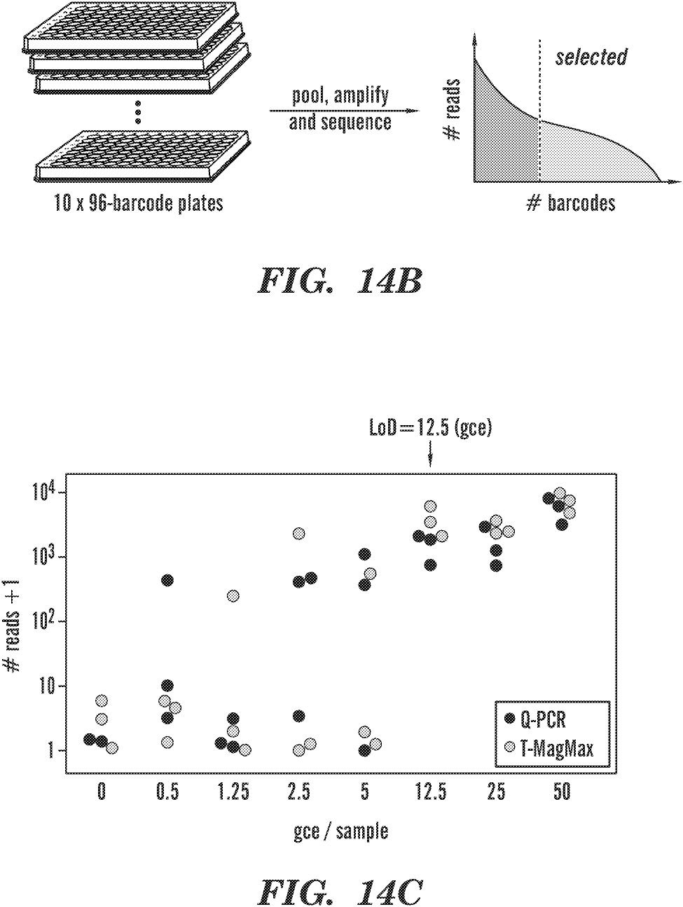

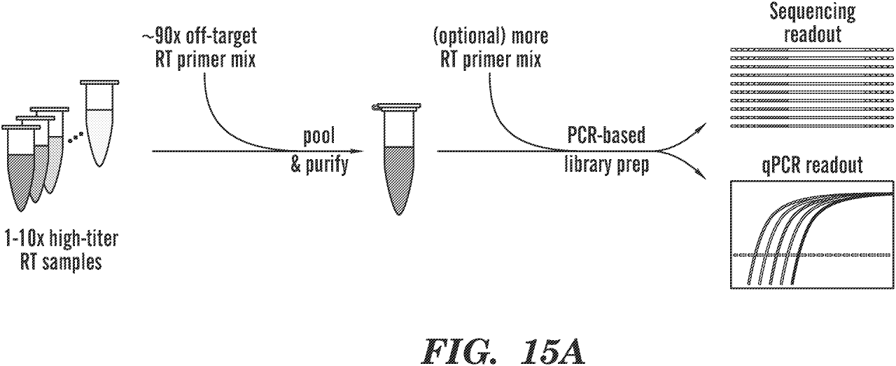

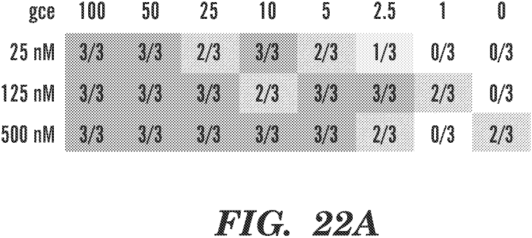

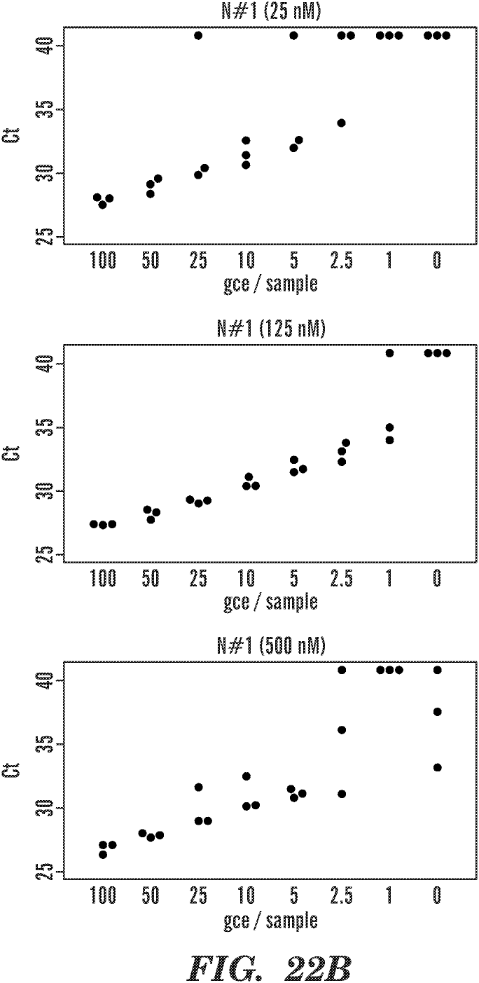

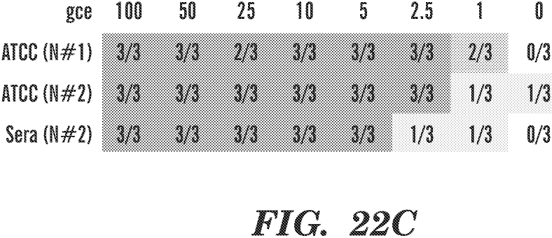

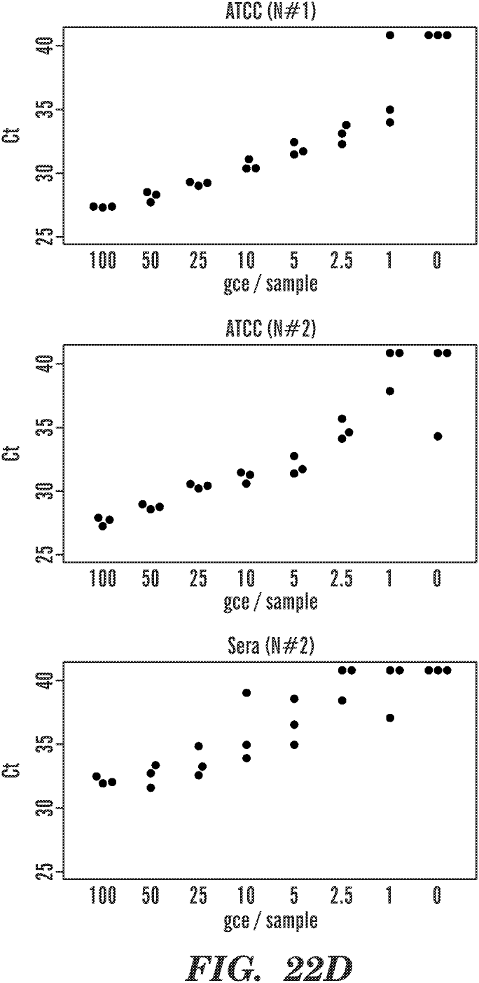

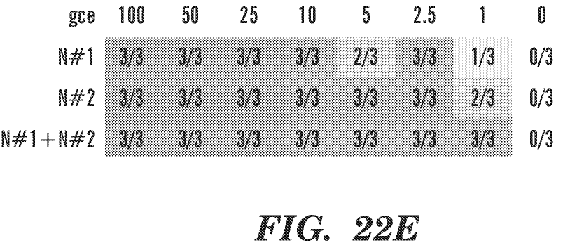

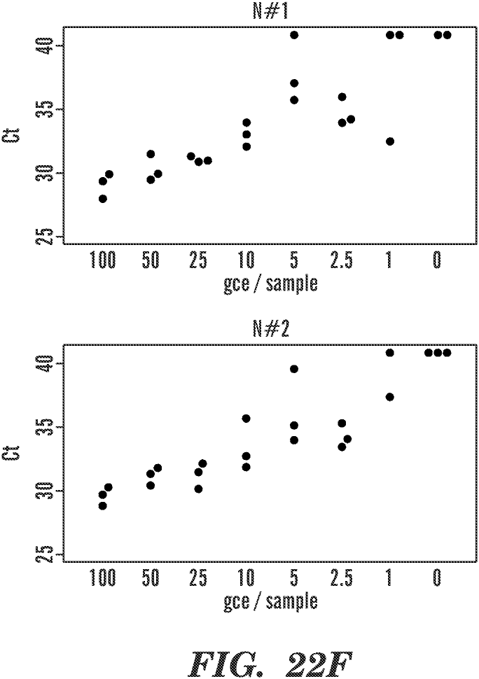

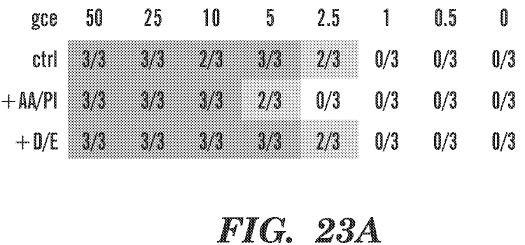

To optimize viral lysis and RNA release, the effect of using detergent was tested; see e.g., Smyrlaki et al., Nat Commun 11, 4812 (Sep. 23, 2020); Srivatsan et al. Preliminary support for a “dry swab, extraction free” protocol for SARS-CoV-2 testing via RT-qPCR (Biorxiv, Apr. 23, 2020); the content of each of which is incorporated herein by reference in its entirety. The addition of mild detergent (Triton X-100) improved the detection sensitivity by ~5x from extraction-free viral samples, from a limit of detection (LoD) = 50 gce to 10 gce (3/3 detection; see e.g., FIG. 13B). Two RT primers were then designed against the SARS-CoV-2 N gene, optimizing thermodynamic parameters and avoiding regions with significant sequence variance or homology to other related viruses (see e.g., Table 4). After optimizing for primer concentration (see e.g., FIG. 14C, FIG. 22), both primers achieved an LoD = 2.5 gce, close to the theoretical maximum sensitivity (see e.g., FIGS. 13B, 13D). The detection limit was further verified with a different source of viral reference standard (SeraCare™), and consistent results were obtained (see e.g., FIG. 13D, FIG. 22).

Multiplexed RT with multiple primers provides the ability for multi-loci and multi-virus monitoring as well as increased detection sensitivity. This effect was tested using the two SARS-CoV-2 N-gene-targeting primers in contrived clinical samples. Indeed, there was a roughly 2-fold higher detection sensitivity (LoD = 1 gce) when signals from both primers were considered (see e.g., FIG. 13E, FIG. 22). Since both primers target different genomic loci (separated by ~800 nt), the detection of these loci can be considered as independent events and thus it is possible to obtain LoD values less than 2 molecular copies.

### One-Seq Sample Stabilization Buffer Preserves Clinical Samples and Allows Sensitive Detection After 24 hr Incubation at Room Temperature

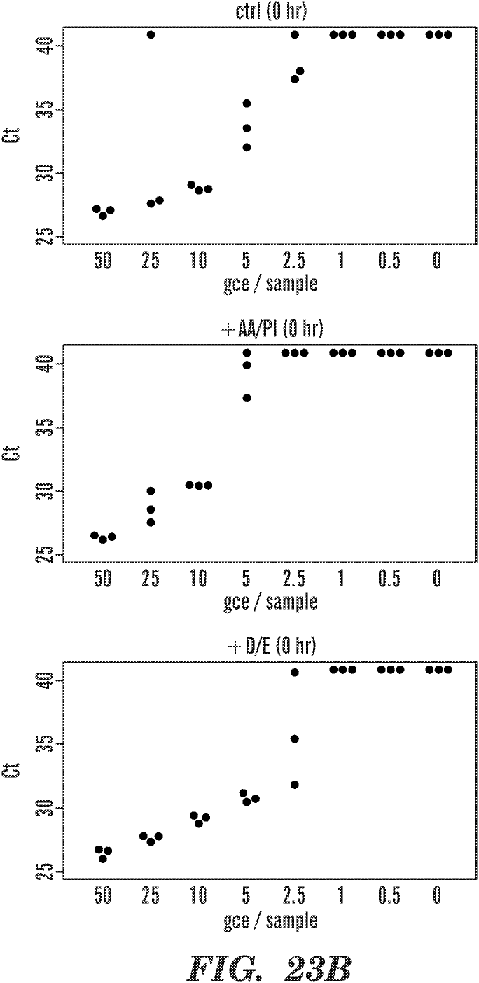

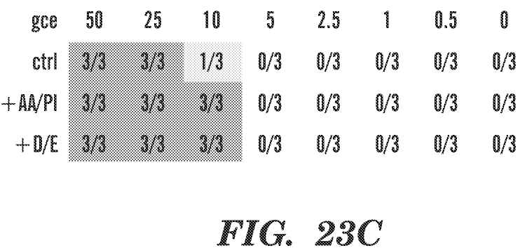

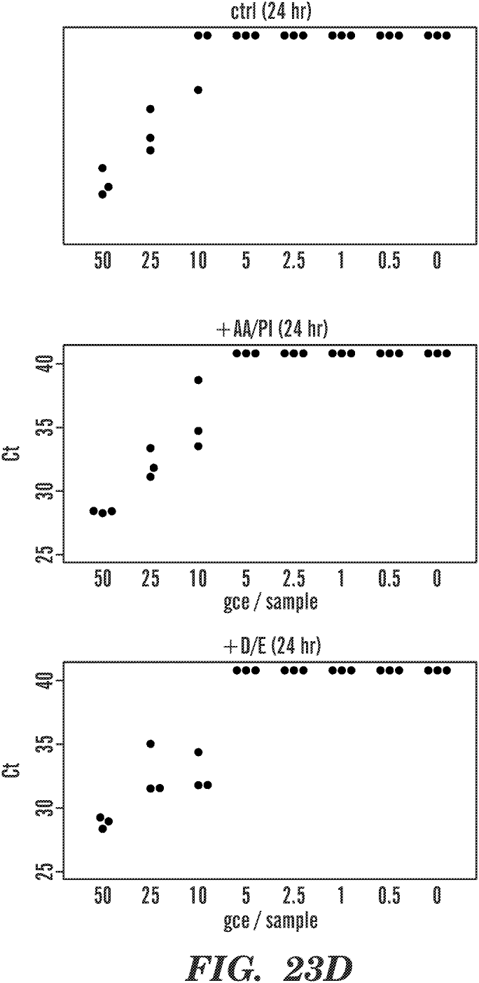

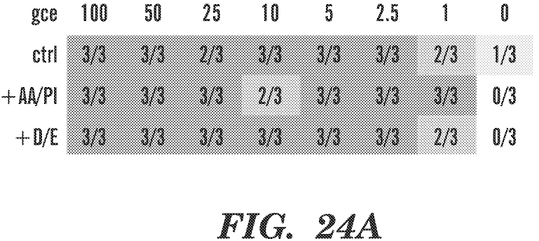

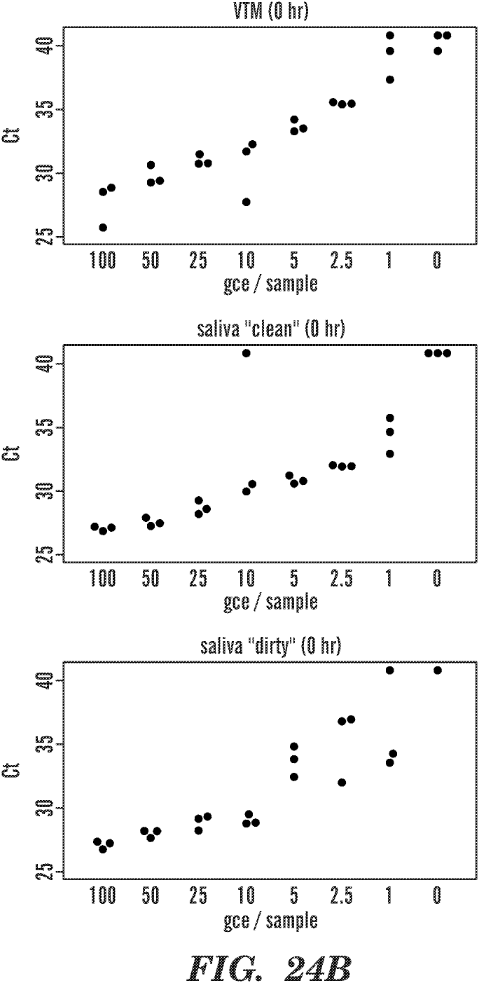

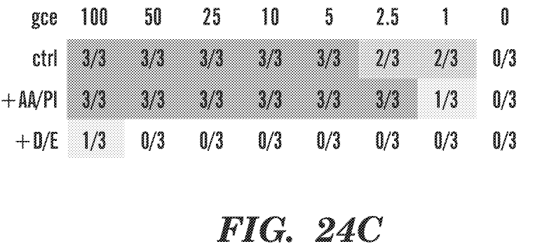

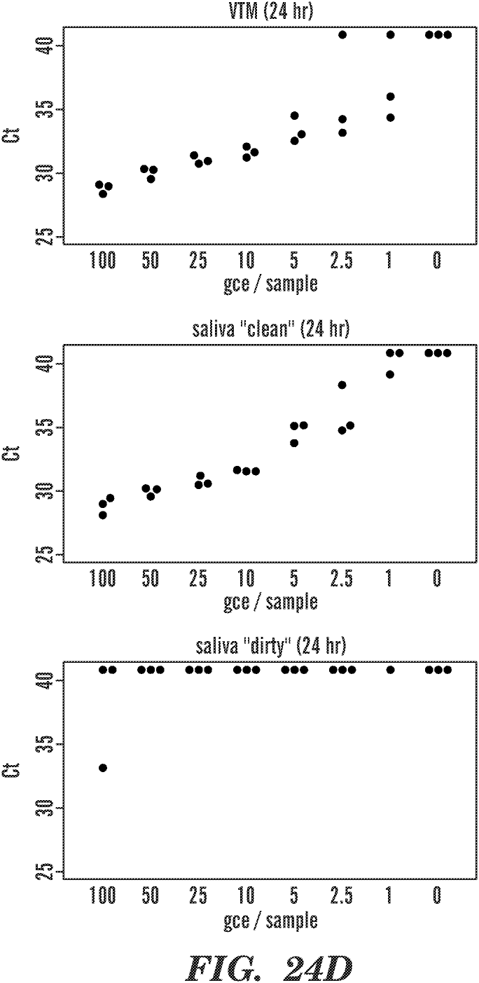

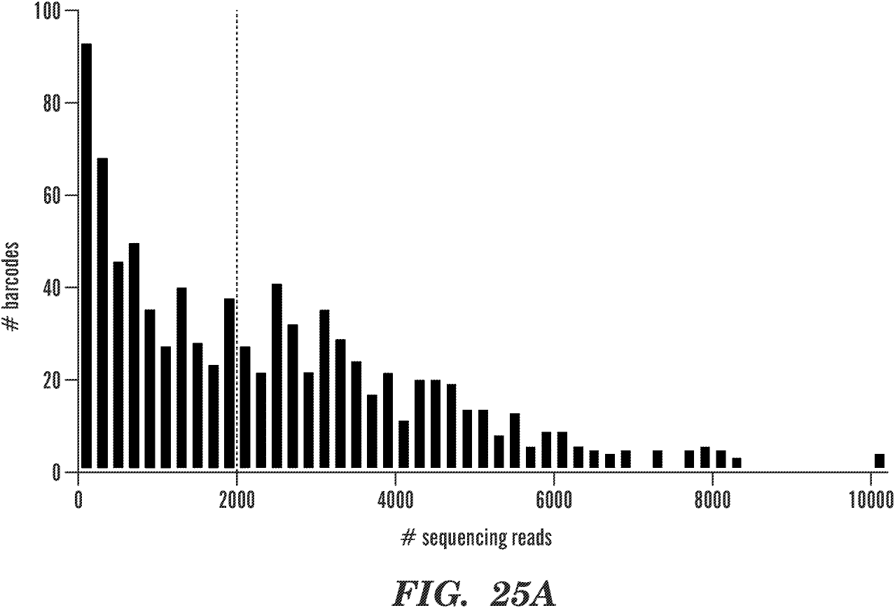

The one-pot reaction system can also stabilize patient samples for up to 24 hr at room temperature, during the delay between sample collection and transport to central testing lab. To work out the parameters, using contrived saliva samples with synthetic RNA spike-in (IVT), a list of stabilization agents were screened for their sample preserving effect, including antibiotics and antimycotics, protease inhibitors, reducing agents and metal chelating agents. The stabilization agents can be grouped into RNA-preserving (e.g., EDTA and DTT) and RT enzyme-preserving (e.g., antibiotic and antimycotic, protease inhibitor) factors. Their effects were tested in contrived clinical VTM samples prepared as above, with inactivated virus spike-in. After 24 hr incubation at room temperature, both groups individually improved RT efficiency by roughly 2-fold (see e.g., FIG. 13F, FIG. 23); together they improved the detection sensitivity significantly (from LoD = 25 gce to 5 gce), only a 2-fold reduction compared with unincubated (0 hr) control (see e.g., FIG. 13G, FIG. 24).

The sample stabilization buffer was also tested in contrived saliva samples (see e.g., FIG. 13G, FIG. 24). For this test, saliva specimens from COVID-19 negative donors were compared, collected with or without careful mouth rinsing before collection (denoted as “clean” and “dirty” saliva samples). To prepare the contrived samples, saliva specimens were pooled for both cases (N=4 and N=9, respectively), and inactivated viral standard (ATCC) was spiked-in (see e.g., FIG. 13A). Without room temperature incubation, both contrived saliva samples allowed highly sensitive detection (LoD <= 2.5 gce). After 24 hr incubation, viral RNA was still detected with high sensitivity (LoD = 2.5 gce) in the “clean” saliva sample, indicating the sample stabilization buffer successfully preserved the viral genetic material without significant degradation (see e.g., FIG. 13G, FIG. 24). Signals were lost in the sample containing “dirty” saliva (with visible food particles and other suspended debris), likely due to the degrading effect of food residues and microbes present in these samples.

### A “Pooling-Before-Amplification”Workflow for High Sensitivity and High Dynamic Range Multiplexed Sequencing

### Barcode Selection and cDNA Purification Allows Efficient Amplification After Sample Pooling

Described herein is a “pooling-before-amplification” workflow for sample pooling and PCR library amplification that not only maintains the high detection sensitivity and preserves signal linearity, but also preserves high sample dynamic range and allows quantitative report of viral load in patient samples.

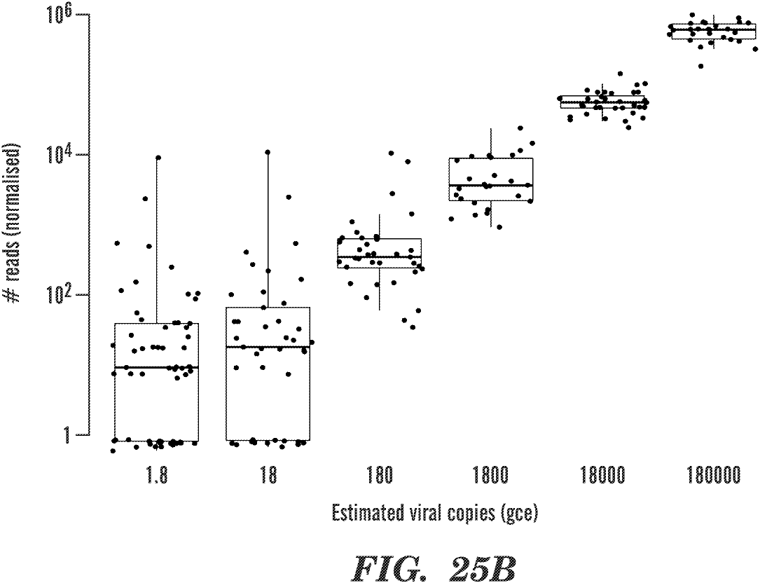

A set of PCR primers were first designed for efficient library amplification (see e.g., Table 4). For each RT target, several different reverse primers were designed and the best one was selected for library amplification efficiency by qPCR and band purity by gel electrophoresis. For sample barcodes, a large set of distinct sample barcodes need to be error-tolerant and color-balanced for Illumina™ sequencing machines. The IDT for Illumina™ unique dual (UD) index set (384 dual index pairs) were concatenated and expanded to 960 unique barcodes by inserting three blocks of sequence tags (see e.g., FIG. 14A). This method ensures a minimum Hamming distance of 12 between any two barcodes, and thus is tolerant to up to 6 nucleotide substitutions and resistant to even a higher level of polymerase errors and/or sequencing errors. To select for barcodes that have low secondary structure and are compatible with our workflow, barcoded RT primers with all 960 barcodes (see e.g., Table 5) were synthesized and pooled 10x in 96-well plates of contrived samples using synthetic viral RNA spike-in. After pooled amplification and sequencing, those barcodes that produced read counts higher than a set threshold were selected and used for subsequent tests (see e.g., FIG. 14B, FIG. 25A).

Amplification efficiency and dynamic range were tested for these selected barcodes, with a 10x dilution series (see e.g., FIG. 25B). For high-load samples, a linear response was observed with a dynamic range of ~10⁴; the detection sensitivity was low, likely due to PCR inhibitors present in pooled RT samples. To improve PCR amplification efficiency (e.g., by removing PCR inhibitors expected to be present in pooled RT samples), spinning column cDNA purification was performed after sample pooling. This step also had the added benefit of reducing sample volume to a manageable level, after pooling a large number of patient samples. After cDNA purification and using 96 selected high-quality barcodes (see e.g., Table 6), the LoD was 12 gce (see e.g., FIG. 14C), which is about 5-fold lower than the qPCR readout, indicating some degree of sample loss and degradation during the cDNA purification, library amplification and sequencing steps.

### Dynamic Strand Displacement With a “Protector” Oligonucleotide Effectively Suppresses Barcode Crosstalk and Preserves Sample Dynamic Range

Suppressing off-target barcode crosstalk and preserving high sample dynamic range are critical for faithful diagnostics, such as COVID-19 since clinical samples have been shown to exhibit a large dynamic range (up to 10⁶to 10⁷) of detectable viral load, and any barcode mis-assignment could result in false positive diagnoses; see e.g., Bar-On et al. SARS-CoV-2 (COVID-19) by the numbers. Elife 9 (Mar. 30, 2020); Arnaout et al., supra. The degree of barcode crosstalk in the workflow was first assayed by pooling 1 or 10 barcoded RT samples prepared with high spiked-in viral load together with 95 or 86 negative samples with other barcodes, and sequencing reads carrying any of the off-target barcodes were tallied (see e.g., FIG. 15A). Without any special treatment, there was a 0.1% barcode crosstalk on average, resulting in an upper limit of 3 logs of detectable sample dynamic range (see e.g., FIG. 15B), much lower than what is required for faithful COVID-19 diagnostics when a high-load sample is present.

A major source of barcode crosstalk in a “pooling-after-amplification” workflow is from cross-hybridization of excess library adapters during the cluster amplification process, which then produces mis-barcoded transcripts; see e.g., Kircher et al, Nucleic Acids Res 40, e3 (2012). A similar mechanism with cross-hybridized excess RT primers during the library amplification step can account for the main source of the 0.1% barcode crosstalk observed in the One-Seq workflow. Methods for minimizing crosstalk using unique dual indices is not compatible with a “pooling-before-amplification” strategy. Described herein is a strategy to reduce this crosstalk by suppressing cross-hybridization of excess RT primers, e.g., during the PCR step (see e.g., FIG. 15C, top panel). To do this a single-stranded “protector” oligonucleotide was designed that comprises the RT primer (without barcode) and an extended sequence complementary to the viral genome downstream. By the principle of dynamic strand displacement, the extended sequence functions as a toehold and provides stable binding of the protector strand to the cDNA, which then competes off any off-target RT primer from cross-hybridization (see e.g., FIG. 15C, top panel).

First, a simple test of this protector strategy was performed using a short DNA amplicon together with an off-target barcoded RT primer, and using qPCR as the readout. The test included several different protector strand designs, including a naive approach using the complement of the RT primer sequence (see e.g., FIG. 15C, bottom panel). The protector strand significantly reduced off-target PCR amplification, and longer toehold lengths (e.g., up to 30 nt) provided more stable binding, leading to more effective suppression (see e.g., FIG. 15D). Increasing protector strand concentration and raising annealing temperature also each improved the suppression effect, as both favor the binding of the protector strand compared to that of the off-target primer. Under optimized conditions, the results showed up to 10⁵-fold suppression of off-target amplification. The effect of the RT primer concentration was also tested (see e.g., FIG. 15E). Lowering RT primer concentration by 100x alone reduced barcode crosstalk by 1,000-fold; and an overall 10⁹-fold suppression was achieved when used in combination with the protector strand.

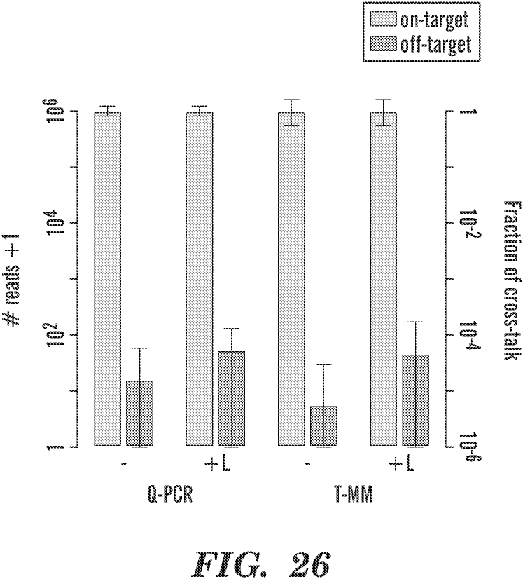

Next, the protector strategy was tested in multiplexed sequencing settings and in contrived clinical samples, following similar test design as above (1-10 high-load sample along with ~90 off-target barcodes) (see e.g., FIG. 15F, FIG. 26). Using the protector strategy significantly reduced the level of barcode crosstalk from 0.03% to 0.0001% (i.e.,300-fold reduction) (see e.g., FIG. 15F). Performance of the protector strategy was then stress tested by supplementing extra off-target RT primer mix into the PCR reaction (see e.g., FIG. 15F). Without adding the protector strand, there was a significantly higher barcode crosstalk (0.1%-6%); with protector strand, the crosstalk level was again significantly suppressed (0.001%-0.01%). To further reduce barcode crosstalk, the effects of RT primer removal by several cDNA purification methods were compared (see e.g., FIG. 15G, FIG. 26). Bead-based purification methods (e.g., Thermo MagMax™ kit) produced a lower level of barcode crosstalk (0.001%) compared to spin column-based purification methods (e.g. QIAquick™ PCR purification kit), likely due to of a sharper size selection cut-off. Since the spiked-in samples have a very high viral load (equivalent to 2x10⁹ gce/ul in patient sample, or Ct=12), a much lower level of barcode crosstalk can occur in practical scenarios, allowing for a dynamic range of 10⁶ to 10⁻⁷, fulfilling the requirement for faithful SARS-CoV-2 detection in patient samples.

### Validation of One-Seq in Clinical Samples

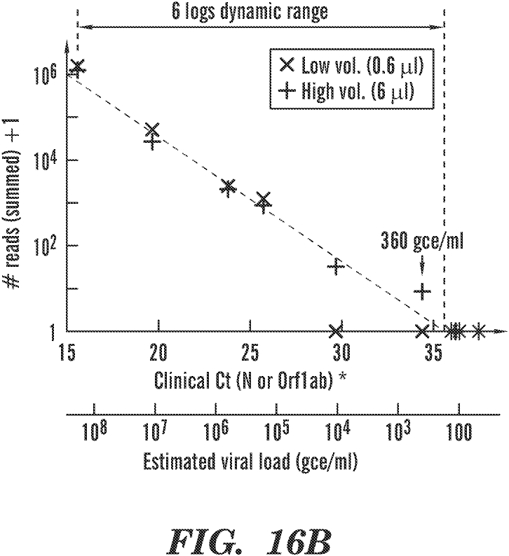

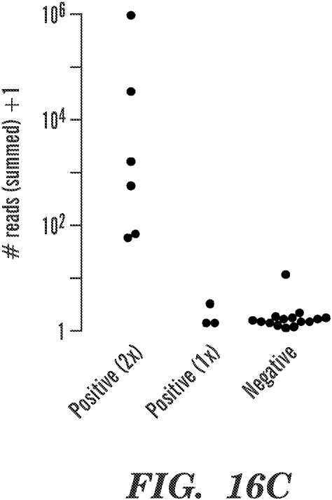

Performance of the method was validated using SARS-CoV-2 positive clinical samples (see e.g., FIG. 16A). To mimic realistic conditions, remnant clinical NP swab samples that had not been heat-inactivated were used, and samples collected in several different viral transport media were compared. Samples collected in most widely used viral transport media were compatible with the One-Seq reaction buffer. Only Hologic Aptima™ swab samples were incompatible with the One-Seq method, generating snow-like aggregates, most likely due to the precipitation of lauryl sulfate in the Aptima™ buffer with potassium ion in the One-Seq buffer.

To test the detection sensitivity as well as dynamic range of our method, a set of representative COVID-19 positive samples (Np swab in VTM) were chosen that spanned a wide range of clinical Ct values (e.g., from 15 to 38), and the samples were subjected to the One-Seq workflow. For this test, three distinct barcodes were mixed together for each sample and their sequencing reads were summed, to maximize the sensitivity and robustness of detection. The first assay tested the detection sensitivity of One-Seq and its dependence on input sample volume (see e.g., FIG. 16B). As expected, higher sample volume allowed higher detection sensitivity. With only 6 ul per sample input, the One-Seq method correctly reported the presence of SARS-CoV-2 RNA in all samples with a clinically determined Ct value <35, and no false positives.

The lowest sample concentration detected was at 360 gce/ul (Ct = 34.39), indicating that One-Seq can detect clinical samples with viral load in the 200-500 gce/ul range, using a single amplicon. There was a linear correlation between the detected sequencing reads and estimated viral load (calculated from clinical Ct values), over the entire range of Ct values (from 15 to 35), demonstrating that One-Seq faithfully reports viral load in a quantitative manner over 6 logs of dynamic range (see e.g., FIG. 16B). There was a slight ratio compression in the sequencing reads, possibly resulting from a decreased RT reaction efficiency in high-load samples, due to the constraints in RT primers and enzymes available. A second test was then performed with both COVID-19 positive and negative samples (NP swabs in VTM, total N=28), and a clear separation was observed between these samples (see e.g., FIG. 16C).

In this test, there were three clinically determined positive samples that were not detected. Notably all three had only one of the two targets detected by RT-qPCR (i.e. either the SARS-CoV-2 N gene or SARS-CoV-2 orflab gene was not detected), and they all had Ct values >36 for the detected target. If these samples were indeed actually positive, they were likely missed by the One-Seq test due to the small sample volume (6 ul) used in this test as compared to a typical RT-qPCR test (300 ul or more); further increasing sample volume can improve the detection sensitivity.

### Multi-Primer Detection and Variant Sequencing

Simultaneous detection using multiple RT primers allows multi-loci, multi-virus diagnostics, with increased viral detection sensitivity. Furthermore, if the RT primers are designed to be in close proximity to mutation hotspots (see e.g., FIG. 17A), it is possible to obtain extra viral sequence information to allow variant identification, without significantly increasing the test turn-around time. The developments in the COVID-19 pandemic indicated that a very useful application of One-Seq is for surveillance of viral variants or simultaneous detection of multiple viruses.

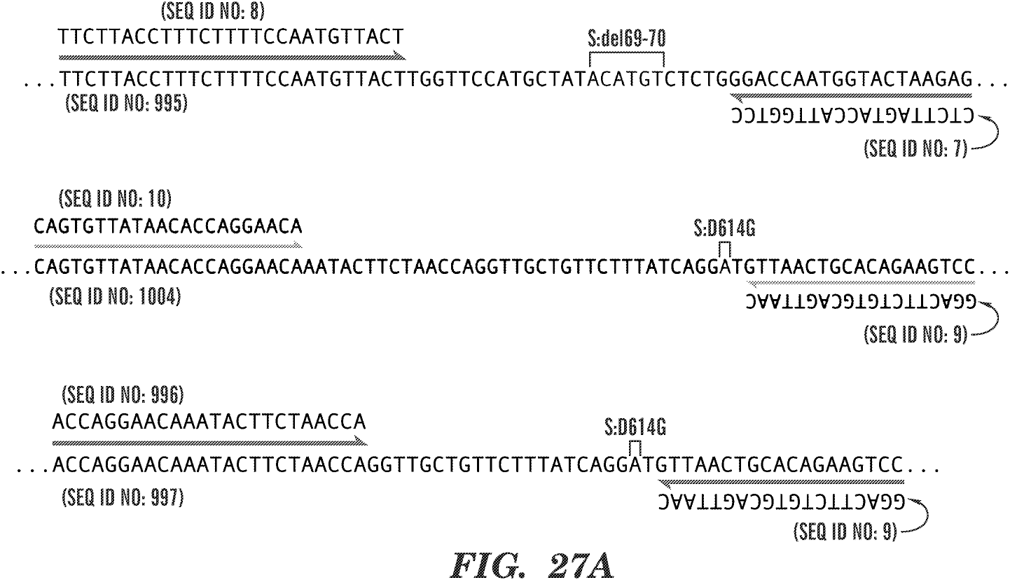

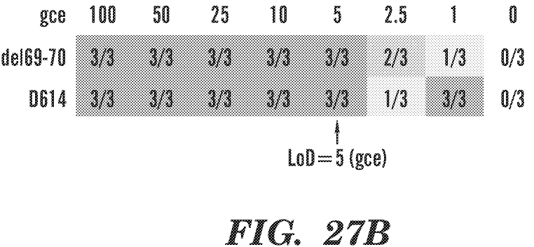

RT primers were designed targeting several characteristic mutations in the SARS-CoV-2 S gene for the reported variant B.1.1.7, including del69-70, dell44, N501Y, D614G and A701V, and dye-based qPCR was used to assay for RT efficiency. It was not always easy to design good RT primers in close proximity to the target mutations, likely due to the presence of strong local secondary structure in the RNA (see e.g., FIG. 17B). As a result, the first batch of primer designs yielded two good candidates with high RT sensitivity (LoD <5 gce) (see e.g., FIG. 27). Sensitivity tests were performed for these two primers in contrived clinical samples and in 96x multiplexed format, and the results indicated limits of detection of 10-30 gce for both primers.

In silico analysis was performed for primer inclusivity and specificity for all designed primer pairs, following FDA guidelines. All primers aligned to all available SARS-CoV-2 genome sequences in the NCBI database (98,765 sequences) with at most 1 base mismatch, and 7 out of the 8 primers showed exact match to >99.4% of all sequences (see e.g., Table 7). Since One-Seq performs RT and PCR in separate steps, cross-reactivity analysis was only performed on RT primers. All four RT primers showed no significant (>80%) homology to genome sequences of common respiratory flora and other related viruses (see e.g., Table 8). In addition, One-Seq reads a short sequence into the viral genome, providing highly specific viral detection.

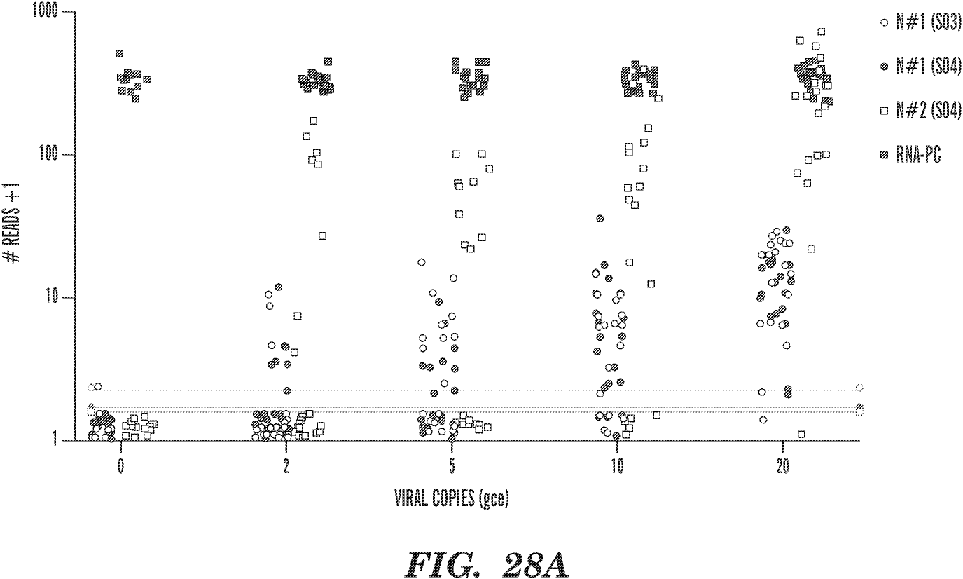

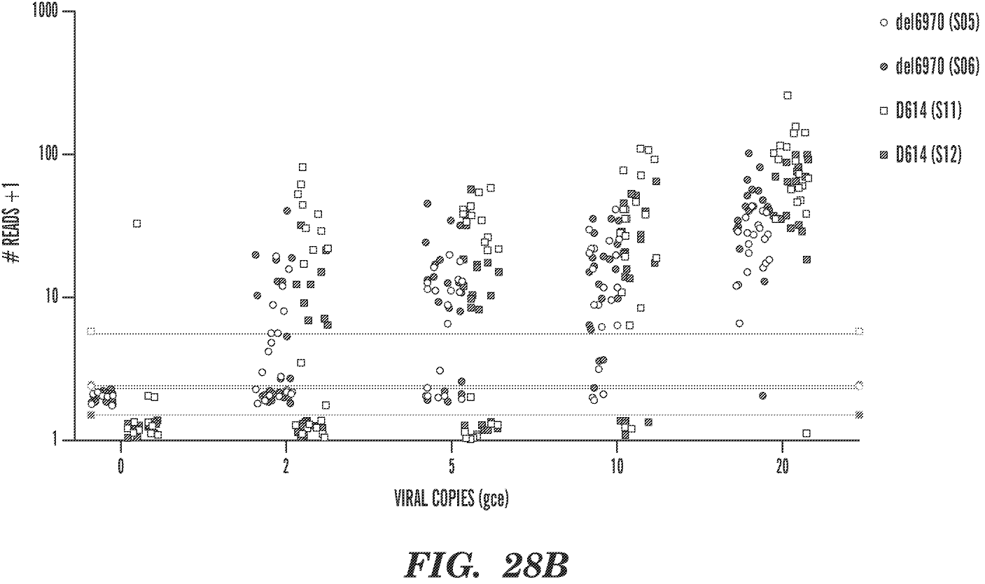

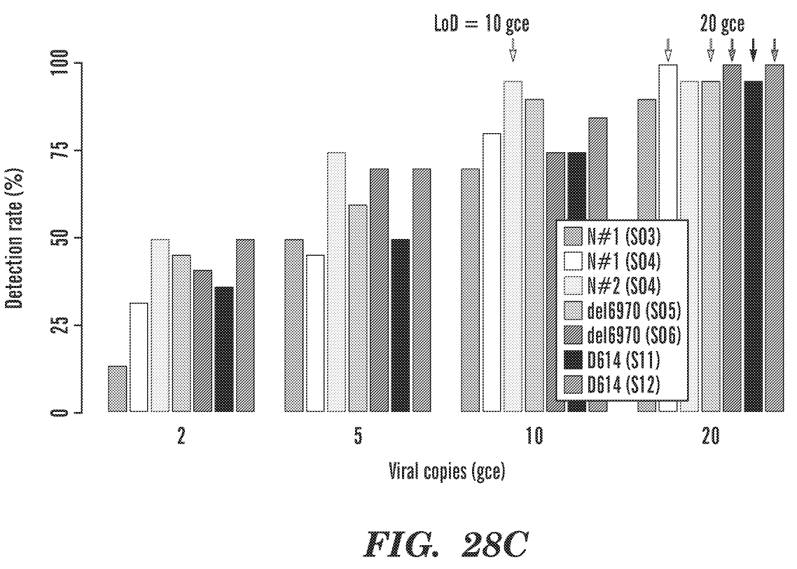

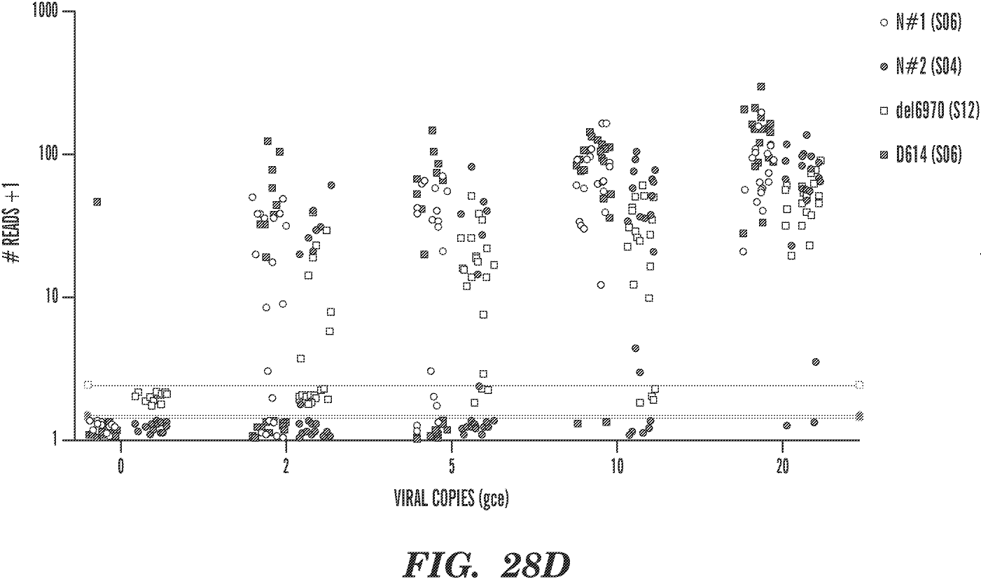

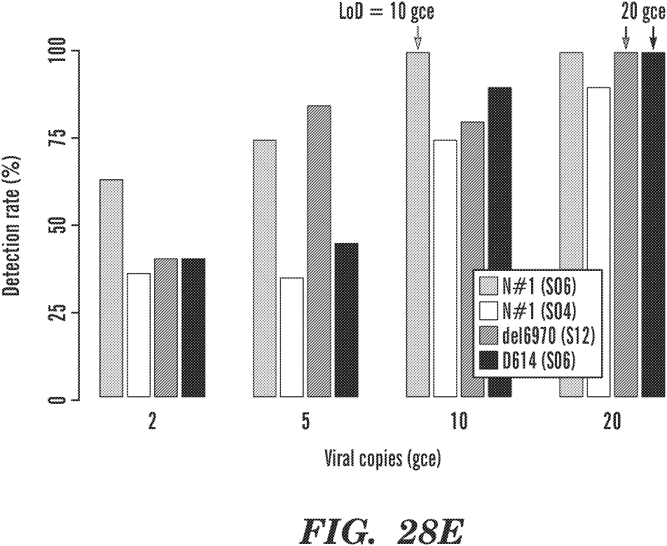

Next, a confirmatory clinical sensitivity test was performed for all designed primer pairs (4 in total) in a similar 96x multiplexed format, in both single-primer and multi-primer settings (see e.g., FIG. 28). For this test, only one unique barcode was used per sample. In single-primer tests, all four primer pairs had an LoD = 20 gce by the 95% detection rate cut-off (see e.g., FIGS. 28A-28C), confirming the results from FIG. 27. In multi-primer tests, three of the four primer pairs performed well and showed an LoD of 10-20 gce (95% cut-off; all four LoD ≤20 when using a 90% cut-off), and primer N#1 showed an even higher sensitivity at LoD = 10 gce (95% cut-off) (see e.g., FIG. 17C, FIGS. 28D-28E). These results indicate that multiplexed RT and library amplification can work well, and there is no significant interference between the designed primers. Another experiment tested if the use of multiple primers can further improve detection sensitivity. Indeed, there was a higher detection rate as more primers are used (see e.g., FIG. 17D). When all four primers were used, there was an LoD of 5 gce (95% cut-off; 2 gce using 90% cut-off).

For a 4-primer multiplexed test with a 20 ul patient sample intake, this result translates to an LoD = 100-250 gce/ml in clinical samples, approaching the detection limit of RT-qPCR tests. Further increasing sample input volume, or using more primers in parallel can both further increase the detection sensitivity in a linear fashion, e.g. taking 300 ul specimen (typical for RT-qPCR tests) can allow an LoD down to 5-10 gce/ml.

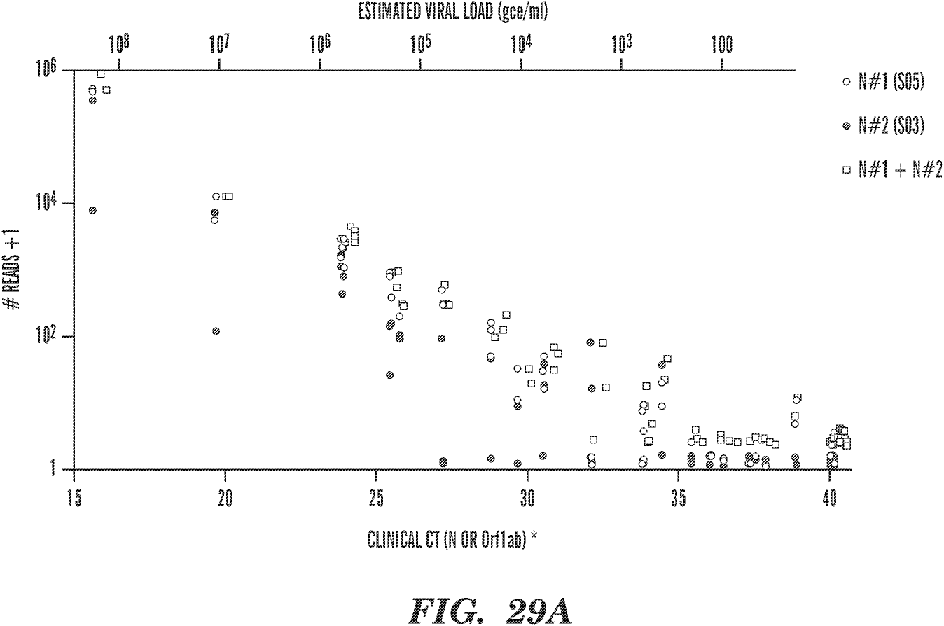

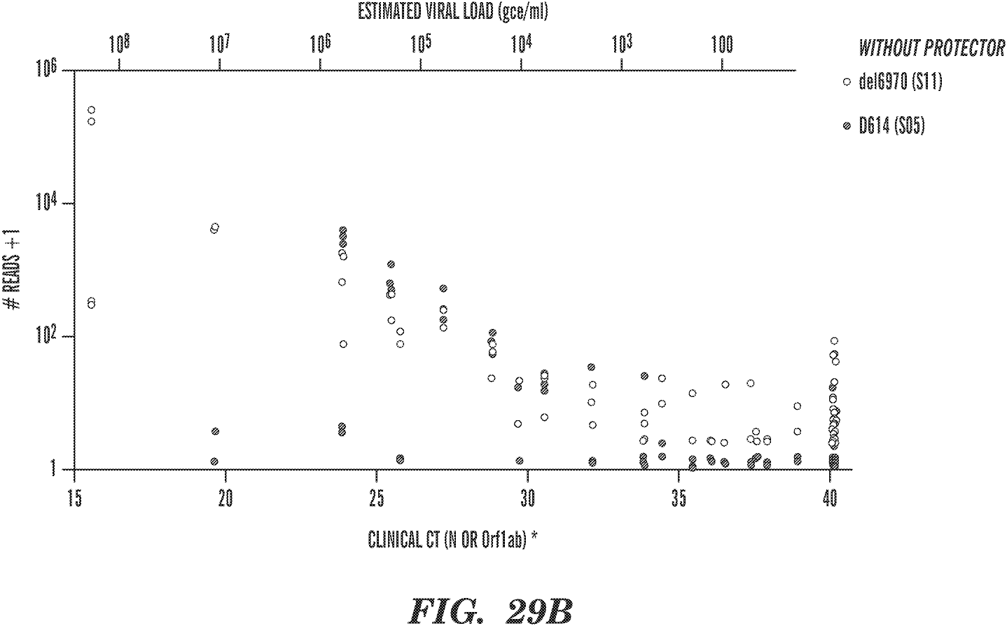

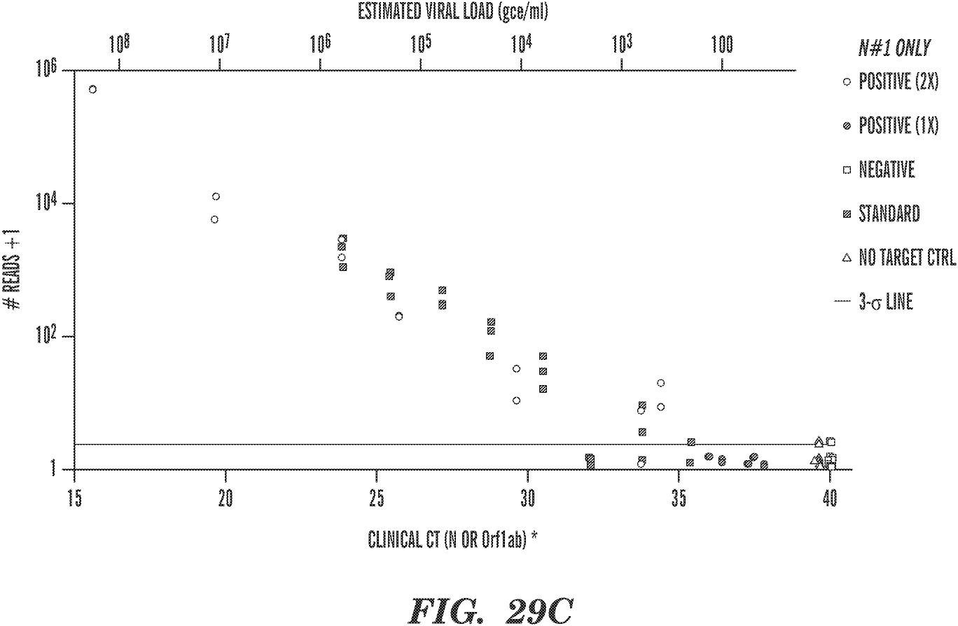

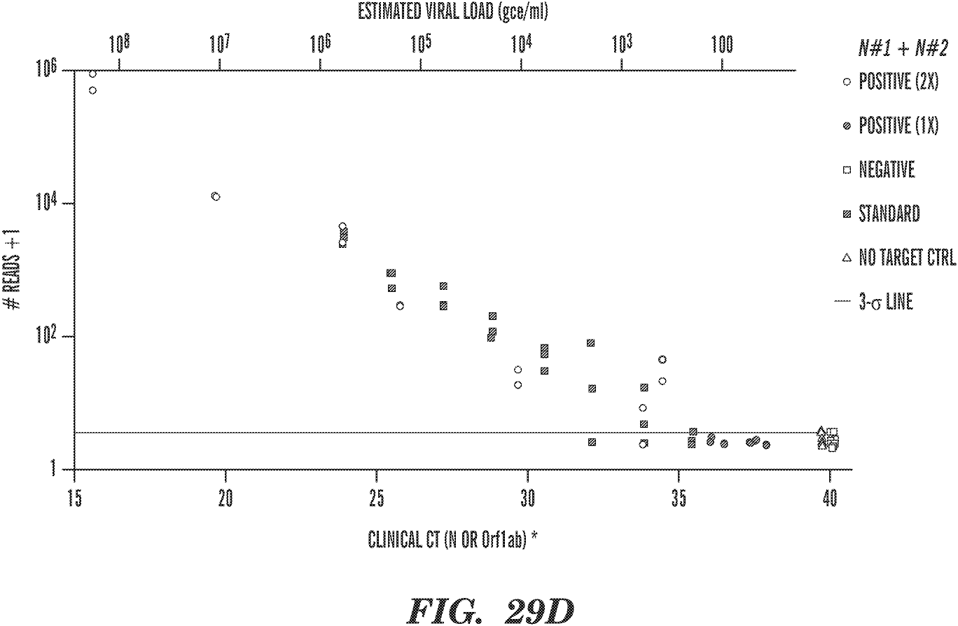

Finally, One-Seq was tested multi-primer detection in clinical samples in a 96x multiplexed format, consisting of 56 COVID-19 clinical samples (two repeats of 28 specimens), 24 contrived standards, and 16 no-target negative controls (see e.g., FIG. 29). All four RT primers designed above were used, two primers for diagnostics targeting the SARS-CoV-2 N gene, and two primers for mutation sequencing targeting the SARS-CoV-2 S gene. Using 5 ul sample volume, One-Seq correctly reported the low viral load sample (360 gce/ul) in both repeats, and again exhibited a linear dynamic range of ~10⁶, allowing quantitative report of viral load. The viral sequences from the two mutation-targeting primers in the SARS-CoV-2 S gene were analyzed (see e.g., FIG. 17E). The D614G mutation was present in all positive clinical samples tested, except the inactivated virus standard (isolate USA-WA1/2020, January 2020), indicating that the D614G mutation was already prevalent in July 2020, when this batch of samples were originally collected. There was no evidence of the del6970 mutation, indicated that none of these samples were related to the later discovered B.1.1.7 variant.

### Discussion

Described herein is a method for viral RNA molecular diagnostics (e.g. SARS-CoV-2) that allows highly scalable central lab testing, achieves high detection sensitivity, and provides sequence information at targeted mutation hotspots, allowing for viral variant identification. To permit such high scalability, the method includes a “pooling-before-amplification” strategy and avoids the high-complexity steps of RNA extraction and PCR thermocycling, thus eliminating current bottlenecks in scalability. To permit early pooling, a one-pot reaction was used for efficient reverse transcription (RT) and upfront barcoding, and a “protector” strategy was used that preserved the high dynamic range of viral load in patient samples. One-Seq can reach a high detection sensitivity in unextracted samples, down to 10 gce (e.g., per 20 uL sample) by multiplexed sequencing for a single primer, and down to 2-5 gce (e.g., per 20 uL sample) for multi-primer detection with four primers. Assuming 20 ul sample intake, this is equivalent to a viral load of 100-250 gce/ml in unextracted patient sample, approaching the maximum sensitivity of extraction-based RT-qPCR assays. Scaling up sample volume can further improve the detection sensitivity linearly. In clinical samples, One-Seq quantitatively reported patient viral load, preserved 6 logs of linear dynamic range of viral load (estimated from clinical Ct values), and detected SARS-CoV-2 positive samples down to ~300 gce/ml in viral load. One-Seq further reports sequences at a number of viral mutation hotspots, allowing for variant identification at equal scalability with no extra cost.

One-Seq can be used with a two-stage barcoding and pooling strategy to test a large number (e.g., 100,000) of patient specimens, without the need to design and manufacture an equally large number of distinct barcodes (see e.g., FIGS. 12B-12C). To implement this strategy, patient specimens can be collected into different “batches” (e.g. by local community, organization, or department). Samples in each batch are pooled and processed together. Each batch is then barcoded on the reverse side during the library amplification step, after which a number of sample batches are pooled together for multiplexed sequencing. This two-stage barcoding strategy provides two benefits. First, it significantly reduces the overhead in barcode design, manufacturing and regulatory approval. Second, it allows the method to be adapted and applied to different application scenarios, for example in an isolated environment (e.g., a cruise ship) where only a limited number of individuals needs to be tested regularly. In such a scenario, One-Seq can be adapted to use the same barcode set but with less second-stage (e.g., post-amplification) pooling, and sequenced on a lower-throughput machine (e.g., Illumina™ NextSeq™ 550).

One-Seq is highly scalable, cost-effective, with a fast turn-around (see e.g., Table 2). Using a high output Illumina™ sequencer such as the NovaSeq™ 6000, a maximum sample throughput is 100,000-160,000 samples per day per machine, allowing an overall throughput of up to 1,000,000 tests per day in a single clinical lab, using multiple sequencers. Further increase in sample throughput as well as cost reduction are possible with other sequencing modalities (e.g. Oxford Nanopore PromethION™ 48 allows 5x lower sequencing reagent cost, and up to 180,000 tests per day at comparable capital cost) (see e.g., Table 2). Depending on the sequencer model used and whether batch pooling and viral sequencing are desired, One-Seq sample turn-around time (TAT) ranges from a minimum of 7.5 hr (for a single batch on a MiSeq™, without viral sequencing) to a maximum of 14.5 hr (for batch pooling on a NovaSeq™ 6000, with viral sequencing), allowing for diagnostic results to be available within 24 hr of sample collection or drop-off (see e.g., Table 9). The cost per sample for the One-Seq method also scales favorably for highly-multiplexed settings. At relatively small scale (e.g., 80 samples per run on a MiSeq™ sequencer) and using off-the-shelf reagents, the cost of the method is at $20 per test; at large scale, (e.g., 40,000 samples per run on a NovaSeq™) sequencing reagent cost is reduced to <$0.5 per sample, and mass production can lower enzyme and reagent cost by 70% or more, bringing the total cost down to $3 (see e.g., Table 10). Due to the minimum sample processing needed for the One-Seq workflow, the consumable cost (e.g., tips, tubes) is also considerably lower, making the total cost per test lower than RT-qPCR or sequencing-based testing methods. In addition to scalability, One-Seq also shows superior performance in comparison with other methods, and offers high detection sensitivity (down to LoD = 100-250 gce/ml), and ability to test unextracted clinical samples (see e.g., Table 3). Taken together, One-Seq offers a technically and economically viable solution for highly-scalable testing on a population scale.

One-Seq also allows detection of viral hotspot mutations and monitoring of their transmission dynamics (see e.g., Table 3). This is especially important as certain mutations can convey higher transmission rate or pathogenicity (e.g. B.1.1.7 of SARS-CoV-2) or evasion from immunity induced by vaccination or prior infection (e.g. E484K of SARS-CoV-2). It has been increasingly appreciated that identifying and tracking viral variants is as critical as diagnostic screening, and sequencing remains the only method available for effective variant identification. Current whole-genome sequencing (WGS) methods (e.g. Illumina™ COVIDSeq) typically require 50-100x sequencing reads for the same sample and are further bottlenecked in throughput by the PCR-limited sample preparation steps. In contrast, One-Seq uses targeted sequencing that requires much fewer reads per sample, and allows much higher scalability and lower amortized cost. Therefore, One-Seq is ideally suited for variant identification and tracking.

One-Seq can be clinically implemented in at least one of two ways to permit highly-scalable viral diagnostics (see e.g., FIG. 18A). First, One-Seq can be directly used in a clinical lab with pre-collected specimens (e.g., swab or saliva in transport media) to achieve extraction-free, highly-scalable diagnostics. Alternatively, patient specimens can be directly collected into purpose-designed collection tubes containing One-Seq reagents and uniquely barcoded RT primers, they can be and pooled immediately after incubation at the testing facility. The latter implementation allows an even higher degree of scalability, as it completely avoids any liquid handling step for individual samples (see e.g., FIG. 18B), and it reduces the logistic complexity from one that scales with the number of samples to one that is largely independent of the number of samples (i.e., from O(N) to O(1)).

Finally, One-Seq is flexible in at least two important ways: it can be continually updated in a matter of days to include RT primers targeting emerging viral mutations as they appear, providing a real-time monitoring of viral evolution and transmission during an ongoing pandemic; and it can be targeted to detect any single-stranded RNA viruses of positive and negative sense, including the common cold, seasonal flu, hepatitis, dengue, Ebola, West Nile, Zika, and more, or a number of them in a multiplexed manner. One-Seq allows for population-scale surveillance with a panel of viruses of special concern, allowing for the reporting of a “bio-weather map” for the early identification and tracking of emerging viral hotspots, in order to help prevent future viral outbreaks.

### Methods

### Clinical Specimen and Reference Materials

All clinical specimen and saliva samples used in the study were deidentified. Remnant clinical nasopharyngeal swab samples were obtained from Boca Biolistics™. None of the clinical specimens were heat-inactivated prior to use, and all operations with clinical specimens were performed inside a biosafety cabinet (BSC) following BL2+ safety protocols. SARS-CoV-2 inactivated virus standard materials were obtained from ATCC (VR-1986HK) or SeraCare™ (AccuPlex™ 0505-0168). In vitro transcribed SARS-CoV-2 viral N gene mRNA were prepared with Invitrogen™ MAXIscript™ T7 transcription kit (ThermoFisher™, AM1312), following manufacturer’s protocol. The template DNA was prepared from N positive control plasmid (IDT, 10006625) with T7 promoter-containing primers, and purified from an agarose gel using QIAquick™ PCR purification kit (QIAGEN, 28104).

### Preparation of Contrived Specimens

For clinical limit of detection studies, pooled confirmed COVID-19 negative remnant nasopharyngeal swab specimens purchased from Boca Biolistics™ (N=15) were used. Pooled clinical samples were then spiked in with ATCC or SeraCare™ inactivated virus standard, or in vitro transcribed viral RNA at various specified concentrations, pre-diluted into viral transport medium (VTM). VTM was prepared with 2% FBS (heat-inactivated at 56° C. for 30 min, Gibco™ 26140079), 1x Antibiotic-Antimycotic (Gibco™, 15240096) and 11 mg/L phenol red, in 1x Hank’s balanced salt solution (Gibco™, 14025092). None of the contrived clinical samples were pre-heat-inactivated before one-pot reverse transcription step.

For reverse transcription efficiency studies, pooled saliva specimen collected from COVID-19 negative donors were used, either with (N=4, “clean”) or without (N=9, “dirty) mouth rinsing before collection. Pooled saliva samples were then spiked with ATCC inactivated virus standard, or in vitro transcribed viral RNA, at specified concentrations, as above.

### Primer, Barcode and Sequencing Construct Designs

Reverse transcription primers were designed following these criteria: (i) Tm (calculated with IDT oligo analyzer, RNA-targeting primer) in range of 54° C.-60° C., strong 3′-end binding (e.g., the presence of G or C bases within the last five bases from the 3′ end of primers (i.e., GC clamp) helps promote specific binding at the 3′ end due to the stronger bonding of G and C bases.), and (ii) high sequence coverage of available SARS-CoV-2 genomes and low homology with SARS, MERS, and related viral sequences. Furthermore, RT primers targeting mutation hotspots were designed to be in close vicinity (e.g., within 5 nt) to the targeted loci, to avoid significantly increasing the sequencing runtime (see e.g., FIG. 17A). Reverse primers for PCR are designed following these criteria: (i) Tm in range of 60° C.-62° C., weak 3′-end binding (e.g., which can be advantageous for multiplex reactions and can reduce off-target reactions), and (ii) high sequence coverage of available SARS-CoV-2 genomes.

960 unique patient barcodes were designed by concatenating the i7 and i5 sequences and further expanding from IDT for Illumina™ Unique Dual Index set (4x96=384 pairs in total; see e.g., FIG. 14A). The following sequences were inserted in between the sequence blocks: ...AC.. .TG...AC... (4x96) (nnnnnACnnnnnTGnnnnnACnnnnn, SEQ ID NO: 998), ... CA... CT... GA... (4x96) (nnnnnCAnnnnnCTnnnnnGAnnnnn, SEQ ID NO: 999), ...AC...AC...TG... (2x96) (nnnnnACnnnnnACnnnnnTGnnnnn, SEQ ID NO: 1000). Such a design ensures a minimum Hamming distance of 12 between any two barcodes, and avoids any homopolymer repeats longer than 3 nucleotides. 12 reverse PCR barcodes for “batch” pooling were selected from the set of IDT8 indices.

Sequencing constructs were designed using custom read primers and PCR adapters. Read primers were designed to be orthogonal to sequencing adapters and have Tm > 70° C. A short PCR adapter sequence, which forms a part of the read 1 primer, was designed to allow for pooled amplification using a common forward primer and also compatible with the protector strand. A detailed illustration of the sequencing construct including example sequences are given in FIG. 20.

A full list of all primers, barcodes and adapters used in this study is provided in Tables 4-6 (Table 4: primers, adapters, batch barcodes; Table 5: 960 sample barcodes; Table 6: 96 selected sample barcodes).

### Synthetic Positive Control RNA

Positive control RNA (e.g., SEQ ID NO: 11) was designed to start with the same RT primer with the SARS-CoV-2 N gene targeting primer N#1, and extended with 8 nt sequence distinct from the viral genome. Synthetic RNA was purchased from IDT, and spiked into all samples at a concentration of 10⁴-10⁵ copies/ul to provide positive control reads.

### One-Pot Sample Processing Reaction

One-pot sample reaction for viral lysis, reverse transcription and sample barcoding was performed with SuperScript™ IV reverse transcriptase (Thermo™, 18090010) in manufacturer provided reaction buffer (without DTT), supplemented with 10% (v/v) murine RNAse inhibitor ( New England Biolabs™, M0314), 0.1% Triton X-100, 1x Antibiotic-Antimycotic (Gibco™, 15240096), 0.5 mM EDTA, 5 mM DTT, cOmplete™ protease inhibitor cocktail (1 tablet into 13.3 ml, Sigma™, 11873580001), 0.5 uM poly-A60 DNA oligonucleotide, 15 ug/ml E. coli tRNA (Sigma™, 10109541001) and 10⁴-10⁵ copies/ul synthetic RNA for positive control, further added with 35-50% (v/v) equivalent of viral transport media or pooled clinical or saliva sample and 125 nM of barcoded RT primer (for each primer). For limit of detection studies, inactivated virus standard from ATCC or SeraCare™ was spiked into the one-pot reaction at specified concentrations. For barcode crosstalk studies, in vitro transcribed viral mRNA was used. For viral lysis and sample preservation studies, different subsets of above components were added to the reaction mix. For primer concentration studies, 25 nM-500 nM of barcoded RT primers were used. For multiplexed sequencing samples, a master mix of above reaction mix without barcoded primer and contrived clinical sample was first prepared and aliquoted into a 96-well plate, then RT primers with unique barcodes and samples was added to each well.

One-pot reactions were assembled on ice-cold blocks. Once assembled, the reaction was incubated at 50° C. for 30 minutes (min), followed by inactivation at 95° C. for 5 min. For tests with contrived samples, incubation was performed in a closed-lid PCR thermocycler; for tests with clinical specimen, incubation was performed in a heat block, and followed by another inactivation session at 95° C. for 5 min in a closed-lid thermocycler once moved out of the BSC. For sample preservation studies, the assembled reaction was left at room temperature and covered for up to 24 hours (hr) before starting the 50° C. incubation.

### qPCR Quantitation

For limit of detection studies for N# 1 and N#2 primers, and RT quality control for clinical sample tests, qPCR was performed after the one-pot sample reaction. 0.5 ul-1.0 ul one-pot reaction sample was added to 40 ul qPCR mix (40x-80x dilution), containing Taq polymerase and standard buffer (New England Biolabs™, M0273), 0.2 mM dNTP mix and CDC SARS-CoV-2 primer and probe set at 0.5 uM equivalent primer concentration (IDT RUO kit, 10006713). Formation of cloudy aggregation was observed in certain clinical samples after the one-pot reaction. In such situation, to ensure adequate sample intake, the one-pot reactions were mixed with pipetting a few times before adding to the qPCR reaction. For limit of detection studies for variant targeting primers, qPCR was performed with dye-based readout, using Luna™ universal qPCR master mix (New England Biolabs™, M3003) and 0.5 uM of both forward and reverse PCR primers.

qPCR samples were run on a Bio-Rad™ C1000 thermal cycler and CFX real-time PCR system for 50 cycles, and optionally with melt curve measurement for dye-based readout. Ct values were determined by manufacturer’s auto-thresholding function when possible. For preliminary clinical sensitivity studies, limit of detection (LoD) was determined to be the lowest viral spike-in concentration at which all 3/3 tests yielded a valid Ct value. For dye-based qPCR, results were interpreted with melt curve analysis instead of Ct values.

### Sample Pooling and cDNA Purification

One-pot reaction samples (20 ul-80 ul each) were pooled by multichannel pipettes from 96-well plate to a single tube and immediately proceeded to cDNA purification using spin column (QIAquick™ PCR purification kit, QIAGEN 28104) or bead-based method (MagMax™ viral/pathogen nucleic acid isolation kit, Thermal™ A42352). The manufacturer’s protocols were adapted for large input sample volume and high sensitivity recovery. For column purification, the sample was added multiple times to the same spin column. For bead purification, large 50 ml conical tubes were used, and centrifugation (e.g., 3,000 rcf for 3 min) was used instead of magnetic attraction for effective collection of the beads. To ensure maximum recovery, only DNA low-bind tubes and pipette tips were used for this step. The purified cDNA library was supplemented with carrier DNA and RNA (e.g., poly-A60 oligonucleotide and E. coli tRNA) to further avoid sample loss on tube walls. For purification method comparison studies, QIAquick™ nucleotide removal kit (QIAGEN, 28304) was also compared to AmPure™ XP beads (Beckman Coulter™, A63880), both following manufacturer’s protocols.

### Library Amplification and Quantitation

The pooled and purified cDNA library was amplified in a dUTP-incorporating PCR reaction, using Luna™ universal qPCR master mix (New England Biolabs™, M3003), supplemented with Uracil-DNA Glycosylase (UDG) enzyme at 25 units/ml (New England Biolabs™, M0372). For single-primer detection, 0.25 uM of both forward and reverse primers were used. For multi-primer detection with 4 primers, 0.5 uM of forward and 0.125 uM of each of the reverse primers were used. For multiplexed sequencing tests on clinical samples, 2 uM protector oligonucleotide was added. For protector concentration studies 0.5 uM-5 uM protector was used. For barcode crosstalk studies, a mixture of 86 or 95 off-target barcoded RT primers was further supplemented into the reaction. Library amplification was run for 40-50 cycles with a custom-optimized thermocycling program: the first two cycles used a low annealing temperature (e.g., 52° C. -58° C.), and the remaining cycles used a high annealing temperature (e.g., 68° C.).

The amplified library samples were within the 200 bp-260 bp range. Since non-specific amplification products can adversely affect loading concentration and sequencing quality, library quality was assessed on agarose gel and the desired band was purified using QIAquick™ PCR purification kit (QIAGEN, 28104). The purified library sample was then normalized using either Qubit™ or Agilent TapeStation™ before proceeding to sequencing run.

### Sequencing Protocol

Sample libraries were sequenced on an Illumina MiSeq™ machine, at a loading concentration of 10 pM (for V2 Micro kit, 300-culec, MS-103-1002) or 20 pM (for V3 kit, 150-cycle, MS-102-3001), supplemented with 15-20% Phi-X control v3 (Illumina™, FC-110-3001). To avoid template carryover contamination between consecutive sequencing runs, two template line washes (e.g., containing sodium hypochlorite solution, Sigma™, 239305) were performed between each run, following Illumina™ protocol.

Since the sequencing construct as well as barcodes were custom designed, custom read primers were spiked into the sequencing kit following Illumina™ protocols (e.g., 2 ul of 100 uM R1 custom read primer into well 12, and 2 ul of R2 primer into well 14). Sequencing was performed for 100+100 bases (for V2 Micro kit, 300-cycle) or 100 \+68 bases (for V3 kit, 150-cycle) with no indexing reads for developing the test; this can be shortened to 40-60 cycles for clinical use.

### Sequencing Analysis

The bioinformatic analysis of sequencing results was performed in a few steps: FASTQ generation and adapter trimming (Illumina™ BaseSpace), sequence alignment (bowtie2™), demultiplexing and read counting (custom scripts in MATLAB and Excel™). Here sequence alignment was performed against sequences from one or multiple RT primers, allowing for ≤2 edit distance between the library and sequencing read. In the case of viral sequencing and mutation identification, the reads were aligned against both original and mutated viral sequences, and the best matched genotype was reported. After alignment, each sample was identified using a combination of a front sample barcode, and a reverse batch barcode. All sequencing read counts were added by 1 to allow easy plotting. The analysis pipeline takes 20-30 min per run. The analysis pipeline involves a fast and user-friendly analysis workflow.

### Analysis of Barcode Crosstalk and Dynamic Range

For barcode crosstalk studies with 1-10 high-load barcoded samples, supplemented with 86-95 off-target RT primers, after sequence alignment, the matched sequence counts for both groups of barcodes (on-target and off-target) were separately tallied. Read counts from the high-load samples were then normalized to 10⁶, and then counts from the off-target barcodes and relative level of crosstalk were determined.

### In Silico Analysis of Primer Specificity and Inclusivity

In silico analysis for RT primer specificity and inclusivity was performed following the FDA guideline (see e.g., Molecular Diagnostic Template for Laboratories, version Jul. 28, 2020). Specifically, inclusivity analysis was performed against all available SARS-CoV-2 genome sequences downloaded from NCBI (98,765 sequences), after excluding incomplete genomes (e.g., sequences with consecutive N’s and sequence fragments less than 20,000 nt in length). Specificity analysis was performed on Blastn against the recommended list of common respiratory flora and other viral pathogens (see e.g., full list available in Table 8), using parameters optimized for detection of short, somewhat similar sequences.

### Confirmatory Clinical Sensitivity Assay With Multiplexed Sequencing

Conformity clinical sensitivity studies were performed in pooled negative remnant clinical specimen background with different concentration of inactivated virus spike-in (ATCC) in a roughly 2x dilution series, based on results from pilot studies. All tests were performed with 96x multiplexed sample processing workflow. Each testing condition was repeated 20-22 times using high-quality, unique barcodes (i.e. not repeated 20-22 times with the same barcode) selected from barcode QC experiment. Each primer was tested multiple times with different batch barcode on the reverse side. Sequencing read threshold values were calculated using 3-σ formula (cut-off = mean + 3x stdev.) and reads obtained from negative control samples. The final limit of detection (LoD) for each target primer pair was determined using 95% detection rate cut-off (e.g., 19/20 or 21/22 detection) or 90% cut-off (when specified).

### Classification of Positive Samples

For sensitivity studies and clinical sample tests by multiplexed sequencing, positive samples can be determined using the 3-σ threshold, e.g., any sample with matched record count higher than mean + 3x stdev of all measurements obtained on the negative control samples were determined to be positive. Here, record count can be measured in one of two ways: either using raw sequencing read count (+1), or using above read count normalised by read count of positive control RNA.

### Tables

Table 2 shows key performance characteristics for scalable diagnostics with One-Seq. “*” indicates column was scaled (2x) to match capital cost as one NovaSeq™ 6000 sequencer; “**” indicates assuming an average of 2.5x10⁵ sequencing reads per sample. “***” indicates the estimated amortized cost with mass production. See e.g., Table 9 for details.

TABLE 2

Key performance characteristics for scalable diagnostics with One-Seq

| | | | | |
|---|---|---|---|---|
| One-Seq specification | MiSeq™ | NextSeq 550™ | NovaSeq 6000™ | PromethION™ 48* |
| Max. samples per run ** | 80 | 1,600 | 40,000 | 60,000 (8 hr) |
| Max. samples per day (diagnostics only) | 480 | 6,400 | 160,000 | 180,000 |
| Max. samples per day (diagnostics and sequencing) | 320 | 4,800 | 100,000 | |
| Sequencing cost | $10.30 | $1.00 | $0.30 | <$0.10 |
| Cost per sample *** | $13.10 | $3.80 | $3.10 | $2.80 |
| Turn-around time | 7.5-10.5 hr | 9-12.5 hr | 10-14.5 hr | 12 hr |

Table 3 compares performance between One-Seq and other methods. “*” indicates that for RNA extraction or PCR limited tests, throughput is estimated assuming sample processing in 96-well formats, and under the assumption that RNA extraction takes 0.5 hr, and PCR thermocycling takes 1.5 hr. PCR throughput is estimated using 384-well plates.

$$″ † ″$$

indicates that it was tested by FDA’s SARS-CoV-2 Reference Panel (see e.g., fda.gov/medical-devices/coronavirus-covid-19-and-medical-devices/sars-cov-2-reference-panel-comparative-data#table2a);

$$″ † † ″$$

indicates projected sensitivity using four primers;

$$″ † † † ″$$

indicates an estimate.

TABLE 3

Performance comparison between One-Seq and other methods

| METHOD | RT-qPCR | | COVIDSeq | Swab-Seq | | LamPORE | One-Seq |
|---|---|---|---|---|---|---|---|
| | With RNA extraction | Without extraction | | With RNA extraction | Without extraction | | |
| Throughput-limiting Step | RNA extraction / RT-PCR | RT-PCR | Sequencing | RNA extraction | PCR * | RNA extraction | Decapping / Sequencing |
| Max. samples per day per limiting instrument* | 1,600 | 1,600 | 1,000 | 4,800 | 6,400 * | 4,800 | 100,000-160,000 |
| Sensitivity (gce / ml) | 180-18,000 † | 2,000+ | 5,400 † | 250 | 1,000-3,000 | 20-200 | 100-250 †† |
| Viral sequencing capability | - | | Whole genome | Targeted (2x) | | Targeted (multiple) | Targeted (multiple) |
| Reagent cost (amortized) | $3-6 | | $20 | $2-4 | | $3-6††† | $3 |
| Turn-around time | 2-4 hr | | 12-24 hr | 8 hr | | 6 hr ††† | 7.5-15 hr |

Table 4 lists One-Seq primers, adapters, and batch barcodes. All Tm values were calculated using IDT oligo analyzer (available on the worldwide web at idtdna.com/calc/analyzer), with qPCR default parameters.

TABLE 4

One-Seq primers, adapters and batch barcodes

| Name | Type | SEQ ID NO | Sequence | Tm |
|---|---|---|---|---|
| N#1_RT | RT primer | 3 | AATTTAAGGTCTTCCTTGC (reverse complement binds to nt 179-197 of SEQ ID NO: 1001, N gene) | 53.8° C. (RNA) |
| N#1_PCR | Reverse PCR primer | 4 | GTTTACCCAATAATACTGCGTCT (identical to nt 131-153 of SEQ ID NO: 1001, N gene) | 60.8° C. |
| N#2_RT | RT primer | 5 | TGTGTAGGTCAACCACG (reverse complement binds to nt 986-1002 of SEQ ID NO: 1001, N gene) | 53.7° C. (RNA) |
| N#2_PCR | Reverse PCR primer | 6 | CAGACAAGGAACTGATTACAAACA (identical to nt 876-899 of SEQ ID NO: 1001, N gene) | 61.5° C. |
| del6970_RT | RT primer | 7 | CTCTTAGTACCATTGGTCC (reverse complement binds to nt 215-233 of SEQ ID NO: 1002, S gene) | 61.4° C. (RNA) |
| del6970_PCR | Reverse PCR primer | 8 | TTCTTACCTTTCTTTTCCAATGTTACT (identical to nt 163-189 of SEQ ID NO: 1002, S gene) | 62.0° C. |
| D614_RT | RT primer | 9 | GGACTTCTGTGCAGTTAAC (reverse complement binds to nt 1843-1861 of SEQ ID NO: 1002, S gene) | 56.5° C. (RNA) |
| D614 PCR | Reverse PCR primer | 10 | CAGTGTTATAACACCAGGAACA (identical to nt 1785-1806 of SEQ ID NO: 1002, S gene) | 60.3° C. |
| RNA_PC | Synthetic RNA control | 11 | CCAAGGTTTACCCAATAATACTGCTGAGGTTGTCACCGCTCTCACGACCACGTGCAAGGAAGACCTTAAATT (bolded text, e.g., nt 54-72 of SEQ ID NO: 11, indicates where SEQ ID NO: 3 (N#1_RT primer) hybridizes; double-underlined text indicates region identical to SEQ ID NO: 12) | |
| RNA_PC_PCR | Reverse PCR primer | 12 | CCAATAATACTGCTGAGGTTGT | 60.5° C. |
| P5xs | Short PCR adapter | 13 | CGCCAGCAGCGAACAA | 62.8° C. |
| P5xe | P5 side adapter and common PCR primer | 14 | AATGATACGGCGACCACCGA GATCTACACAGAACGCCAGCAGCGAACAA (bolded text corresponds to SEQ ID NO: 13; double underlined text corresponds to SEQ ID NO: 15) | 78.4° C. |
| P5xr | Read 1 primer | 15 | CGA GATCTACAC AGAACGCCAGCAGCG | 70.9° C. |
| P7y | P7 side adapter | 16 | CAAGCAGAAGACGGCATACGAGATACGAGCAAGCACAGGACCACAACACG (bolded text corresponds to SEQ ID NO: 17) | 77.4° C. |
| P7yr | Read 2 primer | 17 | ACGAGCAAGCACAGGACCACAACACG | 71.7° C. |
| S01 | Batch barcode | 18 | TGGTACAG | |
| S02 | Batch barcode | 19 | AACCGTTC | |
| S03 | Batch barcode | 20 | TAACCGGT | |
| S04 | Batch barcode | 21 | GAACATCG | |
| S05 | Batch barcode | 22 | CCTTGTAG | |
| S06 | Batch barcode | 23 | TCAGGCTT | |
| S07 | Batch barcode | 24 | GTTCTCGT | |
| S08 | Batch barcode | 25 | AGAACGAG | |
| S09 | Batch barcode | 26 | TGCTTCCA | |
| S10 | Batch barcode | 27 | CTTCGACT | |
| S11 | Batch barcode | 28 | CACCTGTT | |
| S12 | Batch barcode | 29 | TGGTACAG | |

Table 5 lists the 960x unique sample barcodes (e.g., Barcode IDs: UDPX001-960). “#” in the first column indicates SEQ ID NO. “UDPX” in the second column indicates UDPX ID number.

TABLE 5

960x unique sample barcodes (Barcode IDs: UDPX001-960)

| # | UDPX | Barcode sequence | # | UDPX | Barcode sequence |
|---|---|---|---|---|---|
| 30 | 001 | CGCTCACAGTTCTGTCGTGACGAGCG | 510 | 481 | CTGACCACGGCACTCCTGAGATACAA |
| 31 | 002 | TATCTACGACCTTGCTACAACAGATA | 511 | 482 | GAATTCAGAGTGCTTTAAGGATTGTG |
| 32 | 003 | ATATGACAGACGTGTATAGACTAGCT | 512 | 483 | GCGTGCATGAGACTCGGACGAAGTGA |
| 33 | 004 | CTTATACGGAATTGTGCCTACGGTGG | 513 | 484 | TCTCCCAATTGACTGCACTGAACAAC |
| 34 | 005 | TAATCACTCGTCTGACATTACATCCT | 514 | 485 | ACATGCACATATCTTGGTGGACCTGG |
| 35 | 006 | GCGCGACATGTTTGGTCCAACCTTGT | 515 | 486 | CAGGCCAGCCATCTTCCACGAGGCCT |
| 36 | 007 | AGAGCACACTAGTGTGGAAACCAGTA | 516 | 487 | ACATACAACGGACTTTGTAGAGTGTA |
| 37 | 008 | TGCCTACTGATCTGCCTTGACTTAAT | 517 | 488 | TTAATCAAGACCCTCCACGGAACACG |
| 38 | 009 | CTACTACCAGTCTGGTTGAACTAGTG | 518 | 489 | ACGATCATGCTGCTTGTGAGATGTAT |
| 39 | 010 | TCGTCACTGACTTGACCAGACCGACA | 519 | 490 | TTCTACACAGAACTGAGCGGACAATA |
| 40 | Oil | GAACAACTACGGTGCATACACACTGT | 520 | 491 | TATTGCACGTTCCTATCTTGAACTGT |
| 41 | 012 | CCTATACGACTCTGGTGTGACGCGCT | 521 | 492 | CATGACAGTACTCTATGTCGAGTGGT |
| 42 | 013 | TAATGACGCAAGTGATCACACGAAGG | 522 | 493 | TAATTCACTACCCTGTAGCGACATCA |
| 43 | 014 | GTGCCACGCTTCTGCGGCTACCTACT | 523 | 494 | ACGCTCAAATTACTTGGTTGAAAGAA |
| 44 | 015 | CGGCAACATGGATGGAATGACCACGA | 524 | 495 | CCTTGCATTAATCTTGTTGGATTCGT |
| 45 | 016 | GCCGTACAACCGTGAAGACACTATAG | 525 | 496 | GTAGCCACATCACTCCAACGAAACAT |
| 46 | 017 | AACCAACTTCTCTGTCGGCACAGCAA | 526 | 497 | CTTGTCAAATTCCTACCGGGACTCAG |
| 47 | 018 | GGTTGACCCTCTTGCTAATACGATGG | 527 | 498 | TCCAACATTCTACTGTTAAGATCTGA |
| 48 | 019 | CTAATACGATGGTGGGTTGACCCTCT | 528 | 499 | AGAGCCATGCCTCTCGGCTGAAACGT |
| 49 | 020 | TCGGCACCTATCTGCGCACACATGGC | 529 | 500 | CTTCGCACCGATCTTCCAAGAGAATT |
| 50 | 021 | AGTCAACACCATTGGGCCTACGTCCT | 530 | 501 | TCGGTCACACGGCTCCGAAGACGTTG |
| 51 | 022 | GAGCGACCAATATGCTGTGACTTAGG | 531 | 502 | GAACACAAGTATCTTAACCGAGCCGA |
| 52 | 023 | AACAAACGGCGTTGTAAGGACAACGT | 532 | 503 | AATTGCAGCGGACTCTCCGGATGCTG |
| 53 | 024 | GTATGACTAGAATGCTAACACTGTAA | 533 | 504 | GGCCTCAGTCCTCTCATTCGACAGCT |
| 54 | 025 | TTCTAACTGGTTTGGGCGAACGATGG | 534 | 505 | TAGGTCATCTCTCTGGTTAGATGCTA |
| 55 | 026 | CCTCGACCAACCTGAATAGACAGCAA | 535 | 506 | ACACACAATATCCTACCACGAACGGT |
| 56 | 027 | TGGATACGCTTATGTCAATACCCATT | 536 | 507 | TTCCTCAGTACGCTTAGGTGATCTCT |
| 57 | 028 | ATGTCACGTGGTTGTCGTAACTGCGG | 537 | 508 | GGTAACACGCAGCTTATGGGACTCGA |
| 58 | 029 | AGAGTACGCGGCTGTCCGAACCCTCG | 538 | 509 | TCCACCAGGCCTCTCTCGTGAGCGTT |
| 59 | 030 | TGCCTACGGTGGTGCTTATACGGAAT | 539 | 510 | GATACCACTCCTCTCCAGTGATGGCA |
| 60 | 031 | TGCGTACGTCACTGGCTTAACCGGAC | 540 | 511 | CAACGCATCAGCCTTGTTCGAGCATT |
| 61 | 032 | CATACACACTGTTGGAACAACTACGG | 541 | 512 | CGGTTCAATTAGCTAACCGGACATCG |
| 62 | 033 | CGTATACAATCATGGTCGAACTTACA | 542 | 513 | CGCGCCACTAGACTCGAAGGAGTTAA |
| 63 | 034 | TACGCACGGCTGTGACTAGACCCGTG | 543 | 514 | TCTTGCAGCTATCTAGTGCGACACTG |
| 64 | 035 | GCGAGACTTACCTGAAGTTACGGTGA | 544 | 515 | TCACACACCGAACTGAACAGAAGTAT |
| 65 | 036 | TACGGACCCGGTTGTGGCAACATATT | 545 | 516 | AACGTCATACATCTACGATGATGCTG |
| 66 | 037 | GTCGAACTTACATGGATCAACCCGCG | 546 | 517 | CGGCCCATCGTTCTATACCGATGGAT |
| 67 | 038 | CTGTCACTGCACTGTACCAACTCCGT | 547 | 518 | CATAACACACCACTTCCAAGATTCTA |
| 68 | 039 | CAGCCACGATTGTGGCTGTACAGGAA | 548 | 519 | ACAGACAGGCCACTTGAGAGACAGCG |
| 69 | 040 | TGACTACACATATGCGCACACTAATG | 549 | 520 | TGGTGCACCTGGCTACGCTGAAATTA |
| 70 | 041 | ATTGCACCGAGTTGGACAAACCTGAA | 550 | 521 | TAGGACAACCGGCTTATATGATCGAG |
| 71 | 042 | GCCATACTAGACTGAGTGGACTCAGG | 551 | 522 | AATATCATGGCCCTCGGTCGACGATA |
| 72 | 043 | GGCGAACGATGGTGTTCTAACTGGTT | 552 | 523 | ATAGGCATATTCCTACAATGAAGAGT |
| 73 | 044 | TGGCTACCGCAGTGAATCCACGGCCA | 553 | 524 | CCTTCCAACGTACTCGGTTGAATTAG |
| 74 | 045 | TAGAAACTAACGTGCCATAACAGGTT | 554 | 525 | GGCCACAATAAGCTGATAAGACAAGT |
| 75 | 046 | TAATGACGATCTTGATCTCACTACCA | 555 | 526 | CAGTACAGTTGTCTAGTTAGATCACA |
| 76 | 047 | TATCCACAGGACTGCGGTGACGCGAA | 556 | 527 | TTCATCACCAACCTTTCCAGAGGTAA |
| 77 | 048 | AGTGCACCACTGTGTAACAACATAGG | 557 | 528 | CAATTCAGGATTCTCATGTGAAGAGG |
| 78 | 049 | GTGCAACACACTTGCTGGTACACACG | 558 | 529 | GGCCACATCATACTGATTGGATCATA |
| 79 | 050 | ACATGACGTGTCTGTCAACACGTGTA | 559 | 530 | AATTGCACTGCGCTATTCCGAGCTAT |
| 80 | 051 | GACAGACACAGGTGACTGTACTGTGA | 560 | 531 | TAAGGCAAACGTCTGACCGGACTGTG |
| 81 | 052 | TCTTAACCATCATGGTGCGACTCCTT | 561 | 532 | CTATACACGCGGCTTAGGAGAACCGG |
| 82 | 053 | TTACAACATTCCTGAGCACACATCCT | 562 | 533 | ATTCACAGAATCCTAGCGGGATGGAC |
| 83 | 054 | AAGCTACTATGCTGTTCCGACTCGCA | 563 | 534 | GTATTCACTCTACTTATAGGAATTCG |
| 84 | 055 | TATTCACCTCAGTGCTTAAACCCACT | 564 | 535 | CCTGACATACAACTACAGAGAGGCCA |
| 85 | 056 | CTCGTACGCGTTTGGCCTCACGGATA | 565 | 536 | GACCGCACTGTGCTATTCCGATATTG |
| 86 | 057 | TTAGGACATAGATGCGTCGACACTGG | 566 | 537 | TTCAGCACGTGGCTTATTCGACTCAG |
| 87 | 058 | CCGAAACGCGAGTGTACTAACGTCAA | 567 | 538 | AACTCCACGAACCTCGCCTGATCTGA |
| 88 | 059 | GGACCACAACAGTGATAGAACCCGTT | 568 | 539 | ATTCCCAGCTATCTGCGCAGAGAGTA |
| 89 | 060 | TTCCAACGGTAATGACAGTACTCCAG | 569 | 540 | TGAATCAATTGCCTGGCGCGACAATT |
| 90 | 061 | TGATTACAGCCATGAGGCAACTGTAG | 570 | 541 | CGCAACATCTAGCTAGATAGATGGCG |
| 91 | 062 | TAACAACGTGTTTGGCAAGACTCTCA | 571 | 542 | AACCGCACATCGCTCCTGCGATTGGT |
| 92 | 063 | ACCGCACGCAATTGTTGGCACTCCGC | 572 | 543 | CTAGTCACCGGACTGACGAGAACAAT |
| 93 | 064 | GTTCGACCGCCATGAACTGACATACT | 573 | 544 | GCTCCCAGTCACCTTGGCGGAGTCCA |
| 94 | 065 | AGACAACCATTATGGTAAGACGCATA | 574 | 545 | AGATGCAGAATTCTCTTCAGAGTTAC |
| 95 | 066 | GCGTTACGGTATTGAATTGACCTGCG | 575 | 546 | ACACCCAGTTAACTTCCTGGAACCGT |
| 96 | 067 | AG CACACATCCTTGTTACAACATTCC | 576 | 547 | GATAACACAAGTCTCGCGCGACTAGA |
| 97 | 068 | TTGTTACCCGTGTGAACCTACAGCAC | 577 | 548 | CTGGTCAACACGCTAGGATGAAAGTT |
| 98 | 069 | AAGTAACCTCCATGTCTGTACGTGGA | 578 | 549 | CGAAGCAGTTAACTAGGCCGAAGACA |
| 99 | 070 | ACGTCACAATACTGGGAATACTCCAA | 579 | 550 | ATCGCCAATATGCTCCTTGGAAACGG |
| 100 | 071 | GGTGTACACAAGTGAAGCGACCGCTT | 580 | 551 | ATCATCAAGGCTCTCACCAGACCTAC |
| 101 | 072 | CCACCACTGTGTTGTGAGCACGTTGT | 581 | 552 | GATTGCATCATACTTTGCTGATGTAT |
| 102 | 073 | GTTCCACGCAGGTGATCATACAGGCT | 582 | 553 | CCAACCAAACATCTCAATCGATATGA |
| 103 | 074 | ACCTTACATGAATGTGTTAACGAAGG | 583 | 554 | TTGGTCAGGTGCCTTGGTAGACTGAT |
| 104 | 075 | CGCTGACCAGAGTGGATGGACATGTA | 584 | 555 | GCGAACACGCCTCTTTCATGACCAAC |
| 105 | 076 | GTAGAACGTCAGTGACGGCACCGTCA | 585 | 556 | CAACCCAGGAGGCTCATAAGACACCA |
| 106 | 077 | GGATAACCCAGATGCGTTGACCTTAC | 586 | 557 | AGCGGCATGGACCTTCCTAGATTAGC |
| 107 | 078 | CGCACACTAATGTGTGACTACACATA | 587 | 558 | GACGACAACAATCTTCTCTGAAGATT |
| 108 | 079 | TCCTGACACCGTTGCGGCCACTCGTT | 588 | 559 | CCACTCAGGTCCCTCGCGAGAGCCTA |
| 109 | 080 | CTGGCACTTGCCTGCAAGCACATCCG | 589 | 560 | TGTTACAGAAGGCTGATAAGAGCTCT |
| 110 | 081 | ACCAGACCGACATGTCGTCACTGACT | 590 | 561 | TATATCATCGAGCTGAGATGAGTCGA |
| 111 | 082 | TTGTAACACGGTTGCTCATACAGCGA | 591 | 562 | CGCGACACGATCCTCTGGAGATATGT |
| 112 | 083 | GTAAGACGCATATGAGACAACCATTA | 592 | 563 | GCCTCCAGGATACTGGCCAGAATAAG |
| 113 | 084 | GTCCAACCTTGTTGGCGCGACATGTT | 593 | 564 | TGAGACACAGCGCTATTACGATCACC |
| 114 | 085 | TTAGGACTACCATGCATGAACGTACT | 594 | 565 | TGTTCCAGCATTCTAATTGGAGCGGA |
| 115 | 086 | GGAATACTCCAATGACGTCACAATAC | 595 | 566 | TCCAACAGAATTCTTTGTCGAAACTT |
| 116 | 087 | CATGTACAGAGGTGGATACACCTCCT | 596 | 567 | GCTGTCAAGGAACTGGCGAGAATTCT |
| 117 | 088 | TACACACGCTCCTGATCCGACTAAGT | 597 | 568 | ATACCCATGGATCTCAACGGATCAGC |
| 118 | 089 | GCTTAACCGGACTGCGTGTACATCTT | 598 | 569 | GTTGGCAACCGTCTTCTTAGACATCA |
| 119 | 090 | CGCTTACGAAGTTGGAACCACATGAA | 599 | 570 | ACCAACAGTTACCTCGCCAGATACCT |
| 120 | 091 | CGCCTACTCTGATGGGCCAACTCATA | 600 | 571 | GTGTGCAGCGCTCTCTAATGAGTCTT |
| 121 | 092 | ATACCACAACGCTGACATAACCTTCC | 601 | 572 | GGCAGCATAGCACTCAACCGAGGAGG |
| 122 | 093 | CTGGAACTATGTTGTATGTACGCAAT | 602 | 573 | TGCGGCATGTTGCTGGCAGGATAGCA |
| 123 | 094 | CAATCACTATGATGGATTAACAGGTG | 603 | 574 | GATTACAAGGTGCTTTAGGGAATAGA |
| 124 | 095 | GGTGGACAATACTGATGTAACGACAA | 604 | 575 | CAACACATTCAACTCGCAAGATCTAG |
| 125 | 096 | TGGACACGGAGGTGCACATACCGGTG | 605 | 576 | GTGTTCAACCGGCTGAGTTGAGTACT |
| 126 | 097 | CTGACACCGGCATGCCTGAACTACAA | 606 | 577 | TATCACATGAGACTAACACGAGTGGA |
| 127 | 098 | GAATTACGAGTGTGTTAAGACTTGTG | 607 | 578 | CTTGGCACCTCGCTGTGTTGAACCGG |
| 128 | 099 | GCGTGACTGAGATGCGGACACAGTGA | 608 | 579 | GTCTCCAGTGAACTAGATTGAGTTAC |
| 129 | 100 | TCTCCACATTGATGGCACTACACAAC | 609 | 580 | CCATCCACACGCCTTTGACGACAATG |
| 130 | 101 | ACATGACCATATTGTGGTGACCCTGG | 610 | 581 | ACAACCACAGGACTCTGACGACGGCA |
| 131 | 102 | CAGGCACGCCATTGTCCACACGGCCT | 611 | 582 | AGCAGCAAATTACTTCTCAGATCAAT |
| 132 | 103 | ACATAACACGGATGTTGTAACGTGTA | 612 | 583 | CAGTCCAGTGCGCTGGACCGAAACAG |
| 133 | 104 | TTAATACAGACCTGCCACGACACACG | 613 | 584 | GTCTACAACCTCCTAATGTGAATTGC |
| 134 | 105 | ACGATACTGCTGTGTGTGAACTGTAT | 614 | 585 | GAACTCACGGTTCTGATCTGACTGGA |
| 135 | 106 | TTCTAACCAGAATGGAGCGACCAATA | 615 | 586 | AGTTACATCACACTCAGGCGAGCCAT |
| 136 | 107 | TATTGACCGTTCTGATCTTACACTGT | 616 | 587 | GTAGCCAATACTCTTTAATGAAGACC |
| 137 | 108 | CATGAACGTACTTGATGTCACGTGGT | 617 | 588 | CTTCACAGTTACCTGGAGTGACGCGA |
| 138 | 109 | TAATTACCTACCTGGTAGCACCATCA | 618 | 589 | AGTCCCAGAGGACTAACGCGACAGAG |
| 139 | 110 | ACGCTACAATTATGTGGTTACAAGAA | 619 | 590 | ACAGTCATCCAGCTCGTAAGATTAAC |
| 140 | 111 | CCTTGACTTAATTGTGTTGACTTCGT | 620 | 591 | CCGCACATATTCCTACGAGGAACTGA |
| 141 | 112 | GTAGCACCATCATGCCAACACAACAT | 621 | 592 | TTATCCACGATCCTGTATCGAGGCCG |
| 142 | 113 | CTTGTACAATTCTGACCGGACCTCAG | 622 | 593 | ATAGTCACTAGCCTAATACGAGACAT |
| 143 | 114 | TCCAAACTTCTATGGTTAAACTCTGA | 623 | 594 | TATAGCATAGCTCTGTTATGAATGGC |
| 144 | 115 | AGAGCACTGCCTTGCGGCTACAACGT | 624 | 595 | ACTCCCAGGTGGCTGCCTGGACCATG |
| 145 | 116 | CTTCGACCCGATTGTCCAAACGAATT | 625 | 596 | GTGCGCAGTAAGCTTAAGAGACCTAT |
| 146 | 117 | TCGGTACCACGGTGCCGAAACCGTTG | 626 | 597 | GATATCACCTAACTTATACGACATGG |
| 147 | 118 | GAACAACAGTATTGTAACCACGCCGA | 627 | 598 | TCGCGCATATAACTGCCGTGACTGTT |
| 148 | 119 | AATTGACGCGGATGCTCCGACTGCTG | 628 | 599 | ATTCTCAAAGCGCTCAGAGGATGATA |
| 149 | 120 | GGCCTACGTCCTTGCATTCACCAGCT | 629 | 600 | AGCGCCATTCGGCTTGCTAGAACTAT |
| 150 | 121 | TAGGTACTCTCTTGGGTTAACTGCTA | 630 | 601 | GTTGACATAGTGCTTCAGTGATAATG |
| 151 | 122 | ACACAACATATCTGACCACACACGGT | 631 | 602 | AATAGCAAGCAACTGTGACGACTTGA |
| 152 | 123 | TTCCTACGTACGTGTAGGTACTCTCT | 632 | 603 | CTAACCATGTAACTACATGGACATAT |
| 153 | 124 | GGTAAACCGCAGTGTATGGACCTCGA | 633 | 604 | GCGTACACTTAGCTAACATGAACCTA |
| 154 | 125 | TCCACACGGCCTTGCTCGTACGCGTT | 634 | 605 | TACCGCAAACTACTCCATGGATGTAG |
| 155 | 126 | GATACACCTCCTTGCCAGTACTGGCA | 635 | 606 | GTAGTCAAATAGCTGAGTCGATCTCC |
| 156 | 127 | CAACGACTCAGCTGTGTTCACGCATT | 636 | 607 | GGTTACATGCTACTGCTATGAGCGCA |
| 157 | 128 | CGGTTACATTAGTGAACCGACCATCG | 637 | 608 | ACAATCAAGAGTCTATCGCGAATATG |
| 158 | 129 | CGCGCACCTAGATGCGAAGACGTTAA | 638 | 609 | GCTTCCACACTACTAGTACGACTATA |
| 159 | 130 | TCTTGACGCTATTGAGTGCACCACTG | 639 | 610 | AGATACATGGCGCTGACCGGAGAGAT |
| 160 | 131 | TCACAACCCGAATGGAACAACAGTAT | 640 | 611 | AATATCAGAAGCCTCGTTCGAAGCCT |
| 161 | 132 | AACGTACTACATTGACGATACTGCTG | 641 | 612 | TAGCGCACTAGTCTTTACTGATCCTC |
| 162 | 133 | CGGCCACTCGTTTGATACCACTGGAT | 642 | 613 | AGTTACAAGAGCCTCACGTGACCACC |
| 163 | 134 | CATAAACCACCATGTCCAAACTTCTA | 643 | 614 | CAGATCAACCACCTGCTACGATATCT |
| 164 | 135 | ACAGAACGGCCATGTGAGAACCAGCG | 644 | 615 | ACGGCCACGTCACTAGTCAGAACCAT |
| 165 | 136 | TGGTGACCCTGGTGACGCTACAATTA | 645 | 616 | GTAATCATACTGCTCGAGGGACGGTA |
| 166 | 137 | TAGGAACACCGGTGTATATACTCGAG | 646 | 617 | AAGTCCATTGTACTCAGGTGAGTTCA |
| 167 | 138 | AATATACTGGCCTGCGGTCACCGATA | 647 | 618 | GTCACCACACAGCTGACAGGAACAGG |
| 168 | 139 | ATAGGACTATTCTGACAATACAGAGT | 648 | 619 | ATTAGCATGGAGCTTGTACGATTGTT |
| 169 | 140 | CCTTCACACGTATGCGGTTACATTAG | 649 | 620 | TGCTACAACTATCTCTCTAGAAGTAG |
| 170 | 141 | GGCCAACATAAGTGGATAAACCAAGT | 650 | 621 | TAAGACACCTATCTGTCACGACACAG |
| 171 | 142 | CAGTAACGTTGTTGAGTTAACTCACA | 651 | 622 | TGGTTCAAAGAACTTCTACGAATACC |
| 172 | 143 | TTCATACCCAACTGTTCCAACGGTAA | 652 | 623 | ACTCTCATCCTTCTCACGTGATAGGC |
| 173 | 144 | CAATTACGGATTTGCATGTACAGAGG | 653 | 624 | GTCTCCACTTCCCTTGGTGGAAGTCT |
| 174 | 145 | GGCCAACTCATATGGATTGACTCATA | 654 | 625 | TCCGCCAGTTCACTCTTCGGAAAGGA |
| 175 | 146 | AATTGACCTGCGTGATTCCACGCTAT | 655 | 626 | AGGTTCAGCAGGCTGTAGAGAGTCAG |
| 176 | 147 | TAAGGACAACGTTGGACCGACCTGTG | 656 | 627 | GAACCCAATGAACTGACATGATGTCA |
| 177 | 148 | CTATAACCGCGGTGTAGGAACACCGG | 657 | 628 | TTGAGCAAGGATCTTCCGCGAAAGGC |
| 178 | 149 | ATTCAACGAATCTGAGCGGACTGGAC | 658 | 629 | TGGTCCATAGTGCTACTGCGACTTAT |
| 179 | 150 | GTATTACCTCTATGTATAGACATTCG | 659 | 630 | AGTGGCAATAATCTTACGCGAACGTA |
| 180 | 151 | CCTGAACTACAATGACAGAACGGCCA | 660 | 631 | GGCACCAGCCATCTCGCTTGAGAAGT |
| 181 | 152 | GACCGACCTGTGTGATTCCACTATTG | 661 | 632 | GATCTCACTGGACTCTGCAGACTTCA |
| 182 | 153 | TTCAGACCGTGGTGTATTCACCTCAG | 662 | 633 | TGCTGCAGACATCTCAGCGGAGACAA |
| 183 | 154 | AACTCACCGAACTGCGCCTACTCTGA | 663 | 634 | CCGAACACGTTGCTGGATCGACGCAT |
| 184 | 155 | ATTCCACGCTATTGGCGCAACGAGTA | 664 | 635 | ATTAACATACGCCTTGCGGGATGTTG |
| 185 | 156 | TGAATACATTGCTGGGCGCACCAATT | 665 | 636 | TAGTCCAACAACCTACATAGAACGGA |
| 186 | 157 | CGCAAACTCTAGTGAGATAACTGGCG | 666 | 637 | GGTATCATGAGACTGACGTGATCGCG |
| 187 | 158 | AACCGACCATCGTGCCTGCACTTGGT | 667 | 638 | CAAGACATGCTTCTCATTCGAAACAA |
| 188 | 159 | CTAGTACCCGGATGGACGAACACAAT | 668 | 639 | ACGAGCAACTGACTCACGGGAATTAT |
| 189 | 160 | GCTCCACGTCACTGTGGCGACGTCCA | 669 | 640 | TTATCCATTGCACTTTGAGGAGACGG |
| 190 | 161 | AGATGACGAATTTGCTTCAACGTTAC | 670 | 641 | AGATTCAGTTACCTCTCTGGATATAC |
| 191 | 162 | ACACCACGTTAATGTCCTGACACCGT | 671 | 642 | TCTACCACGCTGCTGCAACGAAGGTG |
| 192 | 163 | GATAAACCAAGTTGCGCGCACCTAGA | 672 | 643 | AACGGCATATGACTGGTAAGACGCAG |
| 193 | 164 | CTGGTACACACGTGAGGATACAAGTT | 673 | 644 | CAATGCAGCGCCCTACCGCGAGCAAT |
| 194 | 165 | CGAAGACGTTAATGAGGCCACAGACA | 674 | 645 | CTAATCATCGCTCTAGCCGGAGAACA |
| 195 | 166 | ATCGCACATATGTGCCTTGACAACGG | 675 | 646 | CATGGCATCTAACTTCCTAGAGGAAG |
| 196 | 167 | ATCATACAGGCTTGCACCAACCCTAC | 676 | 647 | ATACTCAGTGTGCTTTGAGGACCTAA |
| 197 | 168 | GATTGACTCATATGTTGCTACTGTAT | 677 | 648 | GCCGACACAAGACTCCACCGATGTGT |
| 198 | 169 | CCAACACAACATTGCAATCACTATGA | 678 | 649 | CGAGGCACGGTACTCCTCGGACAACC |
| 199 | 170 | TTGGTACGGTGCTGTGGTAACCTGAT | 679 | 650 | GATATCAAACAGCTGTATAGAGCTGT |
| 200 | 171 | GCGAAACCGCCTTGTTCATACCCAAC | 680 | 651 | TCGCCCAGGTTACTGCTACGAATTAG |
| 201 | 172 | CAACCACGGAGGTGCATAAACCACCA | 681 | 652 | AGACTCACTCTTCTTACGAGAATCTT |
| 202 | 173 | AGCGGACTGGACTGTCCTAACTTAGC | 682 | 653 | GCTCGCACCTACCTTAGGAGAGCGCA |
| 203 | 174 | GACGAACACAATTGTCTCTACAGATT | 683 | 654 | AGGATCAAAGTTCTGTACTGAGGCGT |
| 204 | 175 | CCACTACGGTCCTGCGCGAACGCCTA | 684 | 655 | GAGACCAATAATCTAGTTAGAAGAGC |
| 205 | 176 | TGTTAACGAAGGTGGATAAACGCTCT | 685 | 656 | AGCTGCATTATACTTCGCGGATATAA |
| 206 | 177 | TATATACTCGAGTGGAGATACGTCGA | 686 | 657 | GTATCCAATTGGCTGAGTGGATGCCG |
| 207 | 178 | CGCGAACCGATCTGCTGGAACTATGT | 687 | 658 | AATAGCAGCCTCCTCTAGTGACCGGA |
| 208 | 179 | GCCTCACGGATATGGGCCAACATAAG | 688 | 659 | CCGCTCATAGCTCTATTAAGATACGC |
| 209 | 180 | TGAGAACCAGCGTGATTACACTCACC | 689 | 660 | TCCTACAGGAAGCTCCTAGGAAGTAT |
| 210 | 181 | TGTTCACGCATTTGAATTGACGCGGA | 690 | 661 | TCACACAGATCGCTTAGGAGAAGACT |
| 211 | 182 | TCCAAACGAATTTGTTGTCACAACTT | 691 | 662 | ACTTGCATCCACCTCCGTGGAGCCTT |
| 212 | 183 | GCTGTACAGGAATGGGCGAACATTCT | 692 | 663 | TGTACCATTGTTCTGGATAGATATCC |
| 213 | 184 | ATACCACTGGATTGCAACGACTCAGC | 693 | 664 | CACTTCAAATCTCTCACCTGACTTGG |
| 214 | 185 | GTTGGACACCGTTGTCTTAACCATCA | 694 | 665 | CAGAGCATGATACTAACGTGATACAT |
| 215 | 186 | ACCAAACGTTACTGCGCCAACTACCT | 695 | 666 | GGCGACAATTCTCTCGGCAGAAGCTC |
| 216 | 187 | GTGTGACGCGCTTGCTAATACGTCTT | 696 | 667 | AGTGGCATCAGGCTTCTTGGAGCTAT |
| 217 | 188 | GGCAGACTAGCATGCAACCACGGAGG | 697 | 668 | CATTCCACAGCTCTACGGAGAATGCG |
| 218 | 189 | TGCGGACTGTTGTGGGCAGACTAGCA | 698 | 669 | CTCGTCATATCACTGTTCCGAGCAGG |
| 219 | 190 | GATTAACAGGTGTGTTAGGACATAGA | 699 | 670 | CCTTACACTATGCTACCAAGAGTTAC |
| 220 | 191 | CAACAACTTCAATGCGCAAACTCTAG | 700 | 671 | AGAAGCACCAATCTTGGCTGACGCAG |
| 221 | 192 | GTGTTACACCGGTGGAGTTACGTACT | 701 | 672 | TAATCCAGGTACCTAACTAGAACGTT |
| 222 | 193 | TATCAACTGAGATGAACACACGTGGA | 702 | 673 | GGAATCATGTTCCTTAGAGGATTGGA |
| 223 | 194 | CTTGGACCCTCGTGGTGTTACACCGG | 703 | 674 | CCGGACACCACACTAGAGCGAACTAG |
| 224 | 195 | GTCTCACGTGAATGAGATTACGTTAC | 704 | 675 | GACTTCAAGAAGCTACTCTGAACAGG |
| 225 | 196 | CCATCACCACGCTGTTGACACCAATG | 705 | 676 | TGGCACAATATTCTCGGTGGAACACC |
| 226 | 197 | ACAACACCAGGATGCTGACACCGGCA | 706 | 677 | GAATGCACACGACTGCGTTGAGGTAT |
| 227 | 198 | AGCAGACAATTATGTCTCAACTCAAT | 707 | 678 | CGTGTCAATCTTCTTGTGCGATAACA |
| 228 | 199 | CAGTCACGTGCGTGGGACCACAACAG | 708 | 679 | ATTCACATTGCACTCCAGAGAAGTAA |
| 229 | 200 | GTCTAACACCTCTGAATGTACATTGC | 709 | 680 | TCCTTCACATAGCTCTTATGAACCTG |
| 230 | 201 | GAACTACCGGTTTGGATCTACCTGGA | 710 | 681 | TCTAGCATCTTCCTACTAGGAAACTT |
| 231 | 202 | AGTTAACTCACATGCAGGCACGCCAT | 711 | 682 | CTCGACACTCCTCTTTAGGGACTTAC |
| 232 | 203 | GTAGCACATACTTGTTAATACAGACC | 712 | 683 | AGTGACAGTGAACTTATCAGATGAGA |
| 233 | 204 | CTTCAACGTTACTGGGAGTACCGCGA | 713 | 684 | GAAGCCAGGACCCTCTCACGAACAAG |
| 234 | 205 | AGTCCACGAGGATGAACGCACCAGAG | 714 | 685 | GCTCTCACGTTGCTGAATTGAGAGTG |
| 235 | 206 | ACAGTACTCCAGTGCGTAAACTTAAC | 715 | 686 | GGACCCATCAATCTCGGATGATATAT |
| 236 | 207 | CCGCAACTATTCTGACGAGACACTGA | 716 | 687 | GAGTCCATCTCCCTTTGAAGAGCAGA |
| 237 | 208 | TTATCACCGATCTGGTATCACGGCCG | 717 | 688 | AACGGCAAGCGGCTTACGGGACGAAG |
| 238 | 209 | ATAGTACCTAGCTGAATACACGACAT | 718 | 689 | TGTGACATGTATCTTCTCCGAATTGA |
| 239 | 210 | TATAGACTAGCTTGGTTATACATGGC | 719 | 690 | AACATCAACCTACTCGAGAGACCAAG |
| 240 | 211 | ACTCCACGGTGGTGGCCTGACCCATG | 720 | 691 | GTGCTCAAGGTGCTTGCTGGAGACAT |
| 241 | 212 | GTGCGACGTAAGTGTAAGAACCCTAT | 721 | 692 | CATACCATTGAACTGATGGGATATCG |
| 242 | 213 | GATATACCCTAATGTATACACCATGG | 722 | 693 | CTTGTCACTTAACTGGCTTGAAATTG |
| 243 | 214 | TCGCGACTATAATGGCCGTACCTGTT | 723 | 694 | AAGAGCAAGGTGCTCTCGAGACTCCT |
| 244 | 215 | ATTCTACAAGCGTGCAGAGACTGATA | 724 | 695 | TGCACCAGAGAACTATACAGACAGAG |
| 245 | 216 | AGCGCACTTCGGTGTGCTAACACTAT | 725 | 696 | ACTTCCACTAGCCTTCTCGGAGACGA |
| 246 | 217 | GTTGAACTAGTGTGTCAGTACTAATG | 726 | 697 | GTGCTCAATTAACTACCACGAGTCTG |
| 247 | 218 | AATAGACAGCAATGGTGACACCTTGA | 727 | 698 | AGCGTCAGAATGCTGTTGTGAACTCA |
| 248 | 219 | CTAACACTGTAATGACATGACCATAT | 728 | 699 | CCTTACAGTGCCCTTCAGGGATCAAC |
| 249 | 220 | GCGTAACCTTAGTGAACATACACCTA | 729 | 700 | TGTACCACGAATCTAGTCCGAGAGGA |
| 250 | 221 | TACCGACAACTATGCCATGACTGTAG | 730 | 701 | GGAGACATTAGTCTCACTTGAAATCT |
| 251 | 222 | GTAGTACAATAGTGGAGTCACTCTCC | 731 | 702 | TACTACAACACACTTACTCGATGTTA |
| 252 | 223 | GGTTAACTGCTATGGCTATACGCGCA | 732 | 703 | TAGGTCACGTTGCTGCGACGATCGAT |
| 253 | 224 | ACAATACAGAGTTGATCGCACATATG | 733 | 704 | ATGCCCAGACCGCTCTAGGGACAAGG |
| 254 | 225 | GCTTCACCACTATGAGTACACCTATA | 734 | 705 | CTAGCCAGTCGACTCCTCTGATCGAA |
| 255 | 226 | AGATAACTGGCGTGGACCGACGAGAT | 735 | 706 | TGCCTCAACGAGCTTCATCGACTCTT |
| 256 | 227 | AATATACGAAGCTGCGTTCACAGCCT | 736 | 707 | ACTAGCAAACTTCTGGTAAGAGATAA |
| 257 | 228 | TAGCGACCTAGTTGTTACTACTCCTC | 737 | 708 | CACCTCACTTGGCTAACGAGAGCCAG |
| 258 | 229 | AGTTAACAGAGCTGCACGTACCCACC | 738 | 709 | AAGCACAGATATCTTAGACGAAATCT |
| 259 | 230 | CAGATACACCACTGGCTACACTATCT | 739 | 710 | GCCAGCAATCCACTCAATGGACTGAA |
| 260 | 231 | ACGGCACCGTCATGAGTCAACACCAT | 740 | 711 | TTGGACATTCAACTGTCACGAGGTGT |
| 261 | 232 | GTAATACTACTGTGCGAGGACCGGTA | 741 | 712 | ACTAGCACCGTGCTGGTGTGAACAAG |
| 262 | 233 | AAGTCACTTGTATGCAGGTACGTTCA | 742 | 713 | CGGCACAAGCTCCTAGGTTGAGCAGG |
| 263 | 234 | GTCACACCACAGTGGACAGACACAGG | 743 | 714 | GAAGCCATAGCTCTTAATAGACGGAG |
| 264 | 235 | ATTAGACTGGAGTGTGTACACTTGTT | 744 | 715 | ACAAGCAGATTGCTCGAAGGAACGCA |
| 265 | 236 | TGCTAACACTATTGCTCTAACAGTAG | 745 | 716 | GCAACCAAGGTGCTATTGAGACACAT |
| 266 | 237 | TAAGAACCCTATTGGTCACACCACAG | 746 | 717 | CAAGGCATGACGCTCAGCCGAGATTG |
| 267 | 238 | TGGTTACAAGAATGTCTACACATACC | 747 | 718 | ACCAGCATCATTCTTCTCAGACGCGT |
| 268 | 239 | ACTCTACTCCTTTGCACGTACTAGGC | 748 | 719 | CCGGACAATCATCTCTCTGGAACGTG |
| 269 | 240 | GTCTCACCTTCCTGTGGTGACAGTCT | 749 | 720 | TTGAGCACCTAACTTCGAAGATGGAA |
| 270 | 241 | TCCGCACGTTCATGCTTCGACAAGGA | 750 | 721 | CCACCCATTACACTAAGGCGACTTGG |
| 271 | 242 | AGGTTACGCAGGTGGTAGAACGTCAG | 751 | 722 | GTTGCCAAGTTGCTTGAACGAGCAAC |
| 272 | 243 | GAACCACATGAATGGACATACTGTCA | 752 | 723 | TCACTCACATGTCTCCGCTGATAGCT |
| 273 | 244 | TTGAGACAGGATTGTCCGCACAAGGC | 753 | 724 | GACTGCAGTTGCCTCACCGGAAGGAA |
| 274 | 245 | TGGTCACTAGTGTGACTGCACCTTAT | 754 | 725 | ATCGTCACGCTCCTCGTATGAAATCA |
| 275 | 246 | AGTGGACATAATTGTACGCACACGTA | 755 | 726 | GGTGCCAGTTCGCTATGACGAAGAAC |
| 276 | 247 | GGCACACGCCATTGCGCTTACGAAGT | 756 | 727 | CGGCGCATAAGACTATTCAGATTGCA |
| 277 | 248 | GATCTACCTGGATGCTGCAACCTTCA | 757 | 728 | GACATCACAGCTCTTCATGGATCCTG |
| 278 | 249 | TGCTGACGACATTGCAGCGACGACAA | 758 | 729 | ACTAACATTCAGCTAATTCGAGATCG |
| 279 | 250 | CCGAAACCGTTGTGGGATCACCGCAT | 759 | 730 | TTCCTCACCTTACTTTCCGGAACATT |
| 280 | 251 | ATTAAACTACGCTGTGCGGACTGTTG | 760 | 731 | TGTGTCAAAGCTCTTGGCAGACGACC |
| 281 | 252 | TAGTCACACAACTGACATAACACGGA | 761 | 732 | GTGGCCATGGTTCTGCCACGAAGCAC |
| 282 | 253 | GGTATACTGAGATGGACGTACTCGCG | 762 | 733 | TCGACCATTAAGCTCAGTAGAGTTGT |
| 283 | 254 | CAAGAACTGCTTTGCATTCACAACAA | 763 | 734 | CACGTCATAGGCCTAGCTCGATCAAG |
| 284 | 255 | ACGAGACACTGATGCACGGACATTAT | 764 | 735 | TGAAGCATAAGTCTTCTGGGAAATTA |
| 285 | 256 | TTATCACTTGCATGTTGAGACGACGG | 765 | 736 | ACGGACAATGCGCTATTAGGATGGAG |
| 286 | 257 | AGATTACGTTACTGCTCTGACTATAC | 766 | 737 | GTGTGCAATATCCTGACTAGATATGT |
| 287 | 258 | TCTACACCGCTGTGGCAACACAGGTG | 767 | 738 | ACACACAGCGCTCTCGTTCGAGGAAC |
| 288 | 259 | AACGGACTATGATGGGTAAACCGCAG | 768 | 739 | AGCGCCAGGTGACTTCGATGAACTAG |
| 289 | 260 | CAATGACGCGCCTGACCGCACGCAAT | 769 | 740 | CAAGGCACTATCCTTACCAGACAATG |
| 290 | 261 | CTAATACTCGCTTGAGCCGACGAACA | 770 | 741 | TGCGTCACCAGGCTTGGTAGATACCA |
| 291 | 262 | CATGGACTCTAATGTCCTAACGGAAG | 771 | 742 | AGGTGCACGTAACTGCTCTGACGTTG |
| 292 | 263 | ATACTACGTGTGTGTTGAGACCCTAA | 772 | 743 | GCAGCCAAACGACTGTCTCGAGTGAA |
| 293 | 264 | GCCGAACCAAGATGCCACCACTGTGT | 773 | 744 | ATCCTCATGTCGCTAAGGCGACACCT |
| 294 | 265 | CGAGGACCGGTATGCCTCGACCAACC | 774 | 745 | GAAGGCATACACCTCTGTGGAAGCTA |
| 295 | 266 | GATATACAACAGTGGTATAACGCTGT | 775 | 746 | TTGGCCACAGGTCTTCACAGAGATCG |
| 296 | 267 | TCGCCACGGTTATGGCTACACATTAG | 776 | 747 | AGGCCCAAGACACTAGAAGGACCAAT |
| 297 | 268 | AGACTACCTCTTTGTACGAACATCTT | 777 | 748 | AGCATCATAACTCTACTGCGAAGCCG |
| 298 | 269 | GCTCGACCCTACTGTAGGAACGCGCA | 778 | 749 | ATTACCATCACCCTAACATGACTAGT |
| 299 | 270 | AGGATACAAGTTTGGTACTACGGCGT | 779 | 750 | GCGCACAGAGTACTCCTTAGACTATG |
| 300 | 271 | GAGACACATAATTGAGTTAACAGAGC | 780 | 751 | CGCCACATACCTCTGTGGCGAGAGAC |
| 301 | 272 | AGCTGACTTATATGTCGCGACTATAA | 781 | 752 | GCAGGCACTGGACTGCCAGGAATCCA |
| 302 | 273 | GTATCACATTGGTGGAGTGACTGCCG | 782 | 753 | GTTATCAATGGCCTACACAGAATATC |
| 303 | 274 | AATAGACGCCTCTGCTAGTACCCGGA | 783 | 754 | CACTCCAGCACTCTTGGAGGAGTAAT |
| 304 | 275 | CCGCTACTAGCTTGATTAAACTACGC | 784 | 755 | ACCGGCACTCAGCTCCTTCGAACGTA |
| 305 | 276 | TCCTAACGGAAGTGCCTAGACAGTAT | 785 | 756 | ATAGACACCGTTCTCTATAGACGCGG |
| 306 | 277 | TCACAACGATCGTGTAGGAACAGACT | 786 | 757 | TGAACCAGCAACCTGTTGCGAAGTTG |
| 307 | 278 | ACTTGACTCCACTGCCGTGACGCCTT | 787 | 758 | GTGGTCATGAAGCTTTATGGACGCCT |
| 308 | 279 | TGTACACTTGTTTGGGATAACTATCC | 788 | 759 | ACTGACAATAGACTTCTCAGAGTACA |
| 309 | 280 | CACTTACAATCTTGCACCTACCTTGG | 789 | 760 | GGACGCATCTTGCTAGTATGAACGGA |
| 310 | 281 | CAGAGACTGATATGAACGTACTACAT | 790 | 761 | GTTGTCAACTCACTACGCTGATGGAC |
| 311 | 282 | GGCGAACATTCTTGCGGCAACAGCTC | 791 | 762 | AGAACCACGCGGCTGGAGTGAAGATT |
| 312 | 283 | AGTGGACTCAGGTGTCTTGACGCTAT | 792 | 763 | CAGTACATCAATCTTACACGAGCTCC |
| 313 | 284 | CATTCACCAGCTTGACGGAACATGCG | 793 | 764 | TCCATCAAATCCCTTCCGAGATAGAG |
| 314 | 285 | CTCGTACTATCATGGTTCCACGCAGG | 794 | 765 | ATGAGCAAACCACTCTCAAGAGGCCG |
| 315 | 286 | CCTTAACCTATGTGACCAAACGTTAC | 795 | 766 | TCGTGCAGTTGACTCAAGTGATCATA |
| 316 | 287 | AGAAGACCCAATTGTGGCTACCGCAG | 796 | 767 | CAAGTCATCATACTAATCCGATTAGG |
| 317 | 288 | TAATCACGGTACTGAACTAACACGTT | 797 | 768 | CTTAACACCACTCTGGTGGGAAATAC |
| 318 | 289 | GGAATACTGTTCTGTAGAGACTTGGA | 798 | 769 | CGCTCACAGTTCACTCGTGTGGAGCG |
| 319 | 290 | CCGGAACCCACATGAGAGCACACTAG | 799 | 770 | TATCTACGACCTACCTACATGAGATA |
| 320 | 291 | GACTTACAGAAGTGACTCTACACAGG | 800 | 771 | ATATGACAGACGACTATAGTGTAGCT |
| 321 | 292 | TGGCAACATATTTGCGGTGACACACC | 801 | 772 | CTTATACGGAATACTGCCTTGGGTGG |
| 322 | 293 | GAATGACCACGATGGCGTTACGGTAT | 802 | 773 | TAATCACTCGTCACACATTTGATCCT |
| 323 | 294 | CGTGTACATCTTTGTGTGCACTAACA | 803 | 774 | GCGCGACATGTTACGTCCATGCTTGT |
| 324 | 295 | ATTCAACTTGCATGCCAGAACAGTAA | 804 | 775 | AGAGCACACTAGACTGGAATGCAGTA |
| 325 | 296 | TCCTTACCATAGTGCTTATACACCTG | 805 | 776 | TGCCTACTGATCACCCTTGTGTTAAT |
| 326 | 297 | TCTAGACTCTTCTGACTAGACAACTT | 806 | 777 | CTACTACCAGTCACGTTGATGTAGTG |
| 327 | 298 | CTCGAACCTCCTTGTTAGGACCTTAC | 807 | 778 | TCGTCACTGACTACACCAGTGCGACA |
| 328 | 299 | AGTGAACGTGAATGTATCAACTGAGA | 808 | 779 | GAACAACTACGGACCATACTGACTGT |
| 329 | 300 | GAAGCACGGACCTGCTCACACACAAG | 809 | 780 | CCTATACGACTCACGTGTGTGGCGCT |
| 330 | 301 | GCTCTACCGTTGTGGAATTACGAGTG | 810 | 781 | TAATGACGCAAGACATCACTGGAAGG |
| 331 | 302 | GGACCACTCAATTGCGGATACTATAT | 811 | 782 | GTGCCACGCTTCACCGGCTTGCTACT |
| 332 | 303 | GAGTCACTCTCCTGTTGAAACGCAGA | 812 | 783 | CGGCAACATGGAACGAATGTGCACGA |
| 333 | 304 | AACGGACAGCGGTGTACGGACCGAAG | 813 | 784 | GCCGTACAACCGACAAGACTGTATAG |
| 334 | 305 | TGTGAACTGTATTGTCTCCACATTGA | 814 | 785 | AACCAACTTCTCACTCGGCTGAGCAA |
| 335 | 306 | AACATACACCTATGCGAGAACCCAAG | 815 | 786 | GGTTGACCCTCTACCTAATTGGATGG |
| 336 | 307 | GTGCTACAGGTGTGTGCTGACGACAT | 816 | 787 | CTAATACGATGGACGGTTGTGCCTCT |
| 337 | 308 | CATACACTTGAATGGATGGACTATCG | 817 | 788 | TCGGCACCTATCACCGCACTGATGGC |
| 338 | 309 | CTTGTACCTTAATGGGCTTACAATTG | 818 | 789 | AGTCAACACCATACGGCCTTGGTCCT |
| 339 | 310 | AAGAGACAGGTGTGCTCGAACCTCCT | 819 | 790 | GAGCGACCAATAACCTGTGTGTTAGG |
| 340 | 311 | TGCACACGAGAATGATACAACCAGAG | 820 | 791 | AACAAACGGCGTACTAAGGTGAACGT |
| 341 | 312 | ACTTCACCTAGCTGTCTCGACGACGA | 821 | 792 | GTATGACTAGAAACCTAACTGTGTAA |
| 342 | 313 | GTGCTACATTAATGACCACACGTCTG | 822 | 793 | TTCTAACTGGTTACGGCGATGGATGG |
| 343 | 314 | AGCGTACGAATGTGGTTGTACACTCA | 823 | 794 | CCTCGACCAACCACAATAGTGAGCAA |
| 344 | 315 | CCTTAACGTGCCTGTCAGGACTCAAC | 824 | 795 | TGGATACGCTTAACTCAATTGCCATT |
| 345 | 316 | TGTACACCGAATTGAGTCCACGAGGA | 825 | 796 | ATGTCACGTGGTACTCGTATGTGCGG |
| 346 | 317 | GGAGAACTTAGTTGCACTTACAATCT | 826 | 797 | AGAGTACGCGGCACTCCGATGCCTCG |
| 347 | 318 | TACTAACACACATGTACTCACTGTTA | 827 | 798 | TGCCTACGGTGGACCTTATTGGGAAT |
| 348 | 319 | TAGGTACCGTTGTGGCGACACTCGAT | 828 | 799 | TGCGTACGTCACACGCTTATGCGGAC |
| 349 | 320 | ATGCCACGACCGTGCTAGGACCAAGG | 829 | 800 | CATACACACTGTACGAACATGTACGG |
| 350 | 321 | CTAGCACGTCGATGCCTCTACTCGAA | 830 | 801 | CGTATACAATCAACGTCGATGTTACA |
| 351 | 322 | TGCCTACACGAGTGTCATCACCTCTT | 831 | 802 | TACGCACGGCTGACACTAGTGCCGTG |
| 352 | 323 | ACTAGACAACTTTGGGTAAACGATAA | 832 | 803 | GCGAGACTTACCACAAGTTTGGGTGA |
| 353 | 324 | CACCTACCTTGGTGAACGAACGCCAG | 833 | 804 | TACGGACCCGGTACTGGCATGATATT |
| 354 | 325 | AAGCAACGATATTGTAGACACAATCT | 834 | 805 | GTCGAACTTACAACGATCATGCCGCG |
| 355 | 326 | GCCAGACATCCATGCAATGACCTGAA | 835 | 806 | CTGTCACTGCACACTACCATGTCCGT |
| 356 | 327 | TTGGAACTTCAATGGTCACACGGTGT | 836 | 807 | CAGCCACGATTGACGCTGTTGAGGAA |
| 357 | 328 | ACTAGACCCGTGTGGGTGTACACAAG | 837 | 808 | TGACTACACATAACCGCACTGTAATG |
| 358 | 329 | CGGCAACAGCTCTGAGGTTACGCAGG | 838 | 809 | ATTGCACCGAGTACGACAATGCTGAA |
| 359 | 330 | GAAGCACTAGCTTGTAATAACCGGAG | 839 | 810 | GCCATACTAGACACAGTGGTGTCAGG |
| 360 | 331 | ACAAGACGATTGTGCGAAGACACGCA | 840 | 811 | GGCGAACGATGGACTTCTATGTGGTT |
| 361 | 332 | GCAACACAGGTGTGATTGAACCACAT | 841 | 812 | TGGCTACCGCAGACAATCCTGGGCCA |
| 362 | 333 | CAAGGACTGACGTGCAGCCACGATTG | 842 | 813 | TAGAAACTAACGACCCATATGAGGTT |
| 363 | 334 | ACCAGACTCATTTGTCTCAACCGCGT | 843 | 814 | TAATGACGATCTACATCTCTGTACCA |
| 364 | 335 | CCGGAACATCATTGCTCTGACACGTG | 844 | 815 | TATCCACAGGACACCGGTGTGGCGAA |
| 365 | 336 | TTGAGACCCTAATGTCGAAACTGGAA | 845 | 816 | AGTGCACCACTGACTAACATGATAGG |
| 366 | 337 | CCACCACTTACATGAAGGCACCTTGG | 846 | 817 | GTGCAACACACTACCTGGTTGACACG |
| 367 | 338 | GTTGCACAGTTGTGTGAACACGCAAC | 847 | 818 | ACATGACGTGTCACTCAACTGGTGTA |
| 368 | 339 | TCACTACCATGTTGCCGCTACTAGCT | 848 | 819 | GACAGACACAGGACACTGTTGTGTGA |
| 369 | 340 | GACTGACGTTGCTGCACCGACAGGAA | 849 | 820 | TCTTAACCATCAACGTGCGTGTCCTT |
| 370 | 341 | ATCGTACCGCTCTGCGTATACAATCA | 850 | 821 | TTACAACATTCCACAGCACTGATCCT |
| 371 | 342 | GGTGCACGTTCGTGATGACACAGAAC | 851 | 822 | AAGCTACTATGCACTTCCGTGTCGCA |
| 372 | 343 | CGGCGACTAAGATGATTCAACTTGCA | 852 | 823 | TATTCACCTCAGACCTTAATGCCACT |
| 373 | 344 | GACATACCAGCTTGTCATGACTCCTG | 853 | 824 | CTCGTACGCGTTACGCCTCTGGGATA |
| 374 | 345 | ACTAAACTTCAGTGAATTCACGATCG | 854 | 825 | TTAGGACATAGAACCGTCGTGACTGG |
| 375 | 346 | TTCCTACCCTTATGTTCCGACACATT | 855 | 826 | CCGAAACGCGAGACTACTATGGTCAA |
| 376 | 347 | TGTGTACAAGCTTGTGGCAACCGACC | 856 | 827 | GGACCACAACAGACATAGATGCCGTT |
| 377 | 348 | GTGGCACTGGTTTGGCCACACAGCAC | 857 | 828 | TTCCAACGGTAAACACAGTTGTCCAG |
| 378 | 349 | TCGACACTTAAGTGCAGTAACGTTGT | 858 | 829 | TGATTACAGCCAACAGGCATGTGTAG |
| 379 | 350 | CACGTACTAGGCTGAGCTCACTCAAG | 859 | 830 | TAACAACGTGTTACGCAAGTGTCTCA |
| 380 | 351 | TGAAGACTAAGTTGTCTGGACAATTA | 860 | 831 | ACCGCACGCAATACTTGGCTGTCCGC |
| 381 | 352 | ACGGAACATGCGTGATTAGACTGGAG | 861 | 832 | GTTCGACCGCCAACAACTGTGATACT |
| 382 | 353 | GTGTGACATATCTGGACTAACTATGT | 862 | 833 | AGACAACCATTAACGTAAGTGGCATA |
| 383 | 354 | ACACAACGCGCTTGCGTTCACGGAAC | 863 | 834 | GCGTTACGGTATACAATTGTGCTGCG |
| 384 | 355 | AGCGCACGGTGATGTCGATACACTAG | 864 | 835 | AGCACACATCCTACTTACATGATTCC |
| 385 | 356 | CAAGGACCTATCTGTACCAACCAATG | 865 | 836 | TTGTTACCCGTGACAACCTTGAGCAC |
| 386 | 357 | TGCGTACCCAGGTGTGGTAACTACCA | 866 | 837 | AAGTAACCTCCAACTCTGTTGGTGGA |
| 387 | 358 | AGGTGACCGTAATGGCTCTACCGTTG | 867 | 838 | ACGTCACAATACACGGAATTGTCCAA |
| 388 | 359 | GCAGCACAACGATGGTCTCACGTGAA | 868 | 839 | GGTGTACACAAGACAAGCGTGCGCTT |
| 389 | 360 | ATCCTACTGTCGTGAAGGCACCACCT | 869 | 840 | CCACCACTGTGTACTGAGCTGGTTGT |
| 390 | 361 | GAAGGACTACACTGCTGTGACAGCTA | 870 | 841 | GTTCCACGCAGGACATCATTGAGGCT |
| 391 | 362 | TTGGCACCAGGTTGTCACAACGATCG | 871 | 842 | ACCTTACATGAAACTGTTATGGAAGG |
| 392 | 363 | AGGCCACAGACATGAGAAGACCCAAT | 872 | 843 | CGCTGACCAGAGACGATGGTGATGTA |
| 393 | 364 | AGCATACTAACTTGACTGCACAGCCG | 873 | 844 | GTAGAACGTCAGACACGGCTGCGTCA |
| 394 | 365 | ATTACACTCACCTGAACATACCTAGT | 874 | 845 | GGATAACCCAGAACCGTTGTGCTTAC |
| 395 | 366 | GCGCAACGAGTATGCCTTAACCTATG | 875 | 846 | CGCACACTAATGACTGACTTGACATA |
| 396 | 367 | CGCCAACTACCTTGGTGGCACGAGAC | 876 | 847 | TCCTGACACCGTACCGGCCTGTCGTT |
| 397 | 368 | GCAGGACCTGGATGGCCAGACATCCA | 877 | 848 | CTGGCACTTGCCACCAAGCTGATCCG |
| 398 | 369 | GTTATACATGGCTGACACAACATATC | 878 | 849 | ACCAGACCGACAACTCGTCTGTGACT |
| 399 | 370 | CACTCACGCACTTGTGGAGACGTAAT | 879 | 850 | TTGTAACACGGTACCTCATTGAGCGA |
| 400 | 371 | ACCGGACCTCAGTGCCTTCACACGTA | 880 | 851 | GTAAGACGCATAACAGACATGCATTA |
| 401 | 372 | ATAGAACCCGTTTGCTATAACCGCGG | 881 | 852 | GTCCAACCTTGTACGCGCGTGATGTT |
| 402 | 373 | TGAACACGCAACTGGTTGCACAGTTG | 882 | 853 | TTAGGACTACCAACCATGATGGTACT |
| 403 | 374 | GTGGTACTGAAGTGTTATGACCGCCT | 883 | 854 | GGAATACTCCAAACACGTCTGAATAC |
| 404 | 375 | ACTGAACATAGATGTCTCAACGTACA | 884 | 855 | CATGTACAGAGGACGATACTGCTCCT |
| 405 | 376 | GGACGACTCTTGTGAGTATACACGGA | 885 | 856 | TACACACGCTCCACATCCGTGTAAGT |
| 406 | 377 | GTTGTACACTCATGACGCTACTGGAC | 886 | 857 | GCTTAACCGGACACCGTGTTGATCTT |
| 407 | 378 | AGAACACCGCGGTGGGAGTACAGATT | 887 | 858 | CGCTTACGAAGTACGAACCTGATGAA |
| 408 | 379 | CAGTAACTCAATTGTACACACGCTCC | 888 | 859 | CGCCTACTCTGAACGGCCATGTCATA |
| 409 | 380 | TCCATACAATCCTGTCCGAACTAGAG | 889 | 860 | ATACCACAACGCACACATATGCTTCC |
| 410 | 381 | ATGAGACAACCATGCTCAAACGGCCG | 890 | 861 | CTGGAACTATGTACTATGTTGGCAAT |
| 411 | 382 | TCGTGACGTTGATGCAAGTACTCATA | 891 | 862 | CAATCACTATGAACGATTATGAGGTG |
| 412 | 383 | CAAGTACTCATATGAATCCACTTAGG | 892 | 863 | GGTGGACAATACACATGTATGGACAA |
| 413 | 384 | CTTAAACCCACTTGGGTGGACAATAC | 893 | 864 | TGGACACGGAGGACCACATTGCGGTG |
| 414 | 385 | CGCTCCAAGTTCCTTCGTGGAGAGCG | 894 | 865 | CTGACACCGGCAACCCTGATGTACAA |
| 415 | 386 | TATCTCAGACCTCTCTACAGAAGATA | 895 | 866 | GAATTACGAGTGACTTAAGTGTTGTG |
| 416 | 387 | ATATGCAAGACGCTTATAGGATAGCT | 896 | 867 | GCGTGACTGAGAACCGGACTGAGTGA |
| 417 | 388 | CTTATCAGGAATCTTGCCTGAGGTGG | 897 | 868 | TCTCCACATTGAACGCACTTGACAAC |
| 418 | 389 | TAATCCATCGTCCTACATTGAATCCT | 898 | 869 | ACATGACCATATACTGGTGTGCCTGG |
| 419 | 390 | GCGCGCAATGTTCTGTCCAGACTTGT | 899 | 870 | CAGGCACGCCATACTCCACTGGGCCT |
| 420 | 391 | AGAGCCAACTAGCTTGGAAGACAGTA | 900 | 871 | ACATAACACGGAACTTGTATGGTGTA |
| 421 | 392 | TGCCTCATGATCCTCCTTGGATTAAT | 901 | 872 | TTAATACAGACCACCCACGTGACACG |
| 422 | 393 | CTACTCACAGTCCTGTTGAGATAGTG | 902 | 873 | ACGATACTGCTGACTGTGATGTGTAT |
| 423 | 394 | TCGTCCATGACTCTACCAGGACGACA | 903 | 874 | TTCTAACCAGAAACGAGCGTGCAATA |
| 424 | 395 | GAACACATACGGCTCATACGAACTGT | 904 | 875 | TATTGACCGTTCACATCTTTGACTGT |
| 425 | 396 | CCTATCAGACTCCTGTGTGGAGCGCT | 905 | 876 | CATGAACGTACTACATGTCTGGTGGT |
| 426 | 397 | TAATGCAGCAAGCTATCACGAGAAGG | 906 | 877 | TAATTACCTACCACGTAGCTGCATCA |
| 427 | 398 | GTGCCCAGCTTCCTCGGCTGACTACT | 907 | 878 | ACGCTACAATTAACTGGTTTGAAGAA |
| 428 | 399 | CGGCACAATGGACTGAATGGACACGA | 908 | 879 | CCTTGACTTAATACTGTTGTGTTCGT |
| 429 | 400 | GCCGTCAAACCGCTAAGACGATATAG | 909 | 880 | GTAGCACCATCAACCCAACTGAACAT |
| 430 | 401 | AACCACATTCTCCTTCGGCGAAGCAA | 910 | 881 | CTTGTACAATTCACACCGGTGCTCAG |
| 431 | 402 | GGTTGCACCTCTCTCTAATGAGATGG | 911 | 882 | TCCAAACTTCTAACGTTAATGTCTGA |
| 432 | 403 | CTAATCAGATGGCTGGTTGGACCTCT | 912 | 883 | AGAGCACTGCCTACCGGCTTGAACGT |
| 433 | 404 | TCGGCCACTATCCTCGCACGAATGGC | 913 | 884 | CTTCGACCCGATACTCCAATGGAATT |
| 434 | 405 | AGTCACAACCATCTGGCCTGAGTCCT | 914 | 885 | TCGGTACCACGGACCCGAATGCGTTG |
| 435 | 406 | GAGCGCACAATACTCTGTGGATTAGG | 915 | 886 | GAACAACAGTATACTAACCTGGCCGA |
| 436 | 407 | AACAACAGGCGTCTTAAGGGAAACGT | 916 | 887 | AATTGACGCGGAACCTCCGTGTGCTG |
| 437 | 408 | GTATGCATAGAACTCTAACGATGTAA | 917 | 888 | GGCCTACGTCCTACCATTCTGCAGCT |
| 438 | 409 | TTCTACATGGTTCTGGCGAGAGATGG | 918 | 889 | TAGGTACTCTCTACGGTTATGTGCTA |
| 439 | 410 | CCTCGCACAACCCTAATAGGAAGCAA | 919 | 890 | ACACAACATATCACACCACTGACGGT |
| 440 | 411 | TGGATCAGCTTACTTCAATGACCATT | 920 | 891 | TTCCTACGTACGACTAGGTTGTCTCT |
| 441 | 412 | ATGTCCAGTGGTCTTCGTAGATGCGG | 921 | 892 | GGTAAACCGCAGACTATGGTGCTCGA |
| 442 | 413 | AGAGTCAGCGGCCTTCCGAGACCTCG | 922 | 893 | TCCACACGGCCTACCTCGTTGGCGTT |
| 443 | 414 | TGCCTCAGGTGGCTCTTATGAGGAAT | 923 | 894 | GATACACCTCCTACCCAGTTGTGGCA |
| 444 | 415 | TGCGTCAGTCACCTGCTTAGACGGAC | 924 | 895 | CAACGACTCAGCACTGTTCTGGCATT |
| 445 | 416 | CATACCAACTGTCTGAACAGATACGG | 925 | 896 | CGGTTACATTAGACAACCGTGCATCG |
| 446 | 417 | CGTATCAAATCACTGTCGAGATTACA | 926 | 897 | CGCGCACCTAGAACCGAAGTGGTTAA |
| 447 | 418 | TACGCCAGGCTGCTACTAGGACCGTG | 927 | 898 | TCTTGACGCTATACAGTGCTGCACTG |
| 448 | 419 | GCGAGCATTACCCTAAGTTGAGGTGA | 928 | 899 | TCACAACCCGAAACGAACATGAGTAT |
| 449 | 420 | TACGGCACCGGTCTTGGCAGAATATT | 929 | 900 | AACGTACTACATACACGATTGTGCTG |
| 450 | 421 | GTCGACATTACACTGATCAGACCGCG | 930 | 901 | CGGCCACTCGTTACATACCTGTGGAT |
| 451 | 422 | CTGTCCATGCACCTTACCAGATCCGT | 931 | 902 | CATAAACCACCAACTCCAATGTTCTA |
| 452 | 423 | CAGCCCAGATTGCTGCTGTGAAGGAA | 932 | 903 | ACAGAACGGCCAACTGAGATGCAGCG |
| 453 | 424 | TGACTCAACATACTCGCACGATAATG | 933 | 904 | TGGTGACCCTGGACACGCTTGAATTA |
| 454 | 425 | ATTGCCACGAGTCTGACAAGACTGAA | 934 | 905 | TAGGAACACCGGACTATATTGTCGAG |
| 455 | 426 | GCCATCATAGACCTAGTGGGATCAGG | 935 | 906 | AATATACTGGCCACCGGTCTGCGATA |
| 456 | 427 | GGCGACAGATGGCTTTCTAGATGGTT | 936 | 907 | ATAGGACTATTCACACAATTGAGAGT |
| 457 | 428 | TGGCTCACGCAGCTAATCCGAGGCCA | 937 | 908 | CCTTCACACGTAACCGGTTTGATTAG |
| 458 | 429 | TAGAACATAACGCTCCATAGAAGGTT | 938 | 909 | GGCCAACATAAGACGATAATGCAAGT |
| 459 | 430 | TAATGCAGATCTCTATCTCGATACCA | 939 | 910 | CAGTAACGTTGTACAGTTATGTCACA |
| 460 | 431 | TATCCCAAGGACCTCGGTGGAGCGAA | 940 | 911 | TTCATACCCAACACTTCCATGGGTAA |
| 461 | 432 | AGTGCCACACTGCTTAACAGAATAGG | 941 | 912 | CAATTACGGATTACCATGTTGAGAGG |
| 462 | 433 | GTGCACAACACTCTCTGGTGAACACG | 942 | 913 | GGCCAACTCATAACGATTGTGTCATA |
| 463 | 434 | ACATGCAGTGTCCTTCAACGAGTGTA | 943 | 914 | AATTGACCTGCGACATTCCTGGCTAT |
| 464 | 435 | GACAGCAACAGGCTACTGTGATGTGA | 944 | 915 | TAAGGACAACGTACGACCGTGCTGTG |
| 465 | 436 | TCTTACACATCACTGTGCGGATCCTT | 945 | 916 | CTATAACCGCGGACTAGGATGACCGG |
| 466 | 437 | TTACACAATTCCCTAGCACGAATCCT | 946 | 917 | ATTCAACGAATCACAGCGGTGTGGAC |
| 467 | 438 | AAGCTCATATGCCTTTCCGGATCGCA | 947 | 918 | GTATTACCTCTAACTATAGTGATTCG |
| 468 | 439 | TATTCCACTCAGCTCTTAAGACCACT | 948 | 919 | CCTGAACTACAAACACAGATGGGCCA |
| 469 | 440 | CTCGTCAGCGTTCTGCCTCGAGGATA | 949 | 920 | GACCGACCTGTGACATTCCTGTATTG |
| 470 | 441 | TTAGGCAATAGACTCGTCGGAACTGG | 950 | 921 | TTCAGACCGTGGACTATTCTGCTCAG |
| 471 | 442 | CCGAACAGCGAGCTTACTAGAGTCAA | 951 | 922 | AACTCACCGAACACCGCCTTGTCTGA |
| 472 | 443 | GGACCCAAACAGCTATAGAGACCGTT | 952 | 923 | ATTCCACGCTATACGCGCATGGAGTA |
| 473 | 444 | TTCCACAGGTAACTACAGTGATCCAG | 953 | 924 | TGAATACATTGCACGGCGCTGCAATT |
| 474 | 445 | TGATTCAAGCCACTAGGCAGATGTAG | 954 | 925 | CGCAAACTCTAGACAGATATGTGGCG |
| 475 | 446 | TAACACAGTGTTCTGCAAGGATCTCA | 955 | 926 | AACCGACCATCGACCCTGCTGTTGGT |
| 476 | 447 | ACCGCCAGCAATCTTTGGCGATCCGC | 956 | 927 | CTAGTACCCGGAACGACGATGACAAT |
| 477 | 448 | GTTCGCACGCCACTAACTGGAATACT | 957 | 928 | GCTCCACGTCACACTGGCGTGGTCCA |
| 478 | 449 | AGACACACATTACTGTAAGGAGCATA | 958 | 929 | AGATGACGAATTACCTTCATGGTTAC |
| 479 | 450 | GCGTTCAGGTATCTAATTGGACTGCG | 959 | 930 | ACACCACGTTAAACTCCTGTGACCGT |
| 480 | 451 | AGCACCAATCCTCTTTACAGAATTCC | 960 | 931 | GATAAACCAAGTACCGCGCTGCTAGA |
| 481 | 452 | TTGTTCACCGTGCTAACCTGAAGCAC | 961 | 932 | CTGGTACACACGACAGGATTGAAGTT |
| 482 | 453 | AAGTACACTCCACTTCTGTGAGTGGA | 962 | 933 | CGAAGACGTTAAACAGGCCTGAGACA |
| 483 | 454 | ACGTCCAAATACCTGGAATGATCCAA | 963 | 934 | ATCGCACATATGACCCTTGTGAACGG |
| 484 | 455 | GGTGTCAACAAGCTAAGCGGACGCTT | 964 | 935 | ATCATACAGGCTACCACCATGCCTAC |
| 485 | 456 | CCACCCATGTGTCTTGAGCGAGTTGT | 965 | 936 | GATTGACTCATAACTTGCTTGTGTAT |
| 486 | 457 | GTTCCCAGCAGGCTATCATGAAGGCT | 966 | 937 | CCAACACAACATACCAATCTGTATGA |
| 487 | 458 | ACCTTCAATGAACTTGTTAGAGAAGG | 967 | 938 | TTGGTACGGTGCACTGGTATGCTGAT |
| 488 | 459 | CGCTGCACAGAGCTGATGGGAATGTA | 968 | 939 | GCGAAACCGCCTACTTCATTGCCAAC |
| 489 | 460 | GTAGACAGTCAGCTACGGCGACGTCA | 969 | 940 | CAACCACGGAGGACCATAATGCACCA |
| 490 | 461 | GGATACACCAGACTCGTTGGACTTAC | 970 | 941 | AGCGGACTGGACACTCCTATGTTAGC |
| 491 | 462 | CGCACCATAATGCTTGACTGAACATA | 971 | 942 | GACGAACACAATACTCTCTTGAGATT |
| 492 | 463 | TCCTGCAACCGTCTCGGCCGATCGTT | 972 | 943 | CCACTACGGTCCACCGCGATGGCCTA |
| 493 | 464 | CTGGCCATTGCCCTCAAGCGAATCCG | 973 | 944 | TGTTAACGAAGGACGATAATGGCTCT |
| 494 | 465 | ACCAGCACGACACTTCGTCGATGACT | 974 | 945 | TATATACTCGAGACGAGATTGGTCGA |
| 495 | 466 | TTGTACAACGGTCTCTCATGAAGCGA | 975 | 946 | CGCGAACCGATCACCTGGATGTATGT |
| 496 | 467 | GTAAGCAGCATACTAGACAGACATTA | 976 | 947 | GCCTCACGGATAACGGCCATGATAAG |
| 497 | 468 | GTCCACACTTGTCTGCGCGGAATGTT | 977 | 948 | TGAGAACCAGCGACATTACTGTCACC |
| 498 | 469 | TTAGGCATACCACTCATGAGAGTACT | 978 | 949 | TGTTCACGCATTACAATTGTGGCGGA |
| 499 | 470 | GGAATCATCCAACTACGTCGAAATAC | 979 | 950 | TCCAAACGAATTACTTGTCTGAACTT |
| 500 | 471 | CATGTCAAGAGGCTGATACGACTCCT | 980 | 951 | GCTGTACAGGAAACGGCGATGATTCT |
| 501 | 472 | TACACCAGCTCCCTATCCGGATAAGT | 981 | 952 | ATACCACTGGATACCAACGTGTCAGC |
| 502 | 473 | GCTTACACGGACCTCGTGTGAATCTT | 982 | 953 | GTTGGACACCGTACTCTTATGCATCA |
| 503 | 474 | CGCTTCAGAAGTCTGAACCGAATGAA | 983 | 954 | ACCAAACGTTACACCGCCATGTACCT |
| 504 | 475 | CGCCTCATCTGACTGGCCAGATCATA | 984 | 955 | GTGTGACGCGCTACCTAATTGGTCTT |
| 505 | 476 | ATACCCAAACGCCTACATAGACTTCC | 985 | 956 | GGCAGACTAGCAACCAACCTGGGAGG |
| 506 | 477 | CTGGACATATGTCTTATGTGAGCAAT | 986 | 957 | TGCGGACTGTTGACGGCAGTGTAGCA |
| 507 | 478 | CAATCCATATGACTGATTAGAAGGTG | 987 | 958 | GATTAACAGGTGACTTAGGTGATAGA |
| 508 | 479 | GGTGGCAAATACCTATGTAGAGACAA | 988 | 959 | CAACAACTTCAAACCGCAATGTCTAG |
| 509 | 480 | TGGACCAGGAGGCTCACATGACGGTG | 989 | 960 | GTGTTACACCGGACGAGTTTGGTACT |

Table 6 lists the 96x selected sample barcodes (Barcode IDs: UDPS001-096).

TABLE 6

96x selected sample barcodes (Barcode IDs: UDPS001-096)

| SEQ ID NO | UDPS ID | UDPX ID | Barcode Sequence |
|---|---|---|---|
| 129 | UDPS001 | UDPX100 | TCTCCACATTGATGGCACTACACAAC |
| 130 | UDPS002 | UDPX101 | ACATGACCATATTGTGGTGACCCTGG |
| 131 | UDPS003 | UDPX102 | CAGGCACGCCATTGTCCACACGGCCT |
| 135 | UDPS004 | UDPX106 | TTCTAACCAGAATGGAGCGACCAATA |
| 136 | UDPS005 | UDPX107 | TATTGACCGTTCTGATCTTACACTGT |
| 359 | UDPS006 | UDPX330 | GAAGCACTAGCTTGTAATAACCGGAG |
| 225 | UDPS007 | UDPX196 | CCATCACCACGCTGTTGACACCAATG |
| 226 | UDPS008 | UDPX197 | ACAACACCAGGATGCTGACACCGGCA |
| 383 | UDPS009 | UDPX354 | ACACAACGCGCTTGCGTTCACGGAAC |
| 231 | UDPS010 | UDPX202 | AGTTAACTCACATGCAGGCACGCCAT |
| 232 | UDPS011 | UDPX203 | GTAGCACATACTTGTTAATACAGACC |
| 233 | UDPS012 | UDPX204 | CTTCAACGTTACTGGGAGTACCGCGA |
| 141 | UDPS013 | UDPX112 | GTAGCACCATCATGCCAACACAACAT |
| 142 | UDPS014 | UDPX113 | CTTGTACAATTCTGACCGGACCTCAG |
| 143 | UDPS015 | UDPX114 | TCCAAACTTCTATGGTTAAACTCTGA |
| 147 | UDPS016 | UDPX118 | GAACAACAGTATTGTAACCACGCCGA |
| 148 | UDPS017 | UDPX119 | AATTGACGCGGATGCTCCGACTGCTG |
| 149 | UDPS018 | UDPX120 | GGCCTACGTCCTTGCATTCACCAGCT |
| 237 | UDPS019 | UDPX208 | TTATCACCGATCTGGTATCACGGCCG |
| 238 | UDPS020 | UDPX209 | ATAGTACCTAGCTGAATACACGACAT |
| 407 | UDPS021 | UDPX378 | AGAACACCGCGGTGGGAGTACAGATT |
| 243 | UDPS022 | UDPX214 | TCGCGACTATAATGGCCGTACCTGTT |
| 244 | UDPS023 | UDPX215 | ATTCTACAAGCGTGCAGAGACTGATA |
| 245 | UDPS024 | UDPX216 | AGCGCACTTCGGTGTGCTAACACTAT |
| 321 | UDPS025 | UDPX292 | TGGCAACATATTTGCGGTGACACACC |
| 154 | UDPS026 | UDPX125 | TCCACACGGCCTTGCTCGTACGCGTT |
| 323 | UDPS027 | UDPX294 | CGTGTACATCTTTGTGTGCACTAACA |
| 159 | UDPS028 | UDPX130 | TCTTGACGCTATTGAGTGCACCACTG |
| 160 | UDPS029 | UDPX131 | TCACAACCCGAATGGAACAACAGTAT |
| 161 | UDPS030 | UDPX132 | AACGTACTACATTGACGATACTGCTG |
| 249 | UDPS031 | UDPX220 | GCGTAACCTTAGTGAACATACACCTA |
| 250 | UDPS032 | UDPX221 | TACCGACAACTATGCCATGACTGTAG |
| 341 | UDPS033 | UDPX312 | ACTTCACCTAGCTGTCTCGACGACGA |
| 405 | UDPS034 | UDPX376 | GGACGACTCTTGTGAGTATACACGGA |
| 256 | UDPS035 | UDPX227 | AATATACGAAGCTGCGTTCACAGCCT |
| 257 | UDPS036 | UDPX228 | TAGCGACCTAGTTGTTACTACTCCTC |
| 165 | UDPS037 | UDPX136 | TGGTGACCCTGGTGACGCTACAATTA |
| 166 | UDPS038 | UDPX137 | TAGGAACACCGGTGTATATACTCGAG |
| 167 | UDPS039 | UDPX138 | AATATACTGGCCTGCGGTCACCGATA |
| 171 | UDPS040 | UDPX142 | CAGTAACGTTGTTGAGTTAACTCACA |
| 172 | UDPS041 | UDPX143 | TTCATACCCAACTGTTCCAACGGTAA |
| 173 | UDPS042 | UDPX144 | CAATTACGGATTTGCATGTACAGAGG |
| 357 | UDPS043 | UDPX328 | ACTAGACCCGTGTGGGTGTACACAAG |
| 382 | UDPS044 | UDPX353 | GTGTGACATATCTGGACTAACTATGT |
| 263 | UDPS045 | UDPX234 | GTCACACCACAGTGGACAGACACAGG |
| 267 | UDPS046 | UDPX238 | TGGTTACAAGAATGTCTACACATACC |
| 268 | UDPS047 | UDPX239 | ACTCTACTCCTTTGCACGTACTAGGC |
| 269 | UDPS048 | UDPX240 | GTCTCACCTTCCTGTGGTGACAGTCT |
| 177 | UDPS049 | UDPX148 | CTATAACCGCGGTGTAGGAACACCGG |
| 178 | UDPS050 | UDPX149 | ATTCAACGAATCTGAGCGGACTGGAC |
| 179 | UDPS051 | UDPX150 | GTATTACCTCTATGTATAGACATTCG |
| 183 | UDPS052 | UDPX154 | AACTCACCGAACTGCGCCTACTCTGA |
| 370 | UDPS053 | UDPX341 | ATCGTACCGCTCTGCGTATACAATCA |
| 185 | UDPS054 | UDPX156 | TGAATACATTGCTGGGCGCACCAATT |
| 369 | UDPS055 | UDPX340 | GACTGACGTTGCTGCACCGACAGGAA |
| 274 | UDPS056 | UDPX245 | TGGTCACTAGTGTGACTGCACCTTAT |
| 353 | UDPS057 | UDPX324 | CACCTACCTTGGTGAACGAACGCCAG |
| 351 | UDPS058 | UDPX322 | TGCCTACACGAGTGTCATCACCTCTT |
| 280 | UDPS059 | UDPX251 | ATTAAACTACGCTGTGCGGACTGTTG |
| 281 | UDPS060 | UDPX252 | TAGTCACACAACTGACATAACACGGA |
| 189 | UDPS061 | UDPX160 | GCTCCACGTCACTGTGGCGACGTCCA |
| 334 | UDPS062 | UDPX305 | TGTGAACTGTATTGTCTCCACATTGA |
| 191 | UDPS063 | UDPX162 | ACACCACGTTAATGTCCTGACACCGT |
| 195 | UDPS064 | UDPX166 | ATCGCACATATGTGCCTTGACAACGG |
| 196 | UDPS065 | UDPX167 | ATCATACAGGCTTGCACCAACCCTAC |
| 371 | UDPS066 | UDPX342 | GGTGCACGTTCGTGATGACACAGAAC |
| 393 | UDPS067 | UDPX364 | AGCATACTAACTTGACTGCACAGCCG |
| 286 | UDPS068 | UDPX257 | AGATTACGTTACTGCTCTGACTATAC |
| 365 | UDPS069 | UDPX336 | TTGAGACCCTAATGTCGAAACTGGAA |
| 291 | UDPS070 | UDPX262 | CATGGACTCTAATGTCCTAACGGAAG |
| 406 | UDPS071 | UDPX377 | GTTGTACACTCATGACGCTACTGGAC |
| 293 | UDPS072 | UDPX264 | GCCGAACCAAGATGCCACCACTGTGT |
| 201 | UDPS073 | UDPX172 | CAACCACGGAGGTGCATAAACCACCA |
| 202 | UDPS074 | UDPX173 | AGCGGACTGGACTGTCCTAACTTAGC |
| 347 | UDPS075 | UDPX318 | TACTAACACACATGTACTCACTGTTA |
| 207 | UDPS076 | UDPX178 | CGCGAACCGATCTGCTGGAACTATGT |
| 208 | UDPS077 | UDPX179 | GCCTCACGGATATGGGCCAACATAAG |
| 209 | UDPS078 | UDPX180 | TGAGAACCAGCGTGATTACACTCACC |
| 297 | UDPS079 | UDPX268 | AGACTACCTCTTTGTACGAACATCTT |
| 298 | UDPS080 | UDPX269 | GCTCGACCCTACTGTAGGAACGCGCA |
| 377 | UDPS081 | UDPX348 | GTGGCACTGGTTTGGCCACACAGCAC |
| 303 | UDPS082 | UDPX274 | AATAGACGCCTCTGCTAGTACCCGGA |
| 304 | UDPS083 | UDPX275 | CCGCTACTAGCTTGATTAAACTACGC |
| 305 | UDPS084 | UDPX276 | TCCTAACGGAAGTGCCTAGACAGTAT |
| 333 | UDPS085 | UDPX304 | AACGGACAGCGGTGTACGGACCGAAG |
| 214 | UDPS086 | UDPX185 | GTTGGACACCGTTGTCTTAACCATCA |
| 215 | UDPS087 | UDPX186 | ACCAAACGTTACTGCGCCAACTACCT |
| 219 | UDPS088 | UDPX190 | GATTAACAGGTGTGTTAGGACATAGA |
| 220 | UDPS089 | UDPX191 | CAACAACTTCAATGCGCAAACTCTAG |
| 221 | UDPS090 | UDPX192 | GTGTTACACCGGTGGAGTTACGTACT |
| 309 | UDPS091 | UDPX280 | CACTTACAATCTTGCACCTACCTTGG |
| 394 | UDPS092 | UDPX365 | ATTACACTCACCTGAACATACCTAGT |
| 311 | UDPS093 | UDPX282 | GGCGAACATTCTTGCGGCAACAGCTC |
| 315 | UDPS094 | UDPX286 | CCTTAACCTATGTGACCAAACGTTAC |
| 352 | UDPS095 | UDPX323 | ACTAGACAACTTTGGGTAAACGATAA |
| 317 | UDPS096 | UDPX288 | TAATCACGGTACTGAACTAACACGTT |

Table 7 shows the inclusivity analysis of primers used.

TABLE 7

Inclusivity analysis of primers used

| Sequence homology | N#1 | | N#2 | | del6970 | | D614 | |
|---|---|---|---|---|---|---|---|---|
| | RT | PCR | RT | PCR | RT | PCR | RT | PCR |
| Exact | 99.7% | 99.8% | 97.8% | 99.5% | 99.6% | 99.4% | 99.6% | 99.9% |
| ≤1 nt mismatch | 100.0% | 100.0% | 100.0% | 100.0% | 100.0% | 100.0% | 100.0% | 100.0% |

Table 8 lists the organisms and taxonomy ID used for cross-reactivity analysis.

TABLE 8

List of organisms and taxonomy ID used for cross-reactivity analysis

| Organism | Taxonomy ID |
|---|---|
| Human adenovirus C1 | 10533 |
| Human adenovirus A | 129875 |
| Adenovirus Ad5 | 28285 |
| Human metapneumovirus | 162145 |
| Human parainfluenza virus 1 | 12730 |
| Human parainfluenza virus 2 | 1979160 |
| Human parainfluenza virus 3 | 11216 |
| Human parainfluenza virus 4 | 11203 |
| Human adenovirus 7 | 10519 |
| Influenza virus type A | 11320 |
| Influenza virus type B | 11520 |
| Human enterovirus EV68 | 42789 |
| Human respiratory syncytial virus | 11250 |
| Rhinovirus | 12059 |
| Chlamydia pneumoniae | 83558 |
| Haemophilus influenzae | 727 |
| Legionella pneumophila | 446 |
| Mycobacterium tuberculosis | 1773 |
| Streptococcus pneumoniae | 1313 |
| Streptococcus pyogenes | 1314 |
| Bordetella pertussis | 520 |
| Mycoplasma pneumoniae | 2104 |
| Pneumocystis jirovecii | 42068 |
| Candida albicans | 5476 |
| Pseudomonas aeruginosa | 287 |
| Staphylococcus epidermidis | 1282 |
| Streptococcus salivarius | 1304 |
| Human coronavirus (STRAIN 229E) | 11137 |
| Human coronavirus (strain OC43) | 31631 |
| Human coronavirus NL63 | 277944 |
| Human coronavirus HKU1 | 290028 |
| MERS | 1335626 |
| HCoV-SARS | 694009 |

Table 9 shows the breakdown of One-Seq processing times

TABLE 9

Breakdown of One-Seq processing times

| One-Seq workflow | | Processing time | | |
|---|---|---|---|---|
| | | MiSeq™ | NextSeq 550™ | NovaSeq 6000™ |
| (1) Diagnostic workflow | | | | |
| Sample incubation (one-pot reaction and inactivation) | | 40 min | | |
| Sample pooling and cDNA purification | | 60 min | | |
| Library amplification | | 90 min | | |
| Purification and quantitation | | 60 min | | |
| Sequencing (diagnostics only) | Cluster generation | 60 min | 150 min | 130 min |
| | Patient barcode (R1, 26 nt) | 120 min | 120 min | 180 min |
| | RT primer ID (R1, 5 nt) | 20 min | 20 min | 30 min |
| | (subtotal) | 200 min | 290 min | 340 min |

(2) Optional - Batch pooling

| | | | | |
|---|---|---|---|---|
| Sequencing (batch pooling) | Paired-end turn-around | 30 min | 60 min | 50 min |
| | Batch barcode (R2, 10 nt) | 45 min | 45 min | 70 min |
| | (subtotal) | 75 min | 105 min | 120 min |

(3) Optional - Variant identification

| | | | | |
|---|---|---|---|---|
| Sequencing (variant ID) | RT primer and mutation hotspot (R1, 20 nt) | 100 min | 100 min | 150 min |

Table 10 shows a breakdown of One-Seq reagent cost. “*” indicates that all costs are estimated for 20 ul patient sample input. “^{∗∗}” indicates that enzyme costs can be significantly reduced when mass produced, estimated as 25% of current off-the-shelf cost.

TABLE 10

Breakdown of One-Seqreagent cost

| Component | Current cost* (off-the-shelf) | Estimated future cost** | Product and manufacturer |
|---|---|---|---|
| RNAse inhibitor | $ 5.0 | $ 1.25 | Murine (New England Biolabs™, M0314) |
| RT enzyme | $ 5.3 | $ 1.33 | SuperScript™ IV (ThermoFisher™, 18090010) |
| Chemicals, oligonucleotides, and other additives | $ <0.3 | $ <0.2 | (various) |
| Total | $ 10.6 | $ 2.8 | |

1. A multiplexed method of detecting at least one target RNA in at least two samples, comprising:
a) contacting the at least two samples with a reverse transcriptase and a first primer or first set of primers comprising at least a first barcode, under conditions permitting the generation of reverse transcription products;
b) combining reverse transcription products from samples in step (a) in one container to form a pooled reverse transcription product mixture;
c) contacting the pooled reverse transcription product mixture with a DNA polymerase and a second set of primers under conditions permitting the generation of amplification products; and
d) sequencing the amplification products, thereby detecting at least one target RNA, if present, in the at least two samples.

2. The method of claim 1, wherein:
step (b) is performed before step (c); and/or
steps (a)-(d) are performed sequentially.

3. (canceled)

4. The method of claim 1, wherein the detection method has:
(a) a limit of detection of at least 500 target RNA copies per mL for a given target RNA; and/or
(b) a dynamic range of at least 3 logs.

5-6. (canceled)

7. The method of claim 1, wherein at least 2 target RNAs in a single sample are detected.

8-9. (canceled)

10. The method of claim 1, wherein at least one target RNA is a viral RNA.

11-13. (canceled)

14. The method of claim 1, wherein target RNAs from at least 50 samples are detected in a single performance of steps (a) - (d).

15. The method of claim 1, wherein prior to step (a), the at least one target RNA is not extracted from the sample.

16. (canceled)

17. The method of claim 1, wherein the first primer or each primer in the first set of primers comprises, from 5′ to 3′:
a) an adaptor region;
b) a first barcode region; and
c) a target-binding region that is complementary or substantially complementary to and permits hybridization to at least one target RNA; or
d) an adaptor region;
e) a first barcode region;
f) a second barcode region; and
g) a target-binding region that is complementary or substantially complementary to and permits hybridization to at least one target RNA.

18-22. (canceled)

23. The method of claim 1, wherein the target-binding region of a primer in the first set of primers binds at most 5 nucleotides away from a variation of interest in the target RNA, wherein the variation of interest is selected from the group consisting of: a single-nucleotide variation; a point mutation; a substitution; an insertion; and a deletion.

24-25. (canceled)

26. The method of claim 1, wherein step (a) further comprises contacting the sample with at least one of the following:
a) a detergent that lyses viral particles or cells in the sample and releases target RNA from the sample, wherein the detergent is a nonionic surfactant;
b) a carrier nucleic acid that reduces loss of the target RNA, wherein the carrier nucleic acid is poly-A60 DNA oligonucleotide or E. coli tRNA;
c) a positive control nucleic acid comprising from 5′ to 3′:
i) an adaptor region;
ii) a first barcode region; and
iii) a target-binding region that is complementary to or substantially complementary to a sample nucleic acid; or
iv) a region that is not identical or substantially identical to any target RNA being assayed; and
v) a region that is identical or substantially identical to at least one target RNA; and/or
d) a stabilization agent that prevents degradation of the RNA target and/or reverse transcriptase for at least 6 hours at room temperature.

27-58. (canceled)

59. The method of claim 1, wherein a forward primer in the second set of primers comprises from 5′ to 3′:
a) an adaptor region; and
b) an adaptor-binding region that is identical or substantially identical to the adaptor region of a primer in the first set of barcoded primers; or
c) an adaptor region;
d) a third barcode region; and
e) an adaptor-binding region that is identical or substantially identical to the adaptor region of a primer in the first set of barcoded primers.

60. (canceled)

61. The method of claim 1, wherein a reverse primer in the second set of primers comprises, from 5′ to 3′:
a) an adaptor region;
b) a second barcode region; and
c) a target-binding region that is identical or substantially identical to at least one target RNA; or
a) an adaptor region; and
b) a region that is identical or substantially identical to at least one target RNA.

62-64. (canceled)

65. The method of claim 1, wherein step (c) further comprises contacting the reverse transcription product with Uracil-DNA Glycosylase (UDG) enzyme.

66. The method of claim 1, wherein step (c) further comprises contacting the reverse transcription product or amplification product thereof with a single stranded DNA protector nucleic acid comprising from 5′ to 3′:
a) a region complementary or substantially complementary to a region of at least one target RNA or amplification product thereof, comprising
i) a 5′ region that is identical or substantially identical to the target-binding region of at least one primer in the first set of primers; and
ii) a 3′ region that is complementary to the target RNA sequence downstream of the target-binding region of at least one primer in the first set of primers; and
b) a 3′ nucleic acid modification that inhibits synthesis of a complementary strand by a polymerase.

67-81. (canceled)

82. The method of claim 1, wherein step (c) comprises a nucleic acid amplification method.

83. The method of claim 82, wherein the amplification method comprises polymerase chain reaction amplification (PCR).

84-98. (canceled)

99. The method of claim 1, wherein the sequencing method is selected from the group consisting of: sequencing by synthesis, dideoxy chain termination sequencing, pyrosequencing, sequencing by ligation and detection, polony sequencing, ion semiconductor sequencing, sequencing by hybridization, and nanopore sequencing.

100-108. (canceled)

109. The method of claim 17, wherein the target RNA is detected in the sample if a first and second barcode region associated with the specific target RNA is detected in the sequencing read of the amplification product; or
wherein the target RNA is not detected in the sample if a first or second barcode region associated with the specific target RNA is not detected in the sequencing read of the amplification product.

110-113. (canceled)

114. A reverse transcription solution comprising:
a) a reverse transcriptase;
b) a first set of primers comprising at least one barcode;
c) a detergent;
d) carrier nucleic acid;
e) at least one positive control nucleic acid;
f) at least one stabilization agent; and/or
g) reverse transcription reaction buffer.

115. (canceled)

116. A kit for detecting a target RNA in a sample, comprising:
a) a reverse transcriptase;
b) a first set of primers comprising at least one barcode;
c) a detergent;
d) a carrier nucleic acid;
e) a positive control nucleic acid;
f) at least one stabilization agent;
g) at least two containers;
h) a DNA polymerase;
i) a second set of primers;
j) Uracil-DNA Glycosylase (UDG) enzyme;
k) a protector nucleic acid; and/or
l) a third set of primers.

117-118. (canceled)


SEQUENCE LISTING (1025 sequences)

SEQ ID NO: 1 - DNA, 64 bp, Artificial Sequence
  Description of Artificial Sequence: Synthetic primer
gatagatcatgaggctaatttaaggtcttccttgccatgttgagtgagagcggtgaaccaagac

SEQ ID NO: 2 - DNA, 79 bp, Artificial Sequence
  Description of Artificial Sequence: Synthetic primer
gcgagggaaacgaccgatagatcatgaggctaatttaaggtcttccttgccatgttgagtgagagcggtgaaccaagac

SEQ ID NO: 3 - DNA, 19 bp, Artificial Sequence
  Description of Artificial Sequence: Synthetic primer
aatttaaggtcttccttgc

SEQ ID NO: 4 - DNA, 23 bp, Artificial Sequence
  Description of Artificial Sequence: Synthetic primer
gtttacccaataatactgcgtct

SEQ ID NO: 5 - DNA, 17 bp, Artificial Sequence
  Description of Artificial Sequence: Synthetic primer
tgtgtaggtcaaccacg

SEQ ID NO: 6 - DNA, 24 bp, Artificial Sequence
  Description of Artificial Sequence: Synthetic primer
cagacaaggaactgattacaaaca

SEQ ID NO: 7 - DNA, 19 bp, Artificial Sequence
  Description of Artificial Sequence: Synthetic primer
ctcttagtaccattggtcc

SEQ ID NO: 8 - DNA, 27 bp, Artificial Sequence
  Description of Artificial Sequence: Synthetic primer
ttcttacctttcttttccaatgttact

SEQ ID NO: 9 - DNA, 19 bp, Artificial Sequence
  Description of Artificial Sequence: Synthetic primer
ggacttctgtgcagttaac

SEQ ID NO: 10 - DNA, 22 bp, Artificial Sequence
  Description of Artificial Sequence: Synthetic primer
cagtgttataacaccaggaaca

SEQ ID NO: 11 - DNA, 72 bp, Artificial Sequence
  Description of Artificial Sequence: Synthetic oligonucleotide
ccaaggtttacccaataatactgctgaggttgtcaccgctctcacgaccacgtgcaaggaagaccttaaatt

SEQ ID NO: 12 - DNA, 22 bp, Artificial Sequence
  Description of Artificial Sequence: Synthetic primer
ccaataatactgctgaggttgt

SEQ ID NO: 13 - DNA, 16 bp, Artificial Sequence
  Description of Artificial Sequence: Synthetic oligonucleotide
cgccagcagcgaacaa

SEQ ID NO: 14 - DNA, 49 bp, Artificial Sequence
  Description of Artificial Sequence: Synthetic primer
aatgatacggcgaccaccgagatctacacagaacgccagcagcgaacaa

SEQ ID NO: 15 - DNA, 27 bp, Artificial Sequence
  Description of Artificial Sequence: Synthetic primer
cgagatctacacagaacgccagcagcg

SEQ ID NO: 16 - DNA, 50 bp, Artificial Sequence
  Description of Artificial Sequence: Synthetic oligonucleotide
caagcagaagacggcatacgagatacgagcaagcacaggaccacaacacg

SEQ ID NO: 17 - DNA, 26 bp, Artificial Sequence
  Description of Artificial Sequence: Synthetic primer
acgagcaagcacaggaccacaacacg

SEQ ID NO: 18 - DNA, 8 bp, Artificial Sequence
  Description of Artificial Sequence: Synthetic oligonucleotide
tggtacag

SEQ ID NO: 19 - DNA, 8 bp, Artificial Sequence
  Description of Artificial Sequence: Synthetic oligonucleotide
aaccgttc

SEQ ID NO: 20 - DNA, 8 bp, Artificial Sequence
  Description of Artificial Sequence: Synthetic oligonucleotide
taaccggt

SEQ ID NO: 21 - DNA, 8 bp, Artificial Sequence
  Description of Artificial Sequence: Synthetic oligonucleotide
gaacatcg

SEQ ID NO: 22 - DNA, 8 bp, Artificial Sequence
  Description of Artificial Sequence: Synthetic oligonucleotide
ccttgtag

SEQ ID NO: 23 - DNA, 8 bp, Artificial Sequence
  Description of Artificial Sequence: Synthetic oligonucleotide
tcaggctt

SEQ ID NO: 24 - DNA, 8 bp, Artificial Sequence
  Description of Artificial Sequence: Synthetic oligonucleotide
gttctcgt

SEQ ID NO: 25 - DNA, 8 bp, Artificial Sequence
  Description of Artificial Sequence: Synthetic oligonucleotide
agaacgag

SEQ ID NO: 26 - DNA, 8 bp, Artificial Sequence
  Description of Artificial Sequence: Synthetic oligonucleotide
tgcttcca

SEQ ID NO: 27 - DNA, 8 bp, Artificial Sequence
  Description of Artificial Sequence: Synthetic oligonucleotide
cttcgact

SEQ ID NO: 28 - DNA, 8 bp, Artificial Sequence
  Description of Artificial Sequence: Synthetic oligonucleotide
cacctgtt

SEQ ID NO: 29 - DNA, 8 bp, Artificial Sequence
  Description of Artificial Sequence: Synthetic oligonucleotide
tggtacag

SEQ ID NO: 30 - DNA, 26 bp, Artificial Sequence
  Description of Artificial Sequence: Synthetic oligonucleotide
cgctcacagttctgtcgtgacgagcg

SEQ ID NO: 31 - DNA, 26 bp, Artificial Sequence
  Description of Artificial Sequence: Synthetic oligonucleotide
tatctacgaccttgctacaacagata

SEQ ID NO: 32 - DNA, 26 bp, Artificial Sequence
  Description of Artificial Sequence: Synthetic oligonucleotide
atatgacagacgtgtatagactagct

SEQ ID NO: 33 - DNA, 26 bp, Artificial Sequence
  Description of Artificial Sequence: Synthetic oligonucleotide
cttatacggaattgtgcctacggtgg

SEQ ID NO: 34 - DNA, 26 bp, Artificial Sequence
  Description of Artificial Sequence: Synthetic oligonucleotide
taatcactcgtctgacattacatcct

SEQ ID NO: 35 - DNA, 26 bp, Artificial Sequence
  Description of Artificial Sequence: Synthetic oligonucleotide
gcgcgacatgtttggtccaaccttgt

SEQ ID NO: 36 - DNA, 26 bp, Artificial Sequence
  Description of Artificial Sequence: Synthetic oligonucleotide
agagcacactagtgtggaaaccagta

SEQ ID NO: 37 - DNA, 26 bp, Artificial Sequence
  Description of Artificial Sequence: Synthetic oligonucleotide
tgcctactgatctgccttgacttaat

SEQ ID NO: 38 - DNA, 26 bp, Artificial Sequence
  Description of Artificial Sequence: Synthetic oligonucleotide
ctactaccagtctggttgaactagtg

SEQ ID NO: 39 - DNA, 26 bp, Artificial Sequence
  Description of Artificial Sequence: Synthetic oligonucleotide
tcgtcactgacttgaccagaccgaca

SEQ ID NO: 40 - DNA, 26 bp, Artificial Sequence
  Description of Artificial Sequence: Synthetic oligonucleotide
gaacaactacggtgcatacacactgt

SEQ ID NO: 41 - DNA, 26 bp, Artificial Sequence
  Description of Artificial Sequence: Synthetic oligonucleotide
cctatacgactctggtgtgacgcgct

SEQ ID NO: 42 - DNA, 26 bp, Artificial Sequence
  Description of Artificial Sequence: Synthetic oligonucleotide
taatgacgcaagtgatcacacgaagg

SEQ ID NO: 43 - DNA, 26 bp, Artificial Sequence
  Description of Artificial Sequence: Synthetic oligonucleotide
gtgccacgcttctgcggctacctact

SEQ ID NO: 44 - DNA, 26 bp, Artificial Sequence
  Description of Artificial Sequence: Synthetic oligonucleotide
cggcaacatggatggaatgaccacga

SEQ ID NO: 45 - DNA, 26 bp, Artificial Sequence
  Description of Artificial Sequence: Synthetic oligonucleotide
gccgtacaaccgtgaagacactatag

SEQ ID NO: 46 - DNA, 26 bp, Artificial Sequence
  Description of Artificial Sequence: Synthetic oligonucleotide
aaccaacttctctgtcggcacagcaa

SEQ ID NO: 47 - DNA, 26 bp, Artificial Sequence
  Description of Artificial Sequence: Synthetic oligonucleotide
ggttgaccctcttgctaatacgatgg

SEQ ID NO: 48 - DNA, 26 bp, Artificial Sequence
  Description of Artificial Sequence: Synthetic oligonucleotide
ctaatacgatggtgggttgaccctct

SEQ ID NO: 49 - DNA, 26 bp, Artificial Sequence
  Description of Artificial Sequence: Synthetic oligonucleotide
tcggcacctatctgcgcacacatggc

SEQ ID NO: 50 - DNA, 26 bp, Artificial Sequence
  Description of Artificial Sequence: Synthetic oligonucleotide
agtcaacaccattgggcctacgtcct

SEQ ID NO: 51 - DNA, 26 bp, Artificial Sequence
  Description of Artificial Sequence: Synthetic oligonucleotide
gagcgaccaatatgctgtgacttagg

SEQ ID NO: 52 - DNA, 26 bp, Artificial Sequence
  Description of Artificial Sequence: Synthetic oligonucleotide
aacaaacggcgttgtaaggacaacgt

SEQ ID NO: 53 - DNA, 26 bp, Artificial Sequence
  Description of Artificial Sequence: Synthetic oligonucleotide
gtatgactagaatgctaacactgtaa

SEQ ID NO: 54 - DNA, 26 bp, Artificial Sequence
  Description of Artificial Sequence: Synthetic oligonucleotide
ttctaactggtttgggcgaacgatgg

SEQ ID NO: 55 - DNA, 26 bp, Artificial Sequence
  Description of Artificial Sequence: Synthetic oligonucleotide
cctcgaccaacctgaatagacagcaa

SEQ ID NO: 56 - DNA, 26 bp, Artificial Sequence
  Description of Artificial Sequence: Synthetic oligonucleotide
tggatacgcttatgtcaatacccatt

SEQ ID NO: 57 - DNA, 26 bp, Artificial Sequence
  Description of Artificial Sequence: Synthetic oligonucleotide
atgtcacgtggttgtcgtaactgcgg

SEQ ID NO: 58 - DNA, 26 bp, Artificial Sequence
  Description of Artificial Sequence: Synthetic oligonucleotide
agagtacgcggctgtccgaaccctcg

SEQ ID NO: 59 - DNA, 26 bp, Artificial Sequence
  Description of Artificial Sequence: Synthetic oligonucleotide
tgcctacggtggtgcttatacggaat

SEQ ID NO: 60 - DNA, 26 bp, Artificial Sequence
  Description of Artificial Sequence: Synthetic oligonucleotide
tgcgtacgtcactggcttaaccggac

SEQ ID NO: 61 - DNA, 26 bp, Artificial Sequence
  Description of Artificial Sequence: Synthetic oligonucleotide
catacacactgttggaacaactacgg

SEQ ID NO: 62 - DNA, 26 bp, Artificial Sequence
  Description of Artificial Sequence: Synthetic oligonucleotide
cgtatacaatcatggtcgaacttaca

SEQ ID NO: 63 - DNA, 26 bp, Artificial Sequence
  Description of Artificial Sequence: Synthetic oligonucleotide
tacgcacggctgtgactagacccgtg

SEQ ID NO: 64 - DNA, 26 bp, Artificial Sequence
  Description of Artificial Sequence: Synthetic oligonucleotide
gcgagacttacctgaagttacggtga

SEQ ID NO: 65 - DNA, 26 bp, Artificial Sequence
  Description of Artificial Sequence: Synthetic oligonucleotide
tacggacccggttgtggcaacatatt

SEQ ID NO: 66 - DNA, 26 bp, Artificial Sequence
  Description of Artificial Sequence: Synthetic oligonucleotide
gtcgaacttacatggatcaacccgcg

SEQ ID NO: 67 - DNA, 26 bp, Artificial Sequence
  Description of Artificial Sequence: Synthetic oligonucleotide
ctgtcactgcactgtaccaactccgt

SEQ ID NO: 68 - DNA, 26 bp, Artificial Sequence
  Description of Artificial Sequence: Synthetic oligonucleotide
cagccacgattgtggctgtacaggaa

SEQ ID NO: 69 - DNA, 26 bp, Artificial Sequence
  Description of Artificial Sequence: Synthetic oligonucleotide
tgactacacatatgcgcacactaatg

SEQ ID NO: 70 - DNA, 26 bp, Artificial Sequence
  Description of Artificial Sequence: Synthetic oligonucleotide
attgcaccgagttggacaaacctgaa

SEQ ID NO: 71 - DNA, 26 bp, Artificial Sequence
  Description of Artificial Sequence: Synthetic oligonucleotide
gccatactagactgagtggactcagg

SEQ ID NO: 72 - DNA, 26 bp, Artificial Sequence
  Description of Artificial Sequence: Synthetic oligonucleotide
ggcgaacgatggtgttctaactggtt

SEQ ID NO: 73 - DNA, 26 bp, Artificial Sequence
  Description of Artificial Sequence: Synthetic oligonucleotide
tggctaccgcagtgaatccacggcca

SEQ ID NO: 74 - DNA, 26 bp, Artificial Sequence
  Description of Artificial Sequence: Synthetic oligonucleotide
tagaaactaacgtgccataacaggtt

SEQ ID NO: 75 - DNA, 26 bp, Artificial Sequence
  Description of Artificial Sequence: Synthetic oligonucleotide
taatgacgatcttgatctcactacca

SEQ ID NO: 76 - DNA, 26 bp, Artificial Sequence
  Description of Artificial Sequence: Synthetic oligonucleotide
tatccacaggactgcggtgacgcgaa

SEQ ID NO: 77 - DNA, 26 bp, Artificial Sequence
  Description of Artificial Sequence: Synthetic oligonucleotide
agtgcaccactgtgtaacaacatagg

SEQ ID NO: 78 - DNA, 26 bp, Artificial Sequence
  Description of Artificial Sequence: Synthetic oligonucleotide
gtgcaacacacttgctggtacacacg

SEQ ID NO: 79 - DNA, 26 bp, Artificial Sequence
  Description of Artificial Sequence: Synthetic oligonucleotide
acatgacgtgtctgtcaacacgtgta

SEQ ID NO: 80 - DNA, 26 bp, Artificial Sequence
  Description of Artificial Sequence: Synthetic oligonucleotide
gacagacacaggtgactgtactgtga

SEQ ID NO: 81 - DNA, 26 bp, Artificial Sequence
  Description of Artificial Sequence: Synthetic oligonucleotide
tcttaaccatcatggtgcgactcctt

SEQ ID NO: 82 - DNA, 26 bp, Artificial Sequence
  Description of Artificial Sequence: Synthetic oligonucleotide
ttacaacattcctgagcacacatcct

SEQ ID NO: 83 - DNA, 26 bp, Artificial Sequence
  Description of Artificial Sequence: Synthetic oligonucleotide
aagctactatgctgttccgactcgca

SEQ ID NO: 84 - DNA, 26 bp, Artificial Sequence
  Description of Artificial Sequence: Synthetic oligonucleotide
tattcacctcagtgcttaaacccact

SEQ ID NO: 85 - DNA, 26 bp, Artificial Sequence
  Description of Artificial Sequence: Synthetic oligonucleotide
ctcgtacgcgtttggcctcacggata

SEQ ID NO: 86 - DNA, 26 bp, Artificial Sequence
  Description of Artificial Sequence: Synthetic oligonucleotide
ttaggacatagatgcgtcgacactgg

SEQ ID NO: 87 - DNA, 26 bp, Artificial Sequence
  Description of Artificial Sequence: Synthetic oligonucleotide
ccgaaacgcgagtgtactaacgtcaa

SEQ ID NO: 88 - DNA, 26 bp, Artificial Sequence
  Description of Artificial Sequence: Synthetic oligonucleotide
ggaccacaacagtgatagaacccgtt

SEQ ID NO: 89 - DNA, 26 bp, Artificial Sequence
  Description of Artificial Sequence: Synthetic oligonucleotide
ttccaacggtaatgacagtactccag

SEQ ID NO: 90 - DNA, 26 bp, Artificial Sequence
  Description of Artificial Sequence: Synthetic oligonucleotide
tgattacagccatgaggcaactgtag

SEQ ID NO: 91 - DNA, 26 bp, Artificial Sequence
  Description of Artificial Sequence: Synthetic oligonucleotide
taacaacgtgtttggcaagactctca

SEQ ID NO: 92 - DNA, 26 bp, Artificial Sequence
  Description of Artificial Sequence: Synthetic oligonucleotide
accgcacgcaattgttggcactccgc

SEQ ID NO: 93 - DNA, 26 bp, Artificial Sequence
  Description of Artificial Sequence: Synthetic oligonucleotide
gttcgaccgccatgaactgacatact

SEQ ID NO: 94 - DNA, 26 bp, Artificial Sequence
  Description of Artificial Sequence: Synthetic oligonucleotide
agacaaccattatggtaagacgcata

SEQ ID NO: 95 - DNA, 26 bp, Artificial Sequence
  Description of Artificial Sequence: Synthetic oligonucleotide
gcgttacggtattgaattgacctgcg

SEQ ID NO: 96 - DNA, 26 bp, Artificial Sequence
  Description of Artificial Sequence: Synthetic oligonucleotide
agcacacatccttgttacaacattcc

SEQ ID NO: 97 - DNA, 26 bp, Artificial Sequence
  Description of Artificial Sequence: Synthetic oligonucleotide
ttgttacccgtgtgaacctacagcac

SEQ ID NO: 98 - DNA, 26 bp, Artificial Sequence
  Description of Artificial Sequence: Synthetic oligonucleotide
aagtaacctccatgtctgtacgtgga

SEQ ID NO: 99 - DNA, 26 bp, Artificial Sequence
  Description of Artificial Sequence: Synthetic oligonucleotide
acgtcacaatactgggaatactccaa

SEQ ID NO: 100 - DNA, 26 bp, Artificial Sequence
  Description of Artificial Sequence: Synthetic oligonucleotide
ggtgtacacaagtgaagcgaccgctt

SEQ ID NO: 101 - DNA, 26 bp, Artificial Sequence
  Description of Artificial Sequence: Synthetic oligonucleotide
ccaccactgtgttgtgagcacgttgt

SEQ ID NO: 102 - DNA, 26 bp, Artificial Sequence
  Description of Artificial Sequence: Synthetic oligonucleotide
gttccacgcaggtgatcatacaggct

SEQ ID NO: 103 - DNA, 26 bp, Artificial Sequence
  Description of Artificial Sequence: Synthetic oligonucleotide
accttacatgaatgtgttaacgaagg

SEQ ID NO: 104 - DNA, 26 bp, Artificial Sequence
  Description of Artificial Sequence: Synthetic oligonucleotide
cgctgaccagagtggatggacatgta

SEQ ID NO: 105 - DNA, 26 bp, Artificial Sequence
  Description of Artificial Sequence: Synthetic oligonucleotide
gtagaacgtcagtgacggcaccgtca

SEQ ID NO: 106 - DNA, 26 bp, Artificial Sequence
  Description of Artificial Sequence: Synthetic oligonucleotide
ggataacccagatgcgttgaccttac

SEQ ID NO: 107 - DNA, 26 bp, Artificial Sequence
  Description of Artificial Sequence: Synthetic oligonucleotide
cgcacactaatgtgtgactacacata

SEQ ID NO: 108 - DNA, 26 bp, Artificial Sequence
  Description of Artificial Sequence: Synthetic oligonucleotide
tcctgacaccgttgcggccactcgtt

SEQ ID NO: 109 - DNA, 26 bp, Artificial Sequence
  Description of Artificial Sequence: Synthetic oligonucleotide
ctggcacttgcctgcaagcacatccg

SEQ ID NO: 110 - DNA, 26 bp, Artificial Sequence
  Description of Artificial Sequence: Synthetic oligonucleotide
accagaccgacatgtcgtcactgact

SEQ ID NO: 111 - DNA, 26 bp, Artificial Sequence
  Description of Artificial Sequence: Synthetic oligonucleotide
ttgtaacacggttgctcatacagcga

SEQ ID NO: 112 - DNA, 26 bp, Artificial Sequence
  Description of Artificial Sequence: Synthetic oligonucleotide
gtaagacgcatatgagacaaccatta

SEQ ID NO: 113 - DNA, 26 bp, Artificial Sequence
  Description of Artificial Sequence: Synthetic oligonucleotide
gtccaaccttgttggcgcgacatgtt

SEQ ID NO: 114 - DNA, 26 bp, Artificial Sequence
  Description of Artificial Sequence: Synthetic oligonucleotide
ttaggactaccatgcatgaacgtact

SEQ ID NO: 115 - DNA, 26 bp, Artificial Sequence
  Description of Artificial Sequence: Synthetic oligonucleotide
ggaatactccaatgacgtcacaatac

SEQ ID NO: 116 - DNA, 26 bp, Artificial Sequence
  Description of Artificial Sequence: Synthetic oligonucleotide
catgtacagaggtggatacacctcct

SEQ ID NO: 117 - DNA, 26 bp, Artificial Sequence
  Description of Artificial Sequence: Synthetic oligonucleotide
tacacacgctcctgatccgactaagt

SEQ ID NO: 118 - DNA, 26 bp, Artificial Sequence
  Description of Artificial Sequence: Synthetic oligonucleotide
gcttaaccggactgcgtgtacatctt

SEQ ID NO: 119 - DNA, 26 bp, Artificial Sequence
  Description of Artificial Sequence: Synthetic oligonucleotide
cgcttacgaagttggaaccacatgaa

SEQ ID NO: 120 - DNA, 26 bp, Artificial Sequence
  Description of Artificial Sequence: Synthetic oligonucleotide
cgcctactctgatgggccaactcata

SEQ ID NO: 121 - DNA, 26 bp, Artificial Sequence
  Description of Artificial Sequence: Synthetic oligonucleotide
ataccacaacgctgacataaccttcc

SEQ ID NO: 122 - DNA, 26 bp, Artificial Sequence
  Description of Artificial Sequence: Synthetic oligonucleotide
ctggaactatgttgtatgtacgcaat

SEQ ID NO: 123 - DNA, 26 bp, Artificial Sequence
  Description of Artificial Sequence: Synthetic oligonucleotide
caatcactatgatggattaacaggtg

SEQ ID NO: 124 - DNA, 26 bp, Artificial Sequence
  Description of Artificial Sequence: Synthetic oligonucleotide
ggtggacaatactgatgtaacgacaa

SEQ ID NO: 125 - DNA, 26 bp, Artificial Sequence
  Description of Artificial Sequence: Synthetic oligonucleotide
tggacacggaggtgcacataccggtg

SEQ ID NO: 126 - DNA, 26 bp, Artificial Sequence
  Description of Artificial Sequence: Synthetic oligonucleotide
ctgacaccggcatgcctgaactacaa

SEQ ID NO: 127 - DNA, 26 bp, Artificial Sequence
  Description of Artificial Sequence: Synthetic oligonucleotide
gaattacgagtgtgttaagacttgtg

SEQ ID NO: 128 - DNA, 26 bp, Artificial Sequence
  Description of Artificial Sequence: Synthetic oligonucleotide
gcgtgactgagatgcggacacagtga

SEQ ID NO: 129 - DNA, 26 bp, Artificial Sequence
  Description of Artificial Sequence: Synthetic oligonucleotide
tctccacattgatggcactacacaac

SEQ ID NO: 130 - DNA, 26 bp, Artificial Sequence
  Description of Artificial Sequence: Synthetic oligonucleotide
acatgaccatattgtggtgaccctgg

SEQ ID NO: 131 - DNA, 26 bp, Artificial Sequence
  Description of Artificial Sequence: Synthetic oligonucleotide
caggcacgccattgtccacacggcct

SEQ ID NO: 132 - DNA, 26 bp, Artificial Sequence
  Description of Artificial Sequence: Synthetic oligonucleotide
acataacacggatgttgtaacgtgta

SEQ ID NO: 133 - DNA, 26 bp, Artificial Sequence
  Description of Artificial Sequence: Synthetic oligonucleotide
ttaatacagacctgccacgacacacg

SEQ ID NO: 134 - DNA, 26 bp, Artificial Sequence
  Description of Artificial Sequence: Synthetic oligonucleotide
acgatactgctgtgtgtgaactgtat

SEQ ID NO: 135 - DNA, 26 bp, Artificial Sequence
  Description of Artificial Sequence: Synthetic oligonucleotide
ttctaaccagaatggagcgaccaata

SEQ ID NO: 136 - DNA, 26 bp, Artificial Sequence
  Description of Artificial Sequence: Synthetic oligonucleotide
tattgaccgttctgatcttacactgt

SEQ ID NO: 137 - DNA, 26 bp, Artificial Sequence
  Description of Artificial Sequence: Synthetic oligonucleotide
catgaacgtacttgatgtcacgtggt

SEQ ID NO: 138 - DNA, 26 bp, Artificial Sequence
  Description of Artificial Sequence: Synthetic oligonucleotide
taattacctacctggtagcaccatca

SEQ ID NO: 139 - DNA, 26 bp, Artificial Sequence
  Description of Artificial Sequence: Synthetic oligonucleotide
acgctacaattatgtggttacaagaa

SEQ ID NO: 140 - DNA, 26 bp, Artificial Sequence
  Description of Artificial Sequence: Synthetic oligonucleotide
ccttgacttaattgtgttgacttcgt

SEQ ID NO: 141 - DNA, 26 bp, Artificial Sequence
  Description of Artificial Sequence: Synthetic oligonucleotide
gtagcaccatcatgccaacacaacat

SEQ ID NO: 142 - DNA, 26 bp, Artificial Sequence
  Description of Artificial Sequence: Synthetic oligonucleotide
cttgtacaattctgaccggacctcag

SEQ ID NO: 143 - DNA, 26 bp, Artificial Sequence
  Description of Artificial Sequence: Synthetic oligonucleotide
tccaaacttctatggttaaactctga

SEQ ID NO: 144 - DNA, 26 bp, Artificial Sequence
  Description of Artificial Sequence: Synthetic oligonucleotide
agagcactgccttgcggctacaacgt

SEQ ID NO: 145 - DNA, 26 bp, Artificial Sequence
  Description of Artificial Sequence: Synthetic oligonucleotide
cttcgacccgattgtccaaacgaatt

SEQ ID NO: 146 - DNA, 26 bp, Artificial Sequence
  Description of Artificial Sequence: Synthetic oligonucleotide
tcggtaccacggtgccgaaaccgttg

SEQ ID NO: 147 - DNA, 26 bp, Artificial Sequence
  Description of Artificial Sequence: Synthetic oligonucleotide
gaacaacagtattgtaaccacgccga

SEQ ID NO: 148 - DNA, 26 bp, Artificial Sequence
  Description of Artificial Sequence: Synthetic oligonucleotide
aattgacgcggatgctccgactgctg

SEQ ID NO: 149 - DNA, 26 bp, Artificial Sequence
  Description of Artificial Sequence: Synthetic oligonucleotide
ggcctacgtccttgcattcaccagct

SEQ ID NO: 150 - DNA, 26 bp, Artificial Sequence
  Description of Artificial Sequence: Synthetic oligonucleotide
taggtactctcttgggttaactgcta

SEQ ID NO: 151 - DNA, 26 bp, Artificial Sequence
  Description of Artificial Sequence: Synthetic oligonucleotide
acacaacatatctgaccacacacggt

SEQ ID NO: 152 - DNA, 26 bp, Artificial Sequence
  Description of Artificial Sequence: Synthetic oligonucleotide
ttcctacgtacgtgtaggtactctct

SEQ ID NO: 153 - DNA, 26 bp, Artificial Sequence
  Description of Artificial Sequence: Synthetic oligonucleotide
ggtaaaccgcagtgtatggacctcga

SEQ ID NO: 154 - DNA, 26 bp, Artificial Sequence
  Description of Artificial Sequence: Synthetic oligonucleotide
tccacacggccttgctcgtacgcgtt

SEQ ID NO: 155 - DNA, 26 bp, Artificial Sequence
  Description of Artificial Sequence: Synthetic oligonucleotide
gatacacctccttgccagtactggca

SEQ ID NO: 156 - DNA, 26 bp, Artificial Sequence
  Description of Artificial Sequence: Synthetic oligonucleotide
caacgactcagctgtgttcacgcatt

SEQ ID NO: 157 - DNA, 26 bp, Artificial Sequence
  Description of Artificial Sequence: Synthetic oligonucleotide
cggttacattagtgaaccgaccatcg

SEQ ID NO: 158 - DNA, 26 bp, Artificial Sequence
  Description of Artificial Sequence: Synthetic oligonucleotide
cgcgcacctagatgcgaagacgttaa

SEQ ID NO: 159 - DNA, 26 bp, Artificial Sequence
  Description of Artificial Sequence: Synthetic oligonucleotide
tcttgacgctattgagtgcaccactg

SEQ ID NO: 160 - DNA, 26 bp, Artificial Sequence
  Description of Artificial Sequence: Synthetic oligonucleotide
tcacaacccgaatggaacaacagtat

SEQ ID NO: 161 - DNA, 26 bp, Artificial Sequence
  Description of Artificial Sequence: Synthetic oligonucleotide
aacgtactacattgacgatactgctg

SEQ ID NO: 162 - DNA, 26 bp, Artificial Sequence
  Description of Artificial Sequence: Synthetic oligonucleotide
cggccactcgtttgataccactggat

SEQ ID NO: 163 - DNA, 26 bp, Artificial Sequence
  Description of Artificial Sequence: Synthetic oligonucleotide
cataaaccaccatgtccaaacttcta

SEQ ID NO: 164 - DNA, 26 bp, Artificial Sequence
  Description of Artificial Sequence: Synthetic oligonucleotide
acagaacggccatgtgagaaccagcg

SEQ ID NO: 165 - DNA, 26 bp, Artificial Sequence
  Description of Artificial Sequence: Synthetic oligonucleotide
tggtgaccctggtgacgctacaatta

SEQ ID NO: 166 - DNA, 26 bp, Artificial Sequence
  Description of Artificial Sequence: Synthetic oligonucleotide
taggaacaccggtgtatatactcgag

SEQ ID NO: 167 - DNA, 26 bp, Artificial Sequence
  Description of Artificial Sequence: Synthetic oligonucleotide
aatatactggcctgcggtcaccgata

SEQ ID NO: 168 - DNA, 26 bp, Artificial Sequence
  Description of Artificial Sequence: Synthetic oligonucleotide
ataggactattctgacaatacagagt

SEQ ID NO: 169 - DNA, 26 bp, Artificial Sequence
  Description of Artificial Sequence: Synthetic oligonucleotide
ccttcacacgtatgcggttacattag

SEQ ID NO: 170 - DNA, 26 bp, Artificial Sequence
  Description of Artificial Sequence: Synthetic oligonucleotide
ggccaacataagtggataaaccaagt

SEQ ID NO: 171 - DNA, 26 bp, Artificial Sequence
  Description of Artificial Sequence: Synthetic oligonucleotide
cagtaacgttgttgagttaactcaca

SEQ ID NO: 172 - DNA, 26 bp, Artificial Sequence
  Description of Artificial Sequence: Synthetic oligonucleotide
ttcatacccaactgttccaacggtaa

SEQ ID NO: 173 - DNA, 26 bp, Artificial Sequence
  Description of Artificial Sequence: Synthetic oligonucleotide
caattacggatttgcatgtacagagg

SEQ ID NO: 174 - DNA, 26 bp, Artificial Sequence
  Description of Artificial Sequence: Synthetic oligonucleotide
ggccaactcatatggattgactcata

SEQ ID NO: 175 - DNA, 26 bp, Artificial Sequence
  Description of Artificial Sequence: Synthetic oligonucleotide
aattgacctgcgtgattccacgctat

SEQ ID NO: 176 - DNA, 26 bp, Artificial Sequence
  Description of Artificial Sequence: Synthetic oligonucleotide
taaggacaacgttggaccgacctgtg

SEQ ID NO: 177 - DNA, 26 bp, Artificial Sequence
  Description of Artificial Sequence: Synthetic oligonucleotide
ctataaccgcggtgtaggaacaccgg

SEQ ID NO: 178 - DNA, 26 bp, Artificial Sequence
  Description of Artificial Sequence: Synthetic oligonucleotide
attcaacgaatctgagcggactggac

SEQ ID NO: 179 - DNA, 26 bp, Artificial Sequence
  Description of Artificial Sequence: Synthetic oligonucleotide
gtattacctctatgtatagacattcg

SEQ ID NO: 180 - DNA, 26 bp, Artificial Sequence
  Description of Artificial Sequence: Synthetic oligonucleotide
cctgaactacaatgacagaacggcca

SEQ ID NO: 181 - DNA, 26 bp, Artificial Sequence
  Description of Artificial Sequence: Synthetic oligonucleotide
gaccgacctgtgtgattccactattg

SEQ ID NO: 182 - DNA, 26 bp, Artificial Sequence
  Description of Artificial Sequence: Synthetic oligonucleotide
ttcagaccgtggtgtattcacctcag

SEQ ID NO: 183 - DNA, 26 bp, Artificial Sequence
  Description of Artificial Sequence: Synthetic oligonucleotide
aactcaccgaactgcgcctactctga

SEQ ID NO: 184 - DNA, 26 bp, Artificial Sequence
  Description of Artificial Sequence: Synthetic oligonucleotide
attccacgctattggcgcaacgagta

SEQ ID NO: 185 - DNA, 26 bp, Artificial Sequence
  Description of Artificial Sequence: Synthetic oligonucleotide
tgaatacattgctgggcgcaccaatt

SEQ ID NO: 186 - DNA, 26 bp, Artificial Sequence
  Description of Artificial Sequence: Synthetic oligonucleotide
cgcaaactctagtgagataactggcg

SEQ ID NO: 187 - DNA, 26 bp, Artificial Sequence
  Description of Artificial Sequence: Synthetic oligonucleotide
aaccgaccatcgtgcctgcacttggt

SEQ ID NO: 188 - DNA, 26 bp, Artificial Sequence
  Description of Artificial Sequence: Synthetic oligonucleotide
ctagtacccggatggacgaacacaat

SEQ ID NO: 189 - DNA, 26 bp, Artificial Sequence
  Description of Artificial Sequence: Synthetic oligonucleotide
gctccacgtcactgtggcgacgtcca

SEQ ID NO: 190 - DNA, 26 bp, Artificial Sequence
  Description of Artificial Sequence: Synthetic oligonucleotide
agatgacgaatttgcttcaacgttac

SEQ ID NO: 191 - DNA, 26 bp, Artificial Sequence
  Description of Artificial Sequence: Synthetic oligonucleotide
acaccacgttaatgtcctgacaccgt

SEQ ID NO: 192 - DNA, 26 bp, Artificial Sequence
  Description of Artificial Sequence: Synthetic oligonucleotide
gataaaccaagttgcgcgcacctaga

SEQ ID NO: 193 - DNA, 26 bp, Artificial Sequence
  Description of Artificial Sequence: Synthetic oligonucleotide
ctggtacacacgtgaggatacaagtt

SEQ ID NO: 194 - DNA, 26 bp, Artificial Sequence
  Description of Artificial Sequence: Synthetic oligonucleotide
cgaagacgttaatgaggccacagaca

SEQ ID NO: 195 - DNA, 26 bp, Artificial Sequence
  Description of Artificial Sequence: Synthetic oligonucleotide
atcgcacatatgtgccttgacaacgg

SEQ ID NO: 196 - DNA, 26 bp, Artificial Sequence
  Description of Artificial Sequence: Synthetic oligonucleotide
atcatacaggcttgcaccaaccctac

SEQ ID NO: 197 - DNA, 26 bp, Artificial Sequence
  Description of Artificial Sequence: Synthetic oligonucleotide
gattgactcatatgttgctactgtat

SEQ ID NO: 198 - DNA, 26 bp, Artificial Sequence
  Description of Artificial Sequence: Synthetic oligonucleotide
ccaacacaacattgcaatcactatga

SEQ ID NO: 199 - DNA, 26 bp, Artificial Sequence
  Description of Artificial Sequence: Synthetic oligonucleotide
ttggtacggtgctgtggtaacctgat

SEQ ID NO: 200 - DNA, 26 bp, Artificial Sequence
  Description of Artificial Sequence: Synthetic oligonucleotide
gcgaaaccgccttgttcatacccaac

SEQ ID NO: 201 - DNA, 26 bp, Artificial Sequence
  Description of Artificial Sequence: Synthetic oligonucleotide
caaccacggaggtgcataaaccacca

SEQ ID NO: 202 - DNA, 26 bp, Artificial Sequence
  Description of Artificial Sequence: Synthetic oligonucleotide
agcggactggactgtcctaacttagc

SEQ ID NO: 203 - DNA, 26 bp, Artificial Sequence
  Description of Artificial Sequence: Synthetic oligonucleotide
gacgaacacaattgtctctacagatt

SEQ ID NO: 204 - DNA, 26 bp, Artificial Sequence
  Description of Artificial Sequence: Synthetic oligonucleotide
ccactacggtcctgcgcgaacgccta

SEQ ID NO: 205 - DNA, 26 bp, Artificial Sequence
  Description of Artificial Sequence: Synthetic oligonucleotide
tgttaacgaaggtggataaacgctct

SEQ ID NO: 206 - DNA, 26 bp, Artificial Sequence
  Description of Artificial Sequence: Synthetic oligonucleotide
tatatactcgagtggagatacgtcga

SEQ ID NO: 207 - DNA, 26 bp, Artificial Sequence
  Description of Artificial Sequence: Synthetic oligonucleotide
cgcgaaccgatctgctggaactatgt

SEQ ID NO: 208 - DNA, 26 bp, Artificial Sequence
  Description of Artificial Sequence: Synthetic oligonucleotide
gcctcacggatatgggccaacataag

SEQ ID NO: 209 - DNA, 26 bp, Artificial Sequence
  Description of Artificial Sequence: Synthetic oligonucleotide
tgagaaccagcgtgattacactcacc

SEQ ID NO: 210 - DNA, 26 bp, Artificial Sequence
  Description of Artificial Sequence: Synthetic oligonucleotide
tgttcacgcatttgaattgacgcgga

SEQ ID NO: 211 - DNA, 26 bp, Artificial Sequence
  Description of Artificial Sequence: Synthetic oligonucleotide
tccaaacgaatttgttgtcacaactt

SEQ ID NO: 212 - DNA, 26 bp, Artificial Sequence
  Description of Artificial Sequence: Synthetic oligonucleotide
gctgtacaggaatgggcgaacattct

SEQ ID NO: 213 - DNA, 26 bp, Artificial Sequence
  Description of Artificial Sequence: Synthetic oligonucleotide
ataccactggattgcaacgactcagc

SEQ ID NO: 214 - DNA, 26 bp, Artificial Sequence
  Description of Artificial Sequence: Synthetic oligonucleotide
gttggacaccgttgtcttaaccatca

SEQ ID NO: 215 - DNA, 26 bp, Artificial Sequence
  Description of Artificial Sequence: Synthetic oligonucleotide
accaaacgttactgcgccaactacct

SEQ ID NO: 216 - DNA, 26 bp, Artificial Sequence
  Description of Artificial Sequence: Synthetic oligonucleotide
gtgtgacgcgcttgctaatacgtctt

SEQ ID NO: 217 - DNA, 26 bp, Artificial Sequence
  Description of Artificial Sequence: Synthetic oligonucleotide
ggcagactagcatgcaaccacggagg

SEQ ID NO: 218 - DNA, 26 bp, Artificial Sequence
  Description of Artificial Sequence: Synthetic oligonucleotide
tgcggactgttgtgggcagactagca

SEQ ID NO: 219 - DNA, 26 bp, Artificial Sequence
  Description of Artificial Sequence: Synthetic oligonucleotide
gattaacaggtgtgttaggacataga

SEQ ID NO: 220 - DNA, 26 bp, Artificial Sequence
  Description of Artificial Sequence: Synthetic oligonucleotide
caacaacttcaatgcgcaaactctag

SEQ ID NO: 221 - DNA, 26 bp, Artificial Sequence
  Description of Artificial Sequence: Synthetic oligonucleotide
gtgttacaccggtggagttacgtact

SEQ ID NO: 222 - DNA, 26 bp, Artificial Sequence
  Description of Artificial Sequence: Synthetic oligonucleotide
tatcaactgagatgaacacacgtgga

SEQ ID NO: 223 - DNA, 26 bp, Artificial Sequence
  Description of Artificial Sequence: Synthetic oligonucleotide
cttggaccctcgtggtgttacaccgg

SEQ ID NO: 224 - DNA, 26 bp, Artificial Sequence
  Description of Artificial Sequence: Synthetic oligonucleotide
gtctcacgtgaatgagattacgttac

SEQ ID NO: 225 - DNA, 26 bp, Artificial Sequence
  Description of Artificial Sequence: Synthetic oligonucleotide
ccatcaccacgctgttgacaccaatg

SEQ ID NO: 226 - DNA, 26 bp, Artificial Sequence
  Description of Artificial Sequence: Synthetic oligonucleotide
acaacaccaggatgctgacaccggca

SEQ ID NO: 227 - DNA, 26 bp, Artificial Sequence
  Description of Artificial Sequence: Synthetic oligonucleotide
agcagacaattatgtctcaactcaat

SEQ ID NO: 228 - DNA, 26 bp, Artificial Sequence
  Description of Artificial Sequence: Synthetic oligonucleotide
cagtcacgtgcgtgggaccacaacag

SEQ ID NO: 229 - DNA, 26 bp, Artificial Sequence
  Description of Artificial Sequence: Synthetic oligonucleotide
gtctaacacctctgaatgtacattgc

SEQ ID NO: 230 - DNA, 26 bp, Artificial Sequence
  Description of Artificial Sequence: Synthetic oligonucleotide
gaactaccggtttggatctacctgga

SEQ ID NO: 231 - DNA, 26 bp, Artificial Sequence
  Description of Artificial Sequence: Synthetic oligonucleotide
agttaactcacatgcaggcacgccat

SEQ ID NO: 232 - DNA, 26 bp, Artificial Sequence
  Description of Artificial Sequence: Synthetic oligonucleotide
gtagcacatacttgttaatacagacc

SEQ ID NO: 233 - DNA, 26 bp, Artificial Sequence
  Description of Artificial Sequence: Synthetic oligonucleotide
cttcaacgttactgggagtaccgcga

SEQ ID NO: 234 - DNA, 26 bp, Artificial Sequence
  Description of Artificial Sequence: Synthetic oligonucleotide
agtccacgaggatgaacgcaccagag

SEQ ID NO: 235 - DNA, 26 bp, Artificial Sequence
  Description of Artificial Sequence: Synthetic oligonucleotide
acagtactccagtgcgtaaacttaac

SEQ ID NO: 236 - DNA, 26 bp, Artificial Sequence
  Description of Artificial Sequence: Synthetic oligonucleotide
ccgcaactattctgacgagacactga

SEQ ID NO: 237 - DNA, 26 bp, Artificial Sequence
  Description of Artificial Sequence: Synthetic oligonucleotide
ttatcaccgatctggtatcacggccg

SEQ ID NO: 238 - DNA, 26 bp, Artificial Sequence
  Description of Artificial Sequence: Synthetic oligonucleotide
atagtacctagctgaatacacgacat

SEQ ID NO: 239 - DNA, 26 bp, Artificial Sequence
  Description of Artificial Sequence: Synthetic oligonucleotide
tatagactagcttggttatacatggc

SEQ ID NO: 240 - DNA, 26 bp, Artificial Sequence
  Description of Artificial Sequence: Synthetic oligonucleotide
actccacggtggtggcctgacccatg

SEQ ID NO: 241 - DNA, 26 bp, Artificial Sequence
  Description of Artificial Sequence: Synthetic oligonucleotide
gtgcgacgtaagtgtaagaaccctat

SEQ ID NO: 242 - DNA, 26 bp, Artificial Sequence
  Description of Artificial Sequence: Synthetic oligonucleotide
gatataccctaatgtatacaccatgg

SEQ ID NO: 243 - DNA, 26 bp, Artificial Sequence
  Description of Artificial Sequence: Synthetic oligonucleotide
tcgcgactataatggccgtacctgtt

SEQ ID NO: 244 - DNA, 26 bp, Artificial Sequence
  Description of Artificial Sequence: Synthetic oligonucleotide
attctacaagcgtgcagagactgata

SEQ ID NO: 245 - DNA, 26 bp, Artificial Sequence
  Description of Artificial Sequence: Synthetic oligonucleotide
agcgcacttcggtgtgctaacactat

SEQ ID NO: 246 - DNA, 26 bp, Artificial Sequence
  Description of Artificial Sequence: Synthetic oligonucleotide
gttgaactagtgtgtcagtactaatg

SEQ ID NO: 247 - DNA, 26 bp, Artificial Sequence
  Description of Artificial Sequence: Synthetic oligonucleotide
aatagacagcaatggtgacaccttga

SEQ ID NO: 248 - DNA, 26 bp, Artificial Sequence
  Description of Artificial Sequence: Synthetic oligonucleotide
ctaacactgtaatgacatgaccatat

SEQ ID NO: 249 - DNA, 26 bp, Artificial Sequence
  Description of Artificial Sequence: Synthetic oligonucleotide
gcgtaaccttagtgaacatacaccta

SEQ ID NO: 250 - DNA, 26 bp, Artificial Sequence
  Description of Artificial Sequence: Synthetic oligonucleotide
taccgacaactatgccatgactgtag

SEQ ID NO: 251 - DNA, 26 bp, Artificial Sequence
  Description of Artificial Sequence: Synthetic oligonucleotide
gtagtacaatagtggagtcactctcc

SEQ ID NO: 252 - DNA, 26 bp, Artificial Sequence
  Description of Artificial Sequence: Synthetic oligonucleotide
ggttaactgctatggctatacgcgca

SEQ ID NO: 253 - DNA, 26 bp, Artificial Sequence
  Description of Artificial Sequence: Synthetic oligonucleotide
acaatacagagttgatcgcacatatg

SEQ ID NO: 254 - DNA, 26 bp, Artificial Sequence
  Description of Artificial Sequence: Synthetic oligonucleotide
gcttcaccactatgagtacacctata

SEQ ID NO: 255 - DNA, 26 bp, Artificial Sequence
  Description of Artificial Sequence: Synthetic oligonucleotide
agataactggcgtggaccgacgagat

SEQ ID NO: 256 - DNA, 26 bp, Artificial Sequence
  Description of Artificial Sequence: Synthetic oligonucleotide
aatatacgaagctgcgttcacagcct

SEQ ID NO: 257 - DNA, 26 bp, Artificial Sequence
  Description of Artificial Sequence: Synthetic oligonucleotide
tagcgacctagttgttactactcctc

SEQ ID NO: 258 - DNA, 26 bp, Artificial Sequence
  Description of Artificial Sequence: Synthetic oligonucleotide
agttaacagagctgcacgtacccacc

SEQ ID NO: 259 - DNA, 26 bp, Artificial Sequence
  Description of Artificial Sequence: Synthetic oligonucleotide
cagatacaccactggctacactatct

SEQ ID NO: 260 - DNA, 26 bp, Artificial Sequence
  Description of Artificial Sequence: Synthetic oligonucleotide
acggcaccgtcatgagtcaacaccat

SEQ ID NO: 261 - DNA, 26 bp, Artificial Sequence
  Description of Artificial Sequence: Synthetic oligonucleotide
gtaatactactgtgcgaggaccggta

SEQ ID NO: 262 - DNA, 26 bp, Artificial Sequence
  Description of Artificial Sequence: Synthetic oligonucleotide
aagtcacttgtatgcaggtacgttca

SEQ ID NO: 263 - DNA, 26 bp, Artificial Sequence
  Description of Artificial Sequence: Synthetic oligonucleotide
gtcacaccacagtggacagacacagg

SEQ ID NO: 264 - DNA, 26 bp, Artificial Sequence
  Description of Artificial Sequence: Synthetic oligonucleotide
attagactggagtgtgtacacttgtt

SEQ ID NO: 265 - DNA, 26 bp, Artificial Sequence
  Description of Artificial Sequence: Synthetic oligonucleotide
tgctaacactattgctctaacagtag

SEQ ID NO: 266 - DNA, 26 bp, Artificial Sequence
  Description of Artificial Sequence: Synthetic oligonucleotide
taagaaccctattggtcacaccacag

SEQ ID NO: 267 - DNA, 26 bp, Artificial Sequence
  Description of Artificial Sequence: Synthetic oligonucleotide
tggttacaagaatgtctacacatacc

SEQ ID NO: 268 - DNA, 26 bp, Artificial Sequence
  Description of Artificial Sequence: Synthetic oligonucleotide
actctactcctttgcacgtactaggc

SEQ ID NO: 269 - DNA, 26 bp, Artificial Sequence
  Description of Artificial Sequence: Synthetic oligonucleotide
gtctcaccttcctgtggtgacagtct

SEQ ID NO: 270 - DNA, 26 bp, Artificial Sequence
  Description of Artificial Sequence: Synthetic oligonucleotide
tccgcacgttcatgcttcgacaagga

SEQ ID NO: 271 - DNA, 26 bp, Artificial Sequence
  Description of Artificial Sequence: Synthetic oligonucleotide
aggttacgcaggtggtagaacgtcag

SEQ ID NO: 272 - DNA, 26 bp, Artificial Sequence
  Description of Artificial Sequence: Synthetic oligonucleotide
gaaccacatgaatggacatactgtca

SEQ ID NO: 273 - DNA, 26 bp, Artificial Sequence
  Description of Artificial Sequence: Synthetic oligonucleotide
ttgagacaggattgtccgcacaaggc

SEQ ID NO: 274 - DNA, 26 bp, Artificial Sequence
  Description of Artificial Sequence: Synthetic oligonucleotide
tggtcactagtgtgactgcaccttat

SEQ ID NO: 275 - DNA, 26 bp, Artificial Sequence
  Description of Artificial Sequence: Synthetic oligonucleotide
agtggacataattgtacgcacacgta

SEQ ID NO: 276 - DNA, 26 bp, Artificial Sequence
  Description of Artificial Sequence: Synthetic oligonucleotide
ggcacacgccattgcgcttacgaagt

SEQ ID NO: 277 - DNA, 26 bp, Artificial Sequence
  Description of Artificial Sequence: Synthetic oligonucleotide
gatctacctggatgctgcaaccttca

SEQ ID NO: 278 - DNA, 26 bp, Artificial Sequence
  Description of Artificial Sequence: Synthetic oligonucleotide
tgctgacgacattgcagcgacgacaa

SEQ ID NO: 279 - DNA, 26 bp, Artificial Sequence
  Description of Artificial Sequence: Synthetic oligonucleotide
ccgaaaccgttgtgggatcaccgcat

SEQ ID NO: 280 - DNA, 26 bp, Artificial Sequence
  Description of Artificial Sequence: Synthetic oligonucleotide
attaaactacgctgtgcggactgttg

SEQ ID NO: 281 - DNA, 26 bp, Artificial Sequence
  Description of Artificial Sequence: Synthetic oligonucleotide
tagtcacacaactgacataacacgga

SEQ ID NO: 282 - DNA, 26 bp, Artificial Sequence
  Description of Artificial Sequence: Synthetic oligonucleotide
ggtatactgagatggacgtactcgcg

SEQ ID NO: 283 - DNA, 26 bp, Artificial Sequence
  Description of Artificial Sequence: Synthetic oligonucleotide
caagaactgctttgcattcacaacaa

SEQ ID NO: 284 - DNA, 26 bp, Artificial Sequence
  Description of Artificial Sequence: Synthetic oligonucleotide
acgagacactgatgcacggacattat

SEQ ID NO: 285 - DNA, 26 bp, Artificial Sequence
  Description of Artificial Sequence: Synthetic oligonucleotide
ttatcacttgcatgttgagacgacgg

SEQ ID NO: 286 - DNA, 26 bp, Artificial Sequence
  Description of Artificial Sequence: Synthetic oligonucleotide
agattacgttactgctctgactatac

SEQ ID NO: 287 - DNA, 26 bp, Artificial Sequence
  Description of Artificial Sequence: Synthetic oligonucleotide
tctacaccgctgtggcaacacaggtg

SEQ ID NO: 288 - DNA, 26 bp, Artificial Sequence
  Description of Artificial Sequence: Synthetic oligonucleotide
aacggactatgatgggtaaaccgcag

SEQ ID NO: 289 - DNA, 26 bp, Artificial Sequence
  Description of Artificial Sequence: Synthetic oligonucleotide
caatgacgcgcctgaccgcacgcaat

SEQ ID NO: 290 - DNA, 26 bp, Artificial Sequence
  Description of Artificial Sequence: Synthetic oligonucleotide
ctaatactcgcttgagccgacgaaca

SEQ ID NO: 291 - DNA, 26 bp, Artificial Sequence
  Description of Artificial Sequence: Synthetic oligonucleotide
catggactctaatgtcctaacggaag

SEQ ID NO: 292 - DNA, 26 bp, Artificial Sequence
  Description of Artificial Sequence: Synthetic oligonucleotide
atactacgtgtgtgttgagaccctaa

SEQ ID NO: 293 - DNA, 26 bp, Artificial Sequence
  Description of Artificial Sequence: Synthetic oligonucleotide
gccgaaccaagatgccaccactgtgt

SEQ ID NO: 294 - DNA, 26 bp, Artificial Sequence
  Description of Artificial Sequence: Synthetic oligonucleotide
cgaggaccggtatgcctcgaccaacc

SEQ ID NO: 295 - DNA, 26 bp, Artificial Sequence
  Description of Artificial Sequence: Synthetic oligonucleotide
gatatacaacagtggtataacgctgt

SEQ ID NO: 296 - DNA, 26 bp, Artificial Sequence
  Description of Artificial Sequence: Synthetic oligonucleotide
tcgccacggttatggctacacattag

SEQ ID NO: 297 - DNA, 26 bp, Artificial Sequence
  Description of Artificial Sequence: Synthetic oligonucleotide
agactacctctttgtacgaacatctt

SEQ ID NO: 298 - DNA, 26 bp, Artificial Sequence
  Description of Artificial Sequence: Synthetic oligonucleotide
gctcgaccctactgtaggaacgcgca

SEQ ID NO: 299 - DNA, 26 bp, Artificial Sequence
  Description of Artificial Sequence: Synthetic oligonucleotide
aggatacaagtttggtactacggcgt

SEQ ID NO: 300 - DNA, 26 bp, Artificial Sequence
  Description of Artificial Sequence: Synthetic oligonucleotide
gagacacataattgagttaacagagc

SEQ ID NO: 301 - DNA, 26 bp, Artificial Sequence
  Description of Artificial Sequence: Synthetic oligonucleotide
agctgacttatatgtcgcgactataa

SEQ ID NO: 302 - DNA, 26 bp, Artificial Sequence
  Description of Artificial Sequence: Synthetic oligonucleotide
gtatcacattggtggagtgactgccg

SEQ ID NO: 303 - DNA, 26 bp, Artificial Sequence
  Description of Artificial Sequence: Synthetic oligonucleotide
aatagacgcctctgctagtacccgga

SEQ ID NO: 304 - DNA, 26 bp, Artificial Sequence
  Description of Artificial Sequence: Synthetic oligonucleotide
ccgctactagcttgattaaactacgc

SEQ ID NO: 305 - DNA, 26 bp, Artificial Sequence
  Description of Artificial Sequence: Synthetic oligonucleotide
tcctaacggaagtgcctagacagtat

SEQ ID NO: 306 - DNA, 26 bp, Artificial Sequence
  Description of Artificial Sequence: Synthetic oligonucleotide
tcacaacgatcgtgtaggaacagact

SEQ ID NO: 307 - DNA, 26 bp, Artificial Sequence
  Description of Artificial Sequence: Synthetic oligonucleotide
acttgactccactgccgtgacgcctt

SEQ ID NO: 308 - DNA, 26 bp, Artificial Sequence
  Description of Artificial Sequence: Synthetic oligonucleotide
tgtacacttgtttgggataactatcc

SEQ ID NO: 309 - DNA, 26 bp, Artificial Sequence
  Description of Artificial Sequence: Synthetic oligonucleotide
cacttacaatcttgcacctaccttgg

SEQ ID NO: 310 - DNA, 26 bp, Artificial Sequence
  Description of Artificial Sequence: Synthetic oligonucleotide
cagagactgatatgaacgtactacat

SEQ ID NO: 311 - DNA, 26 bp, Artificial Sequence
  Description of Artificial Sequence: Synthetic oligonucleotide
ggcgaacattcttgcggcaacagctc

SEQ ID NO: 312 - DNA, 26 bp, Artificial Sequence
  Description of Artificial Sequence: Synthetic oligonucleotide
agtggactcaggtgtcttgacgctat

SEQ ID NO: 313 - DNA, 26 bp, Artificial Sequence
  Description of Artificial Sequence: Synthetic oligonucleotide
cattcaccagcttgacggaacatgcg

SEQ ID NO: 314 - DNA, 26 bp, Artificial Sequence
  Description of Artificial Sequence: Synthetic oligonucleotide
ctcgtactatcatggttccacgcagg

SEQ ID NO: 315 - DNA, 26 bp, Artificial Sequence
  Description of Artificial Sequence: Synthetic oligonucleotide
ccttaacctatgtgaccaaacgttac

SEQ ID NO: 316 - DNA, 26 bp, Artificial Sequence
  Description of Artificial Sequence: Synthetic oligonucleotide
agaagacccaattgtggctaccgcag

SEQ ID NO: 317 - DNA, 26 bp, Artificial Sequence
  Description of Artificial Sequence: Synthetic oligonucleotide
taatcacggtactgaactaacacgtt

SEQ ID NO: 318 - DNA, 26 bp, Artificial Sequence
  Description of Artificial Sequence: Synthetic oligonucleotide
ggaatactgttctgtagagacttgga

SEQ ID NO: 319 - DNA, 26 bp, Artificial Sequence
  Description of Artificial Sequence: Synthetic oligonucleotide
ccggaacccacatgagagcacactag

SEQ ID NO: 320 - DNA, 26 bp, Artificial Sequence
  Description of Artificial Sequence: Synthetic oligonucleotide
gacttacagaagtgactctacacagg

SEQ ID NO: 321 - DNA, 26 bp, Artificial Sequence
  Description of Artificial Sequence: Synthetic oligonucleotide
tggcaacatatttgcggtgacacacc

SEQ ID NO: 322 - DNA, 26 bp, Artificial Sequence
  Description of Artificial Sequence: Synthetic oligonucleotide
gaatgaccacgatggcgttacggtat

SEQ ID NO: 323 - DNA, 26 bp, Artificial Sequence
  Description of Artificial Sequence: Synthetic oligonucleotide
cgtgtacatctttgtgtgcactaaca

SEQ ID NO: 324 - DNA, 26 bp, Artificial Sequence
  Description of Artificial Sequence: Synthetic oligonucleotide
attcaacttgcatgccagaacagtaa

SEQ ID NO: 325 - DNA, 26 bp, Artificial Sequence
  Description of Artificial Sequence: Synthetic oligonucleotide
tccttaccatagtgcttatacacctg

SEQ ID NO: 326 - DNA, 26 bp, Artificial Sequence
  Description of Artificial Sequence: Synthetic oligonucleotide
tctagactcttctgactagacaactt

SEQ ID NO: 327 - DNA, 26 bp, Artificial Sequence
  Description of Artificial Sequence: Synthetic oligonucleotide
ctcgaacctccttgttaggaccttac

SEQ ID NO: 328 - DNA, 26 bp, Artificial Sequence
  Description of Artificial Sequence: Synthetic oligonucleotide
agtgaacgtgaatgtatcaactgaga

SEQ ID NO: 329 - DNA, 26 bp, Artificial Sequence
  Description of Artificial Sequence: Synthetic oligonucleotide
gaagcacggacctgctcacacacaag

SEQ ID NO: 330 - DNA, 26 bp, Artificial Sequence
  Description of Artificial Sequence: Synthetic oligonucleotide
gctctaccgttgtggaattacgagtg

SEQ ID NO: 331 - DNA, 26 bp, Artificial Sequence
  Description of Artificial Sequence: Synthetic oligonucleotide
ggaccactcaattgcggatactatat

SEQ ID NO: 332 - DNA, 26 bp, Artificial Sequence
  Description of Artificial Sequence: Synthetic oligonucleotide
gagtcactctcctgttgaaacgcaga

SEQ ID NO: 333 - DNA, 26 bp, Artificial Sequence
  Description of Artificial Sequence: Synthetic oligonucleotide
aacggacagcggtgtacggaccgaag

SEQ ID NO: 334 - DNA, 26 bp, Artificial Sequence
  Description of Artificial Sequence: Synthetic oligonucleotide
tgtgaactgtattgtctccacattga

SEQ ID NO: 335 - DNA, 26 bp, Artificial Sequence
  Description of Artificial Sequence: Synthetic oligonucleotide
aacatacacctatgcgagaacccaag

SEQ ID NO: 336 - DNA, 26 bp, Artificial Sequence
  Description of Artificial Sequence: Synthetic oligonucleotide
gtgctacaggtgtgtgctgacgacat

SEQ ID NO: 337 - DNA, 26 bp, Artificial Sequence
  Description of Artificial Sequence: Synthetic oligonucleotide
catacacttgaatggatggactatcg

SEQ ID NO: 338 - DNA, 26 bp, Artificial Sequence
  Description of Artificial Sequence: Synthetic oligonucleotide
cttgtaccttaatgggcttacaattg

SEQ ID NO: 339 - DNA, 26 bp, Artificial Sequence
  Description of Artificial Sequence: Synthetic oligonucleotide
aagagacaggtgtgctcgaacctcct

SEQ ID NO: 340 - DNA, 26 bp, Artificial Sequence
  Description of Artificial Sequence: Synthetic oligonucleotide
tgcacacgagaatgatacaaccagag

SEQ ID NO: 341 - DNA, 26 bp, Artificial Sequence
  Description of Artificial Sequence: Synthetic oligonucleotide
acttcacctagctgtctcgacgacga

SEQ ID NO: 342 - DNA, 26 bp, Artificial Sequence
  Description of Artificial Sequence: Synthetic oligonucleotide
gtgctacattaatgaccacacgtctg

SEQ ID NO: 343 - DNA, 26 bp, Artificial Sequence
  Description of Artificial Sequence: Synthetic oligonucleotide
agcgtacgaatgtggttgtacactca

SEQ ID NO: 344 - DNA, 26 bp, Artificial Sequence
  Description of Artificial Sequence: Synthetic oligonucleotide
ccttaacgtgcctgtcaggactcaac

SEQ ID NO: 345 - DNA, 26 bp, Artificial Sequence
  Description of Artificial Sequence: Synthetic oligonucleotide
tgtacaccgaattgagtccacgagga

SEQ ID NO: 346 - DNA, 26 bp, Artificial Sequence
  Description of Artificial Sequence: Synthetic oligonucleotide
ggagaacttagttgcacttacaatct

SEQ ID NO: 347 - DNA, 26 bp, Artificial Sequence
  Description of Artificial Sequence: Synthetic oligonucleotide
tactaacacacatgtactcactgtta

SEQ ID NO: 348 - DNA, 26 bp, Artificial Sequence
  Description of Artificial Sequence: Synthetic oligonucleotide
taggtaccgttgtggcgacactcgat

SEQ ID NO: 349 - DNA, 26 bp, Artificial Sequence
  Description of Artificial Sequence: Synthetic oligonucleotide
atgccacgaccgtgctaggaccaagg

SEQ ID NO: 350 - DNA, 26 bp, Artificial Sequence
  Description of Artificial Sequence: Synthetic oligonucleotide
ctagcacgtcgatgcctctactcgaa

SEQ ID NO: 351 - DNA, 26 bp, Artificial Sequence
  Description of Artificial Sequence: Synthetic oligonucleotide
tgcctacacgagtgtcatcacctctt

SEQ ID NO: 352 - DNA, 26 bp, Artificial Sequence
  Description of Artificial Sequence: Synthetic oligonucleotide
actagacaactttgggtaaacgataa

SEQ ID NO: 353 - DNA, 26 bp, Artificial Sequence
  Description of Artificial Sequence: Synthetic oligonucleotide
cacctaccttggtgaacgaacgccag

SEQ ID NO: 354 - DNA, 26 bp, Artificial Sequence
  Description of Artificial Sequence: Synthetic oligonucleotide
aagcaacgatattgtagacacaatct

SEQ ID NO: 355 - DNA, 26 bp, Artificial Sequence
  Description of Artificial Sequence: Synthetic oligonucleotide
gccagacatccatgcaatgacctgaa

SEQ ID NO: 356 - DNA, 26 bp, Artificial Sequence
  Description of Artificial Sequence: Synthetic oligonucleotide
ttggaacttcaatggtcacacggtgt

SEQ ID NO: 357 - DNA, 26 bp, Artificial Sequence
  Description of Artificial Sequence: Synthetic oligonucleotide
actagacccgtgtgggtgtacacaag

SEQ ID NO: 358 - DNA, 26 bp, Artificial Sequence
  Description of Artificial Sequence: Synthetic oligonucleotide
cggcaacagctctgaggttacgcagg

SEQ ID NO: 359 - DNA, 26 bp, Artificial Sequence
  Description of Artificial Sequence: Synthetic oligonucleotide
gaagcactagcttgtaataaccggag

SEQ ID NO: 360 - DNA, 26 bp, Artificial Sequence
  Description of Artificial Sequence: Synthetic oligonucleotide
acaagacgattgtgcgaagacacgca

SEQ ID NO: 361 - DNA, 26 bp, Artificial Sequence
  Description of Artificial Sequence: Synthetic oligonucleotide
gcaacacaggtgtgattgaaccacat

SEQ ID NO: 362 - DNA, 26 bp, Artificial Sequence
  Description of Artificial Sequence: Synthetic oligonucleotide
caaggactgacgtgcagccacgattg

SEQ ID NO: 363 - DNA, 26 bp, Artificial Sequence
  Description of Artificial Sequence: Synthetic oligonucleotide
accagactcatttgtctcaaccgcgt

SEQ ID NO: 364 - DNA, 26 bp, Artificial Sequence
  Description of Artificial Sequence: Synthetic oligonucleotide
ccggaacatcattgctctgacacgtg

SEQ ID NO: 365 - DNA, 26 bp, Artificial Sequence
  Description of Artificial Sequence: Synthetic oligonucleotide
ttgagaccctaatgtcgaaactggaa

SEQ ID NO: 366 - DNA, 26 bp, Artificial Sequence
  Description of Artificial Sequence: Synthetic oligonucleotide
ccaccacttacatgaaggcaccttgg

SEQ ID NO: 367 - DNA, 26 bp, Artificial Sequence
  Description of Artificial Sequence: Synthetic oligonucleotide
gttgcacagttgtgtgaacacgcaac

SEQ ID NO: 368 - DNA, 26 bp, Artificial Sequence
  Description of Artificial Sequence: Synthetic oligonucleotide
tcactaccatgttgccgctactagct

SEQ ID NO: 369 - DNA, 26 bp, Artificial Sequence
  Description of Artificial Sequence: Synthetic oligonucleotide
gactgacgttgctgcaccgacaggaa

SEQ ID NO: 370 - DNA, 26 bp, Artificial Sequence
  Description of Artificial Sequence: Synthetic oligonucleotide
atcgtaccgctctgcgtatacaatca

SEQ ID NO: 371 - DNA, 26 bp, Artificial Sequence
  Description of Artificial Sequence: Synthetic oligonucleotide
ggtgcacgttcgtgatgacacagaac

SEQ ID NO: 372 - DNA, 26 bp, Artificial Sequence
  Description of Artificial Sequence: Synthetic oligonucleotide
cggcgactaagatgattcaacttgca

SEQ ID NO: 373 - DNA, 26 bp, Artificial Sequence
  Description of Artificial Sequence: Synthetic oligonucleotide
gacataccagcttgtcatgactcctg

SEQ ID NO: 374 - DNA, 26 bp, Artificial Sequence
  Description of Artificial Sequence: Synthetic oligonucleotide
actaaacttcagtgaattcacgatcg

SEQ ID NO: 375 - DNA, 26 bp, Artificial Sequence
  Description of Artificial Sequence: Synthetic oligonucleotide
ttcctacccttatgttccgacacatt

SEQ ID NO: 376 - DNA, 26 bp, Artificial Sequence
  Description of Artificial Sequence: Synthetic oligonucleotide
tgtgtacaagcttgtggcaaccgacc

SEQ ID NO: 377 - DNA, 26 bp, Artificial Sequence
  Description of Artificial Sequence: Synthetic oligonucleotide
gtggcactggtttggccacacagcac

SEQ ID NO: 378 - DNA, 26 bp, Artificial Sequence
  Description of Artificial Sequence: Synthetic oligonucleotide
tcgacacttaagtgcagtaacgttgt

SEQ ID NO: 379 - DNA, 26 bp, Artificial Sequence
  Description of Artificial Sequence: Synthetic oligonucleotide
cacgtactaggctgagctcactcaag

SEQ ID NO: 380 - DNA, 26 bp, Artificial Sequence
  Description of Artificial Sequence: Synthetic oligonucleotide
tgaagactaagttgtctggacaatta

SEQ ID NO: 381 - DNA, 26 bp, Artificial Sequence
  Description of Artificial Sequence: Synthetic oligonucleotide
acggaacatgcgtgattagactggag

SEQ ID NO: 382 - DNA, 26 bp, Artificial Sequence
  Description of Artificial Sequence: Synthetic oligonucleotide
gtgtgacatatctggactaactatgt

SEQ ID NO: 383 - DNA, 26 bp, Artificial Sequence
  Description of Artificial Sequence: Synthetic oligonucleotide
acacaacgcgcttgcgttcacggaac

SEQ ID NO: 384 - DNA, 26 bp, Artificial Sequence
  Description of Artificial Sequence: Synthetic oligonucleotide
agcgcacggtgatgtcgatacactag

SEQ ID NO: 385 - DNA, 26 bp, Artificial Sequence
  Description of Artificial Sequence: Synthetic oligonucleotide
caaggacctatctgtaccaaccaatg

SEQ ID NO: 386 - DNA, 26 bp, Artificial Sequence
  Description of Artificial Sequence: Synthetic oligonucleotide
tgcgtacccaggtgtggtaactacca

SEQ ID NO: 387 - DNA, 26 bp, Artificial Sequence
  Description of Artificial Sequence: Synthetic oligonucleotide
aggtgaccgtaatggctctaccgttg

SEQ ID NO: 388 - DNA, 26 bp, Artificial Sequence
  Description of Artificial Sequence: Synthetic oligonucleotide
gcagcacaacgatggtctcacgtgaa

SEQ ID NO: 389 - DNA, 26 bp, Artificial Sequence
  Description of Artificial Sequence: Synthetic oligonucleotide
atcctactgtcgtgaaggcaccacct

SEQ ID NO: 390 - DNA, 26 bp, Artificial Sequence
  Description of Artificial Sequence: Synthetic oligonucleotide
gaaggactacactgctgtgacagcta

SEQ ID NO: 391 - DNA, 26 bp, Artificial Sequence
  Description of Artificial Sequence: Synthetic oligonucleotide
ttggcaccaggttgtcacaacgatcg

SEQ ID NO: 392 - DNA, 26 bp, Artificial Sequence
  Description of Artificial Sequence: Synthetic oligonucleotide
aggccacagacatgagaagacccaat

SEQ ID NO: 393 - DNA, 26 bp, Artificial Sequence
  Description of Artificial Sequence: Synthetic oligonucleotide
agcatactaacttgactgcacagccg

SEQ ID NO: 394 - DNA, 26 bp, Artificial Sequence
  Description of Artificial Sequence: Synthetic oligonucleotide
attacactcacctgaacatacctagt

SEQ ID NO: 395 - DNA, 26 bp, Artificial Sequence
  Description of Artificial Sequence: Synthetic oligonucleotide
gcgcaacgagtatgccttaacctatg

SEQ ID NO: 396 - DNA, 26 bp, Artificial Sequence
  Description of Artificial Sequence: Synthetic oligonucleotide
cgccaactaccttggtggcacgagac

SEQ ID NO: 397 - DNA, 26 bp, Artificial Sequence
  Description of Artificial Sequence: Synthetic oligonucleotide
gcaggacctggatggccagacatcca

SEQ ID NO: 398 - DNA, 26 bp, Artificial Sequence
  Description of Artificial Sequence: Synthetic oligonucleotide
gttatacatggctgacacaacatatc

SEQ ID NO: 399 - DNA, 26 bp, Artificial Sequence
  Description of Artificial Sequence: Synthetic oligonucleotide
cactcacgcacttgtggagacgtaat

SEQ ID NO: 400 - DNA, 26 bp, Artificial Sequence
  Description of Artificial Sequence: Synthetic oligonucleotide
accggacctcagtgccttcacacgta

SEQ ID NO: 401 - DNA, 26 bp, Artificial Sequence
  Description of Artificial Sequence: Synthetic oligonucleotide
atagaacccgtttgctataaccgcgg

SEQ ID NO: 402 - DNA, 26 bp, Artificial Sequence
  Description of Artificial Sequence: Synthetic oligonucleotide
tgaacacgcaactggttgcacagttg

SEQ ID NO: 403 - DNA, 26 bp, Artificial Sequence
  Description of Artificial Sequence: Synthetic oligonucleotide
gtggtactgaagtgttatgaccgcct

SEQ ID NO: 404 - DNA, 26 bp, Artificial Sequence
  Description of Artificial Sequence: Synthetic oligonucleotide
actgaacatagatgtctcaacgtaca

SEQ ID NO: 405 - DNA, 26 bp, Artificial Sequence
  Description of Artificial Sequence: Synthetic oligonucleotide
ggacgactcttgtgagtatacacgga

SEQ ID NO: 406 - DNA, 26 bp, Artificial Sequence
  Description of Artificial Sequence: Synthetic oligonucleotide
gttgtacactcatgacgctactggac

SEQ ID NO: 407 - DNA, 26 bp, Artificial Sequence
  Description of Artificial Sequence: Synthetic oligonucleotide
agaacaccgcggtgggagtacagatt

SEQ ID NO: 408 - DNA, 26 bp, Artificial Sequence
  Description of Artificial Sequence: Synthetic oligonucleotide
cagtaactcaattgtacacacgctcc

SEQ ID NO: 409 - DNA, 26 bp, Artificial Sequence
  Description of Artificial Sequence: Synthetic oligonucleotide
tccatacaatcctgtccgaactagag

SEQ ID NO: 410 - DNA, 26 bp, Artificial Sequence
  Description of Artificial Sequence: Synthetic oligonucleotide
atgagacaaccatgctcaaacggccg

SEQ ID NO: 411 - DNA, 26 bp, Artificial Sequence
  Description of Artificial Sequence: Synthetic oligonucleotide
tcgtgacgttgatgcaagtactcata

SEQ ID NO: 412 - DNA, 26 bp, Artificial Sequence
  Description of Artificial Sequence: Synthetic oligonucleotide
caagtactcatatgaatccacttagg

SEQ ID NO: 413 - DNA, 26 bp, Artificial Sequence
  Description of Artificial Sequence: Synthetic oligonucleotide
cttaaacccacttgggtggacaatac

SEQ ID NO: 414 - DNA, 26 bp, Artificial Sequence
  Description of Artificial Sequence: Synthetic oligonucleotide
cgctccaagttccttcgtggagagcg

SEQ ID NO: 415 - DNA, 26 bp, Artificial Sequence
  Description of Artificial Sequence: Synthetic oligonucleotide
tatctcagacctctctacagaagata

SEQ ID NO: 416 - DNA, 26 bp, Artificial Sequence
  Description of Artificial Sequence: Synthetic oligonucleotide
atatgcaagacgcttataggatagct

SEQ ID NO: 417 - DNA, 26 bp, Artificial Sequence
  Description of Artificial Sequence: Synthetic oligonucleotide
cttatcaggaatcttgcctgaggtgg

SEQ ID NO: 418 - DNA, 26 bp, Artificial Sequence
  Description of Artificial Sequence: Synthetic oligonucleotide
taatccatcgtcctacattgaatcct

SEQ ID NO: 419 - DNA, 26 bp, Artificial Sequence
  Description of Artificial Sequence: Synthetic oligonucleotide
gcgcgcaatgttctgtccagacttgt

SEQ ID NO: 420 - DNA, 26 bp, Artificial Sequence
  Description of Artificial Sequence: Synthetic oligonucleotide
agagccaactagcttggaagacagta

SEQ ID NO: 421 - DNA, 26 bp, Artificial Sequence
  Description of Artificial Sequence: Synthetic oligonucleotide
tgcctcatgatcctccttggattaat

SEQ ID NO: 422 - DNA, 26 bp, Artificial Sequence
  Description of Artificial Sequence: Synthetic oligonucleotide
ctactcacagtcctgttgagatagtg

SEQ ID NO: 423 - DNA, 26 bp, Artificial Sequence
  Description of Artificial Sequence: Synthetic oligonucleotide
tcgtccatgactctaccaggacgaca

SEQ ID NO: 424 - DNA, 26 bp, Artificial Sequence
  Description of Artificial Sequence: Synthetic oligonucleotide
gaacacatacggctcatacgaactgt

SEQ ID NO: 425 - DNA, 26 bp, Artificial Sequence
  Description of Artificial Sequence: Synthetic oligonucleotide
cctatcagactcctgtgtggagcgct

SEQ ID NO: 426 - DNA, 26 bp, Artificial Sequence
  Description of Artificial Sequence: Synthetic oligonucleotide
taatgcagcaagctatcacgagaagg

SEQ ID NO: 427 - DNA, 26 bp, Artificial Sequence
  Description of Artificial Sequence: Synthetic oligonucleotide
gtgcccagcttcctcggctgactact

SEQ ID NO: 428 - DNA, 26 bp, Artificial Sequence
  Description of Artificial Sequence: Synthetic oligonucleotide
cggcacaatggactgaatggacacga

SEQ ID NO: 429 - DNA, 26 bp, Artificial Sequence
  Description of Artificial Sequence: Synthetic oligonucleotide
gccgtcaaaccgctaagacgatatag

SEQ ID NO: 430 - DNA, 26 bp, Artificial Sequence
  Description of Artificial Sequence: Synthetic oligonucleotide
aaccacattctccttcggcgaagcaa

SEQ ID NO: 431 - DNA, 26 bp, Artificial Sequence
  Description of Artificial Sequence: Synthetic oligonucleotide
ggttgcacctctctctaatgagatgg

SEQ ID NO: 432 - DNA, 26 bp, Artificial Sequence
  Description of Artificial Sequence: Synthetic oligonucleotide
ctaatcagatggctggttggacctct

SEQ ID NO: 433 - DNA, 26 bp, Artificial Sequence
  Description of Artificial Sequence: Synthetic oligonucleotide
tcggccactatcctcgcacgaatggc

SEQ ID NO: 434 - DNA, 26 bp, Artificial Sequence
  Description of Artificial Sequence: Synthetic oligonucleotide
agtcacaaccatctggcctgagtcct

SEQ ID NO: 435 - DNA, 26 bp, Artificial Sequence
  Description of Artificial Sequence: Synthetic oligonucleotide
gagcgcacaatactctgtggattagg

SEQ ID NO: 436 - DNA, 26 bp, Artificial Sequence
  Description of Artificial Sequence: Synthetic oligonucleotide
aacaacaggcgtcttaagggaaacgt

SEQ ID NO: 437 - DNA, 26 bp, Artificial Sequence
  Description of Artificial Sequence: Synthetic oligonucleotide
gtatgcatagaactctaacgatgtaa

SEQ ID NO: 438 - DNA, 26 bp, Artificial Sequence
  Description of Artificial Sequence: Synthetic oligonucleotide
ttctacatggttctggcgagagatgg

SEQ ID NO: 439 - DNA, 26 bp, Artificial Sequence
  Description of Artificial Sequence: Synthetic oligonucleotide
cctcgcacaaccctaataggaagcaa

SEQ ID NO: 440 - DNA, 26 bp, Artificial Sequence
  Description of Artificial Sequence: Synthetic oligonucleotide
tggatcagcttacttcaatgaccatt

SEQ ID NO: 441 - DNA, 26 bp, Artificial Sequence
  Description of Artificial Sequence: Synthetic oligonucleotide
atgtccagtggtcttcgtagatgcgg

SEQ ID NO: 442 - DNA, 26 bp, Artificial Sequence
  Description of Artificial Sequence: Synthetic oligonucleotide
agagtcagcggccttccgagacctcg

SEQ ID NO: 443 - DNA, 26 bp, Artificial Sequence
  Description of Artificial Sequence: Synthetic oligonucleotide
tgcctcaggtggctcttatgaggaat

SEQ ID NO: 444 - DNA, 26 bp, Artificial Sequence
  Description of Artificial Sequence: Synthetic oligonucleotide
tgcgtcagtcacctgcttagacggac

SEQ ID NO: 445 - DNA, 26 bp, Artificial Sequence
  Description of Artificial Sequence: Synthetic oligonucleotide
cataccaactgtctgaacagatacgg

SEQ ID NO: 446 - DNA, 26 bp, Artificial Sequence
  Description of Artificial Sequence: Synthetic oligonucleotide
cgtatcaaatcactgtcgagattaca

SEQ ID NO: 447 - DNA, 26 bp, Artificial Sequence
  Description of Artificial Sequence: Synthetic oligonucleotide
tacgccaggctgctactaggaccgtg

SEQ ID NO: 448 - DNA, 26 bp, Artificial Sequence
  Description of Artificial Sequence: Synthetic oligonucleotide
gcgagcattaccctaagttgaggtga

SEQ ID NO: 449 - DNA, 26 bp, Artificial Sequence
  Description of Artificial Sequence: Synthetic oligonucleotide
tacggcaccggtcttggcagaatatt

SEQ ID NO: 450 - DNA, 26 bp, Artificial Sequence
  Description of Artificial Sequence: Synthetic oligonucleotide
gtcgacattacactgatcagaccgcg

SEQ ID NO: 451 - DNA, 26 bp, Artificial Sequence
  Description of Artificial Sequence: Synthetic oligonucleotide
ctgtccatgcaccttaccagatccgt

SEQ ID NO: 452 - DNA, 26 bp, Artificial Sequence
  Description of Artificial Sequence: Synthetic oligonucleotide
cagcccagattgctgctgtgaaggaa

SEQ ID NO: 453 - DNA, 26 bp, Artificial Sequence
  Description of Artificial Sequence: Synthetic oligonucleotide
tgactcaacatactcgcacgataatg

SEQ ID NO: 454 - DNA, 26 bp, Artificial Sequence
  Description of Artificial Sequence: Synthetic oligonucleotide
attgccacgagtctgacaagactgaa

SEQ ID NO: 455 - DNA, 26 bp, Artificial Sequence
  Description of Artificial Sequence: Synthetic oligonucleotide
gccatcatagacctagtgggatcagg

SEQ ID NO: 456 - DNA, 26 bp, Artificial Sequence
  Description of Artificial Sequence: Synthetic oligonucleotide
ggcgacagatggctttctagatggtt

SEQ ID NO: 457 - DNA, 26 bp, Artificial Sequence
  Description of Artificial Sequence: Synthetic oligonucleotide
tggctcacgcagctaatccgaggcca

SEQ ID NO: 458 - DNA, 26 bp, Artificial Sequence
  Description of Artificial Sequence: Synthetic oligonucleotide
tagaacataacgctccatagaaggtt

SEQ ID NO: 459 - DNA, 26 bp, Artificial Sequence
  Description of Artificial Sequence: Synthetic oligonucleotide
taatgcagatctctatctcgatacca

SEQ ID NO: 460 - DNA, 26 bp, Artificial Sequence
  Description of Artificial Sequence: Synthetic oligonucleotide
tatcccaaggacctcggtggagcgaa

SEQ ID NO: 461 - DNA, 26 bp, Artificial Sequence
  Description of Artificial Sequence: Synthetic oligonucleotide
agtgccacactgcttaacagaatagg

SEQ ID NO: 462 - DNA, 26 bp, Artificial Sequence
  Description of Artificial Sequence: Synthetic oligonucleotide
gtgcacaacactctctggtgaacacg

SEQ ID NO: 463 - DNA, 26 bp, Artificial Sequence
  Description of Artificial Sequence: Synthetic oligonucleotide
acatgcagtgtccttcaacgagtgta

SEQ ID NO: 464 - DNA, 26 bp, Artificial Sequence
  Description of Artificial Sequence: Synthetic oligonucleotide
gacagcaacaggctactgtgatgtga

SEQ ID NO: 465 - DNA, 26 bp, Artificial Sequence
  Description of Artificial Sequence: Synthetic oligonucleotide
tcttacacatcactgtgcggatcctt

SEQ ID NO: 466 - DNA, 26 bp, Artificial Sequence
  Description of Artificial Sequence: Synthetic oligonucleotide
ttacacaattccctagcacgaatcct

SEQ ID NO: 467 - DNA, 26 bp, Artificial Sequence
  Description of Artificial Sequence: Synthetic oligonucleotide
aagctcatatgcctttccggatcgca

SEQ ID NO: 468 - DNA, 26 bp, Artificial Sequence
  Description of Artificial Sequence: Synthetic oligonucleotide
tattccactcagctcttaagaccact

SEQ ID NO: 469 - DNA, 26 bp, Artificial Sequence
  Description of Artificial Sequence: Synthetic oligonucleotide
ctcgtcagcgttctgcctcgaggata

SEQ ID NO: 470 - DNA, 26 bp, Artificial Sequence
  Description of Artificial Sequence: Synthetic oligonucleotide
ttaggcaatagactcgtcggaactgg

SEQ ID NO: 471 - DNA, 26 bp, Artificial Sequence
  Description of Artificial Sequence: Synthetic oligonucleotide
ccgaacagcgagcttactagagtcaa

SEQ ID NO: 472 - DNA, 26 bp, Artificial Sequence
  Description of Artificial Sequence: Synthetic oligonucleotide
ggacccaaacagctatagagaccgtt

SEQ ID NO: 473 - DNA, 26 bp, Artificial Sequence
  Description of Artificial Sequence: Synthetic oligonucleotide
ttccacaggtaactacagtgatccag

SEQ ID NO: 474 - DNA, 26 bp, Artificial Sequence
  Description of Artificial Sequence: Synthetic oligonucleotide
tgattcaagccactaggcagatgtag

SEQ ID NO: 475 - DNA, 26 bp, Artificial Sequence
  Description of Artificial Sequence: Synthetic oligonucleotide
taacacagtgttctgcaaggatctca

SEQ ID NO: 476 - DNA, 26 bp, Artificial Sequence
  Description of Artificial Sequence: Synthetic oligonucleotide
accgccagcaatctttggcgatccgc

SEQ ID NO: 477 - DNA, 26 bp, Artificial Sequence
  Description of Artificial Sequence: Synthetic oligonucleotide
gttcgcacgccactaactggaatact

SEQ ID NO: 478 - DNA, 26 bp, Artificial Sequence
  Description of Artificial Sequence: Synthetic oligonucleotide
agacacacattactgtaaggagcata

SEQ ID NO: 479 - DNA, 26 bp, Artificial Sequence
  Description of Artificial Sequence: Synthetic oligonucleotide
gcgttcaggtatctaattggactgcg

SEQ ID NO: 480 - DNA, 26 bp, Artificial Sequence
  Description of Artificial Sequence: Synthetic oligonucleotide
agcaccaatcctctttacagaattcc

SEQ ID NO: 481 - DNA, 26 bp, Artificial Sequence
  Description of Artificial Sequence: Synthetic oligonucleotide
ttgttcaccgtgctaacctgaagcac

SEQ ID NO: 482 - DNA, 26 bp, Artificial Sequence
  Description of Artificial Sequence: Synthetic oligonucleotide
aagtacactccacttctgtgagtgga

SEQ ID NO: 483 - DNA, 26 bp, Artificial Sequence
  Description of Artificial Sequence: Synthetic oligonucleotide
acgtccaaatacctggaatgatccaa

SEQ ID NO: 484 - DNA, 26 bp, Artificial Sequence
  Description of Artificial Sequence: Synthetic oligonucleotide
ggtgtcaacaagctaagcggacgctt

SEQ ID NO: 485 - DNA, 26 bp, Artificial Sequence
  Description of Artificial Sequence: Synthetic oligonucleotide
ccacccatgtgtcttgagcgagttgt

SEQ ID NO: 486 - DNA, 26 bp, Artificial Sequence
  Description of Artificial Sequence: Synthetic oligonucleotide
gttcccagcaggctatcatgaaggct

SEQ ID NO: 487 - DNA, 26 bp, Artificial Sequence
  Description of Artificial Sequence: Synthetic oligonucleotide
accttcaatgaacttgttagagaagg

SEQ ID NO: 488 - DNA, 26 bp, Artificial Sequence
  Description of Artificial Sequence: Synthetic oligonucleotide
cgctgcacagagctgatgggaatgta

SEQ ID NO: 489 - DNA, 26 bp, Artificial Sequence
  Description of Artificial Sequence: Synthetic oligonucleotide
gtagacagtcagctacggcgacgtca

SEQ ID NO: 490 - DNA, 26 bp, Artificial Sequence
  Description of Artificial Sequence: Synthetic oligonucleotide
ggatacaccagactcgttggacttac

SEQ ID NO: 491 - DNA, 26 bp, Artificial Sequence
  Description of Artificial Sequence: Synthetic oligonucleotide
cgcaccataatgcttgactgaacata

SEQ ID NO: 492 - DNA, 26 bp, Artificial Sequence
  Description of Artificial Sequence: Synthetic oligonucleotide
tcctgcaaccgtctcggccgatcgtt

SEQ ID NO: 493 - DNA, 26 bp, Artificial Sequence
  Description of Artificial Sequence: Synthetic oligonucleotide
ctggccattgccctcaagcgaatccg

SEQ ID NO: 494 - DNA, 26 bp, Artificial Sequence
  Description of Artificial Sequence: Synthetic oligonucleotide
accagcacgacacttcgtcgatgact

SEQ ID NO: 495 - DNA, 26 bp, Artificial Sequence
  Description of Artificial Sequence: Synthetic oligonucleotide
ttgtacaacggtctctcatgaagcga

SEQ ID NO: 496 - DNA, 26 bp, Artificial Sequence
  Description of Artificial Sequence: Synthetic oligonucleotide
gtaagcagcatactagacagacatta

SEQ ID NO: 497 - DNA, 26 bp, Artificial Sequence
  Description of Artificial Sequence: Synthetic oligonucleotide
gtccacacttgtctgcgcggaatgtt

SEQ ID NO: 498 - DNA, 26 bp, Artificial Sequence
  Description of Artificial Sequence: Synthetic oligonucleotide
ttaggcataccactcatgagagtact

SEQ ID NO: 499 - DNA, 26 bp, Artificial Sequence
  Description of Artificial Sequence: Synthetic oligonucleotide
ggaatcatccaactacgtcgaaatac

SEQ ID NO: 500 - DNA, 26 bp, Artificial Sequence
  Description of Artificial Sequence: Synthetic oligonucleotide
catgtcaagaggctgatacgactcct

SEQ ID NO: 501 - DNA, 26 bp, Artificial Sequence
  Description of Artificial Sequence: Synthetic oligonucleotide
tacaccagctccctatccggataagt

SEQ ID NO: 502 - DNA, 26 bp, Artificial Sequence
  Description of Artificial Sequence: Synthetic oligonucleotide
gcttacacggacctcgtgtgaatctt

SEQ ID NO: 503 - DNA, 26 bp, Artificial Sequence
  Description of Artificial Sequence: Synthetic oligonucleotide
cgcttcagaagtctgaaccgaatgaa

SEQ ID NO: 504 - DNA, 26 bp, Artificial Sequence
  Description of Artificial Sequence: Synthetic oligonucleotide
cgcctcatctgactggccagatcata

SEQ ID NO: 505 - DNA, 26 bp, Artificial Sequence
  Description of Artificial Sequence: Synthetic oligonucleotide
atacccaaacgcctacatagacttcc

SEQ ID NO: 506 - DNA, 26 bp, Artificial Sequence
  Description of Artificial Sequence: Synthetic oligonucleotide
ctggacatatgtcttatgtgagcaat

SEQ ID NO: 507 - DNA, 26 bp, Artificial Sequence
  Description of Artificial Sequence: Synthetic oligonucleotide
caatccatatgactgattagaaggtg

SEQ ID NO: 508 - DNA, 26 bp, Artificial Sequence
  Description of Artificial Sequence: Synthetic oligonucleotide
ggtggcaaatacctatgtagagacaa

SEQ ID NO: 509 - DNA, 26 bp, Artificial Sequence
  Description of Artificial Sequence: Synthetic oligonucleotide
tggaccaggaggctcacatgacggtg

SEQ ID NO: 510 - DNA, 26 bp, Artificial Sequence
  Description of Artificial Sequence: Synthetic oligonucleotide
ctgaccacggcactcctgagatacaa

SEQ ID NO: 511 - DNA, 26 bp, Artificial Sequence
  Description of Artificial Sequence: Synthetic oligonucleotide
gaattcagagtgctttaaggattgtg

SEQ ID NO: 512 - DNA, 26 bp, Artificial Sequence
  Description of Artificial Sequence: Synthetic oligonucleotide
gcgtgcatgagactcggacgaagtga

SEQ ID NO: 513 - DNA, 26 bp, Artificial Sequence
  Description of Artificial Sequence: Synthetic oligonucleotide
tctcccaattgactgcactgaacaac

SEQ ID NO: 514 - DNA, 26 bp, Artificial Sequence
  Description of Artificial Sequence: Synthetic oligonucleotide
acatgcacatatcttggtggacctgg

SEQ ID NO: 515 - DNA, 26 bp, Artificial Sequence
  Description of Artificial Sequence: Synthetic oligonucleotide
caggccagccatcttccacgaggcct

SEQ ID NO: 516 - DNA, 26 bp, Artificial Sequence
  Description of Artificial Sequence: Synthetic oligonucleotide
acatacaacggactttgtagagtgta

SEQ ID NO: 517 - DNA, 26 bp, Artificial Sequence
  Description of Artificial Sequence: Synthetic oligonucleotide
ttaatcaagaccctccacggaacacg

SEQ ID NO: 518 - DNA, 26 bp, Artificial Sequence
  Description of Artificial Sequence: Synthetic oligonucleotide
acgatcatgctgcttgtgagatgtat

SEQ ID NO: 519 - DNA, 26 bp, Artificial Sequence
  Description of Artificial Sequence: Synthetic oligonucleotide
ttctacacagaactgagcggacaata

SEQ ID NO: 520 - DNA, 26 bp, Artificial Sequence
  Description of Artificial Sequence: Synthetic oligonucleotide
tattgcacgttcctatcttgaactgt

SEQ ID NO: 521 - DNA, 26 bp, Artificial Sequence
  Description of Artificial Sequence: Synthetic oligonucleotide
catgacagtactctatgtcgagtggt

SEQ ID NO: 522 - DNA, 26 bp, Artificial Sequence
  Description of Artificial Sequence: Synthetic oligonucleotide
taattcactaccctgtagcgacatca

SEQ ID NO: 523 - DNA, 26 bp, Artificial Sequence
  Description of Artificial Sequence: Synthetic oligonucleotide
acgctcaaattacttggttgaaagaa

SEQ ID NO: 524 - DNA, 26 bp, Artificial Sequence
  Description of Artificial Sequence: Synthetic oligonucleotide
ccttgcattaatcttgttggattcgt

SEQ ID NO: 525 - DNA, 26 bp, Artificial Sequence
  Description of Artificial Sequence: Synthetic oligonucleotide
gtagccacatcactccaacgaaacat

SEQ ID NO: 526 - DNA, 26 bp, Artificial Sequence
  Description of Artificial Sequence: Synthetic oligonucleotide
cttgtcaaattcctaccgggactcag

SEQ ID NO: 527 - DNA, 26 bp, Artificial Sequence
  Description of Artificial Sequence: Synthetic oligonucleotide
tccaacattctactgttaagatctga

SEQ ID NO: 528 - DNA, 26 bp, Artificial Sequence
  Description of Artificial Sequence: Synthetic oligonucleotide
agagccatgcctctcggctgaaacgt

SEQ ID NO: 529 - DNA, 26 bp, Artificial Sequence
  Description of Artificial Sequence: Synthetic oligonucleotide
cttcgcaccgatcttccaagagaatt

SEQ ID NO: 530 - DNA, 26 bp, Artificial Sequence
  Description of Artificial Sequence: Synthetic oligonucleotide
tcggtcacacggctccgaagacgttg

SEQ ID NO: 531 - DNA, 26 bp, Artificial Sequence
  Description of Artificial Sequence: Synthetic oligonucleotide
gaacacaagtatcttaaccgagccga

SEQ ID NO: 532 - DNA, 26 bp, Artificial Sequence
  Description of Artificial Sequence: Synthetic oligonucleotide
aattgcagcggactctccggatgctg

SEQ ID NO: 533 - DNA, 26 bp, Artificial Sequence
  Description of Artificial Sequence: Synthetic oligonucleotide
ggcctcagtcctctcattcgacagct

SEQ ID NO: 534 - DNA, 26 bp, Artificial Sequence
  Description of Artificial Sequence: Synthetic oligonucleotide
taggtcatctctctggttagatgcta

SEQ ID NO: 535 - DNA, 26 bp, Artificial Sequence
  Description of Artificial Sequence: Synthetic oligonucleotide
acacacaatatcctaccacgaacggt

SEQ ID NO: 536 - DNA, 26 bp, Artificial Sequence
  Description of Artificial Sequence: Synthetic oligonucleotide
ttcctcagtacgcttaggtgatctct

SEQ ID NO: 537 - DNA, 26 bp, Artificial Sequence
  Description of Artificial Sequence: Synthetic oligonucleotide
ggtaacacgcagcttatgggactcga

SEQ ID NO: 538 - DNA, 26 bp, Artificial Sequence
  Description of Artificial Sequence: Synthetic oligonucleotide
tccaccaggcctctctcgtgagcgtt

SEQ ID NO: 539 - DNA, 26 bp, Artificial Sequence
  Description of Artificial Sequence: Synthetic oligonucleotide
gataccactcctctccagtgatggca

SEQ ID NO: 540 - DNA, 26 bp, Artificial Sequence
  Description of Artificial Sequence: Synthetic oligonucleotide
caacgcatcagccttgttcgagcatt

SEQ ID NO: 541 - DNA, 26 bp, Artificial Sequence
  Description of Artificial Sequence: Synthetic oligonucleotide
cggttcaattagctaaccggacatcg

SEQ ID NO: 542 - DNA, 26 bp, Artificial Sequence
  Description of Artificial Sequence: Synthetic oligonucleotide
cgcgccactagactcgaaggagttaa

SEQ ID NO: 543 - DNA, 26 bp, Artificial Sequence
  Description of Artificial Sequence: Synthetic oligonucleotide
tcttgcagctatctagtgcgacactg

SEQ ID NO: 544 - DNA, 26 bp, Artificial Sequence
  Description of Artificial Sequence: Synthetic oligonucleotide
tcacacaccgaactgaacagaagtat

SEQ ID NO: 545 - DNA, 26 bp, Artificial Sequence
  Description of Artificial Sequence: Synthetic oligonucleotide
aacgtcatacatctacgatgatgctg

SEQ ID NO: 546 - DNA, 26 bp, Artificial Sequence
  Description of Artificial Sequence: Synthetic oligonucleotide
cggcccatcgttctataccgatggat

SEQ ID NO: 547 - DNA, 26 bp, Artificial Sequence
  Description of Artificial Sequence: Synthetic oligonucleotide
cataacacaccacttccaagattcta

SEQ ID NO: 548 - DNA, 26 bp, Artificial Sequence
  Description of Artificial Sequence: Synthetic oligonucleotide
acagacaggccacttgagagacagcg

SEQ ID NO: 549 - DNA, 26 bp, Artificial Sequence
  Description of Artificial Sequence: Synthetic oligonucleotide
tggtgcacctggctacgctgaaatta

SEQ ID NO: 550 - DNA, 26 bp, Artificial Sequence
  Description of Artificial Sequence: Synthetic oligonucleotide
taggacaaccggcttatatgatcgag

SEQ ID NO: 551 - DNA, 26 bp, Artificial Sequence
  Description of Artificial Sequence: Synthetic oligonucleotide
aatatcatggccctcggtcgacgata

SEQ ID NO: 552 - DNA, 26 bp, Artificial Sequence
  Description of Artificial Sequence: Synthetic oligonucleotide
ataggcatattcctacaatgaagagt

SEQ ID NO: 553 - DNA, 26 bp, Artificial Sequence
  Description of Artificial Sequence: Synthetic oligonucleotide
ccttccaacgtactcggttgaattag

SEQ ID NO: 554 - DNA, 26 bp, Artificial Sequence
  Description of Artificial Sequence: Synthetic oligonucleotide
ggccacaataagctgataagacaagt

SEQ ID NO: 555 - DNA, 26 bp, Artificial Sequence
  Description of Artificial Sequence: Synthetic oligonucleotide
cagtacagttgtctagttagatcaca

SEQ ID NO: 556 - DNA, 26 bp, Artificial Sequence
  Description of Artificial Sequence: Synthetic oligonucleotide
ttcatcaccaacctttccagaggtaa

SEQ ID NO: 557 - DNA, 26 bp, Artificial Sequence
  Description of Artificial Sequence: Synthetic oligonucleotide
caattcaggattctcatgtgaagagg

SEQ ID NO: 558 - DNA, 26 bp, Artificial Sequence
  Description of Artificial Sequence: Synthetic oligonucleotide
ggccacatcatactgattggatcata

SEQ ID NO: 559 - DNA, 26 bp, Artificial Sequence
  Description of Artificial Sequence: Synthetic oligonucleotide
aattgcactgcgctattccgagctat

SEQ ID NO: 560 - DNA, 26 bp, Artificial Sequence
  Description of Artificial Sequence: Synthetic oligonucleotide
taaggcaaacgtctgaccggactgtg

SEQ ID NO: 561 - DNA, 26 bp, Artificial Sequence
  Description of Artificial Sequence: Synthetic oligonucleotide
ctatacacgcggcttaggagaaccgg

SEQ ID NO: 562 - DNA, 26 bp, Artificial Sequence
  Description of Artificial Sequence: Synthetic oligonucleotide
attcacagaatcctagcgggatggac

SEQ ID NO: 563 - DNA, 26 bp, Artificial Sequence
  Description of Artificial Sequence: Synthetic oligonucleotide
gtattcactctacttataggaattcg

SEQ ID NO: 564 - DNA, 26 bp, Artificial Sequence
  Description of Artificial Sequence: Synthetic oligonucleotide
cctgacatacaactacagagaggcca

SEQ ID NO: 565 - DNA, 26 bp, Artificial Sequence
  Description of Artificial Sequence: Synthetic oligonucleotide
gaccgcactgtgctattccgatattg

SEQ ID NO: 566 - DNA, 26 bp, Artificial Sequence
  Description of Artificial Sequence: Synthetic oligonucleotide
ttcagcacgtggcttattcgactcag

SEQ ID NO: 567 - DNA, 26 bp, Artificial Sequence
  Description of Artificial Sequence: Synthetic oligonucleotide
aactccacgaacctcgcctgatctga

SEQ ID NO: 568 - DNA, 26 bp, Artificial Sequence
  Description of Artificial Sequence: Synthetic oligonucleotide
attcccagctatctgcgcagagagta

SEQ ID NO: 569 - DNA, 26 bp, Artificial Sequence
  Description of Artificial Sequence: Synthetic oligonucleotide
tgaatcaattgcctggcgcgacaatt

SEQ ID NO: 570 - DNA, 26 bp, Artificial Sequence
  Description of Artificial Sequence: Synthetic oligonucleotide
cgcaacatctagctagatagatggcg

SEQ ID NO: 571 - DNA, 26 bp, Artificial Sequence
  Description of Artificial Sequence: Synthetic oligonucleotide
aaccgcacatcgctcctgcgattggt

SEQ ID NO: 572 - DNA, 26 bp, Artificial Sequence
  Description of Artificial Sequence: Synthetic oligonucleotide
ctagtcaccggactgacgagaacaat

SEQ ID NO: 573 - DNA, 26 bp, Artificial Sequence
  Description of Artificial Sequence: Synthetic oligonucleotide
gctcccagtcaccttggcggagtcca

SEQ ID NO: 574 - DNA, 26 bp, Artificial Sequence
  Description of Artificial Sequence: Synthetic oligonucleotide
agatgcagaattctcttcagagttac

SEQ ID NO: 575 - DNA, 26 bp, Artificial Sequence
  Description of Artificial Sequence: Synthetic oligonucleotide
acacccagttaacttcctggaaccgt

SEQ ID NO: 576 - DNA, 26 bp, Artificial Sequence
  Description of Artificial Sequence: Synthetic oligonucleotide
gataacacaagtctcgcgcgactaga

SEQ ID NO: 577 - DNA, 26 bp, Artificial Sequence
  Description of Artificial Sequence: Synthetic oligonucleotide
ctggtcaacacgctaggatgaaagtt

SEQ ID NO: 578 - DNA, 26 bp, Artificial Sequence
  Description of Artificial Sequence: Synthetic oligonucleotide
cgaagcagttaactaggccgaagaca

SEQ ID NO: 579 - DNA, 26 bp, Artificial Sequence
  Description of Artificial Sequence: Synthetic oligonucleotide
atcgccaatatgctccttggaaacgg

SEQ ID NO: 580 - DNA, 26 bp, Artificial Sequence
  Description of Artificial Sequence: Synthetic oligonucleotide
atcatcaaggctctcaccagacctac

SEQ ID NO: 581 - DNA, 26 bp, Artificial Sequence
  Description of Artificial Sequence: Synthetic oligonucleotide
gattgcatcatactttgctgatgtat

SEQ ID NO: 582 - DNA, 26 bp, Artificial Sequence
  Description of Artificial Sequence: Synthetic oligonucleotide
ccaaccaaacatctcaatcgatatga

SEQ ID NO: 583 - DNA, 26 bp, Artificial Sequence
  Description of Artificial Sequence: Synthetic oligonucleotide
ttggtcaggtgccttggtagactgat

SEQ ID NO: 584 - DNA, 26 bp, Artificial Sequence
  Description of Artificial Sequence: Synthetic oligonucleotide
gcgaacacgcctctttcatgaccaac

SEQ ID NO: 585 - DNA, 26 bp, Artificial Sequence
  Description of Artificial Sequence: Synthetic oligonucleotide
caacccaggaggctcataagacacca

SEQ ID NO: 586 - DNA, 26 bp, Artificial Sequence
  Description of Artificial Sequence: Synthetic oligonucleotide
agcggcatggaccttcctagattagc

SEQ ID NO: 587 - DNA, 26 bp, Artificial Sequence
  Description of Artificial Sequence: Synthetic oligonucleotide
gacgacaacaatcttctctgaagatt

SEQ ID NO: 588 - DNA, 26 bp, Artificial Sequence
  Description of Artificial Sequence: Synthetic oligonucleotide
ccactcaggtccctcgcgagagccta

SEQ ID NO: 589 - DNA, 26 bp, Artificial Sequence
  Description of Artificial Sequence: Synthetic oligonucleotide
tgttacagaaggctgataagagctct

SEQ ID NO: 590 - DNA, 26 bp, Artificial Sequence
  Description of Artificial Sequence: Synthetic oligonucleotide
tatatcatcgagctgagatgagtcga

SEQ ID NO: 591 - DNA, 26 bp, Artificial Sequence
  Description of Artificial Sequence: Synthetic oligonucleotide
cgcgacacgatcctctggagatatgt

SEQ ID NO: 592 - DNA, 26 bp, Artificial Sequence
  Description of Artificial Sequence: Synthetic oligonucleotide
gcctccaggatactggccagaataag

SEQ ID NO: 593 - DNA, 26 bp, Artificial Sequence
  Description of Artificial Sequence: Synthetic oligonucleotide
tgagacacagcgctattacgatcacc

SEQ ID NO: 594 - DNA, 26 bp, Artificial Sequence
  Description of Artificial Sequence: Synthetic oligonucleotide
tgttccagcattctaattggagcgga

SEQ ID NO: 595 - DNA, 26 bp, Artificial Sequence
  Description of Artificial Sequence: Synthetic oligonucleotide
tccaacagaattctttgtcgaaactt

SEQ ID NO: 596 - DNA, 26 bp, Artificial Sequence
  Description of Artificial Sequence: Synthetic oligonucleotide
gctgtcaaggaactggcgagaattct

SEQ ID NO: 597 - DNA, 26 bp, Artificial Sequence
  Description of Artificial Sequence: Synthetic oligonucleotide
atacccatggatctcaacggatcagc

SEQ ID NO: 598 - DNA, 26 bp, Artificial Sequence
  Description of Artificial Sequence: Synthetic oligonucleotide
gttggcaaccgtcttcttagacatca

SEQ ID NO: 599 - DNA, 26 bp, Artificial Sequence
  Description of Artificial Sequence: Synthetic oligonucleotide
accaacagttacctcgccagatacct

SEQ ID NO: 600 - DNA, 26 bp, Artificial Sequence
  Description of Artificial Sequence: Synthetic oligonucleotide
gtgtgcagcgctctctaatgagtctt

SEQ ID NO: 601 - DNA, 26 bp, Artificial Sequence
  Description of Artificial Sequence: Synthetic oligonucleotide
ggcagcatagcactcaaccgaggagg

SEQ ID NO: 602 - DNA, 26 bp, Artificial Sequence
  Description of Artificial Sequence: Synthetic oligonucleotide
tgcggcatgttgctggcaggatagca

SEQ ID NO: 603 - DNA, 26 bp, Artificial Sequence
  Description of Artificial Sequence: Synthetic oligonucleotide
gattacaaggtgctttagggaataga

SEQ ID NO: 604 - DNA, 26 bp, Artificial Sequence
  Description of Artificial Sequence: Synthetic oligonucleotide
caacacattcaactcgcaagatctag

SEQ ID NO: 605 - DNA, 26 bp, Artificial Sequence
  Description of Artificial Sequence: Synthetic oligonucleotide
gtgttcaaccggctgagttgagtact

SEQ ID NO: 606 - DNA, 26 bp, Artificial Sequence
  Description of Artificial Sequence: Synthetic oligonucleotide
tatcacatgagactaacacgagtgga

SEQ ID NO: 607 - DNA, 26 bp, Artificial Sequence
  Description of Artificial Sequence: Synthetic oligonucleotide
cttggcacctcgctgtgttgaaccgg

SEQ ID NO: 608 - DNA, 26 bp, Artificial Sequence
  Description of Artificial Sequence: Synthetic oligonucleotide
gtctccagtgaactagattgagttac

SEQ ID NO: 609 - DNA, 26 bp, Artificial Sequence
  Description of Artificial Sequence: Synthetic oligonucleotide
ccatccacacgcctttgacgacaatg

SEQ ID NO: 610 - DNA, 26 bp, Artificial Sequence
  Description of Artificial Sequence: Synthetic oligonucleotide
acaaccacaggactctgacgacggca

SEQ ID NO: 611 - DNA, 26 bp, Artificial Sequence
  Description of Artificial Sequence: Synthetic oligonucleotide
agcagcaaattacttctcagatcaat

SEQ ID NO: 612 - DNA, 26 bp, Artificial Sequence
  Description of Artificial Sequence: Synthetic oligonucleotide
cagtccagtgcgctggaccgaaacag

SEQ ID NO: 613 - DNA, 26 bp, Artificial Sequence
  Description of Artificial Sequence: Synthetic oligonucleotide
gtctacaacctcctaatgtgaattgc

SEQ ID NO: 614 - DNA, 26 bp, Artificial Sequence
  Description of Artificial Sequence: Synthetic oligonucleotide
gaactcacggttctgatctgactgga

SEQ ID NO: 615 - DNA, 26 bp, Artificial Sequence
  Description of Artificial Sequence: Synthetic oligonucleotide
agttacatcacactcaggcgagccat

SEQ ID NO: 616 - DNA, 26 bp, Artificial Sequence
  Description of Artificial Sequence: Synthetic oligonucleotide
gtagccaatactctttaatgaagacc

SEQ ID NO: 617 - DNA, 26 bp, Artificial Sequence
  Description of Artificial Sequence: Synthetic oligonucleotide
cttcacagttacctggagtgacgcga

SEQ ID NO: 618 - DNA, 26 bp, Artificial Sequence
  Description of Artificial Sequence: Synthetic oligonucleotide
agtcccagaggactaacgcgacagag

SEQ ID NO: 619 - DNA, 26 bp, Artificial Sequence
  Description of Artificial Sequence: Synthetic oligonucleotide
acagtcatccagctcgtaagattaac

SEQ ID NO: 620 - DNA, 26 bp, Artificial Sequence
  Description of Artificial Sequence: Synthetic oligonucleotide
ccgcacatattcctacgaggaactga

SEQ ID NO: 621 - DNA, 26 bp, Artificial Sequence
  Description of Artificial Sequence: Synthetic oligonucleotide
ttatccacgatcctgtatcgaggccg

SEQ ID NO: 622 - DNA, 26 bp, Artificial Sequence
  Description of Artificial Sequence: Synthetic oligonucleotide
atagtcactagcctaatacgagacat

SEQ ID NO: 623 - DNA, 26 bp, Artificial Sequence
  Description of Artificial Sequence: Synthetic oligonucleotide
tatagcatagctctgttatgaatggc

SEQ ID NO: 624 - DNA, 26 bp, Artificial Sequence
  Description of Artificial Sequence: Synthetic oligonucleotide
actcccaggtggctgcctggaccatg

SEQ ID NO: 625 - DNA, 26 bp, Artificial Sequence
  Description of Artificial Sequence: Synthetic oligonucleotide
gtgcgcagtaagcttaagagacctat

SEQ ID NO: 626 - DNA, 26 bp, Artificial Sequence
  Description of Artificial Sequence: Synthetic oligonucleotide
gatatcacctaacttatacgacatgg

SEQ ID NO: 627 - DNA, 26 bp, Artificial Sequence
  Description of Artificial Sequence: Synthetic oligonucleotide
tcgcgcatataactgccgtgactgtt

SEQ ID NO: 628 - DNA, 26 bp, Artificial Sequence
  Description of Artificial Sequence: Synthetic oligonucleotide
attctcaaagcgctcagaggatgata

SEQ ID NO: 629 - DNA, 26 bp, Artificial Sequence
  Description of Artificial Sequence: Synthetic oligonucleotide
agcgccattcggcttgctagaactat

SEQ ID NO: 630 - DNA, 26 bp, Artificial Sequence
  Description of Artificial Sequence: Synthetic oligonucleotide
gttgacatagtgcttcagtgataatg

SEQ ID NO: 631 - DNA, 26 bp, Artificial Sequence
  Description of Artificial Sequence: Synthetic oligonucleotide
aatagcaagcaactgtgacgacttga

SEQ ID NO: 632 - DNA, 26 bp, Artificial Sequence
  Description of Artificial Sequence: Synthetic oligonucleotide
ctaaccatgtaactacatggacatat

SEQ ID NO: 633 - DNA, 26 bp, Artificial Sequence
  Description of Artificial Sequence: Synthetic oligonucleotide
gcgtacacttagctaacatgaaccta

SEQ ID NO: 634 - DNA, 26 bp, Artificial Sequence
  Description of Artificial Sequence: Synthetic oligonucleotide
taccgcaaactactccatggatgtag

SEQ ID NO: 635 - DNA, 26 bp, Artificial Sequence
  Description of Artificial Sequence: Synthetic oligonucleotide
gtagtcaaatagctgagtcgatctcc

SEQ ID NO: 636 - DNA, 26 bp, Artificial Sequence
  Description of Artificial Sequence: Synthetic oligonucleotide
ggttacatgctactgctatgagcgca

SEQ ID NO: 637 - DNA, 26 bp, Artificial Sequence
  Description of Artificial Sequence: Synthetic oligonucleotide
acaatcaagagtctatcgcgaatatg

SEQ ID NO: 638 - DNA, 26 bp, Artificial Sequence
  Description of Artificial Sequence: Synthetic oligonucleotide
gcttccacactactagtacgactata

SEQ ID NO: 639 - DNA, 26 bp, Artificial Sequence
  Description of Artificial Sequence: Synthetic oligonucleotide
agatacatggcgctgaccggagagat

SEQ ID NO: 640 - DNA, 26 bp, Artificial Sequence
  Description of Artificial Sequence: Synthetic oligonucleotide
aatatcagaagcctcgttcgaagcct

SEQ ID NO: 641 - DNA, 26 bp, Artificial Sequence
  Description of Artificial Sequence: Synthetic oligonucleotide
tagcgcactagtctttactgatcctc

SEQ ID NO: 642 - DNA, 26 bp, Artificial Sequence
  Description of Artificial Sequence: Synthetic oligonucleotide
agttacaagagcctcacgtgaccacc

SEQ ID NO: 643 - DNA, 26 bp, Artificial Sequence
  Description of Artificial Sequence: Synthetic oligonucleotide
cagatcaaccacctgctacgatatct

SEQ ID NO: 644 - DNA, 26 bp, Artificial Sequence
  Description of Artificial Sequence: Synthetic oligonucleotide
acggccacgtcactagtcagaaccat

SEQ ID NO: 645 - DNA, 26 bp, Artificial Sequence
  Description of Artificial Sequence: Synthetic oligonucleotide
gtaatcatactgctcgagggacggta

SEQ ID NO: 646 - DNA, 26 bp, Artificial Sequence
  Description of Artificial Sequence: Synthetic oligonucleotide
aagtccattgtactcaggtgagttca

SEQ ID NO: 647 - DNA, 26 bp, Artificial Sequence
  Description of Artificial Sequence: Synthetic oligonucleotide
gtcaccacacagctgacaggaacagg

SEQ ID NO: 648 - DNA, 26 bp, Artificial Sequence
  Description of Artificial Sequence: Synthetic oligonucleotide
attagcatggagcttgtacgattgtt

SEQ ID NO: 649 - DNA, 26 bp, Artificial Sequence
  Description of Artificial Sequence: Synthetic oligonucleotide
tgctacaactatctctctagaagtag

SEQ ID NO: 650 - DNA, 26 bp, Artificial Sequence
  Description of Artificial Sequence: Synthetic oligonucleotide
taagacacctatctgtcacgacacag

SEQ ID NO: 651 - DNA, 26 bp, Artificial Sequence
  Description of Artificial Sequence: Synthetic oligonucleotide
tggttcaaagaacttctacgaatacc

SEQ ID NO: 652 - DNA, 26 bp, Artificial Sequence
  Description of Artificial Sequence: Synthetic oligonucleotide
actctcatccttctcacgtgataggc

SEQ ID NO: 653 - DNA, 26 bp, Artificial Sequence
  Description of Artificial Sequence: Synthetic oligonucleotide
gtctccacttcccttggtggaagtct

SEQ ID NO: 654 - DNA, 26 bp, Artificial Sequence
  Description of Artificial Sequence: Synthetic oligonucleotide
tccgccagttcactcttcggaaagga

SEQ ID NO: 655 - DNA, 26 bp, Artificial Sequence
  Description of Artificial Sequence: Synthetic oligonucleotide
aggttcagcaggctgtagagagtcag

SEQ ID NO: 656 - DNA, 26 bp, Artificial Sequence
  Description of Artificial Sequence: Synthetic oligonucleotide
gaacccaatgaactgacatgatgtca

SEQ ID NO: 657 - DNA, 26 bp, Artificial Sequence
  Description of Artificial Sequence: Synthetic oligonucleotide
ttgagcaaggatcttccgcgaaaggc

SEQ ID NO: 658 - DNA, 26 bp, Artificial Sequence
  Description of Artificial Sequence: Synthetic oligonucleotide
tggtccatagtgctactgcgacttat

SEQ ID NO: 659 - DNA, 26 bp, Artificial Sequence
  Description of Artificial Sequence: Synthetic oligonucleotide
agtggcaataatcttacgcgaacgta

SEQ ID NO: 660 - DNA, 26 bp, Artificial Sequence
  Description of Artificial Sequence: Synthetic oligonucleotide
ggcaccagccatctcgcttgagaagt

SEQ ID NO: 661 - DNA, 26 bp, Artificial Sequence
  Description of Artificial Sequence: Synthetic oligonucleotide
gatctcactggactctgcagacttca

SEQ ID NO: 662 - DNA, 26 bp, Artificial Sequence
  Description of Artificial Sequence: Synthetic oligonucleotide
tgctgcagacatctcagcggagacaa

SEQ ID NO: 663 - DNA, 26 bp, Artificial Sequence
  Description of Artificial Sequence: Synthetic oligonucleotide
ccgaacacgttgctggatcgacgcat

SEQ ID NO: 664 - DNA, 26 bp, Artificial Sequence
  Description of Artificial Sequence: Synthetic oligonucleotide
attaacatacgccttgcgggatgttg

SEQ ID NO: 665 - DNA, 26 bp, Artificial Sequence
  Description of Artificial Sequence: Synthetic oligonucleotide
tagtccaacaacctacatagaacgga

SEQ ID NO: 666 - DNA, 26 bp, Artificial Sequence
  Description of Artificial Sequence: Synthetic oligonucleotide
ggtatcatgagactgacgtgatcgcg

SEQ ID NO: 667 - DNA, 26 bp, Artificial Sequence
  Description of Artificial Sequence: Synthetic oligonucleotide
caagacatgcttctcattcgaaacaa

SEQ ID NO: 668 - DNA, 26 bp, Artificial Sequence
  Description of Artificial Sequence: Synthetic oligonucleotide
acgagcaactgactcacgggaattat

SEQ ID NO: 669 - DNA, 26 bp, Artificial Sequence
  Description of Artificial Sequence: Synthetic oligonucleotide
ttatccattgcactttgaggagacgg

SEQ ID NO: 670 - DNA, 26 bp, Artificial Sequence
  Description of Artificial Sequence: Synthetic oligonucleotide
agattcagttacctctctggatatac

SEQ ID NO: 671 - DNA, 26 bp, Artificial Sequence
  Description of Artificial Sequence: Synthetic oligonucleotide
tctaccacgctgctgcaacgaaggtg

SEQ ID NO: 672 - DNA, 26 bp, Artificial Sequence
  Description of Artificial Sequence: Synthetic oligonucleotide
aacggcatatgactggtaagacgcag

SEQ ID NO: 673 - DNA, 26 bp, Artificial Sequence
  Description of Artificial Sequence: Synthetic oligonucleotide
caatgcagcgccctaccgcgagcaat

SEQ ID NO: 674 - DNA, 26 bp, Artificial Sequence
  Description of Artificial Sequence: Synthetic oligonucleotide
ctaatcatcgctctagccggagaaca

SEQ ID NO: 675 - DNA, 26 bp, Artificial Sequence
  Description of Artificial Sequence: Synthetic oligonucleotide
catggcatctaacttcctagaggaag

SEQ ID NO: 676 - DNA, 26 bp, Artificial Sequence
  Description of Artificial Sequence: Synthetic oligonucleotide
atactcagtgtgctttgaggacctaa

SEQ ID NO: 677 - DNA, 26 bp, Artificial Sequence
  Description of Artificial Sequence: Synthetic oligonucleotide
gccgacacaagactccaccgatgtgt

SEQ ID NO: 678 - DNA, 26 bp, Artificial Sequence
  Description of Artificial Sequence: Synthetic oligonucleotide
cgaggcacggtactcctcggacaacc

SEQ ID NO: 679 - DNA, 26 bp, Artificial Sequence
  Description of Artificial Sequence: Synthetic oligonucleotide
gatatcaaacagctgtatagagctgt

SEQ ID NO: 680 - DNA, 26 bp, Artificial Sequence
  Description of Artificial Sequence: Synthetic oligonucleotide
tcgcccaggttactgctacgaattag

SEQ ID NO: 681 - DNA, 26 bp, Artificial Sequence
  Description of Artificial Sequence: Synthetic oligonucleotide
agactcactcttcttacgagaatctt

SEQ ID NO: 682 - DNA, 26 bp, Artificial Sequence
  Description of Artificial Sequence: Synthetic oligonucleotide
gctcgcacctaccttaggagagcgca

SEQ ID NO: 683 - DNA, 26 bp, Artificial Sequence
  Description of Artificial Sequence: Synthetic oligonucleotide
aggatcaaagttctgtactgaggcgt

SEQ ID NO: 684 - DNA, 26 bp, Artificial Sequence
  Description of Artificial Sequence: Synthetic oligonucleotide
gagaccaataatctagttagaagagc

SEQ ID NO: 685 - DNA, 26 bp, Artificial Sequence
  Description of Artificial Sequence: Synthetic oligonucleotide
agctgcattatacttcgcggatataa

SEQ ID NO: 686 - DNA, 26 bp, Artificial Sequence
  Description of Artificial Sequence: Synthetic oligonucleotide
gtatccaattggctgagtggatgccg

SEQ ID NO: 687 - DNA, 26 bp, Artificial Sequence
  Description of Artificial Sequence: Synthetic oligonucleotide
aatagcagcctcctctagtgaccgga

SEQ ID NO: 688 - DNA, 26 bp, Artificial Sequence
  Description of Artificial Sequence: Synthetic oligonucleotide
ccgctcatagctctattaagatacgc

SEQ ID NO: 689 - DNA, 26 bp, Artificial Sequence
  Description of Artificial Sequence: Synthetic oligonucleotide
tcctacaggaagctcctaggaagtat

SEQ ID NO: 690 - DNA, 26 bp, Artificial Sequence
  Description of Artificial Sequence: Synthetic oligonucleotide
tcacacagatcgcttaggagaagact

SEQ ID NO: 691 - DNA, 26 bp, Artificial Sequence
  Description of Artificial Sequence: Synthetic oligonucleotide
acttgcatccacctccgtggagcctt

SEQ ID NO: 692 - DNA, 26 bp, Artificial Sequence
  Description of Artificial Sequence: Synthetic oligonucleotide
tgtaccattgttctggatagatatcc

SEQ ID NO: 693 - DNA, 26 bp, Artificial Sequence
  Description of Artificial Sequence: Synthetic oligonucleotide
cacttcaaatctctcacctgacttgg

SEQ ID NO: 694 - DNA, 26 bp, Artificial Sequence
  Description of Artificial Sequence: Synthetic oligonucleotide
cagagcatgatactaacgtgatacat

SEQ ID NO: 695 - DNA, 26 bp, Artificial Sequence
  Description of Artificial Sequence: Synthetic oligonucleotide
ggcgacaattctctcggcagaagctc

SEQ ID NO: 696 - DNA, 26 bp, Artificial Sequence
  Description of Artificial Sequence: Synthetic oligonucleotide
agtggcatcaggcttcttggagctat

SEQ ID NO: 697 - DNA, 26 bp, Artificial Sequence
  Description of Artificial Sequence: Synthetic oligonucleotide
cattccacagctctacggagaatgcg

SEQ ID NO: 698 - DNA, 26 bp, Artificial Sequence
  Description of Artificial Sequence: Synthetic oligonucleotide
ctcgtcatatcactgttccgagcagg

SEQ ID NO: 699 - DNA, 26 bp, Artificial Sequence
  Description of Artificial Sequence: Synthetic oligonucleotide
ccttacactatgctaccaagagttac

SEQ ID NO: 700 - DNA, 26 bp, Artificial Sequence
  Description of Artificial Sequence: Synthetic oligonucleotide
agaagcaccaatcttggctgacgcag

SEQ ID NO: 701 - DNA, 26 bp, Artificial Sequence
  Description of Artificial Sequence: Synthetic oligonucleotide
taatccaggtacctaactagaacgtt

SEQ ID NO: 702 - DNA, 26 bp, Artificial Sequence
  Description of Artificial Sequence: Synthetic oligonucleotide
ggaatcatgttccttagaggattgga

SEQ ID NO: 703 - DNA, 26 bp, Artificial Sequence
  Description of Artificial Sequence: Synthetic oligonucleotide
ccggacaccacactagagcgaactag

SEQ ID NO: 704 - DNA, 26 bp, Artificial Sequence
  Description of Artificial Sequence: Synthetic oligonucleotide
gacttcaagaagctactctgaacagg

SEQ ID NO: 705 - DNA, 26 bp, Artificial Sequence
  Description of Artificial Sequence: Synthetic oligonucleotide
tggcacaatattctcggtggaacacc

SEQ ID NO: 706 - DNA, 26 bp, Artificial Sequence
  Description of Artificial Sequence: Synthetic oligonucleotide
gaatgcacacgactgcgttgaggtat

SEQ ID NO: 707 - DNA, 26 bp, Artificial Sequence
  Description of Artificial Sequence: Synthetic oligonucleotide
cgtgtcaatcttcttgtgcgataaca

SEQ ID NO: 708 - DNA, 26 bp, Artificial Sequence
  Description of Artificial Sequence: Synthetic oligonucleotide
attcacattgcactccagagaagtaa

SEQ ID NO: 709 - DNA, 26 bp, Artificial Sequence
  Description of Artificial Sequence: Synthetic oligonucleotide
tccttcacatagctcttatgaacctg

SEQ ID NO: 710 - DNA, 26 bp, Artificial Sequence
  Description of Artificial Sequence: Synthetic oligonucleotide
tctagcatcttcctactaggaaactt

SEQ ID NO: 711 - DNA, 26 bp, Artificial Sequence
  Description of Artificial Sequence: Synthetic oligonucleotide
ctcgacactcctctttagggacttac

SEQ ID NO: 712 - DNA, 26 bp, Artificial Sequence
  Description of Artificial Sequence: Synthetic oligonucleotide
agtgacagtgaacttatcagatgaga

SEQ ID NO: 713 - DNA, 26 bp, Artificial Sequence
  Description of Artificial Sequence: Synthetic oligonucleotide
gaagccaggaccctctcacgaacaag

SEQ ID NO: 714 - DNA, 26 bp, Artificial Sequence
  Description of Artificial Sequence: Synthetic oligonucleotide
gctctcacgttgctgaattgagagtg

SEQ ID NO: 715 - DNA, 26 bp, Artificial Sequence
  Description of Artificial Sequence: Synthetic oligonucleotide
ggacccatcaatctcggatgatatat

SEQ ID NO: 716 - DNA, 26 bp, Artificial Sequence
  Description of Artificial Sequence: Synthetic oligonucleotide
gagtccatctccctttgaagagcaga

SEQ ID NO: 717 - DNA, 26 bp, Artificial Sequence
  Description of Artificial Sequence: Synthetic oligonucleotide
aacggcaagcggcttacgggacgaag

SEQ ID NO: 718 - DNA, 26 bp, Artificial Sequence
  Description of Artificial Sequence: Synthetic oligonucleotide
tgtgacatgtatcttctccgaattga

SEQ ID NO: 719 - DNA, 26 bp, Artificial Sequence
  Description of Artificial Sequence: Synthetic oligonucleotide
aacatcaacctactcgagagaccaag

SEQ ID NO: 720 - DNA, 26 bp, Artificial Sequence
  Description of Artificial Sequence: Synthetic oligonucleotide
gtgctcaaggtgcttgctggagacat

SEQ ID NO: 721 - DNA, 26 bp, Artificial Sequence
  Description of Artificial Sequence: Synthetic oligonucleotide
cataccattgaactgatgggatatcg

SEQ ID NO: 722 - DNA, 26 bp, Artificial Sequence
  Description of Artificial Sequence: Synthetic oligonucleotide
cttgtcacttaactggcttgaaattg

SEQ ID NO: 723 - DNA, 26 bp, Artificial Sequence
  Description of Artificial Sequence: Synthetic oligonucleotide
aagagcaaggtgctctcgagactcct

SEQ ID NO: 724 - DNA, 26 bp, Artificial Sequence
  Description of Artificial Sequence: Synthetic oligonucleotide
tgcaccagagaactatacagacagag

SEQ ID NO: 725 - DNA, 26 bp, Artificial Sequence
  Description of Artificial Sequence: Synthetic oligonucleotide
acttccactagccttctcggagacga

SEQ ID NO: 726 - DNA, 26 bp, Artificial Sequence
  Description of Artificial Sequence: Synthetic oligonucleotide
gtgctcaattaactaccacgagtctg

SEQ ID NO: 727 - DNA, 26 bp, Artificial Sequence
  Description of Artificial Sequence: Synthetic oligonucleotide
agcgtcagaatgctgttgtgaactca

SEQ ID NO: 728 - DNA, 26 bp, Artificial Sequence
  Description of Artificial Sequence: Synthetic oligonucleotide
ccttacagtgcccttcagggatcaac

SEQ ID NO: 729 - DNA, 26 bp, Artificial Sequence
  Description of Artificial Sequence: Synthetic oligonucleotide
tgtaccacgaatctagtccgagagga

SEQ ID NO: 730 - DNA, 26 bp, Artificial Sequence
  Description of Artificial Sequence: Synthetic oligonucleotide
ggagacattagtctcacttgaaatct

SEQ ID NO: 731 - DNA, 26 bp, Artificial Sequence
  Description of Artificial Sequence: Synthetic oligonucleotide
tactacaacacacttactcgatgtta

SEQ ID NO: 732 - DNA, 26 bp, Artificial Sequence
  Description of Artificial Sequence: Synthetic oligonucleotide
taggtcacgttgctgcgacgatcgat

SEQ ID NO: 733 - DNA, 26 bp, Artificial Sequence
  Description of Artificial Sequence: Synthetic oligonucleotide
atgcccagaccgctctagggacaagg

SEQ ID NO: 734 - DNA, 26 bp, Artificial Sequence
  Description of Artificial Sequence: Synthetic oligonucleotide
ctagccagtcgactcctctgatcgaa

SEQ ID NO: 735 - DNA, 26 bp, Artificial Sequence
  Description of Artificial Sequence: Synthetic oligonucleotide
tgcctcaacgagcttcatcgactctt

SEQ ID NO: 736 - DNA, 26 bp, Artificial Sequence
  Description of Artificial Sequence: Synthetic oligonucleotide
actagcaaacttctggtaagagataa

SEQ ID NO: 737 - DNA, 26 bp, Artificial Sequence
  Description of Artificial Sequence: Synthetic oligonucleotide
cacctcacttggctaacgagagccag

SEQ ID NO: 738 - DNA, 26 bp, Artificial Sequence
  Description of Artificial Sequence: Synthetic oligonucleotide
aagcacagatatcttagacgaaatct

SEQ ID NO: 739 - DNA, 26 bp, Artificial Sequence
  Description of Artificial Sequence: Synthetic oligonucleotide
gccagcaatccactcaatggactgaa

SEQ ID NO: 740 - DNA, 26 bp, Artificial Sequence
  Description of Artificial Sequence: Synthetic oligonucleotide
ttggacattcaactgtcacgaggtgt

SEQ ID NO: 741 - DNA, 26 bp, Artificial Sequence
  Description of Artificial Sequence: Synthetic oligonucleotide
actagcaccgtgctggtgtgaacaag

SEQ ID NO: 742 - DNA, 26 bp, Artificial Sequence
  Description of Artificial Sequence: Synthetic oligonucleotide
cggcacaagctcctaggttgagcagg

SEQ ID NO: 743 - DNA, 26 bp, Artificial Sequence
  Description of Artificial Sequence: Synthetic oligonucleotide
gaagccatagctcttaatagacggag

SEQ ID NO: 744 - DNA, 26 bp, Artificial Sequence
  Description of Artificial Sequence: Synthetic oligonucleotide
acaagcagattgctcgaaggaacgca

SEQ ID NO: 745 - DNA, 26 bp, Artificial Sequence
  Description of Artificial Sequence: Synthetic oligonucleotide
gcaaccaaggtgctattgagacacat

SEQ ID NO: 746 - DNA, 26 bp, Artificial Sequence
  Description of Artificial Sequence: Synthetic oligonucleotide
caaggcatgacgctcagccgagattg

SEQ ID NO: 747 - DNA, 26 bp, Artificial Sequence
  Description of Artificial Sequence: Synthetic oligonucleotide
accagcatcattcttctcagacgcgt

SEQ ID NO: 748 - DNA, 26 bp, Artificial Sequence
  Description of Artificial Sequence: Synthetic oligonucleotide
ccggacaatcatctctctggaacgtg

SEQ ID NO: 749 - DNA, 26 bp, Artificial Sequence
  Description of Artificial Sequence: Synthetic oligonucleotide
ttgagcacctaacttcgaagatggaa

SEQ ID NO: 750 - DNA, 26 bp, Artificial Sequence
  Description of Artificial Sequence: Synthetic oligonucleotide
ccacccattacactaaggcgacttgg

SEQ ID NO: 751 - DNA, 26 bp, Artificial Sequence
  Description of Artificial Sequence: Synthetic oligonucleotide
gttgccaagttgcttgaacgagcaac

SEQ ID NO: 752 - DNA, 26 bp, Artificial Sequence
  Description of Artificial Sequence: Synthetic oligonucleotide
tcactcacatgtctccgctgatagct

SEQ ID NO: 753 - DNA, 26 bp, Artificial Sequence
  Description of Artificial Sequence: Synthetic oligonucleotide
gactgcagttgcctcaccggaaggaa

SEQ ID NO: 754 - DNA, 26 bp, Artificial Sequence
  Description of Artificial Sequence: Synthetic oligonucleotide
atcgtcacgctcctcgtatgaaatca

SEQ ID NO: 755 - DNA, 26 bp, Artificial Sequence
  Description of Artificial Sequence: Synthetic oligonucleotide
ggtgccagttcgctatgacgaagaac

SEQ ID NO: 756 - DNA, 26 bp, Artificial Sequence
  Description of Artificial Sequence: Synthetic oligonucleotide
cggcgcataagactattcagattgca

SEQ ID NO: 757 - DNA, 26 bp, Artificial Sequence
  Description of Artificial Sequence: Synthetic oligonucleotide
gacatcacagctcttcatggatcctg

SEQ ID NO: 758 - DNA, 26 bp, Artificial Sequence
  Description of Artificial Sequence: Synthetic oligonucleotide
actaacattcagctaattcgagatcg

SEQ ID NO: 759 - DNA, 26 bp, Artificial Sequence
  Description of Artificial Sequence: Synthetic oligonucleotide
ttcctcaccttactttccggaacatt

SEQ ID NO: 760 - DNA, 26 bp, Artificial Sequence
  Description of Artificial Sequence: Synthetic oligonucleotide
tgtgtcaaagctcttggcagacgacc

SEQ ID NO: 761 - DNA, 26 bp, Artificial Sequence
  Description of Artificial Sequence: Synthetic oligonucleotide
gtggccatggttctgccacgaagcac

SEQ ID NO: 762 - DNA, 26 bp, Artificial Sequence
  Description of Artificial Sequence: Synthetic oligonucleotide
tcgaccattaagctcagtagagttgt

SEQ ID NO: 763 - DNA, 26 bp, Artificial Sequence
  Description of Artificial Sequence: Synthetic oligonucleotide
cacgtcataggcctagctcgatcaag

SEQ ID NO: 764 - DNA, 26 bp, Artificial Sequence
  Description of Artificial Sequence: Synthetic oligonucleotide
tgaagcataagtcttctgggaaatta

SEQ ID NO: 765 - DNA, 26 bp, Artificial Sequence
  Description of Artificial Sequence: Synthetic oligonucleotide
acggacaatgcgctattaggatggag

SEQ ID NO: 766 - DNA, 26 bp, Artificial Sequence
  Description of Artificial Sequence: Synthetic oligonucleotide
gtgtgcaatatcctgactagatatgt

SEQ ID NO: 767 - DNA, 26 bp, Artificial Sequence
  Description of Artificial Sequence: Synthetic oligonucleotide
acacacagcgctctcgttcgaggaac

SEQ ID NO: 768 - DNA, 26 bp, Artificial Sequence
  Description of Artificial Sequence: Synthetic oligonucleotide
agcgccaggtgacttcgatgaactag

SEQ ID NO: 769 - DNA, 26 bp, Artificial Sequence
  Description of Artificial Sequence: Synthetic oligonucleotide
caaggcactatccttaccagacaatg

SEQ ID NO: 770 - DNA, 26 bp, Artificial Sequence
  Description of Artificial Sequence: Synthetic oligonucleotide
tgcgtcaccaggcttggtagatacca

SEQ ID NO: 771 - DNA, 26 bp, Artificial Sequence
  Description of Artificial Sequence: Synthetic oligonucleotide
aggtgcacgtaactgctctgacgttg

SEQ ID NO: 772 - DNA, 26 bp, Artificial Sequence
  Description of Artificial Sequence: Synthetic oligonucleotide
gcagccaaacgactgtctcgagtgaa

SEQ ID NO: 773 - DNA, 26 bp, Artificial Sequence
  Description of Artificial Sequence: Synthetic oligonucleotide
atcctcatgtcgctaaggcgacacct

SEQ ID NO: 774 - DNA, 26 bp, Artificial Sequence
  Description of Artificial Sequence: Synthetic oligonucleotide
gaaggcatacacctctgtggaagcta

SEQ ID NO: 775 - DNA, 26 bp, Artificial Sequence
  Description of Artificial Sequence: Synthetic oligonucleotide
ttggccacaggtcttcacagagatcg

SEQ ID NO: 776 - DNA, 26 bp, Artificial Sequence
  Description of Artificial Sequence: Synthetic oligonucleotide
aggcccaagacactagaaggaccaat

SEQ ID NO: 777 - DNA, 26 bp, Artificial Sequence
  Description of Artificial Sequence: Synthetic oligonucleotide
agcatcataactctactgcgaagccg

SEQ ID NO: 778 - DNA, 26 bp, Artificial Sequence
  Description of Artificial Sequence: Synthetic oligonucleotide
attaccatcaccctaacatgactagt

SEQ ID NO: 779 - DNA, 26 bp, Artificial Sequence
  Description of Artificial Sequence: Synthetic oligonucleotide
gcgcacagagtactccttagactatg

SEQ ID NO: 780 - DNA, 26 bp, Artificial Sequence
  Description of Artificial Sequence: Synthetic oligonucleotide
cgccacatacctctgtggcgagagac

SEQ ID NO: 781 - DNA, 26 bp, Artificial Sequence
  Description of Artificial Sequence: Synthetic oligonucleotide
gcaggcactggactgccaggaatcca

SEQ ID NO: 782 - DNA, 26 bp, Artificial Sequence
  Description of Artificial Sequence: Synthetic oligonucleotide
gttatcaatggcctacacagaatatc

SEQ ID NO: 783 - DNA, 26 bp, Artificial Sequence
  Description of Artificial Sequence: Synthetic oligonucleotide
cactccagcactcttggaggagtaat

SEQ ID NO: 784 - DNA, 26 bp, Artificial Sequence
  Description of Artificial Sequence: Synthetic oligonucleotide
accggcactcagctccttcgaacgta

SEQ ID NO: 785 - DNA, 26 bp, Artificial Sequence
  Description of Artificial Sequence: Synthetic oligonucleotide
atagacaccgttctctatagacgcgg

SEQ ID NO: 786 - DNA, 26 bp, Artificial Sequence
  Description of Artificial Sequence: Synthetic oligonucleotide
tgaaccagcaacctgttgcgaagttg

SEQ ID NO: 787 - DNA, 26 bp, Artificial Sequence
  Description of Artificial Sequence: Synthetic oligonucleotide
gtggtcatgaagctttatggacgcct

SEQ ID NO: 788 - DNA, 26 bp, Artificial Sequence
  Description of Artificial Sequence: Synthetic oligonucleotide
actgacaatagacttctcagagtaca

SEQ ID NO: 789 - DNA, 26 bp, Artificial Sequence
  Description of Artificial Sequence: Synthetic oligonucleotide
ggacgcatcttgctagtatgaacgga

SEQ ID NO: 790 - DNA, 26 bp, Artificial Sequence
  Description of Artificial Sequence: Synthetic oligonucleotide
gttgtcaactcactacgctgatggac

SEQ ID NO: 791 - DNA, 26 bp, Artificial Sequence
  Description of Artificial Sequence: Synthetic oligonucleotide
agaaccacgcggctggagtgaagatt

SEQ ID NO: 792 - DNA, 26 bp, Artificial Sequence
  Description of Artificial Sequence: Synthetic oligonucleotide
cagtacatcaatcttacacgagctcc

SEQ ID NO: 793 - DNA, 26 bp, Artificial Sequence
  Description of Artificial Sequence: Synthetic oligonucleotide
tccatcaaatcccttccgagatagag

SEQ ID NO: 794 - DNA, 26 bp, Artificial Sequence
  Description of Artificial Sequence: Synthetic oligonucleotide
atgagcaaaccactctcaagaggccg

SEQ ID NO: 795 - DNA, 26 bp, Artificial Sequence
  Description of Artificial Sequence: Synthetic oligonucleotide
tcgtgcagttgactcaagtgatcata

SEQ ID NO: 796 - DNA, 26 bp, Artificial Sequence
  Description of Artificial Sequence: Synthetic oligonucleotide
caagtcatcatactaatccgattagg

SEQ ID NO: 797 - DNA, 26 bp, Artificial Sequence
  Description of Artificial Sequence: Synthetic oligonucleotide
cttaacaccactctggtgggaaatac

SEQ ID NO: 798 - DNA, 26 bp, Artificial Sequence
  Description of Artificial Sequence: Synthetic oligonucleotide
cgctcacagttcactcgtgtggagcg

SEQ ID NO: 799 - DNA, 26 bp, Artificial Sequence
  Description of Artificial Sequence: Synthetic oligonucleotide
tatctacgacctacctacatgagata

SEQ ID NO: 800 - DNA, 26 bp, Artificial Sequence
  Description of Artificial Sequence: Synthetic oligonucleotide
atatgacagacgactatagtgtagct

SEQ ID NO: 801 - DNA, 26 bp, Artificial Sequence
  Description of Artificial Sequence: Synthetic oligonucleotide
cttatacggaatactgccttgggtgg

SEQ ID NO: 802 - DNA, 26 bp, Artificial Sequence
  Description of Artificial Sequence: Synthetic oligonucleotide
taatcactcgtcacacatttgatcct

SEQ ID NO: 803 - DNA, 26 bp, Artificial Sequence
  Description of Artificial Sequence: Synthetic oligonucleotide
gcgcgacatgttacgtccatgcttgt

SEQ ID NO: 804 - DNA, 26 bp, Artificial Sequence
  Description of Artificial Sequence: Synthetic oligonucleotide
agagcacactagactggaatgcagta

SEQ ID NO: 805 - DNA, 26 bp, Artificial Sequence
  Description of Artificial Sequence: Synthetic oligonucleotide
tgcctactgatcacccttgtgttaat

SEQ ID NO: 806 - DNA, 26 bp, Artificial Sequence
  Description of Artificial Sequence: Synthetic oligonucleotide
ctactaccagtcacgttgatgtagtg

SEQ ID NO: 807 - DNA, 26 bp, Artificial Sequence
  Description of Artificial Sequence: Synthetic oligonucleotide
tcgtcactgactacaccagtgcgaca

SEQ ID NO: 808 - DNA, 26 bp, Artificial Sequence
  Description of Artificial Sequence: Synthetic oligonucleotide
gaacaactacggaccatactgactgt

SEQ ID NO: 809 - DNA, 26 bp, Artificial Sequence
  Description of Artificial Sequence: Synthetic oligonucleotide
cctatacgactcacgtgtgtggcgct

SEQ ID NO: 810 - DNA, 26 bp, Artificial Sequence
  Description of Artificial Sequence: Synthetic oligonucleotide
taatgacgcaagacatcactggaagg

SEQ ID NO: 811 - DNA, 26 bp, Artificial Sequence
  Description of Artificial Sequence: Synthetic oligonucleotide
gtgccacgcttcaccggcttgctact

SEQ ID NO: 812 - DNA, 26 bp, Artificial Sequence
  Description of Artificial Sequence: Synthetic oligonucleotide
cggcaacatggaacgaatgtgcacga

SEQ ID NO: 813 - DNA, 26 bp, Artificial Sequence
  Description of Artificial Sequence: Synthetic oligonucleotide
gccgtacaaccgacaagactgtatag

SEQ ID NO: 814 - DNA, 26 bp, Artificial Sequence
  Description of Artificial Sequence: Synthetic oligonucleotide
aaccaacttctcactcggctgagcaa

SEQ ID NO: 815 - DNA, 26 bp, Artificial Sequence
  Description of Artificial Sequence: Synthetic oligonucleotide
ggttgaccctctacctaattggatgg

SEQ ID NO: 816 - DNA, 26 bp, Artificial Sequence
  Description of Artificial Sequence: Synthetic oligonucleotide
ctaatacgatggacggttgtgcctct

SEQ ID NO: 817 - DNA, 26 bp, Artificial Sequence
  Description of Artificial Sequence: Synthetic oligonucleotide
tcggcacctatcaccgcactgatggc

SEQ ID NO: 818 - DNA, 26 bp, Artificial Sequence
  Description of Artificial Sequence: Synthetic oligonucleotide
agtcaacaccatacggccttggtcct

SEQ ID NO: 819 - DNA, 26 bp, Artificial Sequence
  Description of Artificial Sequence: Synthetic oligonucleotide
gagcgaccaataacctgtgtgttagg

SEQ ID NO: 820 - DNA, 26 bp, Artificial Sequence
  Description of Artificial Sequence: Synthetic oligonucleotide
aacaaacggcgtactaaggtgaacgt

SEQ ID NO: 821 - DNA, 26 bp, Artificial Sequence
  Description of Artificial Sequence: Synthetic oligonucleotide
gtatgactagaaacctaactgtgtaa

SEQ ID NO: 822 - DNA, 26 bp, Artificial Sequence
  Description of Artificial Sequence: Synthetic oligonucleotide
ttctaactggttacggcgatggatgg

SEQ ID NO: 823 - DNA, 26 bp, Artificial Sequence
  Description of Artificial Sequence: Synthetic oligonucleotide
cctcgaccaaccacaatagtgagcaa

SEQ ID NO: 824 - DNA, 26 bp, Artificial Sequence
  Description of Artificial Sequence: Synthetic oligonucleotide
tggatacgcttaactcaattgccatt

SEQ ID NO: 825 - DNA, 26 bp, Artificial Sequence
  Description of Artificial Sequence: Synthetic oligonucleotide
atgtcacgtggtactcgtatgtgcgg

SEQ ID NO: 826 - DNA, 26 bp, Artificial Sequence
  Description of Artificial Sequence: Synthetic oligonucleotide
agagtacgcggcactccgatgcctcg

SEQ ID NO: 827 - DNA, 26 bp, Artificial Sequence
  Description of Artificial Sequence: Synthetic oligonucleotide
tgcctacggtggaccttattgggaat

SEQ ID NO: 828 - DNA, 26 bp, Artificial Sequence
  Description of Artificial Sequence: Synthetic oligonucleotide
tgcgtacgtcacacgcttatgcggac

SEQ ID NO: 829 - DNA, 26 bp, Artificial Sequence
  Description of Artificial Sequence: Synthetic oligonucleotide
catacacactgtacgaacatgtacgg

SEQ ID NO: 830 - DNA, 26 bp, Artificial Sequence
  Description of Artificial Sequence: Synthetic oligonucleotide
cgtatacaatcaacgtcgatgttaca

SEQ ID NO: 831 - DNA, 26 bp, Artificial Sequence
  Description of Artificial Sequence: Synthetic oligonucleotide
tacgcacggctgacactagtgccgtg

SEQ ID NO: 832 - DNA, 26 bp, Artificial Sequence
  Description of Artificial Sequence: Synthetic oligonucleotide
gcgagacttaccacaagtttgggtga

SEQ ID NO: 833 - DNA, 26 bp, Artificial Sequence
  Description of Artificial Sequence: Synthetic oligonucleotide
tacggacccggtactggcatgatatt

SEQ ID NO: 834 - DNA, 26 bp, Artificial Sequence
  Description of Artificial Sequence: Synthetic oligonucleotide
gtcgaacttacaacgatcatgccgcg

SEQ ID NO: 835 - DNA, 26 bp, Artificial Sequence
  Description of Artificial Sequence: Synthetic oligonucleotide
ctgtcactgcacactaccatgtccgt

SEQ ID NO: 836 - DNA, 26 bp, Artificial Sequence
  Description of Artificial Sequence: Synthetic oligonucleotide
cagccacgattgacgctgttgaggaa

SEQ ID NO: 837 - DNA, 26 bp, Artificial Sequence
  Description of Artificial Sequence: Synthetic oligonucleotide
tgactacacataaccgcactgtaatg

SEQ ID NO: 838 - DNA, 26 bp, Artificial Sequence
  Description of Artificial Sequence: Synthetic oligonucleotide
attgcaccgagtacgacaatgctgaa

SEQ ID NO: 839 - DNA, 26 bp, Artificial Sequence
  Description of Artificial Sequence: Synthetic oligonucleotide
gccatactagacacagtggtgtcagg

SEQ ID NO: 840 - DNA, 26 bp, Artificial Sequence
  Description of Artificial Sequence: Synthetic oligonucleotide
ggcgaacgatggacttctatgtggtt

SEQ ID NO: 841 - DNA, 26 bp, Artificial Sequence
  Description of Artificial Sequence: Synthetic oligonucleotide
tggctaccgcagacaatcctgggcca

SEQ ID NO: 842 - DNA, 26 bp, Artificial Sequence
  Description of Artificial Sequence: Synthetic oligonucleotide
tagaaactaacgacccatatgaggtt

SEQ ID NO: 843 - DNA, 26 bp, Artificial Sequence
  Description of Artificial Sequence: Synthetic oligonucleotide
taatgacgatctacatctctgtacca

SEQ ID NO: 844 - DNA, 26 bp, Artificial Sequence
  Description of Artificial Sequence: Synthetic oligonucleotide
tatccacaggacaccggtgtggcgaa

SEQ ID NO: 845 - DNA, 26 bp, Artificial Sequence
  Description of Artificial Sequence: Synthetic oligonucleotide
agtgcaccactgactaacatgatagg

SEQ ID NO: 846 - DNA, 26 bp, Artificial Sequence
  Description of Artificial Sequence: Synthetic oligonucleotide
gtgcaacacactacctggttgacacg

SEQ ID NO: 847 - DNA, 26 bp, Artificial Sequence
  Description of Artificial Sequence: Synthetic oligonucleotide
acatgacgtgtcactcaactggtgta

SEQ ID NO: 848 - DNA, 26 bp, Artificial Sequence
  Description of Artificial Sequence: Synthetic oligonucleotide
gacagacacaggacactgttgtgtga

SEQ ID NO: 849 - DNA, 26 bp, Artificial Sequence
  Description of Artificial Sequence: Synthetic oligonucleotide
tcttaaccatcaacgtgcgtgtcctt

SEQ ID NO: 850 - DNA, 26 bp, Artificial Sequence
  Description of Artificial Sequence: Synthetic oligonucleotide
ttacaacattccacagcactgatcct

SEQ ID NO: 851 - DNA, 26 bp, Artificial Sequence
  Description of Artificial Sequence: Synthetic oligonucleotide
aagctactatgcacttccgtgtcgca

SEQ ID NO: 852 - DNA, 26 bp, Artificial Sequence
  Description of Artificial Sequence: Synthetic oligonucleotide
tattcacctcagaccttaatgccact

SEQ ID NO: 853 - DNA, 26 bp, Artificial Sequence
  Description of Artificial Sequence: Synthetic oligonucleotide
ctcgtacgcgttacgcctctgggata

SEQ ID NO: 854 - DNA, 26 bp, Artificial Sequence
  Description of Artificial Sequence: Synthetic oligonucleotide
ttaggacatagaaccgtcgtgactgg

SEQ ID NO: 855 - DNA, 26 bp, Artificial Sequence
  Description of Artificial Sequence: Synthetic oligonucleotide
ccgaaacgcgagactactatggtcaa

SEQ ID NO: 856 - DNA, 26 bp, Artificial Sequence
  Description of Artificial Sequence: Synthetic oligonucleotide
ggaccacaacagacatagatgccgtt

SEQ ID NO: 857 - DNA, 26 bp, Artificial Sequence
  Description of Artificial Sequence: Synthetic oligonucleotide
ttccaacggtaaacacagttgtccag

SEQ ID NO: 858 - DNA, 26 bp, Artificial Sequence
  Description of Artificial Sequence: Synthetic oligonucleotide
tgattacagccaacaggcatgtgtag

SEQ ID NO: 859 - DNA, 26 bp, Artificial Sequence
  Description of Artificial Sequence: Synthetic oligonucleotide
taacaacgtgttacgcaagtgtctca

SEQ ID NO: 860 - DNA, 26 bp, Artificial Sequence
  Description of Artificial Sequence: Synthetic oligonucleotide
accgcacgcaatacttggctgtccgc

SEQ ID NO: 861 - DNA, 26 bp, Artificial Sequence
  Description of Artificial Sequence: Synthetic oligonucleotide
gttcgaccgccaacaactgtgatact

SEQ ID NO: 862 - DNA, 26 bp, Artificial Sequence
  Description of Artificial Sequence: Synthetic oligonucleotide
agacaaccattaacgtaagtggcata

SEQ ID NO: 863 - DNA, 26 bp, Artificial Sequence
  Description of Artificial Sequence: Synthetic oligonucleotide
gcgttacggtatacaattgtgctgcg

SEQ ID NO: 864 - DNA, 26 bp, Artificial Sequence
  Description of Artificial Sequence: Synthetic oligonucleotide
agcacacatcctacttacatgattcc

SEQ ID NO: 865 - DNA, 26 bp, Artificial Sequence
  Description of Artificial Sequence: Synthetic oligonucleotide
ttgttacccgtgacaaccttgagcac

SEQ ID NO: 866 - DNA, 26 bp, Artificial Sequence
  Description of Artificial Sequence: Synthetic oligonucleotide
aagtaacctccaactctgttggtgga

SEQ ID NO: 867 - DNA, 26 bp, Artificial Sequence
  Description of Artificial Sequence: Synthetic oligonucleotide
acgtcacaatacacggaattgtccaa

SEQ ID NO: 868 - DNA, 26 bp, Artificial Sequence
  Description of Artificial Sequence: Synthetic oligonucleotide
ggtgtacacaagacaagcgtgcgctt

SEQ ID NO: 869 - DNA, 26 bp, Artificial Sequence
  Description of Artificial Sequence: Synthetic oligonucleotide
ccaccactgtgtactgagctggttgt

SEQ ID NO: 870 - DNA, 26 bp, Artificial Sequence
  Description of Artificial Sequence: Synthetic oligonucleotide
gttccacgcaggacatcattgaggct

SEQ ID NO: 871 - DNA, 26 bp, Artificial Sequence
  Description of Artificial Sequence: Synthetic oligonucleotide
accttacatgaaactgttatggaagg

SEQ ID NO: 872 - DNA, 26 bp, Artificial Sequence
  Description of Artificial Sequence: Synthetic oligonucleotide
cgctgaccagagacgatggtgatgta

SEQ ID NO: 873 - DNA, 26 bp, Artificial Sequence
  Description of Artificial Sequence: Synthetic oligonucleotide
gtagaacgtcagacacggctgcgtca

SEQ ID NO: 874 - DNA, 26 bp, Artificial Sequence
  Description of Artificial Sequence: Synthetic oligonucleotide
ggataacccagaaccgttgtgcttac

SEQ ID NO: 875 - DNA, 26 bp, Artificial Sequence
  Description of Artificial Sequence: Synthetic oligonucleotide
cgcacactaatgactgacttgacata

SEQ ID NO: 876 - DNA, 26 bp, Artificial Sequence
  Description of Artificial Sequence: Synthetic oligonucleotide
tcctgacaccgtaccggcctgtcgtt

SEQ ID NO: 877 - DNA, 26 bp, Artificial Sequence
  Description of Artificial Sequence: Synthetic oligonucleotide
ctggcacttgccaccaagctgatccg

SEQ ID NO: 878 - DNA, 26 bp, Artificial Sequence
  Description of Artificial Sequence: Synthetic oligonucleotide
accagaccgacaactcgtctgtgact

SEQ ID NO: 879 - DNA, 26 bp, Artificial Sequence
  Description of Artificial Sequence: Synthetic oligonucleotide
ttgtaacacggtacctcattgagcga

SEQ ID NO: 880 - DNA, 26 bp, Artificial Sequence
  Description of Artificial Sequence: Synthetic oligonucleotide
gtaagacgcataacagacatgcatta

SEQ ID NO: 881 - DNA, 26 bp, Artificial Sequence
  Description of Artificial Sequence: Synthetic oligonucleotide
gtccaaccttgtacgcgcgtgatgtt

SEQ ID NO: 882 - DNA, 26 bp, Artificial Sequence
  Description of Artificial Sequence: Synthetic oligonucleotide
ttaggactaccaaccatgatggtact

SEQ ID NO: 883 - DNA, 26 bp, Artificial Sequence
  Description of Artificial Sequence: Synthetic oligonucleotide
ggaatactccaaacacgtctgaatac

SEQ ID NO: 884 - DNA, 26 bp, Artificial Sequence
  Description of Artificial Sequence: Synthetic oligonucleotide
catgtacagaggacgatactgctcct

SEQ ID NO: 885 - DNA, 26 bp, Artificial Sequence
  Description of Artificial Sequence: Synthetic oligonucleotide
tacacacgctccacatccgtgtaagt

SEQ ID NO: 886 - DNA, 26 bp, Artificial Sequence
  Description of Artificial Sequence: Synthetic oligonucleotide
gcttaaccggacaccgtgttgatctt

SEQ ID NO: 887 - DNA, 26 bp, Artificial Sequence
  Description of Artificial Sequence: Synthetic oligonucleotide
cgcttacgaagtacgaacctgatgaa

SEQ ID NO: 888 - DNA, 26 bp, Artificial Sequence
  Description of Artificial Sequence: Synthetic oligonucleotide
cgcctactctgaacggccatgtcata

SEQ ID NO: 889 - DNA, 26 bp, Artificial Sequence
  Description of Artificial Sequence: Synthetic oligonucleotide
ataccacaacgcacacatatgcttcc

SEQ ID NO: 890 - DNA, 26 bp, Artificial Sequence
  Description of Artificial Sequence: Synthetic oligonucleotide
ctggaactatgtactatgttggcaat

SEQ ID NO: 891 - DNA, 26 bp, Artificial Sequence
  Description of Artificial Sequence: Synthetic oligonucleotide
caatcactatgaacgattatgaggtg

SEQ ID NO: 892 - DNA, 26 bp, Artificial Sequence
  Description of Artificial Sequence: Synthetic oligonucleotide
ggtggacaatacacatgtatggacaa

SEQ ID NO: 893 - DNA, 26 bp, Artificial Sequence
  Description of Artificial Sequence: Synthetic oligonucleotide
tggacacggaggaccacattgcggtg

SEQ ID NO: 894 - DNA, 26 bp, Artificial Sequence
  Description of Artificial Sequence: Synthetic oligonucleotide
ctgacaccggcaaccctgatgtacaa

SEQ ID NO: 895 - DNA, 26 bp, Artificial Sequence
  Description of Artificial Sequence: Synthetic oligonucleotide
gaattacgagtgacttaagtgttgtg

SEQ ID NO: 896 - DNA, 26 bp, Artificial Sequence
  Description of Artificial Sequence: Synthetic oligonucleotide
gcgtgactgagaaccggactgagtga

SEQ ID NO: 897 - DNA, 26 bp, Artificial Sequence
  Description of Artificial Sequence: Synthetic oligonucleotide
tctccacattgaacgcacttgacaac

SEQ ID NO: 898 - DNA, 26 bp, Artificial Sequence
  Description of Artificial Sequence: Synthetic oligonucleotide
acatgaccatatactggtgtgcctgg

SEQ ID NO: 899 - DNA, 26 bp, Artificial Sequence
  Description of Artificial Sequence: Synthetic oligonucleotide
caggcacgccatactccactgggcct

SEQ ID NO: 900 - DNA, 26 bp, Artificial Sequence
  Description of Artificial Sequence: Synthetic oligonucleotide
acataacacggaacttgtatggtgta

SEQ ID NO: 901 - DNA, 26 bp, Artificial Sequence
  Description of Artificial Sequence: Synthetic oligonucleotide
ttaatacagaccacccacgtgacacg

SEQ ID NO: 902 - DNA, 26 bp, Artificial Sequence
  Description of Artificial Sequence: Synthetic oligonucleotide
acgatactgctgactgtgatgtgtat

SEQ ID NO: 903 - DNA, 26 bp, Artificial Sequence
  Description of Artificial Sequence: Synthetic oligonucleotide
ttctaaccagaaacgagcgtgcaata

SEQ ID NO: 904 - DNA, 26 bp, Artificial Sequence
  Description of Artificial Sequence: Synthetic oligonucleotide
tattgaccgttcacatctttgactgt

SEQ ID NO: 905 - DNA, 26 bp, Artificial Sequence
  Description of Artificial Sequence: Synthetic oligonucleotide
catgaacgtactacatgtctggtggt

SEQ ID NO: 906 - DNA, 26 bp, Artificial Sequence
  Description of Artificial Sequence: Synthetic oligonucleotide
taattacctaccacgtagctgcatca

SEQ ID NO: 907 - DNA, 26 bp, Artificial Sequence
  Description of Artificial Sequence: Synthetic oligonucleotide
acgctacaattaactggtttgaagaa

SEQ ID NO: 908 - DNA, 26 bp, Artificial Sequence
  Description of Artificial Sequence: Synthetic oligonucleotide
ccttgacttaatactgttgtgttcgt

SEQ ID NO: 909 - DNA, 26 bp, Artificial Sequence
  Description of Artificial Sequence: Synthetic oligonucleotide
gtagcaccatcaacccaactgaacat

SEQ ID NO: 910 - DNA, 26 bp, Artificial Sequence
  Description of Artificial Sequence: Synthetic oligonucleotide
cttgtacaattcacaccggtgctcag

SEQ ID NO: 911 - DNA, 26 bp, Artificial Sequence
  Description of Artificial Sequence: Synthetic oligonucleotide
tccaaacttctaacgttaatgtctga

SEQ ID NO: 912 - DNA, 26 bp, Artificial Sequence
  Description of Artificial Sequence: Synthetic oligonucleotide
agagcactgcctaccggcttgaacgt

SEQ ID NO: 913 - DNA, 26 bp, Artificial Sequence
  Description of Artificial Sequence: Synthetic oligonucleotide
cttcgacccgatactccaatggaatt

SEQ ID NO: 914 - DNA, 26 bp, Artificial Sequence
  Description of Artificial Sequence: Synthetic oligonucleotide
tcggtaccacggacccgaatgcgttg

SEQ ID NO: 915 - DNA, 26 bp, Artificial Sequence
  Description of Artificial Sequence: Synthetic oligonucleotide
gaacaacagtatactaacctggccga

SEQ ID NO: 916 - DNA, 26 bp, Artificial Sequence
  Description of Artificial Sequence: Synthetic oligonucleotide
aattgacgcggaacctccgtgtgctg

SEQ ID NO: 917 - DNA, 26 bp, Artificial Sequence
  Description of Artificial Sequence: Synthetic oligonucleotide
ggcctacgtcctaccattctgcagct

SEQ ID NO: 918 - DNA, 26 bp, Artificial Sequence
  Description of Artificial Sequence: Synthetic oligonucleotide
taggtactctctacggttatgtgcta

SEQ ID NO: 919 - DNA, 26 bp, Artificial Sequence
  Description of Artificial Sequence: Synthetic oligonucleotide
acacaacatatcacaccactgacggt

SEQ ID NO: 920 - DNA, 26 bp, Artificial Sequence
  Description of Artificial Sequence: Synthetic oligonucleotide
ttcctacgtacgactaggttgtctct

SEQ ID NO: 921 - DNA, 26 bp, Artificial Sequence
  Description of Artificial Sequence: Synthetic oligonucleotide
ggtaaaccgcagactatggtgctcga

SEQ ID NO: 922 - DNA, 26 bp, Artificial Sequence
  Description of Artificial Sequence: Synthetic oligonucleotide
tccacacggcctacctcgttggcgtt

SEQ ID NO: 923 - DNA, 26 bp, Artificial Sequence
  Description of Artificial Sequence: Synthetic oligonucleotide
gatacacctcctacccagttgtggca

SEQ ID NO: 924 - DNA, 26 bp, Artificial Sequence
  Description of Artificial Sequence: Synthetic oligonucleotide
caacgactcagcactgttctggcatt

SEQ ID NO: 925 - DNA, 26 bp, Artificial Sequence
  Description of Artificial Sequence: Synthetic oligonucleotide
cggttacattagacaaccgtgcatcg

SEQ ID NO: 926 - DNA, 26 bp, Artificial Sequence
  Description of Artificial Sequence: Synthetic oligonucleotide
cgcgcacctagaaccgaagtggttaa

SEQ ID NO: 927 - DNA, 26 bp, Artificial Sequence
  Description of Artificial Sequence: Synthetic oligonucleotide
tcttgacgctatacagtgctgcactg

SEQ ID NO: 928 - DNA, 26 bp, Artificial Sequence
  Description of Artificial Sequence: Synthetic oligonucleotide
tcacaacccgaaacgaacatgagtat

SEQ ID NO: 929 - DNA, 26 bp, Artificial Sequence
  Description of Artificial Sequence: Synthetic oligonucleotide
aacgtactacatacacgattgtgctg

SEQ ID NO: 930 - DNA, 26 bp, Artificial Sequence
  Description of Artificial Sequence: Synthetic oligonucleotide
cggccactcgttacatacctgtggat

SEQ ID NO: 931 - DNA, 26 bp, Artificial Sequence
  Description of Artificial Sequence: Synthetic oligonucleotide
cataaaccaccaactccaatgttcta

SEQ ID NO: 932 - DNA, 26 bp, Artificial Sequence
  Description of Artificial Sequence: Synthetic oligonucleotide
acagaacggccaactgagatgcagcg

SEQ ID NO: 933 - DNA, 26 bp, Artificial Sequence
  Description of Artificial Sequence: Synthetic oligonucleotide
tggtgaccctggacacgcttgaatta

SEQ ID NO: 934 - DNA, 26 bp, Artificial Sequence
  Description of Artificial Sequence: Synthetic oligonucleotide
taggaacaccggactatattgtcgag

SEQ ID NO: 935 - DNA, 26 bp, Artificial Sequence
  Description of Artificial Sequence: Synthetic oligonucleotide
aatatactggccaccggtctgcgata

SEQ ID NO: 936 - DNA, 26 bp, Artificial Sequence
  Description of Artificial Sequence: Synthetic oligonucleotide
ataggactattcacacaattgagagt

SEQ ID NO: 937 - DNA, 26 bp, Artificial Sequence
  Description of Artificial Sequence: Synthetic oligonucleotide
ccttcacacgtaaccggtttgattag

SEQ ID NO: 938 - DNA, 26 bp, Artificial Sequence
  Description of Artificial Sequence: Synthetic oligonucleotide
ggccaacataagacgataatgcaagt

SEQ ID NO: 939 - DNA, 26 bp, Artificial Sequence
  Description of Artificial Sequence: Synthetic oligonucleotide
cagtaacgttgtacagttatgtcaca

SEQ ID NO: 940 - DNA, 26 bp, Artificial Sequence
  Description of Artificial Sequence: Synthetic oligonucleotide
ttcatacccaacacttccatgggtaa

SEQ ID NO: 941 - DNA, 26 bp, Artificial Sequence
  Description of Artificial Sequence: Synthetic oligonucleotide
caattacggattaccatgttgagagg

SEQ ID NO: 942 - DNA, 26 bp, Artificial Sequence
  Description of Artificial Sequence: Synthetic oligonucleotide
ggccaactcataacgattgtgtcata

SEQ ID NO: 943 - DNA, 26 bp, Artificial Sequence
  Description of Artificial Sequence: Synthetic oligonucleotide
aattgacctgcgacattcctggctat

SEQ ID NO: 944 - DNA, 26 bp, Artificial Sequence
  Description of Artificial Sequence: Synthetic oligonucleotide
taaggacaacgtacgaccgtgctgtg

SEQ ID NO: 945 - DNA, 26 bp, Artificial Sequence
  Description of Artificial Sequence: Synthetic oligonucleotide
ctataaccgcggactaggatgaccgg

SEQ ID NO: 946 - DNA, 26 bp, Artificial Sequence
  Description of Artificial Sequence: Synthetic oligonucleotide
attcaacgaatcacagcggtgtggac

SEQ ID NO: 947 - DNA, 26 bp, Artificial Sequence
  Description of Artificial Sequence: Synthetic oligonucleotide
gtattacctctaactatagtgattcg

SEQ ID NO: 948 - DNA, 26 bp, Artificial Sequence
  Description of Artificial Sequence: Synthetic oligonucleotide
cctgaactacaaacacagatgggcca

SEQ ID NO: 949 - DNA, 26 bp, Artificial Sequence
  Description of Artificial Sequence: Synthetic oligonucleotide
gaccgacctgtgacattcctgtattg

SEQ ID NO: 950 - DNA, 26 bp, Artificial Sequence
  Description of Artificial Sequence: Synthetic oligonucleotide
ttcagaccgtggactattctgctcag

SEQ ID NO: 951 - DNA, 26 bp, Artificial Sequence
  Description of Artificial Sequence: Synthetic oligonucleotide
aactcaccgaacaccgccttgtctga

SEQ ID NO: 952 - DNA, 26 bp, Artificial Sequence
  Description of Artificial Sequence: Synthetic oligonucleotide
attccacgctatacgcgcatggagta

SEQ ID NO: 953 - DNA, 26 bp, Artificial Sequence
  Description of Artificial Sequence: Synthetic oligonucleotide
tgaatacattgcacggcgctgcaatt

SEQ ID NO: 954 - DNA, 26 bp, Artificial Sequence
  Description of Artificial Sequence: Synthetic oligonucleotide
cgcaaactctagacagatatgtggcg

SEQ ID NO: 955 - DNA, 26 bp, Artificial Sequence
  Description of Artificial Sequence: Synthetic oligonucleotide
aaccgaccatcgaccctgctgttggt

SEQ ID NO: 956 - DNA, 26 bp, Artificial Sequence
  Description of Artificial Sequence: Synthetic oligonucleotide
ctagtacccggaacgacgatgacaat

SEQ ID NO: 957 - DNA, 26 bp, Artificial Sequence
  Description of Artificial Sequence: Synthetic oligonucleotide
gctccacgtcacactggcgtggtcca

SEQ ID NO: 958 - DNA, 26 bp, Artificial Sequence
  Description of Artificial Sequence: Synthetic oligonucleotide
agatgacgaattaccttcatggttac

SEQ ID NO: 959 - DNA, 26 bp, Artificial Sequence
  Description of Artificial Sequence: Synthetic oligonucleotide
acaccacgttaaactcctgtgaccgt

SEQ ID NO: 960 - DNA, 26 bp, Artificial Sequence
  Description of Artificial Sequence: Synthetic oligonucleotide
gataaaccaagtaccgcgctgctaga

SEQ ID NO: 961 - DNA, 26 bp, Artificial Sequence
  Description of Artificial Sequence: Synthetic oligonucleotide
ctggtacacacgacaggattgaagtt

SEQ ID NO: 962 - DNA, 26 bp, Artificial Sequence
  Description of Artificial Sequence: Synthetic oligonucleotide
cgaagacgttaaacaggcctgagaca

SEQ ID NO: 963 - DNA, 26 bp, Artificial Sequence
  Description of Artificial Sequence: Synthetic oligonucleotide
atcgcacatatgacccttgtgaacgg

SEQ ID NO: 964 - DNA, 26 bp, Artificial Sequence
  Description of Artificial Sequence: Synthetic oligonucleotide
atcatacaggctaccaccatgcctac

SEQ ID NO: 965 - DNA, 26 bp, Artificial Sequence
  Description of Artificial Sequence: Synthetic oligonucleotide
gattgactcataacttgcttgtgtat

SEQ ID NO: 966 - DNA, 26 bp, Artificial Sequence
  Description of Artificial Sequence: Synthetic oligonucleotide
ccaacacaacataccaatctgtatga

SEQ ID NO: 967 - DNA, 26 bp, Artificial Sequence
  Description of Artificial Sequence: Synthetic oligonucleotide
ttggtacggtgcactggtatgctgat

SEQ ID NO: 968 - DNA, 26 bp, Artificial Sequence
  Description of Artificial Sequence: Synthetic oligonucleotide
gcgaaaccgcctacttcattgccaac

SEQ ID NO: 969 - DNA, 26 bp, Artificial Sequence
  Description of Artificial Sequence: Synthetic oligonucleotide
caaccacggaggaccataatgcacca

SEQ ID NO: 970 - DNA, 26 bp, Artificial Sequence
  Description of Artificial Sequence: Synthetic oligonucleotide
agcggactggacactcctatgttagc

SEQ ID NO: 971 - DNA, 26 bp, Artificial Sequence
  Description of Artificial Sequence: Synthetic oligonucleotide
gacgaacacaatactctcttgagatt

SEQ ID NO: 972 - DNA, 26 bp, Artificial Sequence
  Description of Artificial Sequence: Synthetic oligonucleotide
ccactacggtccaccgcgatggccta

SEQ ID NO: 973 - DNA, 26 bp, Artificial Sequence
  Description of Artificial Sequence: Synthetic oligonucleotide
tgttaacgaaggacgataatggctct

SEQ ID NO: 974 - DNA, 26 bp, Artificial Sequence
  Description of Artificial Sequence: Synthetic oligonucleotide
tatatactcgagacgagattggtcga

SEQ ID NO: 975 - DNA, 26 bp, Artificial Sequence
  Description of Artificial Sequence: Synthetic oligonucleotide
cgcgaaccgatcacctggatgtatgt

SEQ ID NO: 976 - DNA, 26 bp, Artificial Sequence
  Description of Artificial Sequence: Synthetic oligonucleotide
gcctcacggataacggccatgataag

SEQ ID NO: 977 - DNA, 26 bp, Artificial Sequence
  Description of Artificial Sequence: Synthetic oligonucleotide
tgagaaccagcgacattactgtcacc

SEQ ID NO: 978 - DNA, 26 bp, Artificial Sequence
  Description of Artificial Sequence: Synthetic oligonucleotide
tgttcacgcattacaattgtggcgga

SEQ ID NO: 979 - DNA, 26 bp, Artificial Sequence
  Description of Artificial Sequence: Synthetic oligonucleotide
tccaaacgaattacttgtctgaactt

SEQ ID NO: 980 - DNA, 26 bp, Artificial Sequence
  Description of Artificial Sequence: Synthetic oligonucleotide
gctgtacaggaaacggcgatgattct

SEQ ID NO: 981 - DNA, 26 bp, Artificial Sequence
  Description of Artificial Sequence: Synthetic oligonucleotide
ataccactggataccaacgtgtcagc

SEQ ID NO: 982 - DNA, 26 bp, Artificial Sequence
  Description of Artificial Sequence: Synthetic oligonucleotide
gttggacaccgtactcttatgcatca

SEQ ID NO: 983 - DNA, 26 bp, Artificial Sequence
  Description of Artificial Sequence: Synthetic oligonucleotide
accaaacgttacaccgccatgtacct

SEQ ID NO: 984 - DNA, 26 bp, Artificial Sequence
  Description of Artificial Sequence: Synthetic oligonucleotide
gtgtgacgcgctacctaattggtctt

SEQ ID NO: 985 - DNA, 26 bp, Artificial Sequence
  Description of Artificial Sequence: Synthetic oligonucleotide
ggcagactagcaaccaacctgggagg

SEQ ID NO: 986 - DNA, 26 bp, Artificial Sequence
  Description of Artificial Sequence: Synthetic oligonucleotide
tgcggactgttgacggcagtgtagca

SEQ ID NO: 987 - DNA, 26 bp, Artificial Sequence
  Description of Artificial Sequence: Synthetic oligonucleotide
gattaacaggtgacttaggtgataga

SEQ ID NO: 988 - DNA, 26 bp, Artificial Sequence
  Description of Artificial Sequence: Synthetic oligonucleotide
caacaacttcaaaccgcaatgtctag

SEQ ID NO: 989 - DNA, 26 bp, Artificial Sequence
  Description of Artificial Sequence: Synthetic oligonucleotide
gtgttacaccggacgagtttggtact

SEQ ID NO: 990 - DNA, 203 bp, Artificial Sequence
  Description of Artificial Sequence: Synthetic polynucleotide
aatgatacggcgaccaccgagatctacacagaacgccagcagcgaacaaccgctcacagttctgtcgtgacgagcgaatttaaggtcttccttgccatgttgagtgagagcggtgaaccaagacgcagtattattgggtaaacgcgcatattgcgtgttgtggtcctgtgcttgctcgtatctcgtatgccgtcttctgcttg

SEQ ID NO: 991 - DNA, 25 bp, Artificial Sequence
  Description of Artificial Sequence: Synthetic oligonucleotide
catgttgagtgagagcggtgaacca

SEQ ID NO: 992 - DNA, 10 bp, Artificial Sequence
  Description of Artificial Sequence: Synthetic oligonucleotide
gcgcatattg

SEQ ID NO: 993 - DNA, 70 bp, Artificial Sequence
  Description of Artificial Sequence: Synthetic oligonucleotide
cgctcacagttctgtcgtgacgagcgaatttaaggtcttccttgccatgttgagtgagagcggtgaacca

SEQ ID NO: 994 - DNA, 58 bp, Artificial Sequence
  Description of Artificial Sequence: Synthetic oligonucleotide
caatatgcgcgtttacccaataatactgcgtcttggttcaccgctctcactcaacatg

SEQ ID NO: 995 - DNA, 71 bp, Severe acute respiratory syndrome coronavirus 2
ttcttacctttcttttccaatgttacttggttccatgctatacatgtctctgggaccaatggtactaagag

SEQ ID NO: 996 - DNA, 24 bp, Artificial Sequence
  Description of Artificial Sequence: Synthetic oligonucleotide
accaggaacaaatacttctaacca

SEQ ID NO: 997 - DNA, 65 bp, Severe acute respiratory syndrome coronavirus 2
accaggaacaaatacttctaaccaggttgctgttctttatcaggatgttaactgcacagaagtcc

SEQ ID NO: 998 - DNA, 26 bp, Artificial Sequence
  Description of Artificial Sequence: Synthetic oligonucleotide
  modified_base (1)..(5): a, c, t, g, unknown or other
  modified_base (8)..(12): a, c, t, g, unknown or other
  modified_base (15)..(19): a, c, t, g, unknown or other
  modified_base (22)..(26): a, c, t, g, unknown or other
nnnnnacnnnnntgnnnnnacnnnnn

SEQ ID NO: 999 - DNA, 26 bp, Artificial Sequence
  Description of Artificial Sequence: Synthetic oligonucleotide
  modified_base (1)..(5): a, c, t, g, unknown or other
  modified_base (8)..(12): a, c, t, g, unknown or other
  modified_base (15)..(19): a, c, t, g, unknown or other
  modified_base (22)..(26): a, c, t, g, unknown or other
nnnnncannnnnctnnnnngannnnn

SEQ ID NO: 1000 - DNA, 26 bp, Artificial Sequence
  Description of Artificial Sequence: Synthetic oligonucleotide
  modified_base (1)..(5): a, c, t, g, unknown or other
  modified_base (8)..(12): a, c, t, g, unknown or other
  modified_base (15)..(19): a, c, t, g, unknown or other
  modified_base (22)..(26): a, c, t, g, unknown or other
nnnnnacnnnnnacnnnnntgnnnnn

SEQ ID NO: 1001 - DNA, 1260 bp, Severe acute respiratory syndrome coronavirus 2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 1002 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 1003 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 1004 - DNA, 77 bp, Severe acute respiratory syndrome coronavirus 2
cagtgttataacaccaggaacaaatacttctaaccaggttgctgttctttatcaggatgttaactgcacagaagtcc

SEQ ID NO: 1005 - DNA, 61 bp, Artificial Sequence
  Description of Artificial Sequence: Synthetic primer
cgccagcagcgaacaacgctcacagttctgtcgtgacgagcgaatttaaggtcttccttgc

SEQ ID NO: 1006 - DNA, 4251 bp, Homo sapiens
atgggacttcagcatggcggtgtttgcagatttggacctgcgagcgggttctgacctgaaggctctgcgcggacttgtggagacagccgctcaccttggctattcagttgttgctatcaatcatatcgttgactttaaggaaaagaaacaggaaattgaaaaaccagtagctgtttctgaactcttcacaactttgccaattgtacagggaaaatcaagaccaattaaaattttaactagattaacaattattgtctcggatccatctcactgcaatgttttgagagcaacttcttcaagggcccggctctatgatgttgttgcagtttttccaaagacagaaaagctttttcatattgcttgcacacatttagatgtggatttagtctgcataactgtaacagagaaactaccattttacttcaaaagacctcctattaatgtggcgattgaccgaggcctggcttttgaacttgtctatagccctgctatcaaagactccacaatgagaaggtatacaatttccagtgccctcaatttgatgcaaatctgcaaaggaaagaatgtaattatatctagtgctgcagaaaggcctttagaaataagagggccatatgacgtggcaaatctaggcttgctgtttgggctctctgaaagtgacgccaaggctgcggtgtccaccaactgccgagcagcgcttctccatggagaaactagaaaaactgcttttggaattatctctacagtgaagaaacctcggccatcagaaggagatgaagattgtcttccagcttccaagaaagccaagtggtctcactctgtcacccaggctggagtgcagtggcacaatctcggctcactgcaacctttgcctcttgggctcaagccatcctcccacctcagcctcccaagaactagaattcaacaaagacaacttttgatctctcatcagagagatcatactcccaagaacaggctttgacccttctttaaaagaggattgtcctgggctgatgagagtcactttacctgaagatacctgggaagttttgtctcctctgaggttggcccatggccagtgactgatgcaggacattacagcctggcccactggcctcggtggtacaatttatgctcctgagcaccccatgggattacgctgtgtgtggatttctcctgaaaccacatatttgcctcttctgccctgttctgttttttctcattcccttatagaggactctcagtaagtcacttacacaagaatcctaatttgaaatgctgcttccaggagacctgacttagaaaattggacaaataaagttgatttttttaaatgtccagtaacatgaagatgctgaactttcctagtcatttagggggaaatcaccacaaatatatctggctgatcaggttgaaagttaaaagaaaaaaagatttataaagtgggtattttcaagatggtgtggagggagatacaatttgatgcaagtcttatacttttgatgtcaatttattcttcagaaataactggttaattataaagggtggatggataaggatattcactgcaacaatgtcttaaatgtgaaaatggaaacaacctaaatacccaataataacaggattaaataattcattgtacattaaaagaatactgtctataaagatgtctagaataagttgttcagttgaagttgtaaagctaaatacacaatacccataatgtgcacagaaataatcagaatgtcatgaaaccaggattattggtgatgtgttgcttcctttgttactcatttctgtattggcataatgagtattgggtgttcaagaggagggggaaggaagtatgacagatgttatggggaaaaagcaaagtacaacaggaagacaccttgggggaactaatagaatctaaggactcaaggatggcttcctggaggaaatacagctagaacaaaggggaggaatgagaagtgatgtgatggcgtggagtgggctgtagtgggaagaagagtcttccagggagctggcacagtatgtgaaaacagtaaagcaagtgcctggattttttaaggaactgaaaatttagttgagttgaaatttagagtttggctaggaaggttatgagagataagaataaagagttaacagcagccagattttaaggattttataagacatttttaggagtttttatttcatcctgagagaaatgtgaagccatcgaagggttgaaagaggagagtgagttgatcagcattgcattttagaaaaatccctctatctgcaacttgaaaaacattctggaggtaagcaagcctggaggccaggagcctaggagggctatttgatccagatgagaagtaatggtgacctgaactagggcagaggcacctaggattggaaaacatggacagatcacagcactacttatgtagtatacttggtaagacctggttgtttaaaagagaaggatgagggaaagaaggtcaaaaacaacttctaggactctccattggccagtgtggtgtgcccttcactgaagagcaaacacaaaatgaaagattgtgggcaaagagttgagtcagtgagaaggcaaggagagaaccttataaaaaaattgactatgtgattaaaaacttaaaaatttcccccaacgtgtttatcttttccattagcagaaataactaagagttgtcttaattctaatgggatttattccatattgtctctcatgccctctacctagttattagtgcaaatatttatatgtggcaacataaaactttttaactctttattctcttctctcgtgtaccctcccagctctttaggggaggtggatttgaggcagataccataaagaaaagttggtcacatggtggtaacacgttgaagttatgccacatgagacatcagcactggcaagagaaatgtctgtgttgtagatgtttcacttggaagaaattgaaggaccctgagccttaaaagtctgacaaacttaagccaggacccctgtggggaaggtagaggggccaacaaacaagattgggagtcagagagataacaatgaaatccccaatgcctgtgggaggtggactccctggattagtactagacagaaaaggtacaaaaatatttcaaaccattctcacaactctatatgtgtctatgaccagataactggaagaccttctggttatggactatgcgtatacactctcccagatagttagaggcatatctaagaggttaacatatatgatcttatccaaaatgggtctcttggtgctagtgttttacatcagacttcactggctttcatgtatttccacaagtgccaaacatttctcatatccttgctgtattccatagagcagtgttcctgctacctggaacacttgattcttgaataactcctgtttacctttcagacaaaccctaaaggttaccacctcaaagaagtctttatagaagcctcatcatcttagacactctgtattgtttccttcatcgtatttacaacagacagatactgtgcacttactgcctcacttaacgacagggatacgttctgaaaggtgcatcattaggcggttttgttgtgtgaacatcacagagtgttacttacacaaacctaaatgatacagcctactaaacacctaggctatatgagcaatacagcctattgctcttaggcttcaaacttgtacgacatgtcactgtactgaatactgtaggcaactataacacagtggtaagtatattgtgtatctaaacaaacatagaaaaggtaatgcactgtactatgatgttacaacagctaggatgttgctatcaatagaaatttttcagcttcattttatttttatgggaccacctttgtatatgtggttcattgttggccgaaacaccattctgtggcacatgactatgtatttattcctcattattcctttaatattcatctcttccaggagggcatgtcatggacaatctctttttcttaccacaggtcttaggacctggcctagcacctggccaagaactactggcatacctccttttattgtgcttcaatttattgtgctttgcaaatactgaattttttacaagttgaagatttgtggcacctctgtaaccagcaagtctattggtgccattttttcaacatcatgtgcctgtttcctgtctcgctcatgtcacattttggtaattttcacaatattaaaaactttttcattattatta

SEQ ID NO: 1007 - protein, 832 aa, Thermus aquaticus [sequence omitted]

SEQ ID NO: 1008 - DNA, 85 bp, Artificial Sequence
  Description of Artificial Sequence: Synthetic primer
caagcagaagacggcatacgagatacgagcaagcacaggaccacaacacgcaatatatgcgcgtttacccaataatactgcgtct

SEQ ID NO: 1009 - DNA, 67 bp, Artificial Sequence
  Description of Artificial Sequence: Synthetic oligonucleotide
gtttacccaataatactgcgtcttggttcaccgctctcactcaacatggcaaggaagaccttaaatt

SEQ ID NO: 1010 - DNA, 127 bp, Artificial Sequence
  Description of Artificial Sequence: Synthetic polynucleotide
cagacaaggaactgattacaaacattggccgcaaattgcacaatttgcccccagcgcttcagcgttcttcggaatgtcgcgcattggcatggaagtcacaccttcgggaacgtggttgacctacaca

SEQ ID NO: 1011 - DNA, 71 bp, Artificial Sequence; identical to SEQ ID NO: 995
  Description of Artificial Sequence: Synthetic oligonucleotide

SEQ ID NO: 1012 - DNA, 77 bp, Artificial Sequence; identical to SEQ ID NO: 1004
  Description of Artificial Sequence: Synthetic oligonucleotide

SEQ ID NO: 1013 - DNA, 39 bp, Artificial Sequence
  Description of Artificial Sequence: Synthetic oligonucleotide
aatttaaggtcttccttgccatgttgagtgagagcggtg

SEQ ID NO: 1014 - DNA, 37 bp, Artificial Sequence
  Description of Artificial Sequence: Synthetic oligonucleotide
tgtgtaggtcaaccacgttcccgaaggtgtgacttcc

SEQ ID NO: 1015 - DNA, 39 bp, Artificial Sequence
  Description of Artificial Sequence: Synthetic oligonucleotide
ctcttagtaccattggtcccagagacatgtatagcatgg

SEQ ID NO: 1016 - DNA, 39 bp, Artificial Sequence
  Description of Artificial Sequence: Synthetic oligonucleotide
ggacttctgtgcagttaacatcctgataaagaacagcaa

SEQ ID NO: 1017 - DNA, 18 bp, Artificial Sequence
  Description of Artificial Sequence: Synthetic oligonucleotide
tttttttttttttttttt

SEQ ID NO: 1018 - DNA, 21290 bp, Severe acute respiratory syndrome coronavirus 2
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 1019 - DNA, 20 bp, Artificial Sequence
  Description of Artificial Sequence: Synthetic primer
gagcggctgtctccacaagt

SEQ ID NO: 1020 - DNA, 20 bp, Artificial Sequence
  Description of Artificial Sequence: Synthetic primer
gtgtttgcagatttggacct

SEQ ID NO: 1021 - DNA, 49 bp, Artificial Sequence
  Description of Artificial Sequence: Synthetic oligonucleotide
aatttaaggtcttccttgccatgttgagtgagagcggtgaaccaagacg

SEQ ID NO: 1022 - DNA, 47 bp, Artificial Sequence
  Description of Artificial Sequence: Synthetic oligonucleotide
tgtgtaggtcaaccacgttcccgaaggtgtgacttccatgccaatgc

SEQ ID NO: 1023 - DNA, 49 bp, Artificial Sequence
  Description of Artificial Sequence: Synthetic oligonucleotide
ctcttagtaccattggtcccagagacatgtatagcatggaaccaagtaa

SEQ ID NO: 1024 - DNA, 49 bp, Artificial Sequence
  Description of Artificial Sequence: Synthetic oligonucleotide
ggacttctgtgcagttaacatcctgataaagaacagcaacctggttaga

SEQ ID NO: 1025 - DNA, 60 bp, Artificial Sequence
  Description of Artificial Sequence: Synthetic oligonucleotide
  See specification as filed for detailed description of substitutions and preferred embodiments
aaaaaaaaaaaaaaaaaaaaaaaaaaaaaaaaaaaaaaaaaaaaaaaaaaaaaaaaaaaa